(19) United States
(12) Patent Application Publication
(10) Pub. No.: US 2013/0072523 A1
(43) Pub. Date: Mar. 21, 2013

(54) ANALOGUES FOR THE TREATMENT OR PREVENTION OF FLAVIVIRUS INFECTIONS

(71) Applicants: Bingcan Liu, Montreal (CA); Carl Poisson, Montreal (CA); Laval Chan Chun Kong, Kirkland (CA); Sanjoy Kumar Das, Pierrefonds (CA); Constantin Yannopoulos, Notre-Dame de I'lle Perrot (CA); Caroline Cadilhac, Montreal (CA); T. Jagadeeswar Reddy, Pierrefonds (CA); Louis Vaillancourt, Mascouche (CA); Guy Falardeau, Laval (CA); Oswy Z. Pereira, Kirkland (CA); Monica Bubenik, Mascouche (CA); John Maxwell, Hingham, MA (US)

(72) Inventors: Bingcan Liu, Montreal (CA); Carl Poisson, Montreal (CA); Laval Chan Chun Kong, Kirkland (CA); Sanjoy Kumar Das, Pierrefonds (CA); Constantin Yannopoulos, Notre-Dame de I'lle Perrot (CA); Caroline Cadilhac, Montreal (CA); T. Jagadeeswar Reddy, Pierrefonds (CA); Louis Vaillancourt, Mascouche (CA); Guy Falardeau, Laval (CA); Oswy Z. Pereira, Kirkland (CA); Monica Bubenik, Mascouche (CA); John Maxwell, Hingham, MA (US)

(73) Assignee: VERTEX PHARMACEUTICALS INCORPORATED, Cambridge, MA (US)

(21) Appl. No.: 13/518,059

(22) PCT Filed: Dec. 27, 2010
(86) PCT No.: PCT/US10/62168

§ 371 Date: Dec. 3, 2012

Related U.S. Application Data
(60) Provisional application No. 61/290,030, filed on Dec. 24, 2009.
(60) Provisional application No. 61/316,998, filed on Mar. 24, 2010.

(51) Int. Cl. C07D 403/14; C07D 401/14; C07D 495/04; C07D 413/14; C07D 405/14; C07D 409/14

(52) U.S. Cl. CPC C07D 403/14; C07D 405/14; C07D 409/14; C07D 495/04; C07D 413/14; C07D 401/14
USPC 514/314; 548/313.1; 514/397; 546/175; 546/272.7; 514/341

(57) ABSTRACT

Compounds represented by Formula I: (I) or pharmaceutically acceptable salts thereof, wherein A, B, B′, X, Y, R1, R₂, R₂′, R₃, R₃′, R₄, R₄′, R_{5′}, R_{5′}m, n, or p are as defined herein, are useful for treating flaviviridae viral infections.

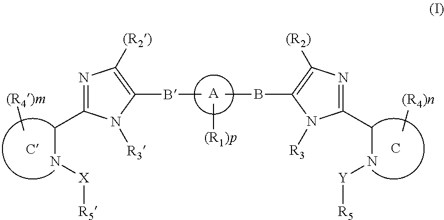

The present application claims the benefit under 35 U.S.C. §119(e) of U.S. Provisional Application No. 61/290,030, filed Dec. 24, 2009, and U.S. Provisional Application No. 61/316,998, filed Mar. 24, 2010, both of which are hereby incorporated by reference in their entirety.

The present invention relates to novel compounds and a method for the treatment or prevention of Flavivirus infections using novel compounds.

Hepatitis is a disease occurring throughout the world. It is generally of viral nature, although there are other causes known. Viral hepatitis is by far the most common form of hepatitis. Nearly 750,000 Americans are affected by hepatitis each year, and out of those, more than 150,000 are infected with the hepatitis C virus (“HCV”).

HCV is a positive-stranded RNA virus belonging to the Flaviviridae family and has close relationship to the pestiviruses that include hog cholera virus and bovine viral diarrhea virus (BVDV). HCV is believed to replicate through the production of a complementary negative-strand RNA template. Due to the lack of efficient culture replication system for the virus, HCV particles were isolated from pooled human plasma and shown, by electron microscopy, to have a diameter of about 50-60 nm. The HCV genome is a single-stranded, positive-sense RNA of about 9,600 bp coding for a polyprotein of 3009-3030 amino-acids, which is cleaved co- and post-translationally into mature viral proteins (core, E1, E2, p7, NS2, NS3, NS4A, NS4B, NS5A, NS5B). It is believed that the structural glycoproteins, E1 and E2, are embedded into a viral lipid envelope and form stable heterodimers. It is also believed that the structural core protein interacts with the viral RNA genome to form the nucleocapsid. The nonstructural proteins designated NS2 to NS5 include proteins with enzymatic functions involved in virus replication and protein processing including a polymerase, protease and helicase.

The main source of contamination with HCV is blood. The magnitude of the HCV infection as a health problem is illustrated by the prevalence among high-risk groups. For example, 60% to 90% of hemophiliacs and more than 80% of intravenous drug abusers in western countries are chronically infected with HCV. For intravenous drug abusers, the prevalence varies from about 28% to 70% depending on the population studied. The proportion of new HCV infections associated with post-transfusion has been markedly reduced lately due to advances in diagnostic tools used to screen blood donors.

Combination of pegylated interferon plus ribavirin is the treatment of choice for chronic HCV infection. This treatment does not provide sustained viral response (SVR) in a majority of patients infected with the most prevalent genotype (1a and 1b). Furthermore, significant side effects prevent compliance to the current regimen and may require dose reduction or discontinuation in some patients.

There is therefore a great need for the development of anti-viral agents for use in treating or preventing Flavivirus infections.

In one aspect, the present invention provides a compound of formula (I):

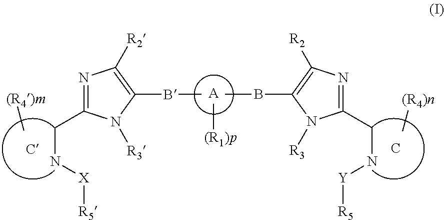

wherein
A is C₆₋₁₄ aryl, 4-12 membered heterocycle, C₃₋₁₀ cycloalkyl, 5-12 membered heteroaryl, or a bond;
B and B′ are each independently C₁₋₆ alkyl, C₂₋₆ alkenyl, or C₂₋₆ alkynyl;
C and C′ are each independently a 4-7 membered heterocycle;
R₁ is H, halogen, —ORₐ, —NRₐR_{b}, —C(═O)ORₐ, —C(O)NRₐR_{b}, —C(═O)OH, —C(═O)Rₐ, —C(═NOR_{c}) Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, —P(═O)ORₐOR_{b}, C₁₋₆ alkyl, C₂₋₆ alkenyl, C₂₋₆ alkynyl, or C₁₋₆ halogenated alkyl, or any two occurrences of R₁ can be taken together with the atoms to which they are attached to form a 5-7 cycloalkyl which is unsubstituted or substituted one or more times by R¹⁰ or a 5-7 membered heterocycle which is unsubstituted or substituted one or more times by R¹², wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
R₂ and R₂′ are each independently H, halogen, C₁₋₆ alkyl, —(CH₂)₁₋₃OH, —ORₐ, —C(═O)ORₐ, —C(O)NRₐR_{b}, —C(═O)OH, C₆₋₁₂ aryl, or 5-12 membered heteroaryl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
R₃ and R₃′ are each independently H, C₁₋₆ alkyl, C₂₋₆ alkenyl, or C₂₋₆ alkynyl;
R₄ and R₄′ are each independently H, halogen, C₁₋₆ alkyl, hydroxyl, C₆₋₁₄ aryl, or C₁₋₄ alkoxy;
wherein two occurrence of R₄ can be taken together with the atoms to which they are attached to form a 3-7 cycloalkyl which is unsubstituted or substituted one or more times by R¹⁰ or a 4-7 membered heterocycle which is unsubstituted or substituted one or more times by R¹²;
wherein two occurrence of R₄′ can be taken together with the atoms to which they are attached to form a 3-7 cycloalkyl which is unsubstituted or substituted one or more times by R¹⁰ or a 4-7 membered heterocycle which is unsubstituted or substituted one or more times by R¹²;
X and Y are each independently

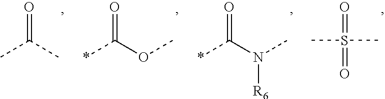

or a bond;
wherein the bond marked with an asterisk (*) indicates the attachment to the nitrogen of ring C or C′;
R₅ and R₅′ are each independently H, C₁₋₁₂ alkyl which is unsubstituted or substituted one or more times by R¹⁰, C₂₋₁₂ alkenyl which is unsubstituted or substituted one or more times by R¹⁰, C₂₋₁₂ alkynyl which is unsubstituted or substituted one or more times by R¹⁰, C₆₋₁₄ aryl which is unsubstituted or substituted one or more times by R¹¹, C₇₋₁₆ aralkyl which is unsubstituted or substituted one or more times by R¹¹, 5-12 membered heteroaryl which is unsubstituted or substituted one or more times by R¹¹, 6-18 membered heteroaralkyl which is unsubstituted or substituted one or more times by R¹¹, 3-12 membered heterocycle which is unsubstituted or substituted one or more times by R¹², or 4-18 membered heterocycle-alkyl which is unsubstituted or substituted one or more times by R¹²;
R₆ is H, C₁₋₆ alkyl, or halogenated C₁₋₆ alkyl, or can be merged with R₆ or R₆′ to form a 3-12 membered heterocycle;
m and n, are each independently 0, 1, 2, 3 or 4;
p is 0, 1, 2, 3 or 4;
R¹⁰ is halogen, —ORₐ, OXO, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
R¹¹ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
R¹² is halogen, —ORₐ, OXO, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
wherein as valency allows in B, B′, Rₐ-R_{d}, R₁, R₂, R₂′, R₃, R₃′, R₄, R₄′, R¹⁰, R¹¹ and R¹² each of alkyl, alkenyl, alkynyl, alkoxy, aryl, aralkyl, heteroaryl, heteroaralkyl, heterocycle, or heterocycle-alkyl is independently unsubstituted or substituted one or more times by halogen, —OR_{a′}, oxo, —NR_{a′R}_{b′}, —C(═O)OR_{a′}, —C(O)NR_{a′}R_{b′}, —C(═O)OH, —C(═O)R_{a′}, —C(═NOR_{c′})R_{a′}, —C(═NR_{c′})NR_{a′}R_{b′}, —NR_{d′}C(═O)NR_{a′}R_{b′}, —NR_{b′}C(═O)R_{a′}, —NR_{d′}C(═NR_{c′})NR_{a′}R_{b′}, —NR_{b′}C(═O)OR_{a′}, —OC(═O)NR_{a′}R_{b′}, —OC(═O)R_{a′}, —OC(═O)OR_{a′}, hydroxyl, nitro, azido, cyano, —S(O)₀₋₃R_{a′, —SO}₂NR_{a′}R_{b′}, or —NR_{b′}SO₂R_{a′}; wherein R_{a′}, R_{b′}, R_{c′}, and R_{d′} are each independently H or C₁₋₁₂ alkyl; or
   a pharmaceutically acceptable salt thereof.

In one aspect, the present invention provides a compound of formula (IA):

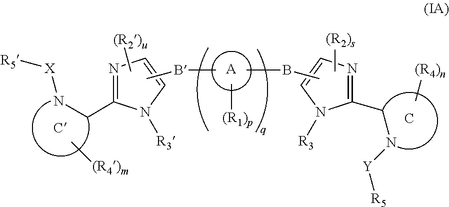

or a pharmaceutically acceptable salt thereof, wherein
each A is independently C₆₋₁₄ aryl, 4-12 membered heterocycle, C₃₋₁₀ cycloalkyl, or 5-12 membered heteroaryl containing at least one heteroatom selected from the group consisting of O and S; wherein when q is 2 then both A rings are not phenyl;
B and B′ are each independently absent, C₁₋₆ alkyl, C₂₋₆ alkenyl, or C₂₋₆ alkynyl; wherein when q is 1 then at least one of B and B′ is absent;
C and C′ are each independently a 4-7 membered heterocycle;
R₁ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, —P(═O)ORₐOR_{b}, C₁₋₆ alkyl which is unsubstituted or substituted one or more times by R¹⁰, C₂₋₆ alkenyl which is unsubstituted or substituted one or more times by R¹⁰, C₂₋₆ alkynyl which is unsubstituted or substituted one or more times by R¹⁰, or any two occurrences of R₁ can be taken together with the atoms to which they are attached to form a 5-7 cycloalkyl which is unsubstituted or substituted one or more times by R¹¹ or a 5-7 membered heterocycle which is unsubstituted or substituted one or more times by R¹²;
Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
R_{2′} and R₂ are independently halogen, C₁₋₁₀ alkyl, C₁₋₆ halogenated alkyl, —(CH₂)₁₋₆OH, —NR_{b}C(═O)Rₐ, C₆₋₁₂ aryl, or 5-12 membered heteroaryl;
R₃ and R₃′ are each independently H, C₁₋₆ alkyl, —(CH₂)₁₋₆OH, C₂₋₆ alkenyl, or C₂₋₆ alkynyl;
R₄ and R₄′ are each independently halogen, —NRₐR_{b}, —C(O)NRₐR_{b}, —(CH₂)₁₋₆OH, C₁₋₆ alkyl, C₁₋₆ halogenated alkyl, hydroxyl, C₆₋₁₄ aryl, or C₁₋₆ alkoxy; wherein two occurrence of R₄ can be taken together with the atoms to which they are attached to form a C₁₋₆ alkenyl which is unsubstituted or substituted one or more times by R¹⁰, a 3-7 cycloalkyl which is unsubstituted or substituted one or more times by R¹¹ or a 4-7 membered heterocycle which is unsubstituted or substituted one or more times by R¹²; wherein two occurrence of R₄′ can be taken together with the atoms to which they are attached to form a C₁₋₆ alkenyl which is unsubstituted or substituted one or more times by R¹⁰, a 3-7 cycloalkyl which is unsubstituted or substituted one or more times by R¹¹ or a 4-7 membered heterocycle which is unsubstituted or substituted one or more times by R¹²;
X and Y are each independently

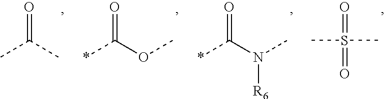

or a bond;
wherein the asterisk (*) indicates the point of attachment to the nitrogen of ring C or C′;
R₅ and R₅′ are each independently H, C₁₋₁₈ alkyl which is unsubstituted or substituted one or more times by R¹⁰, C₂₋₁₂ alkenyl which is unsubstituted or substituted one or more times by R¹⁰, C₂₋₁₂ alkynyl which is unsubstituted or substituted one or more times by R¹⁰, C₆₋₁₄ aryl which is unsubstituted or substituted one or more times by R¹¹, C₇₋₁₆ aralkyl which is unsubstituted or substituted one or more times by R¹¹, 5-12 membered heteroaryl which is unsubstituted or substituted one or more times by R¹¹, 6-18 membered heteroaralkyl which is unsubstituted or substituted one or more times by R¹¹, 3-12 membered heterocycle which is unsubstituted or substituted one or more times by R¹², or 4-18 membered heterocycle-alkyl which is unsubstituted or substituted one or more times by R¹²;
R₆ is H, C₁₋₆ alkyl, or halogenated C₁₋₆ alkyl;
m and n, are each independently 0, 1, 2, 3 or 4;
p is 0, 1, 2, 3 or 4;
q is 1 or 2;
u is 0 or 1;
s is 0 or 1;
R¹⁰ is halogen, —ORₐ, OXO, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b};
R¹¹ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and
R¹² is halogen, —ORₐ, OXO, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment for formula (I) or (IA), the distance between C and C′ is between about 16 Å and about 24 Å in length.

In another aspect, there is provided a method for treating or preventing a Flaviviridae viral infection in a patient comprising administering to the patient a therapeutically effective amount of a compound, composition or combination of the invention.

In another aspect, there is provided a pharmaceutical composition comprising at least one compound of the invention and at least one pharmaceutically acceptable carrier or excipient.

In another aspect, there is provided a combination comprising a compound of the invention and one or more additional agents chosen from viral serine protease inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

In a further aspect, there is provided the use of a compound, composition or combination of the invention for treating or preventing a Flaviviridae viral infection in a human.

In still another aspect, there is provided the use of a compound, composition or combination of the invention for the manufacture of a medicament for treating or preventing a viral Flaviviridae infection in a human.

In one embodiment, compounds of the present invention comprise those wherein the following embodiments are present, either independently or in combination.

In accordance with a further embodiment, the compounds of the present invention are represented by formula (II):

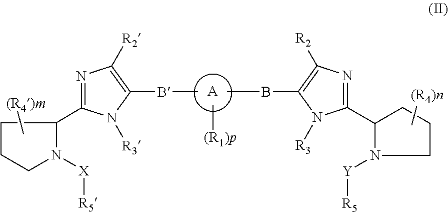

or a pharmaceutically acceptable salt thereof, wherein each of A, B, B′, R¹, p, R₂, R_{2′}, R₃, R_{3′}, R₄, R_{4′}, R₅, R_{5′}, X, and Y, are as defined for formula (I).

In accordance with a further embodiment, the compounds of the present invention are represented by formula (IIA):

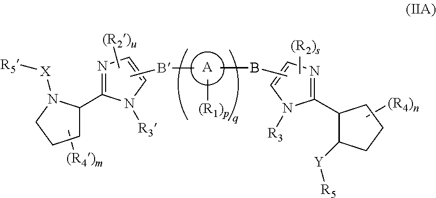

or a pharmaceutically acceptable salt thereof, wherein each of q, u, s, A, B, B′, R¹, p, R₂, R_{2′}, R₃, R₃′, R₄, R_{4′}, R₅, R_{5′}, m, n, X, and Y, are as defined for formula (IA).

In accordance with a further embodiment, the compounds of the present invention are represented by formula (III):

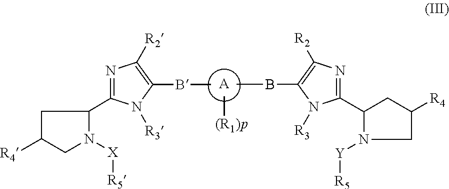

or a pharmaceutically acceptable salt thereof, wherein each of A, B, B′, R¹, p, R₂, R_{2′}, R₃, R_{3′}, R₄, R_{4′}, R₅, R_{5′}, X, and Y, are as defined for formula (I).

In accordance with a further embodiment, the compounds of the present invention are represented by formula (IIIA):

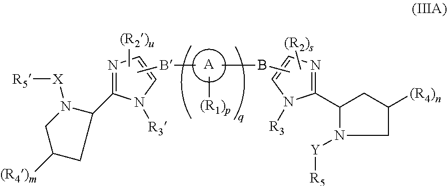

or a pharmaceutically acceptable salt thereof, wherein
each A is independently C₆₋₁₄ aryl, 4-12 membered heterocycle, C₃₋₁₀ cycloalkyl, or 5-12 membered heteroaryl;
B and B′ are each independently absent, C₁₋₆ alkyl, C₂₋₆ alkenyl, or C₂₋₆ alkynyl; wherein when q is 1 then at least one of B and B′ is absent;
R₁ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, —P(═O)ORₐOR_{b}, C₁₋₆ alkyl which is unsubstituted or substituted one or more times by R¹⁰, C₂₋₆ alkenyl which is unsubstituted or substituted one or more times by R¹⁰, C₂₋₆ alkynyl which is unsubstituted or substituted one or more times by R¹⁰, or any two occurrences of R₁ can be taken together with the atoms to which they are attached to form a 5-7 cycloalkyl which is unsubstituted or substituted one or more times by R¹¹ or a 5-7 membered heterocycle which is unsubstituted or substituted one or more times by R¹²;
Rₐ, R_{b}, R_{c}, and R_{d}Rₐ-R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
R_{2′} and R₂ are independently H, halogen, C₁₋₁₀ alkyl, C₁₋₆ halogenated alkyl, —(CH₂)₁₋₆OH, —NR_{b}C(═O)Rₐ C₆₋₁₂ aryl, or 5-12 membered heteroaryl;
R₃ and R₃′ are each independently H, C₁₋₆ alkyl, —(CH₂)₁₋₆OH, C₂₋₆ alkenyl, or C₂₋₆ alkynyl;
R₄ and R₄′ are each independently —NRₐR_{b}, —C(O)NRₐR_{b}, —(CH₂)₁₋₆OH, C₁₋₆ alkyl, C₁₋₆ halogenated alkyl, C₆₋₁₄ aryl, or C₁₋₆ alkoxy; wherein two occurrence of R₄ can be taken together with the atoms to which they are attached to form a C₁₋₆ alkenyl which is unsubstituted or substituted one or more times by R¹⁰, a 4-7 cycloalkyl which is unsubstituted or substituted one or more times by R¹¹ or a 4-7 membered heterocycle which is unsubstituted or substituted one or more times by R¹²; wherein two occurrence of R₄′ can be taken together with the atoms to which they are attached to form a C₁₋₆ alkenyl which is unsubstituted or substituted one or more times by R¹⁰, a 3-7 cycloalkyl which is unsubstituted or substituted one or more times by R¹¹ or a 4-7 membered heterocycle which is unsubstituted or substituted one or more times by R¹²; wherein Rₐ-R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
X and Y are each independently

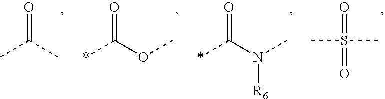

or a bond;
wherein the asterisk (*) indicates the point of attachment to the nitrogen of ring C or C′;
R₅ and R₅′ are each independently H, C₁₋₁₈ alkyl which is unsubstituted or substituted one or more times by R¹⁰, C₂₋₁₂ alkenyl which is unsubstituted or substituted one or more times by R¹⁰, C₂₋₁₂ alkynyl which is unsubstituted or substituted one or more times by R¹⁰, C₆₋₁₄ aryl which is unsubstituted or substituted one or more times by R¹¹, C₇₋₁₆ aralkyl which is unsubstituted or substituted one or more times by R¹¹, 5-12 membered heteroaryl which is unsubstituted or substituted one or more times by R¹¹, 6-18 membered heteroaralkyl which is unsubstituted or substituted one or more times by R¹¹, 3-12 membered heterocycle which is unsubstituted or substituted one or more times by R¹², or 4-18 membered heterocycle-alkyl which is unsubstituted or substituted one or more times by R¹²;
R₆ is H, C₁₋₆ alkyl, or halogenated C₁₋₆ alkyl;
m and n are a positive integer and when combined are 1, 2, 3, or 4; provided that each of m and n are not 3 or 4;
p is 0, 1, 2, 3 or 4;
q is 1 or 2;
u is or 1;
s is 0 or 1;
R¹⁰ is halogen, —ORₐ, OXO, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b};
R¹¹ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and
R¹² is halogen, —ORₐ, OXO, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In accordance with a further embodiment, the compounds of the present invention are represented by formula (IIIB):

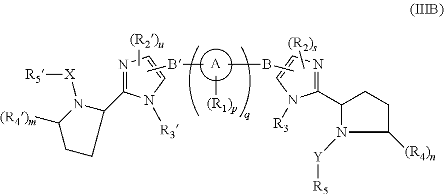

or a pharmaceutically acceptable salt thereof, wherein
each A is independently C₆₋₁₄ aryl, 4-12 membered heterocycle, C₃₋₁₀ cycloalkyl, or 5-12 membered heteroaryl wherein when q is 2 then both A rings are not phenyl;
B and B′ are each independently absent, C₁₋₆ alkyl, C₂₋₆ alkenyl, or C₂₋₆ alkynyl; wherein q is 1 then at least one of B and B′ is absent;
R₁ is halogen, —ORₐ, —NRₐR_{b}, —C(═O)ORₐ, —C(O)NRₐR_{b}, C(═O)OH, —C(═O)Rₐ, —C(═NOR_{c})Rₐ, —C(═NR_{c})NRₐR_{b}, NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, —P(═O)ORₐOR_{b}, C₁₋₆ alkyl which is unsubstituted or substituted one or more times by R¹⁰, C₂₋₆ alkenyl which is unsubstituted or substituted one or more times by R¹⁰, C₂₋₆ alkynyl which is unsubstituted or substituted one or more times by R¹⁰, or any two occurrences of R₁ can be taken together with the atoms to which they are attached to form a 5-7 cycloalkyl which is unsubstituted or substituted one or more times by R¹¹ or a 5-7 membered heterocycle which is unsubstituted or substituted one or more times by R¹²;
Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
R_{2′} and R₂ are independently H, halogen, C₁₋₁₀ alkyl, C₁₋₆ halogenated alkyl, —(CH₂)₁₋₆OH, —NR_{b}C(═O)RₐC₆₋₁₂ aryl, or 5-12 membered heteroaryl;
R₃ and R₃′ are each independently H, C₁₋₆ alkyl, —(CH₂)₁₋₆OH, C₂₋₆ alkenyl, or C₂₋₆ alkynyl;
R₄ and R₄′ are each independently halogen, —NRₐR_{b}, —C(O)NRₐR_{b}, —(CH₂)₁₋₆OH, C₁₋₆ alkyl, C₁₋₆ halogenated alkyl, hydroxyl, or C₁₋₆ alkoxy; wherein two occurrence of R₄ can be taken together with the atoms to which they are attached to form a C₁₋₆ alkenyl which is unsubstituted or substituted one or more times by R¹⁰, a 3-7 cycloalkyl which is unsubstituted or substituted one or more times by R¹¹ or a 4-7 membered heterocycle which is unsubstituted or substituted one or more times by R¹²; wherein two occurrence of R₄′ can be taken together with the atoms to which they are attached to form a C₁₋₆ alkenyl which is unsubstituted or substituted one or more times by R¹⁰, a 3-7 cycloalkyl which is unsubstituted or substituted one or more times by R¹¹ or a 4-7 membered heterocycle which is unsubstituted or substituted one or more times by R¹²; wherein Rₐ-R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
X and Y are each independently

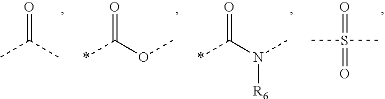

or a bond;
wherein the asterisk (*) indicates the point of attachment to the nitrogen of ring C or C′;
R₅ and R₅′ are each independently H, C₁₋₁₈ alkyl which is unsubstituted or substituted one or more times by R¹⁰, C₂₋₁₂ alkenyl which is unsubstituted or substituted one or more times by R¹⁰, C₂₋₁₂ alkynyl which is unsubstituted or substituted one or more times by R¹⁰, C₆₋₁₄ aryl which is unsubstituted or substituted one or more times by R¹¹, C₇₋₁₆ aralkyl which is unsubstituted or substituted one or more times by R¹¹, 5-12 membered heteroaryl which is unsubstituted or substituted one or more times by R¹¹, 6-18 membered heteroaralkyl which is unsubstituted or substituted one or more times by R¹¹, 3-12 membered heterocycle which is unsubstituted or substituted one or more times by R¹², or 4-18 membered heterocycle-alkyl which is unsubstituted or substituted one or more times by R¹²;
R₆ is H, C₁₋₆ alkyl, or halogenated C₁₋₆ alkyl;
m and n combined are each independently 1, 2, 3, or 4;
p is 0, 1, 2, 3 or 4;
q is 1 or 2;
u is 0 or 1;
s is 0 or 1;
R¹⁰ is halogen, —ORₐ, OXO, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b};
R¹¹ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and
R¹² is halogen, —ORₐ, OXO, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

It is to be understood that unless specifically stated otherwise, each of the further embodiments disclosed herein for the variables A, B, B′, R₁, p, q, R₂, R_{2′}, s, u, R₃, R_{3′}, R₄, R_{4′}, m, n, R₅, R_{5′}, X, Y, Rₐ, R_{b}, R_{c}, R_{d}, R₁₀, R₁₁, and R₁₂, applies to any and all of the structural formulas in which the variable appears.

In accordance with a further embodiment, each A is independently cyclopropyl, cyclohexyl, pyrrolidinyl, pyrazolidinyl, imidazolidinyl, piperazinyl, piperadinyl, phenyl, naphthalenyl, thienyl, furanyl, pyrrolyl, pyrazolyl, imidazolyl, thiazolyl, thiadiazolyl, oxazolyl, oxadiazolyl, pyridyl, pyrimidyl, pyrazinyl, pyridazinyl, indolyl, indazolyl, benzimidazolyl, benzoxazolyl, benzodioxolyl, benzothiazolyl, benzothiadiazolyl, dihydrobenzodioxine, thienofuranyl, thienothienyl, thienopyrrolyl, quinolinyl, quinoxalinyl, quinazolinyl, cinnolinyl, or triazolyl; and wherein each A is independently substituted with (R₁)ₚ.

In accordance with a further embodiment, each A is independently cyclopropyl, cyclohexyl, phenyl, or naphthalenyl, wherein each A is independently substituted with (R₁)ₚ.

In accordance with a further embodiment, A is independently selected from the group consisting of:

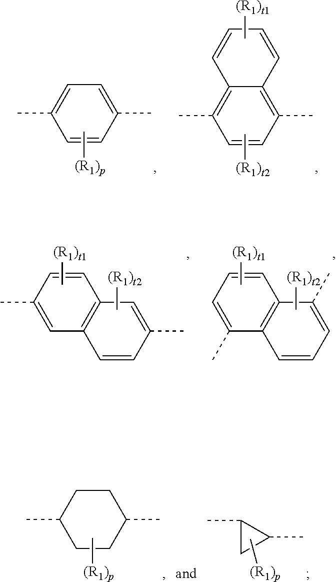

and t1+t2=p.

In accordance with a further embodiment, A is independently selected from the group consisting of:

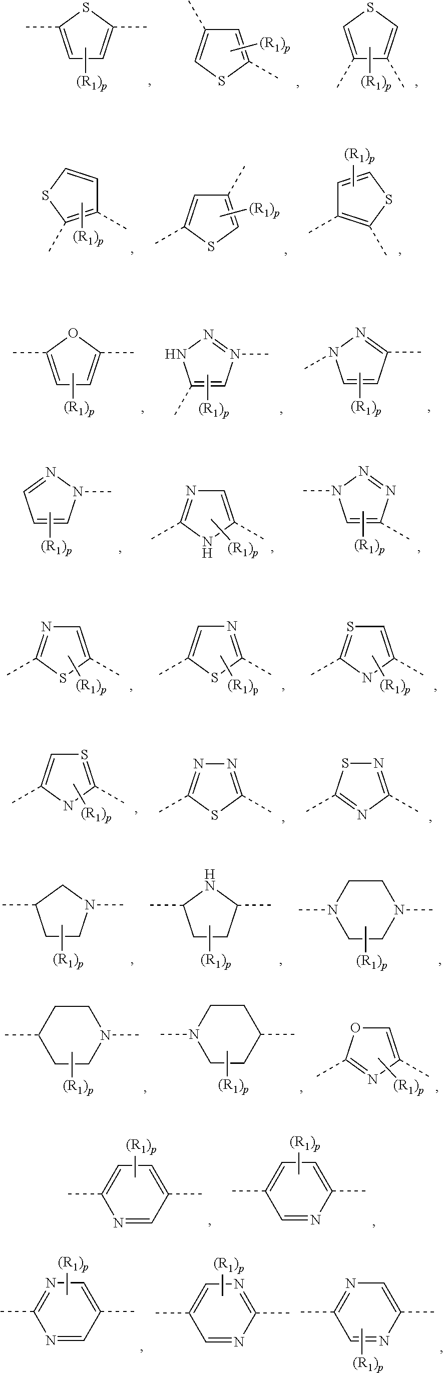

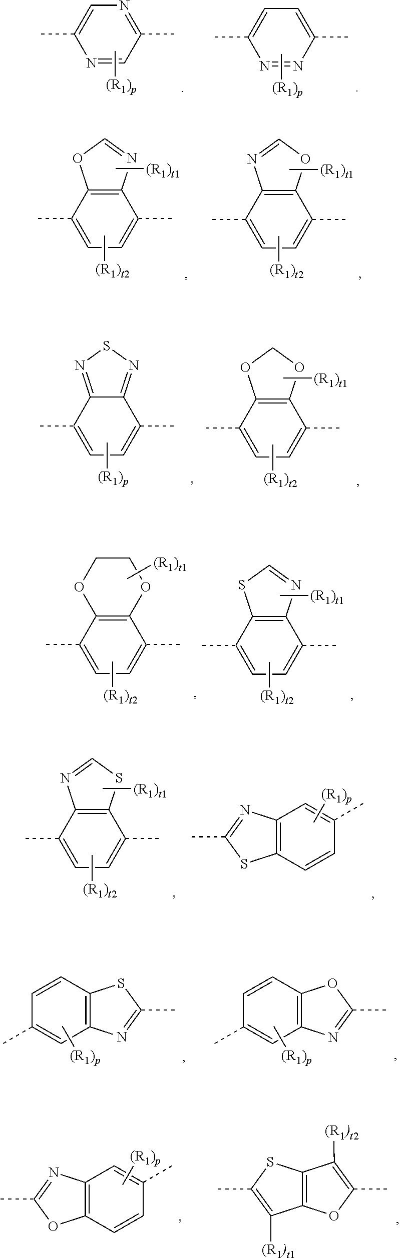

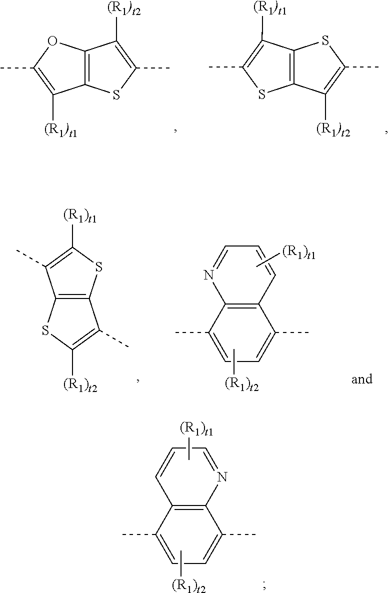

and
t1+t2=p.

In accordance with a further embodiment, each A is independently a 5-12 membered heteroaryl wherein the heteroatom(s) are selected from the group consisting of oxygen and sulphur; wherein each A is independently substituted with (R₁)ₚ.

In accordance with a further embodiment, A is independently piperazinyl, piperadinyl, thienyl, furanyl, pyrrolyl, pyrazolyl, imidazolyl, thiazolyl, oxazolyl, thiadiazolyl, pyrrolidinyl, pyridyl, pyrimidinyl, pyrazinyl, pyridazinyl, benzoxazolyl, benzodioxolyl, benzothiazolyl, benzothiadiazolyl, dihydrobenzodioxinyl, thienofuranyl, thienothienyl, quinolinyl, or triazolyl.

According to a further embodiment, A is phenyl, thiophene, pyridine, pyrimidine, triazole, naphthalene, thieno[3,2-b]thiophene, benzo[c][1,2,5]thiadiazole, quinoline, or benzo[b]thiophene.

According to a further embodiment, A is phenyl, thiophene, thieno[3,2-b]thiophene, pyridine, pyrimidine, naphthyl, benzo[1,3]dioxole, benzooxazole, or triazole.

According to a further embodiment, A is phenyl, thiophene, thieno[3,2-b]thiophene, naphtyl, benzo[1,3]dioxole, or benzooxazole.

According to a further embodiment, A is phenyl, thiophene, pyridine, pyrimidine, or triazole.

According to a further embodiment, A is phenyl or thieno[3,2-b]thiophene.

According to a further embodiment, A is phenyl or thiophene.

In accordance with a further embodiment, A is:

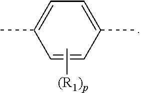

According to a further embodiment, A is

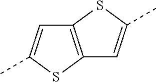

According to a further embodiment, A is

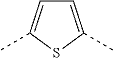

According to a further embodiment, A is

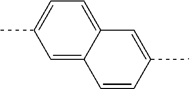

According to a further embodiment, A is

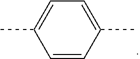

According to a further embodiment, A in formula (I), (II), (III), (IV) or (V) is a bond.

According to a further embodiment, B and B′ in formula (IA), (IIA), (IIIA), (IIIB), (IVA), or (VA) are independently absent, C₁₋₆ alkyl or C₂₋₆ alkynyl.

According to a further embodiment, B and B′ in formula (IA), (IIA), (IIIA), (IIIB), (IVA), or (VA) are independently absent, —(CH₂)₂— or —(C≡C)—.

According to a further embodiment, B and B′ in formula (IA), (IIA), (IIIA), (IIIB), (IVA), or (VA) are independently absent or —(C≡C)—.

According to a further embodiment,

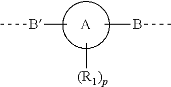

in formula (I), (II), (III), (IV) or (V) is selected from the group consisting of:

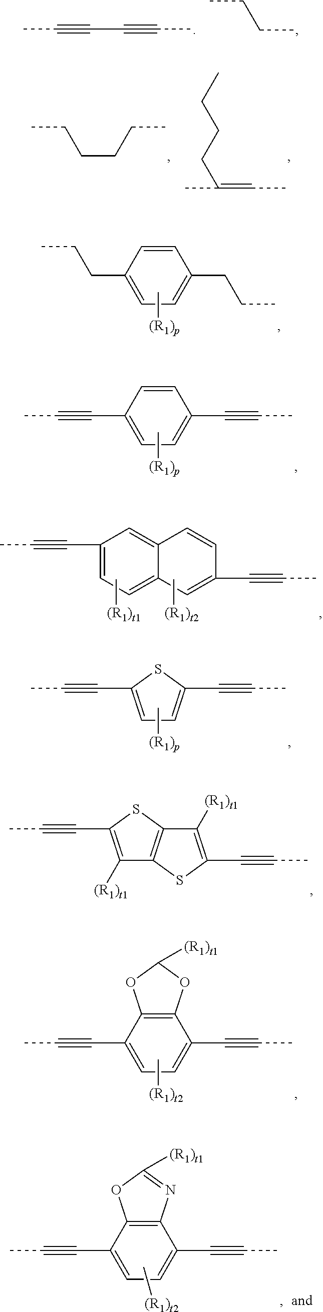

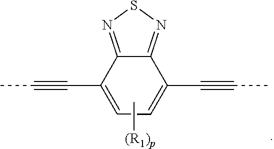

According to a further embodiment,

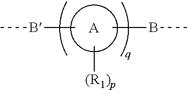

in formula (IA), (IIA), (IIIA), (IIIB), (IVA), or (VA) is selected from the group consisting of:

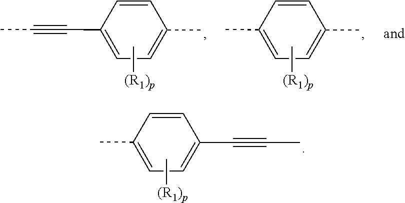

According to a further embodiment,

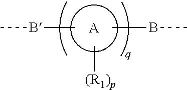

in formula (IA), (IIA), (IIIA), (IIIB), (IVA), or (VA) is selected from the group consisting of:

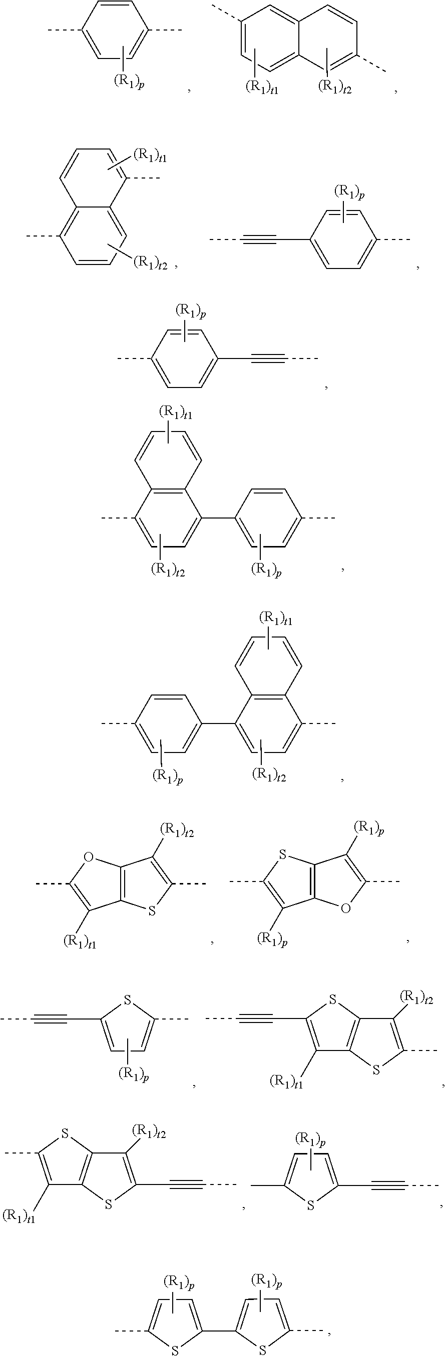

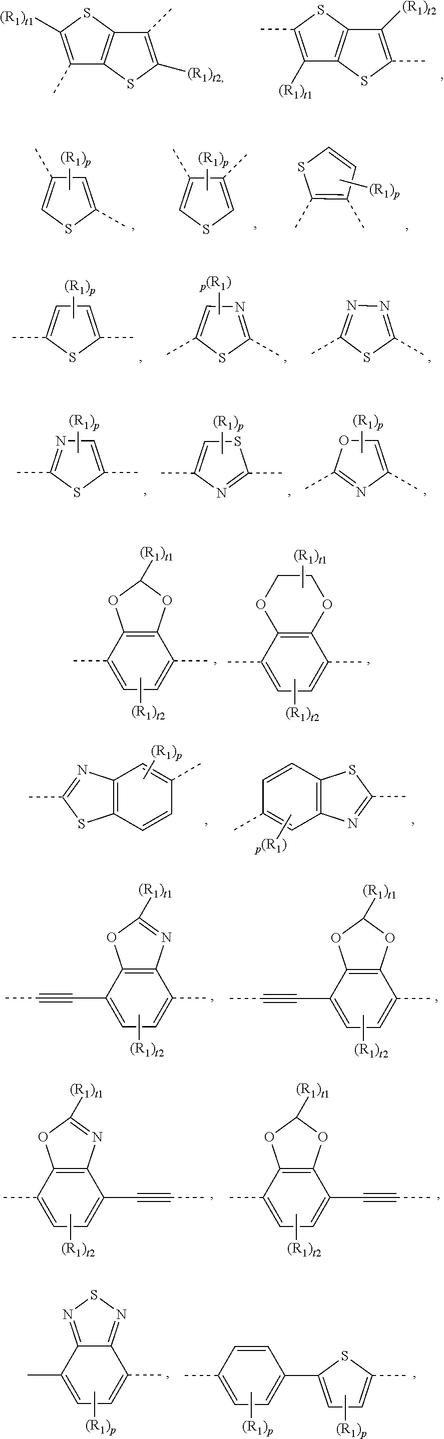

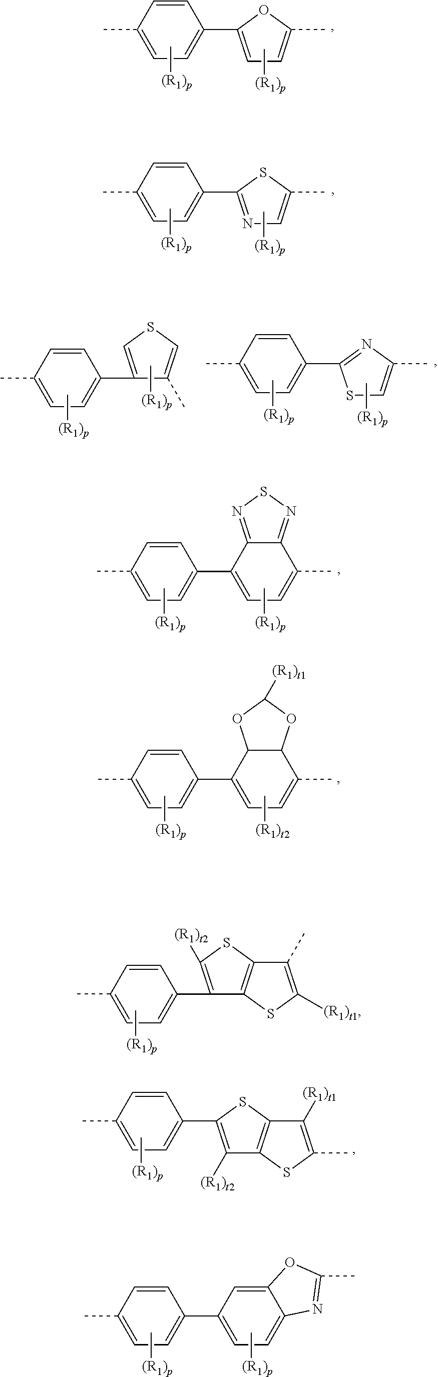

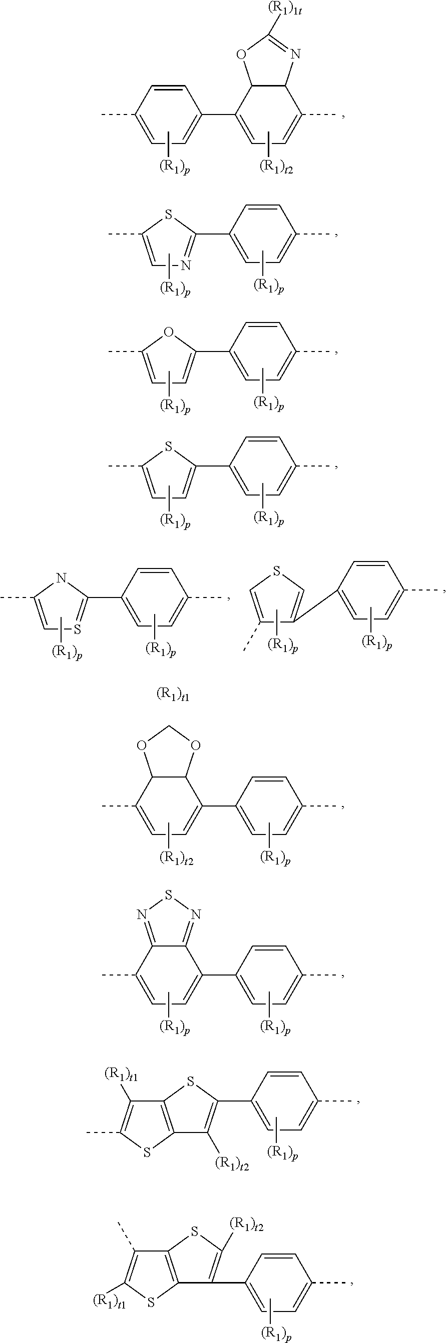

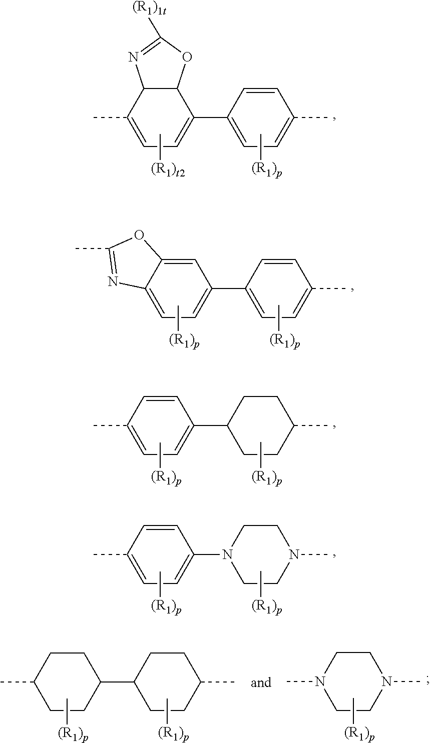

and
t1+t2=p.

According to a further embodiment,

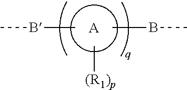

in formula (IA), (IIA), (IIIA), (IIIB), (IVA), or (VA) is selected from the group consisting of:

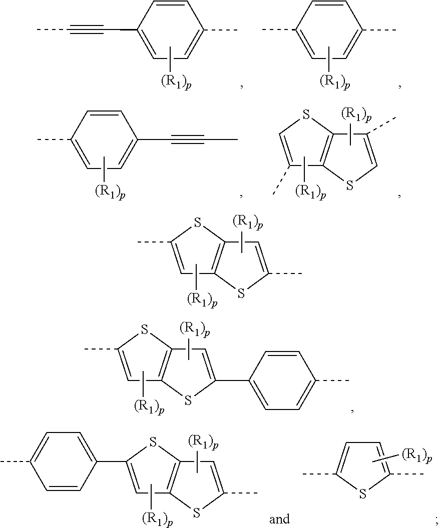

an d
t1+t2=p.

According to a further embodiment,

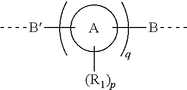

in formula (IA), (IIA), (IIIA), (IIIB), (IVA), or (VA) is selected from the group consisting of:

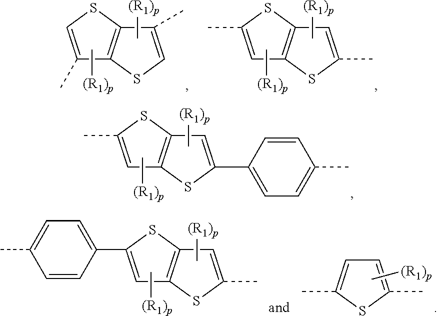

According to a further embodiment,

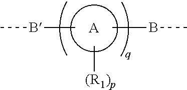

in formula (IA), (IIA), (IIIA), (IIIB), (IVA), or (VA) is selected from the group consisting of:

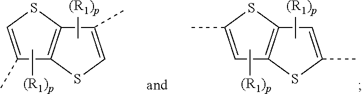

and
t1+t2=p.

According to a further embodiment,

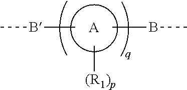

in formula (IA), (IIA), (IIIA), (IIIB), (IVA), or (VA) is:

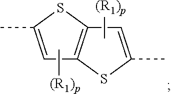

and
t1+t2=p.

According to a further embodiment,

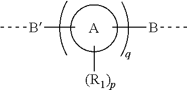

in formula (IA), (IIA), (IIIA), (IIIB), (IVA), or (VA) is:

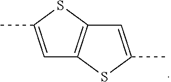

According to a further embodiment,

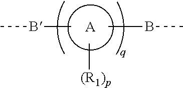

in formula (IA), (IIA), (IIIA), (IIIB), (IVA), or (VA) is:

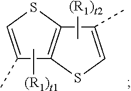

and
t1+t2=p.

According to a further embodiment,

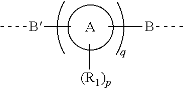

in formula (IA), (IIA), (IIIA), (IIIB), (IVA), or (VA) is:

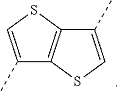

According to a further embodiment, B and B′ are each independently C₂₋₆ alkynyl or C₁₋₆ alkyl.

According to a further embodiment, B and B′ are each independently —(C≡C)— or —(CH₂)₂—.

According to a further embodiment, B and B′ are each —(CH₂)₂—.

According to a further embodiment, B and B′ are each —(C≡C)—.

According to a further embodiment, m or n is 2.

According to a further embodiment, m or n is 1.

According to a further embodiment, m and n are 1.

According to a further embodiment, one of m or n is 1 and the other of m and n are independently 1 or 2.

According to a further embodiment, p is 2.

According to a further embodiment, p is 1.

According to a further embodiment, X and Y are

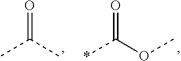

or a bond.

According to a further embodiment, X and Y are each

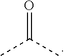

According to a further embodiment, X and Y are each

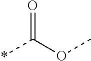

wherein the bond marked with an asterisk (*) indicates the attachment to the nitrogen.

According to a further embodiment, R₄ and R₄′ in formula (I), (IA), (II), (IIA), or (IIIB) are each independently halogen, —NRₐR_{b}, —C(O)NRₐR_{b}, —(CH₂)₁₋₆OH, C₁₋₆ alkyl, C₁₋₆ halogenated alkyl, hydroxyl, C₆₋₁₄ aryl, or C₁₋₆ alkoxy; wherein two occurrence of R₄ can be taken together with the atoms to which they are attached to form a C₁₋₆ alkenyl which is unsubstituted or substituted one or more times by R¹⁰, a 3-7 cycloalkyl which is unsubstituted or substituted one or more times by R¹¹ or a 4-7 membered heterocycle which is unsubstituted or substituted one or more times by R¹²; wherein two occurrence of R₄′ can be taken together with the atoms to which they are attached to form a C₁₋₆ alkenyl which is unsubstituted or substituted one or more times by R¹⁰, a 3-7 cycloalkyl which is unsubstituted or substituted one or more times by R¹¹ or a 4-7 membered heterocycle which is unsubstituted or substituted one or more times by R¹².

According to a further embodiment, R₄ and R₄′ in formula (I), (IA), (II), or (IIA) are each independently halogen, methyl, ethyl, isopropyl, di-fluoromethyl, di-fluoroethyl, trifluoromethyl, tri-fluoroethyl, —CH₂OH, —NRₐN_{b}, t-butoxy-, or hydroxyl; or two R₄ groups together with the atoms to which they are attached form fused cyclopropyl, spiro cyclopropyl or

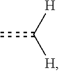

two R₄′ groups together with the atoms to which they are attached form fused cyclopropyl, spiro cyclopropyl or

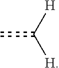

According to a further embodiment, R₄ and R₄′ in formula (I), (IA), (II), (IIA), or (IIIB) are each independently halogen, methyl, ethyl, isopropyl, di-fluoromethyl, di-fluoroethyl, trifluoromethyl, tri-fluoroethyl, —CH₂OH, —NRₐN_{b}, t-butoxy-, or hydroxyl; or two R₄ groups together with the atoms to which they are attached form spiro cyclopropyl or

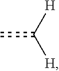

two R₄′ groups together with the atoms to which they are attached form spiro cyclopropyl or

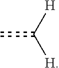

According to a further embodiment, R₄ and R₄′ in formula (I), (IA), (II), (IIA), or (IIIB) are each independently methyl, ethyl, isopropyl, di-fluoromethyl, di-fluoroethyl, trifluoromethyl, tri-fluoroethyl, —CH₂OH, —NRₐN_{b} or t-butoxy-; or two R₄ groups together with the atoms to which they are attached form spiro cyclopropyl or

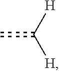

two R₄′ groups together with the atoms to which they are attached form spiro cyclopropyl or

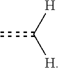

According to a further embodiment, R₄ and R₄′ in formula (I), (IA), (II), (IIA), (IIIB), (IVA), or (VA) are each independently methyl, ethyl, isopropyl, di-fluoromethyl, di-fluoroethyl, trifluoromethyl, tri-fluoroethyl, —CH₂OH, —NRₐN_{b} or t-butoxy-; or two R₄ groups together with the atoms to which they are attached form

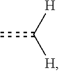

or two R₄′ groups together with the atoms to which they are attached form

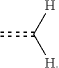

According to a further embodiment, in formula (I), (IA), (II), (IIA), (IIIA), (IIIB), (IVA), or (VA) two R₄ groups together with the atoms to which they are attached form

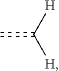

and two R₄′ groups together with the atoms to which they are attached form

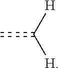

According to a further embodiment, R₄ and R₄′ in formula (I), (IA), (II), or (IIA) are each independently methyl, ethyl, methoxy, di-fluoromethyl, trifluoromethyl, or two R₄ groups together with the atoms to which they are attached form fused cyclopropyl or spiro cyclopropyl or two R₄′ groups together with the atoms to which they are attached form fused cyclopropyl or spiro cyclopropyl.

According to a further embodiment, R₄ and R₄′ in formula (I), (IA), (II), (IIA), or (IIIB) are each independently methyl, ethyl, methoxy, di-fluoromethyl, trifluoromethyl, or two R₄ groups together with the atoms to which they are attached form spiro cyclopropyl or two R₄′ groups together with the atoms to which they are attached form spiro cyclopropyl.

According to a further embodiment, R₄ and R₄′ are each independently H, halogen, C₁₋₆ alkyl, hydroxyl, phenyl, or C₁₋₄ alkoxy.

According to a further embodiment, R₄ and R₄′ are each independently —NRₐR_{b}, —C(O)NRₐR_{b}, —(CH₂)₁₋₆OH, C₁₋₆ alkyl, C₁₋₆ halogenated alkyl, C₆₋₁₄ aryl, or C₁₋₆ alkoxy.

According to a further embodiment, R₄ and R₄′ are each independently halogen, methyl, ethyl, isopropyl, di-fluoromethyl, di-fluoroethyl, trifluoromethyl, tri-fluoroethyl, —CH₂OH, —NRₐN_{b}, t-butoxy-, or hydroxyl.

According to a further embodiment, R₄ and R₄′ are each independently halogen, methyl, ethyl, isopropyl, di-fluoromethyl, di-fluoroethyl, trifluoromethyl, tri-fluoroethyl, —CH₂OH, t-butoxy-, or hydroxyl.

According to a further embodiment, R₄ and R₄′ are each independently methyl, ethyl, isopropyl, di-fluoromethyl, di-fluoroethyl, trifluoromethyl, tri-fluoroethyl, —CH₂OH, —NRₐN_{b}, or t-butoxy.

According to a further embodiment, R₄ and R₄′ are each independently methyl, ethyl, isopropyl, di-fluoromethyl, di-fluoroethyl, trifluoromethyl, tri-fluoroethyl, or —CH₂OH.

According to a further embodiment, R₄ and R₄′ are each independently methyl, ethyl, methoxy, di-fluoromethyl, or trifluoromethyl.

According to a further embodiment, R₄ and R₄′ are each independently H, halogen, methyl, ethyl, t-butoxy-, or hydroxyl.

According to a further embodiment, R₄ and R₄′ are each H.

According to a further embodiment, R₄ and R₄′ are each fluoro.

According to a further embodiment, R₄ and R₄′ are C₁₋₆ alkyl.

According to a further embodiment, R₄ and R₄′ are each C₁₋₆ haloalkyl.

According to a further embodiment, R₄ and R₄′ are methyl or ethyl.

According to a further embodiment, R₄ and R₄′ are each methyl.

According to a further embodiment, R₄ and R₄′ are each ethyl.

According to a further embodiment, R₄ and R₄′ are each methoxy.

According to a further embodiment, R₄ and R₄′ are each independently halogen, C₁₋₆ alkyl, or C₁₋₆ alkoxy.

According to a further embodiment, one of R₄ and R₄′ is hydrogen and the other of R₄ and R₄′ is C₁₋₆ alkyl.

According to a further embodiment, R₃ and R₃′ are H or methyl.

According to a further embodiment, R₃ and R₃′ are each H.

According to a further embodiment, R₃ and R₃′ are methyl.

According to a further embodiment, R₁ is H, halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, —P(═O)ORₐOR_{b}, C₁₋₆ alkyl, C₂₋₆ alkenyl, C₂₋₆ alkynyl, or C₁₋₆ halogenated alkyl or any two occurrences of R₁ can be taken together with the atoms to which they are attached to form a 5-7 cycloalkyl which is unsubstituted or substituted one or more times by R¹⁰ or a 5-7 membered heterocycle which is unsubstituted or substituted one or more times by R₁₂.

According to a further embodiment, R₁ is H, halogen, —ORₐ, —NRₐR_{b}, —C(═O)ORₐ, —C(O)NRₐR_{b}, —C(═O)OH, —NR_{b}C(═O)Rₐ, -hydroxyl, nitro, cyano, —S(O)₀₋₃Rₐ, C₁₋₆ alkyl, C₂₋₆ alkenyl, C₂₋₆ alkynyl, or C₁₋₆ halogenated alkyl.

According to a further embodiment, R₁ is halogen, C₁₋₃ alkyl, hydroxyl, cyano, or C₁₋₃ alkoxy, or methoxycarbonyl.

According to a further embodiment, R₁ is halogen, C₁₋₄ alkyl which is unsubstituted or substituted one or more times by R¹⁰, —C(═O)ORₐ, —C(O)NRₐR_{b}, hydroxyl, cyano, or C₁₋₃ alkoxy.

According to a further embodiment, R₁ is chloro, fluoro, bromo, methyl, ethyl, propyl, butyl, —CH₂OH, difluoromethyl, trifluoromethyl, hydroxyl, cyano, methoxy, or methoxycarbonyl.

According to a further embodiment, R₁ is chloro, fluoro, methyl, hydroxyl, cyano, trifluoromethyl, methoxy, or methoxycarbonyl.

According to a further embodiment, R₁ is halogen, C₁₋₃ alkyl, hydroxyl, cyano, or C₁₋₃ alkoxy.

According to a further embodiment, R₁ is chloro, fluoro, methyl, hydroxyl, cyano, or methoxy.

According to a further embodiment, R₁ is methyl

According to a further embodiment, R₁ is H.

According to a further embodiment, R₂ and R₂′ are each independently H, halogen, C₁₋₆ alkyl, —(CH₂)₁₋₃OH, —ORₐ, —C(═O)ORₐ, —C(O)NRₐR_{b}, —C(═O)OH, C₆₋₁₂ aryl, or 5-12 membered heteroaryl, wherein Rₐ, R_{b}, R_{c}, and R_{d} are each independently H, C₁₋₁₂ alkyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R₂ and R₂′ are each independently H, halogen, C₁₋₆ alkyl, —(CH₂)₁₋₃OH, —ORₐ, —C(═O)ORₐ, —C(O)NRₐR_{b}, —C(═O)OH, phenyl, or 5-6 membered heteroaryl, wherein Rₐ, R_{b}, R_{c}, and R_{d} are each independently H, C₁₋₁₂ alkyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R₂ and R₂′ are each independently H, iodo, methyl, hydroxymethyl, trifluoromethyl, or thienothienyl.

According to a further embodiment, R₂′ is independently methyl, trifluoromethyl, iodo, CH₂OH, or NHC(O)CH₃.

According to a further embodiment, R₂ and R₂′ are each methyl.

According to a further embodiment, R₂ and R₂′ are each iodo.

According to a further embodiment, s is 0.

According to a further embodiment, u is 0.

According to a further embodiment, R₂ and R₂′ are each H.

According to a further embodiment, R₆ is H or C₁₋₃ alkyl.

According to a further embodiment, R₅ and R₅′ are each independently C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, C₇₋₈ aralkyl which is unsubstituted or substituted one or more times by R¹¹, 5-6 membered heteroaryl which is unsubstituted or substituted one or more times by R¹¹, 6-8 membered heteroaralkyl which is unsubstituted or substituted one or more times by R¹¹, 3-6 membered heterocycle which is unsubstituted or substituted one or more times by R¹², or 4-8 membered heterocycle-alkyl which is unsubstituted or substituted one or more times by R¹².

According to a further embodiment, R₅ and R₅′ are each independently C₁₋₆ alkyl which is unsubstituted or substituted one or more times by R¹⁰, C₂₋₆ alkenyl which is unsubstituted or substituted one or more times by R¹⁰, C₂₋₆ alkynyl which is unsubstituted or substituted one or more times by R¹⁰, phenyl which is unsubstituted or substituted one or more times by R¹¹, benzyl which is unsubstituted or substituted one or more times by R¹¹, 5-6 membered heteroaryl which is unsubstituted or substituted one or more times by R¹¹, 6-7 membered heteroaralkyl which is unsubstituted or substituted one or more times by R¹¹, 5-6 membered heterocycle which is unsubstituted or substituted one or more times by R¹², or 6-7 membered heterocycle-alkyl which is unsubstituted or substituted one or more times by R¹².

According to a further embodiment, R₅ and R₅′ are each independently C₁₋₆ alkyl which is unsubstituted or substituted one or more times by R¹⁰, C₂₋₆ alkenyl which is unsubstituted or substituted one or more times by R¹⁰, or C₂₋₆ alkynyl which is unsubstituted or substituted one or more times by R¹⁰.

According to a further embodiment, R₅ and R₅′ are each independently C₁₋₁₂ alkyl which is unsubstituted or substituted one or more times by R¹⁰.

According to a further embodiment, R₅ and R₅′ are each independently methyl, ethyl, propyl, isopropyl, butyl, sec-butyl, tert-butyl, pentyl, 2-methylbutane, 3-methylbutane, cyclopropyl, cyclobutyl, cyclopentyl, cyclohexyl, or cyclohexyl(CH₂)—, which in each case is unsubstituted or substituted one or more times by R¹⁰.

According to a further embodiment, R₅ and R₅′ are each independently methyl, ethyl, propyl, isopropyl, butyl, sec-butyl, tert-butyl, pentyl, 2-methylbutane, 3-methylbutane, cyclopropyl, cyclobutyl, cyclopentyl, cyclohexyl, or cyclohexyl(CH₂)—.

According to a further embodiment, R₅ and R₅′ are each independently isopropyl which is unsubstituted or substituted one or more times by R¹⁰.

According to a further embodiment, R₅ and R₅′ are each independently isopropyl which is unsubstituted or substituted one or more times by —OCH₃.

According to a further embodiment, R₅ and R₅′ are each isopropyl.

According to a further embodiment, R₅ and R₅′ are each H or tert-butyl.

According to a further embodiment, R₅ and R₅′ are each independently phenyl which is unsubstituted or substituted one or more times by R¹¹.

According to a further embodiment, R₅ and R₅′ are each independently benzyl which is unsubstituted or substituted one or more times by R¹¹.

According to a further embodiment, R¹⁰ is halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, or —NR_{b}SO₂NRₐR_{b}, wherein Rₐ-R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹⁰ is —NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —NR_{b}SO₂Rₐ, or —NR_{b}SO₂NRₐR_{b}, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹⁰ is —NRₐR_{b}, —NR_{d}C(═O)NRₐR_{b}, —NR_{b}C(═O)Rₐ, —NR_{b}C(═O)ORₐ, or —NR_{b}SO₂Rₐ, wherein Rₐ, R_{b}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹⁰ is —NRₐR_{b} or —NR_{d}C(═O)NRₐR_{b}, wherein Rₐ and R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹⁰ is —NR_{d}C(═O)NRₐR_{b}, wherein Rₐ, R_{b}, are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹⁰ is halogen, —ORₐ, oxo, —C(═O)ORₐ, —C(O)NRₐR_{b}, —C(═O)OH, —C(═O)Rₐ, —OC(═O)NRₐR_{b}, —OC(═O)Rₐ, —OC(═O)OR_{c}, hydroxyl, cyano, wherein Rₐ-R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹⁰ is halogen, —OR_{c}, oxo, —C(═O)OR_{c}, —C(O)NRₐR_{b}, —C(═O)OH, —OC(═O)NRₐR_{b}, hydroxyl, or cyano, wherein Rₐ-R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹⁰ is halogen, C₁₋₆ alkoxy, hydroxyl, or NH₂.

According to a further embodiment, R¹⁰ is halogen, hydroxyl, or NH₂.

According to a further embodiment, R¹⁰ is halogen.

According to a further embodiment, R¹¹ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, or —NR_{b}SO₂NRₐR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹¹ is halogen, —ORₐ, —NRₐR_{b}, —C(═O)ORₐ, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, —OC(═O)Rₐ, —OC(═O)ORₐ, hydroxyl, cyano, —SO₂NRₐR_{b}, —NR_{b}SO₂Rₐ, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹¹ is halogen, —ORₐ, —NRₐR_{b}, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, hydroxyl, cyano, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹¹ is halogen, —ORₐ, —NRₐR_{b}, hydroxyl, cyano, or C₁₋₆ alkyl, wherein Rₐ-R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹¹ is halogen, hydroxyl, cyano, or NH₂.

According to a further embodiment, R¹¹ is halogen.

According to a further embodiment, R¹² is halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹² is halogen, —OR_{a, OxO, —NR}ₐR_{b}, —C(═O)ORₐ, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, —OC(═O)Rₐ, —OC(═O)ORₐ, hydroxyl, cyano, —SO₂NRₐR_{b}, —NR_{b}SO₂Rₐ, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹² is halogen, —OR_{a, OxO, —NR}ₐR_{b}, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, hydroxyl, cyano, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹² is halogen, —OR_{a, OxO, —NR}ₐR_{b}, hydroxyl, cyano, or C₁₋₆ alkyl, wherein Rₐ-R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹² is halogen.

According to a further embodiment, Rₐ, R_{b}, R_{c}, and R_{d} are each independently H, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl.

According to a further embodiment, Rₐ and R_{c} are each independently H, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, and R_{b}, and R_{d} are each independently H or C₁₋₃ alkyl.

According to a further embodiment, Rₐ and R_{c} are each independently H, C₁₋₆ alkyl, C₂₋₆ alkenyl, C₂₋₆ alkynyl, phenyl, benzyl, 5-6 membered heteroaryl, 6-8 membered heteroaralkyl, 5-6 membered heterocycle, or 6-8 membered heterocycle-alkyl, and R_{b}, and R_{d} are each independently H or C₁₋₃ alkyl.

According to a further embodiment, Rₐ, R_{b}, R_{c}, and R_{d} are each independently H or C₁₋₃ alkyl.

In accordance with a further embodiment, the compounds of the present invention are represented by formula (IV):

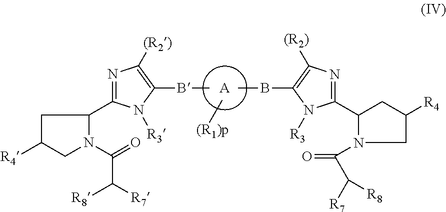

or a pharmaceutically acceptable salt thereof,
wherein A, B, B′, R₁, p, R₂, R_{2′}, R₃, R_{3′}, R₄, and R_{4′} are as defined for formula (I), and
R₇ and R₇′ are each independently C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, benzyl which is unsubstituted or substituted one or more times by R¹¹, 5-6 membered heteroaryl which is unsubstituted or substituted one or more times by R¹¹, 6-7 membered heteroaralkyl which is unsubstituted or substituted one or more times by R¹¹, 3-6 membered heterocycle which is unsubstituted or substituted one or more times by R¹², or 4-7 membered heterocycle-alkyl which is unsubstituted or substituted one or more times by R¹²; and
R₈ and R₈′ are each independently —NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —NR_{b}SO₂Rₐ, —NR_{b}SO₂NRₐR_{b}, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In accordance with a further embodiment, the compounds of the present invention are represented by formula (IVA):

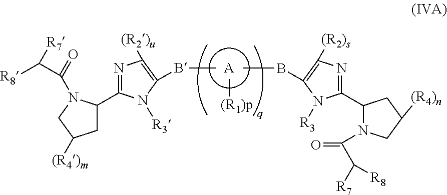

or a pharmaceutically acceptable salt thereof,
wherein q, u, s, A, B, B′, R¹, p, R₂, R_{2′}, R₃, R_{3′}, R₄, R_{4′}, m, and n, are as defined for formula (IIIA)
R₇ and R₇′ are each independently C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, benzyl which is unsubstituted or substituted one or more times by R¹¹, 5-6 membered heteroaryl which is unsubstituted or substituted one or more times by R¹¹, 6-7 membered heteroaralkyl which is unsubstituted or substituted one or more times by R¹¹, 3-6 membered heterocycle which is unsubstituted or substituted one or more times by R¹², or 4-7 membered heterocycle-alkyl which is unsubstituted or substituted one or more times by R¹²;
R₈ and R₈′ are each independently —NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —NR_{b}SO₂Rₐ, or —NR_{b}SO₂NRₐR_{b}, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and
m and n combined are 0, 1, 2, 3 or 4.

According to a further embodiment, R₈ and R₈′ are each independently —NRₐR_{b}, —NR_{b}C(═O)Rₐ, or —NR_{b}C(═O)ORₐ, wherein Rₐ-R_{b} are each independently H, C₁₋₆ alkyl, phenyl, benzyl, 5-6 membered heteroaryl, 6-8 membered heteroaralkyl, 5-6 membered heterocycle, or 6-8 membered heterocycle-alkyl.

According to a further embodiment, R₈ and R₈′ are each independently —NRₐR_{b} or —NR_{b}C(═O)ORₐ, wherein Rₐ-R_{b} are each independently H, C₁₋₆ alkyl, phenyl, benzyl, 5-6 membered heteroaryl, 6-8 membered heteroaralkyl, 5-6 membered heterocycle, or 6-8 membered heterocycle-alkyl.

According to a further embodiment, R₈ and R₈′ are each independently —NR_{b}C(═O)ORₐ, wherein Rₐ-R_{b} are each independently H, C₁₋₆ alkyl, phenyl, benzyl, 5-6 membered heteroaryl, 6-8 membered heteroaralkyl, 5-6 membered heterocycle, or 6-8 membered heterocycle-alkyl.

According to a further embodiment, R₈ and R₈′ are each independently —NR_{b}C(═O)ORₐ, wherein Rₐ-R_{b} are each independently H, C₁₋₆ alkyl, phenyl, tetrahydrofuran, or benzyl.

According to a further embodiment, R₈ and R₈′ are each independently —NR_{b}C(═O)ORₐ, wherein Rₐ is C₁₋₆ alkyl and R_{b} is H or methyl.

According to a further embodiment, R₈ and R₈′ are each independently —NR_{b}C(═O)ORₐ, wherein Rₐ is C₁₋₆ alkyl and R_{b} is H.

According to a further embodiment, R₈ and R₈′ are each independently —NR_{b}C(═O)ORₐ, wherein Rₐ is methyl and R_{b} is H.

According to a further embodiment, R₇ and R₇′ are each independently C₁₋₈ alkyl, C₂₋₈ alkenyl, C₂₋₈ alkynyl, phenyl, benzyl, 5-6 membered heteroaryl, 6-7 membered heteroaralkyl, 3-6 membered heterocycle, or 4-7 membered heterocycle-alkyl;

According to a further embodiment, R₇ and R₇′ are each independently phenyl.

According to a further embodiment, R₇ and R₇′ are each independently C₁₋₆ alkyl.

According to a further embodiment, R₇ and R₇′ are each independently C₁₋₆ alkyl which is unsubstituted or substituted one or more times by R¹⁰.

According to a further embodiment, R₇ and R₇′ are each independently methyl, ethyl, propyl, isopropyl, methoxyisopropyl, butyl, sec-butyl, tert-butyl, pentyl, 2-methylbutane, 3-methylbutane, cyclopropyl, cyclobutyl, cyclopentyl, or cyclohexyl.

According to a further embodiment, R₇ and R₇′ are each independently methyl, ethyl, propyl, isopropyl, butyl, sec-butyl, tert-butyl, pentyl, 2-methylbutane, 3-methylbutane, cyclopropyl, cyclobutyl, cyclopentyl, or cyclohexyl.

According to a further embodiment, R₇ and R₈ or R_{7′} and R_{8′} together with the carbon to which they are attached are each independently:

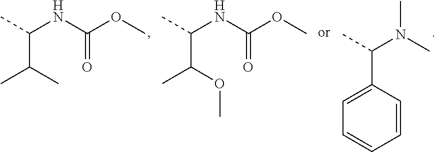

According to a further embodiment, R₇ and R₇′ are each isopropyl.

In accordance with a further embodiment, the compounds of the present invention are represented by formula (V):

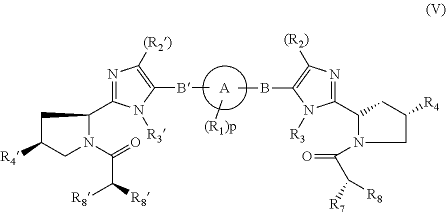

or a pharmaceutically acceptable salt thereof, wherein each of A, B, B′, R¹, p, R₂, R_{2′}, R₃, R_{3′}, R₄, R_{4′}, R₇, R_{7′}, R₈, and R_{8′}, are as defined for formula (I).

In accordance with a further embodiment, the compounds of the present invention are represented by formula (VA):

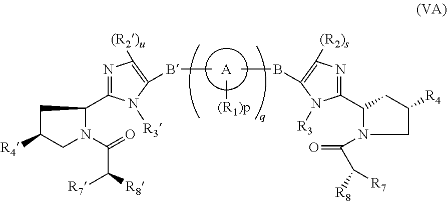

or a pharmaceutically acceptable salt thereof, wherein each of q, u, s, A, B, B′, R¹, p, R₂, R_{2′}, R₃, R_{3′}, R₄, R_{4′}, R₇, R_{7′}, R₈, and R_{8′}, are as defined for formula (IIIA).

According to a further embodiment, as valency allows in B, B′, Rₐ, R_{b}, R_{c}, and R_{d}, R₁, R₂, R₂′, R₃, R₃′, R₄, R₄′, R¹⁰, R¹¹ and R¹² each of alkyl, alkenyl, alkynyl, alkoxy, aryl, aralkyl, heteroaryl, heteroaralkyl, heterocycle, or heterocycle-alkyl is independently unsubstituted or substituted one or more times by halogen, —OR_{a′}, —NR_{a′}R_{b′}, C(═O)OR_{a′}, —C(O)NR_{a′}R_{b′}, —C(═O)OH, hydroxyl, nitro, azido, or cyano, wherein R_{a′}, R_{b′}, R_{c′}, and R_{d′} are each independently H, C₁₋₁₂ alkyl.

According to a further embodiment, as valency allows in B, B′, Rₐ, R_{b}, R_{c}, and R_{d}, R₁, R₂, R₂′, R₃, R₃′, R₄, R₄′, R¹⁰, R¹¹ and R¹² each of alkyl, alkenyl, alkynyl, alkoxy, aryl, aralkyl, heteroaryl, heteroaralkyl, heterocycle, or heterocycle-alkyl is independently unsubstituted or substituted one time by halogen.

According to a further embodiment, as valency allows in B, B′, Rₐ, R_{b}, R_{c}, and R_{d}, R₁, R₂, R₂′, R₃, R₃′, R₄, R₄′, R¹⁰, R¹¹ and R¹² each of alkyl, alkenyl, alkynyl, alkoxy, aryl, aralkyl, heteroaryl, heteroaralkyl, heterocycle, or heterocycle-alkyl is independently unsubstituted or substituted one time by fluoro.

In accordance with the present invention, the compounds are selected from compounds as defined in the formulas wherein:
A is C₆₋₁₄ aryl, 5-12 membered heteroaryl, or a bond;
B and B′ are each independently —(C≡C)— or —(CH₂)₂—;
R₁ is H, halogen, —ORₐ, —NRₐR_{b}, —C(═O)ORₐ, —C(O)NRₐR_{b}, —C(═O)OH, —NR_{b}C(═O)Rₐ, hydroxyl, nitro, cyano, —S(O)₀₋₃Rₐ, —C₁₋₆ alkyl, C₂₋₆ alkenyl, C₂₋₆ alkynyl, or C₁₋₆ halogenated alkyl;
R₂ and R₂′ are each independently H, methyl, or iodo;
m and n are each independently 0, 1 or 2;
p is 0, 1 or 2;
R₃ and R₃′ are H;
R₄ and R₄′ are each independently H, halogen, C₁₋₆ alkyl, hydroxyl, phenyl, or C₁₋₄alkoxy;
X and Y are

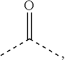

R₅ and R₅′ are each independently C₁₋₁₂ alkyl which is unsubstituted or substituted one or more times by R^{w}.

In accordance with the present invention, the compounds are selected from compounds as defined in the formulas wherein:
A is C₆₋₁₄ aryl, 5-12 membered heteroaryl, or a bond;
B and B′ are each independently —(C≡C)— or —(CH₂)₂—;
R₁ is H or methyl;
R₂ and R₂′ are each independently H, methyl or iodo;
m and n are each independently 0, 1 or 2;
p is 0, 1 or 2;
R₃ and R₃′ are H;
R₄ and R₄′ are each independently H, halogen, C₁₋₆ alkyl, hydroxyl, phenyl, or C₁₋₄ alkoxy;
X and Y are

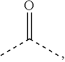

R₅ and R₅′ are each independently C₁₋₁₂ alkyl which is unsubstituted or substituted one or more times by R¹⁰.

In accordance with the present invention, the compounds are selected from compounds as defined in the formulas wherein:
A is phenyl, thiophene, thieno[3,2-b]thiophene, pyridine, pyrimidine, naphthyl, benzo[1,3]dioxole, benzooxazole, or triazole;
B and B′ are each independently —(C≡C)— or —(CH₂)₂—;
R₁ is H or methyl;
R₂ and R₂′ are each independently H, methyl or iodo;
m and n are each independently 0, 1 or 2;
p is 0, 1 or 2;
R₃ and R₃′ are H;
R₄ and R₄′ are each independently H, halogen, C₁₋₆ alkyl, hydroxyl, phenyl, or C₁₋₄ alkoxy;
X and Y are

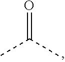

R₅ and R₅′ are each independently C₁₋₁₂ alkyl which is unsubstituted or substituted one or more times by R¹⁰.

In accordance with the present invention, the compounds are selected from compounds as defined in the formulas wherein:
A is phenyl, thiophene, thieno[3,2-b]thiophene, naphthyl, benzo[1,3]dioxole, or benzooxazole;
B and B′ are each independently —(C≡C)— or —(CH₂)₂—;
R₁ is H, halogen, —ORₐ, —NRₐR_{b}, —C(═O)ORₐ, —C(O)NRₐR_{b}, —C(═O)OH, —NR_{b}C(═O)Rₐ, hydroxyl, nitro, cyano, —S(O)₀₋₃Rₐ, —C₁₋₆ alkyl, C₂₋₆ alkenyl, C₂₋₆ alkynyl, or C₁₋₆ halogenated alkyl;
R₂ and R₂′ are each independently H, methyl or iodo;
m and n are each independently 0, 1 or 2;
p is 0, 1 or 2;
R₃ and R₃′ are H;
R₄ and R₄′ are each independently H, halogen, C₁₋₆ alkyl, hydroxyl, phenyl, or C₁₋₄ alkoxy;
X and Y are each

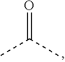

R₅ and R₅′ are each independently C₁₋₁₂ alkyl which is unsubstituted or substituted one or more times by R¹⁰;
R₇ and R₇′ are each independently C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, benzyl which is unsubstituted or substituted one or more times by R¹¹, 5-6 membered heteroaryl which is unsubstituted or substituted one or more times by R¹¹, 6-7 membered heteroaralkyl which is unsubstituted or substituted one or more times by R¹¹, 3-6 membered heterocycle which is unsubstituted or substituted one or more times by R¹², or 4-7 membered heterocycle-alkyl which is unsubstituted or substituted one or more times by R¹²; and
R₈ and R₈′ are each independently —NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —NR_{b}SO₂Rₐ, —NR_{b}SO₂NRₐR_{b}, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In some embodiments, the compounds of this invention are represented in Table 1A. In some embodiments, the compounds of this invention are represented in Table 1B. In certain embodiments, the variables used herein are as defined in the specific embodiments as shown in the tables below.

In one embodiment in the compounds of the present invention R₁ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, —P(═O)ORₐOR_{b}, C₁₋₆ alkyl which is unsubstituted or substituted one or more times by R¹⁰, C₂₋₆ alkenyl which is unsubstituted or substituted one or more times by R¹⁰, C₂₋₆ alkynyl which is unsubstituted or substituted one or more times by R¹⁰;

In one embodiment in the compounds of the present invention, herein as valency allows in B, B′, Rₐ, R_{b}, R_{c}, and R_{d}, R₁, R₂, R₂′, R₃, R₃′, R₄, R₄′, R¹⁰, R¹¹ and R¹² each of alkyl, alkenyl, alkynyl, alkoxy, aryl, aralkyl, heteroaryl, heteroaralkyl, heterocycle, or heterocycle-alkyl is independently unsubstituted or substituted one or more times by halogen, —OR_{a′}, oxo, —NRₐ, R_{b′}, —C(═O)OR_{a′}, —C(O)NR_{a′}, R_{b′}, —C(═O)OH, —C(═O)R_{a′}, —C(═NOR_{c′})R_{a′}, —C(═NR_{c′})NR_{a′}R_{b′}, —NR_{d′}C(═O)NR_{a′}R_{b′}, —NR_{b′}C(═O)R_{a′}, —NR_{d′}C(═NR_{c′})NR_{a′}R_{b′}, —NR_{b′}C(═O)OR_{a′}, —OC(═O)NR_{a′}R_{b′}, —OC(═O)R_{a′}, —OC(═O)OR_{a′}, hydroxyl, nitro, azido, cyano, —S(O)₀₋₃R_{a′}, —SO₂NR_{a′}R_{b′}, —NR_{b′}SO₂R_{a′}; wherein R_{a′}-R_{d′} are each independently H, C₁₋₁₂ alkyl.

In one embodiment in the compounds of the present invention p is 0, 1 or 2.

In one embodiment in the compounds of the present invention p is 0 or 1.

In one embodiment in the compounds of the present invention p is 0.

In one embodiment in the compounds of the present invention p is 2.

In one embodiment in the compounds of the present invention R₄ and R₄′ are H.

In one embodiment in the compounds of the present invention R₁ is halogen, C₁₋₃ alkyl, hydroxyl, cyano, or C₁₋₃ alkoxy.

In one embodiment in the compounds of the present invention R₁ is chloro, fluoro, methyl, hydroxyl, cyano, or methoxy.

In one embodiment in the compounds of the present invention n R₁ is H.

In one embodiment R¹⁰ is halogen, —ORₐ, oxo, —C(═O)ORₐ, —C(O)NRₐR_{b}, —C(═O)OH, —C(═O)Rₐ, —OC(═O)NRₐR_{b}, —OC(═O)Rₐ, —OC(═O)ORₐ, hydroxyl, cyano, wherein Rₐ-R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹¹ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, or —NR_{b}SO₂NRₐR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹¹ is halogen, —ORₐ, —NRₐR_{b}, —C(═O)ORₐ, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, —OC(═O)Rₐ, —OC(═O)ORₐ, hydroxyl, cyano, —SO₂NRₐR_{b}, —NR_{b}SO₂Rₐ, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹¹ is halogen, —ORₐ, —NRₐR_{b}, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, hydroxyl, cyano, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹¹ is halogen, —ORₐ, —NRₐR_{b}, hydroxyl, cyano, C₁₋₆ alkyl, wherein Rₐ-R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹² is halogen, —ORₐ, OXO, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹² is halogen, —ORₐ, OXO, —NRₐR_{b}, —C(═O)ORₐ, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, —OC(═O)Rₐ, —OC(═O)ORₐ, hydroxyl, cyano, —SO₂NRₐR_{b}, —NR_{b}SO₂Rₐ, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹² is halogen, —ORₐ, OXO, —NRₐR_{b}, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, hydroxyl, cyano, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹² is halogen, —ORₐ, oxo, —NRₐR_{b}, hydroxyl, cyano, C₁₋₆ alkyl, wherein Rₐ-R_{b} are are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-membered heteroaralkyl, 3-12 membered heterocycle, or 4-18 membered heterocycle-alkyl.

In one embodiment in the compounds of the present invention wherein as valency allows in B, B′, Rₐ, R_{b}, R_{c}, and R_{d}, R₁, R₂, R₂′, R₃, R₃′, R₄, R₄′, R¹⁰, R¹¹ and R¹² each of alkyl, alkenyl, alkynyl, alkoxy, aryl, aralkyl, heteroaryl, heteroaralkyl, heterocycle, or heterocycle-alkyl is independently unsubstituted or substituted one or more times by halogen, —OR_{a′}—NR_{a′}R_{b′, C(═O)OR}_{a′}, —C(O)NR_{a′}R_{b′}, —C(═O)OH, hydroxyl, nitro, azido, cyano; wherein R_{a′}-R_{d′} are each independently H, C₁₋₁₂ alkyl.

In one embodiment in the compounds of the present invention wherein as valency allows in B, B′, Rₐ, R_{b}, R_{c}, and R_{d}, R₁, R₂, R₂′, R₃, R₃′, R₄, R₄′, R¹⁰, R¹¹ and R¹² each of alkyl, alkenyl, alkynyl, alkoxy, aryl, aralkyl, heteroaryl, heteroaralkyl, heterocycle, or heterocycle-alkyl is independently unsubstituted or substituted one time by halogen.

In one embodiment in the compounds of the present invention wherein as valency allows in B, B′, Rₐ, R_{b}, R_{c}, and R_{d}, R₁, R₂, R₂′, R₃, R₃′, R₄, R₄′, R¹⁰, R¹¹ and R¹² each of alkyl, alkenyl, alkynyl, alkoxy, aryl, aralkyl, heteroaryl, heteroaralkyl, heterocycle, or heterocycle-alkyl is independently unsubstituted or substituted one time by fluoro.

The use of a compound of the present invention for treating an Hepatitis C viral infection in a human. The use of a compound of the present invention further comprising administering at least one additional agent. The use of a compound of the present invention wherein said at least one additional agent is selected from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES).

The use of a compound of the present invention, wherein said at least one additional agent is selected from ribavirin and interferon-α.

The use of a compound of the present invention for the manufacture of a medicament.

A pharmaceutical formulation comprising at least one compound of the present invention and at least one pharmaceutically acceptable carrier or excipient.

The use of a compound of the present invention for treating an Hepatitis C viral infection in a human. The use of a compound of the present invention further comprising administering at least one additional agent. The use of a compound of the present invention wherein said at least one additional agent is selected from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES). The use of a compound of the present invention wherein said at least one additional agent is selected from ribavirin and interferon-α.

The use of a compound of the present invention for the manufacture of a medicament.

A pharmaceutical formulation comprising at least one compound of the present invention and at least one pharmaceutically acceptable carrier or excipient.

According to an aspect of the invention, the compounds of the invention are selected from Table 1A:

TABLE 1A

| No. | Structure |
|---|---|
| 1 | 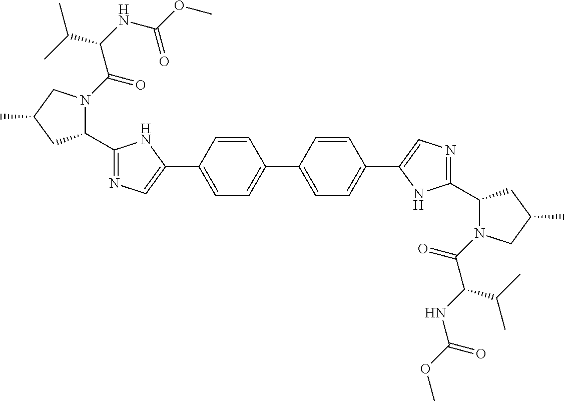 |
| 4 | 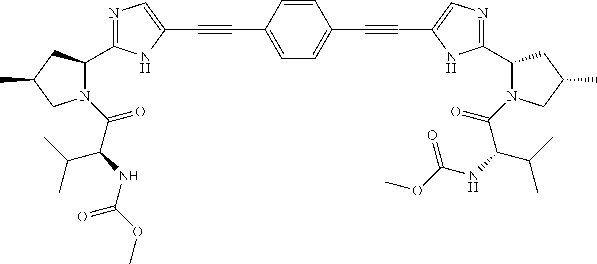 |
| 5 | 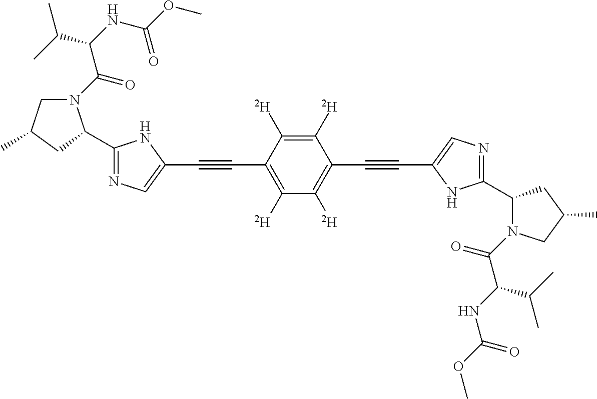 |
| 6 | 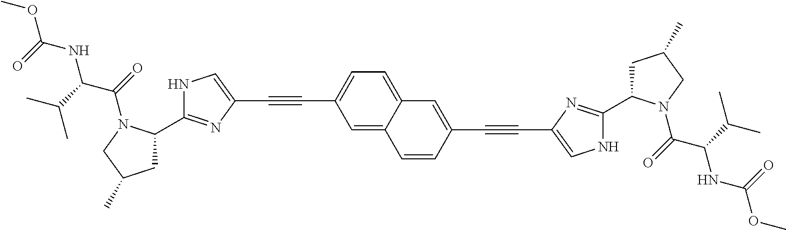 |
| 7 | 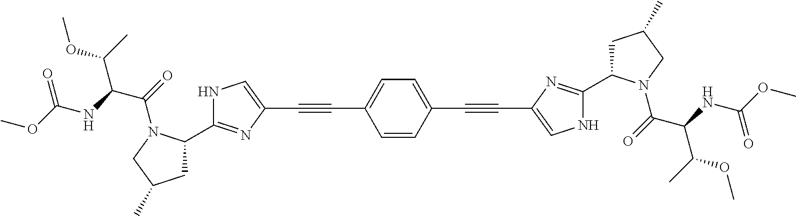 |
| 8 | 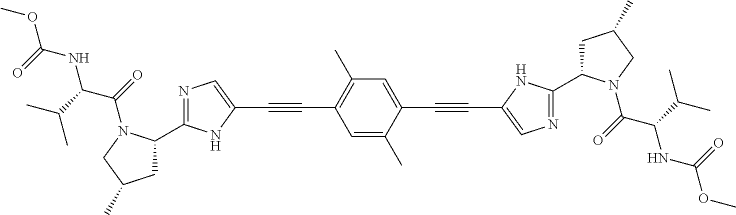 |
| 9 | 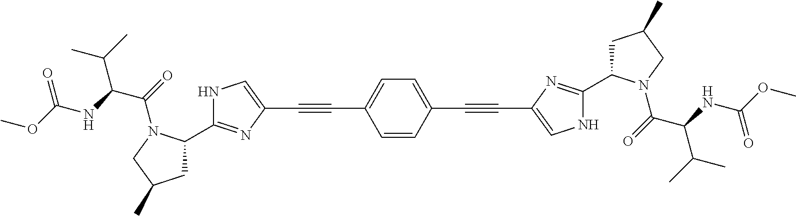 |
| 10 | 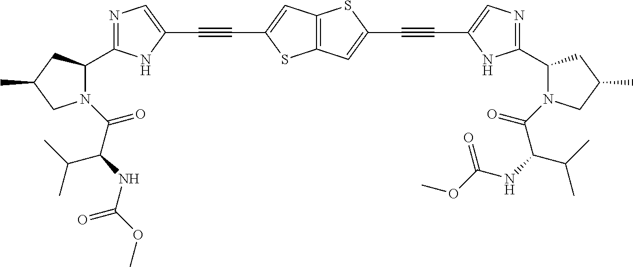 |
| 11 | 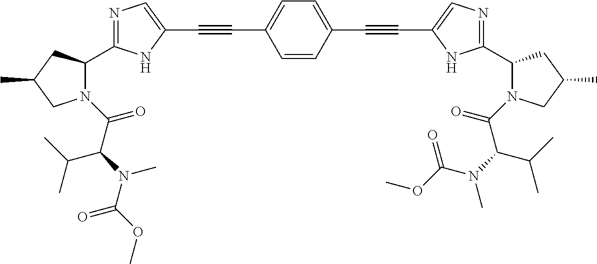 |
| 12 | 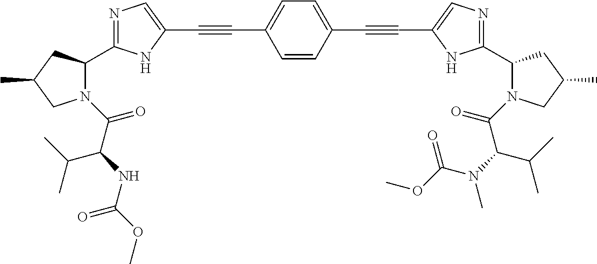 |
| 16 | 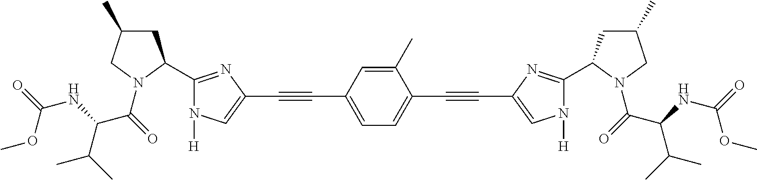 |
| 17 | 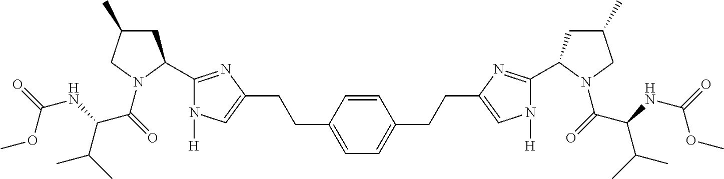 |
| 18 | 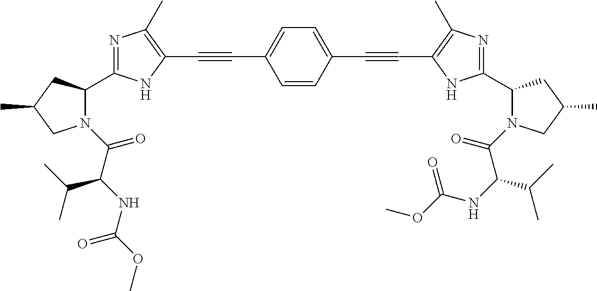 |
| 20 | 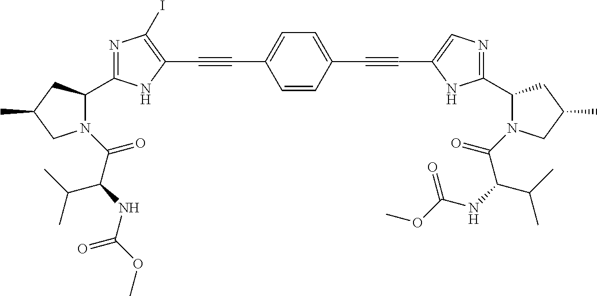 |
| 21 | 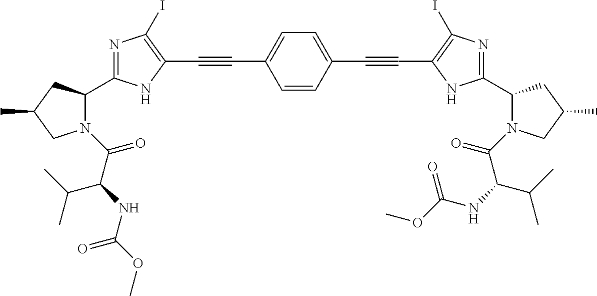 |
| 22 | 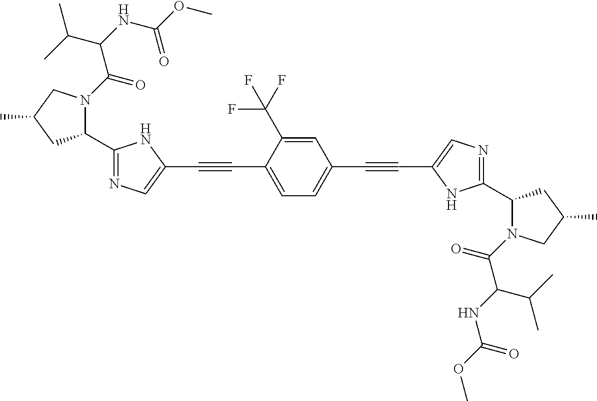 |
| \23 | 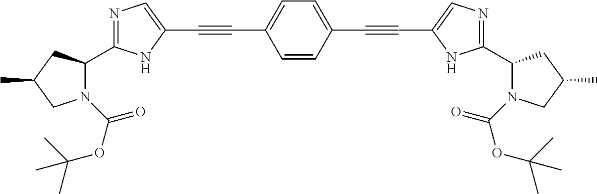 |
| 24 | 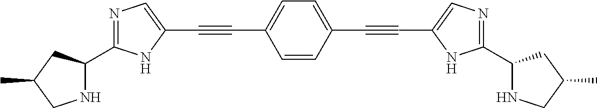 |
| 26 | 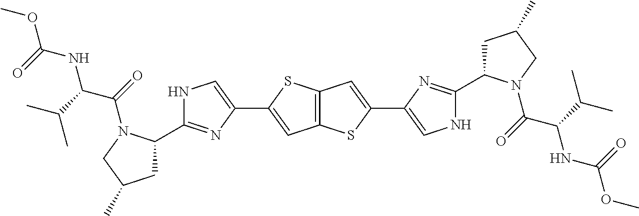 |
| 27 | 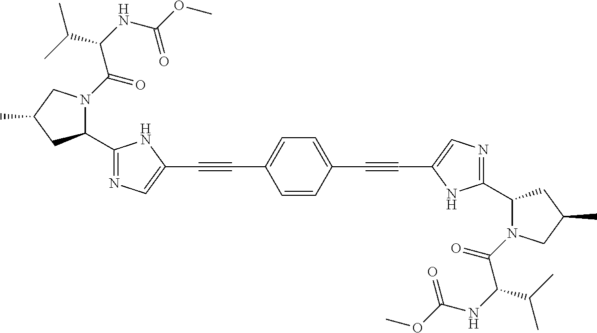 |
| 28 | 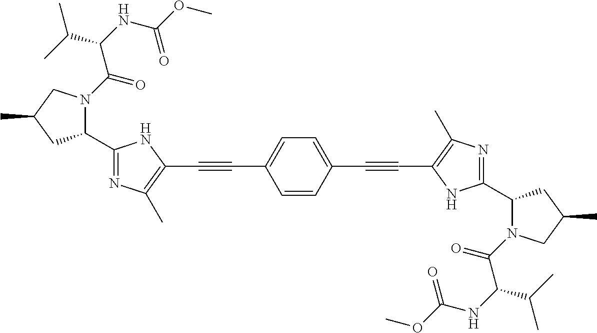 |
| 29 | 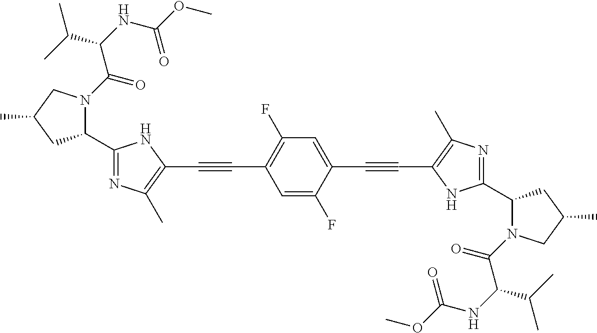 |
| 31 | 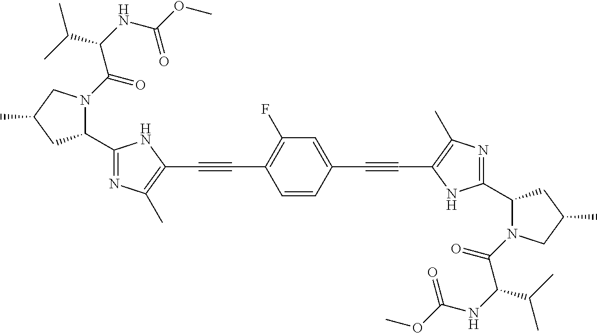 |
| 32 | 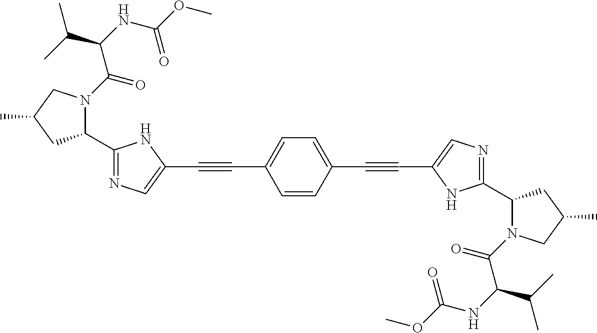 |
| 33 | 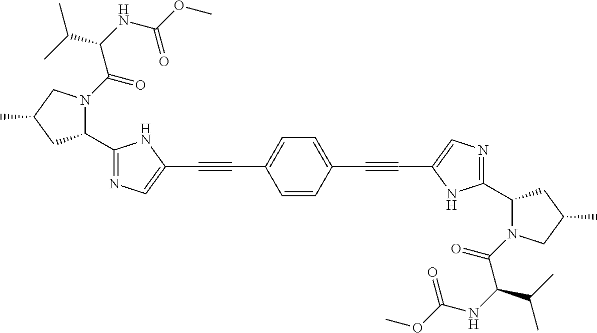 |
| 34 | 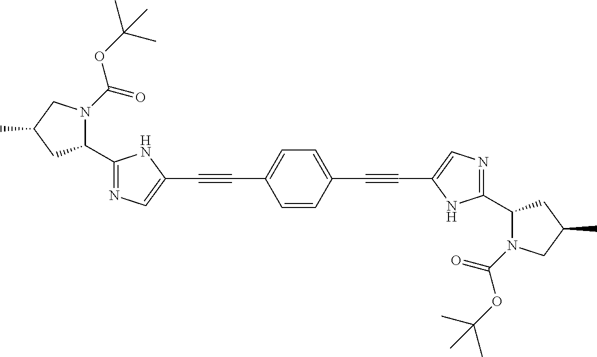 |
| 35 | 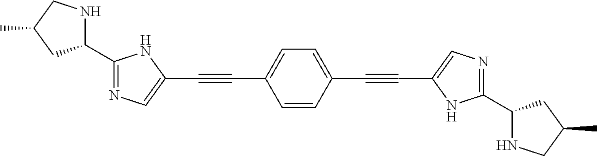 |
| 36 | 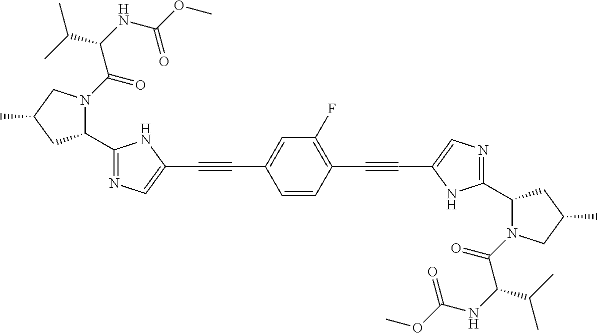 |
| 37 | 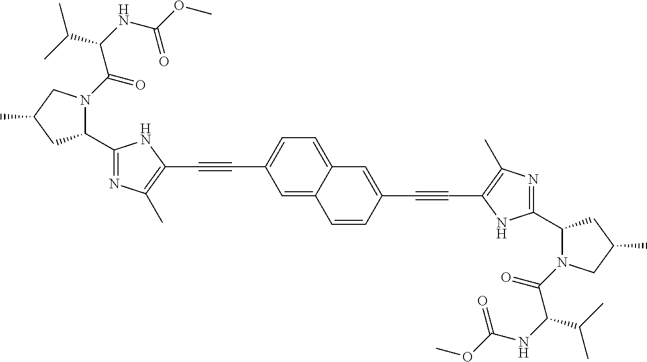 |
| 38 | 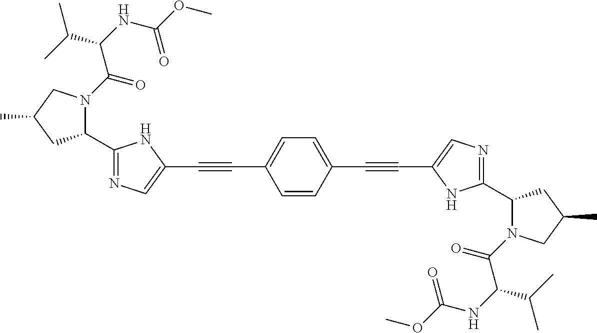 |
| 39 | 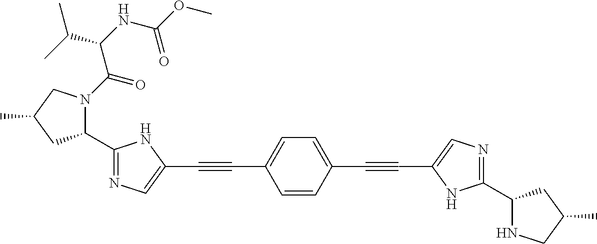 |
| 40 | 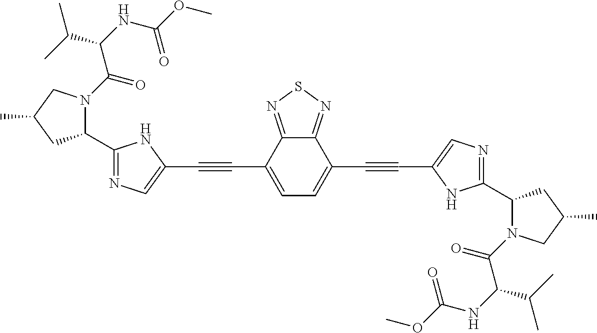 |
| 41 | 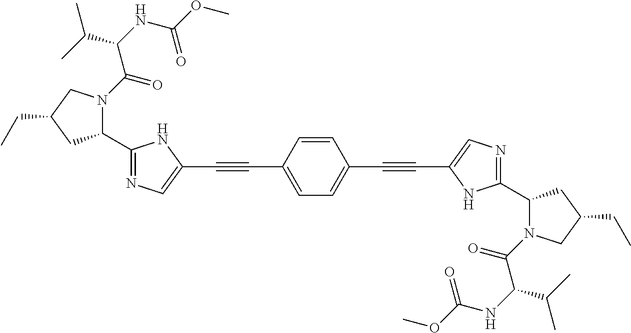 [SMILES: CC[C@H]1C[C@@H](c2ncc(C#Cc3ccc(C#Cc4cnc([C@@H]5C[C@H](CC)CN5C(=O)[C@@H](NC(=O)OC)C(C)C)[nH]4)cc3)[nH]2)N(C(=O)[C@@H](NC(=O)OC)C(C)C)C1] |
| 44 | 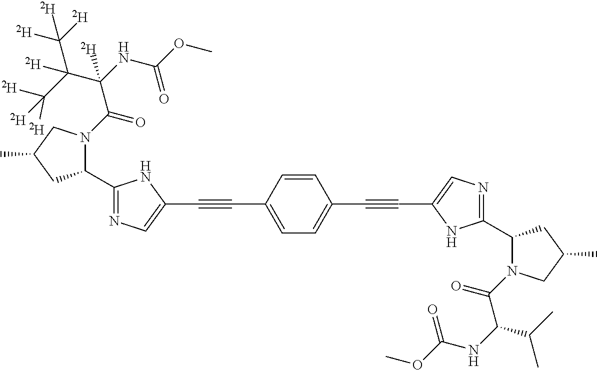 |
| 45 | 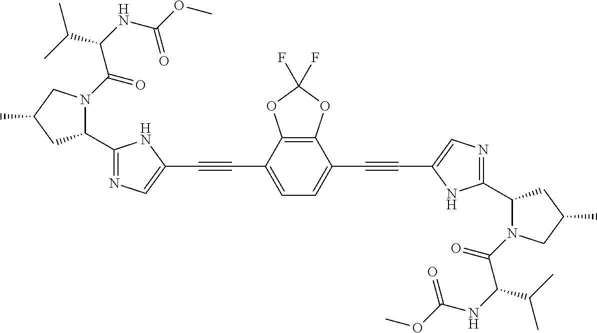 |
| 46 | 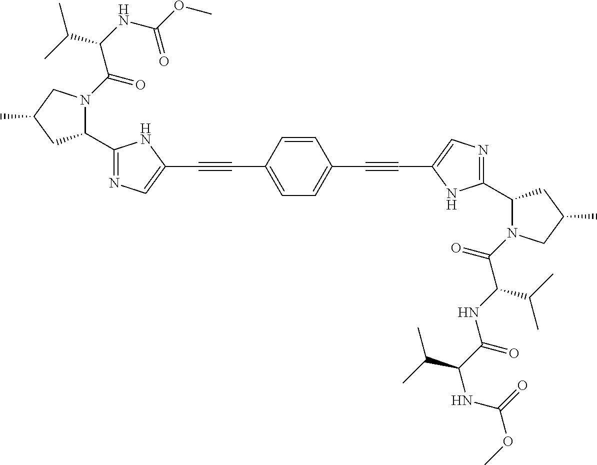 |
| 47 | 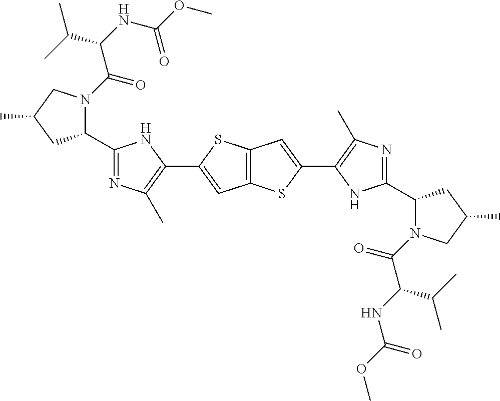 |
| 48 | 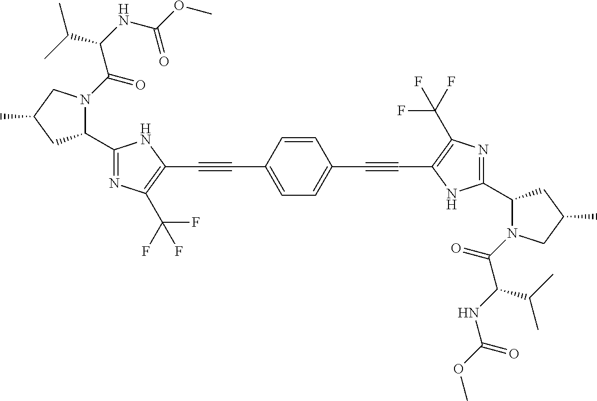 |
| 49 | 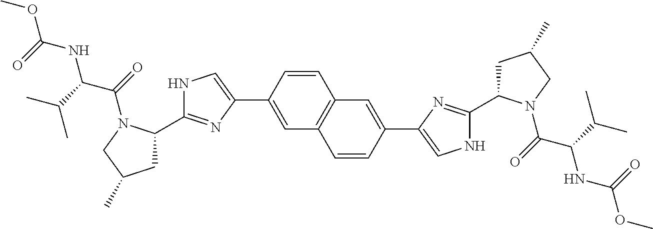 |
| 50 | 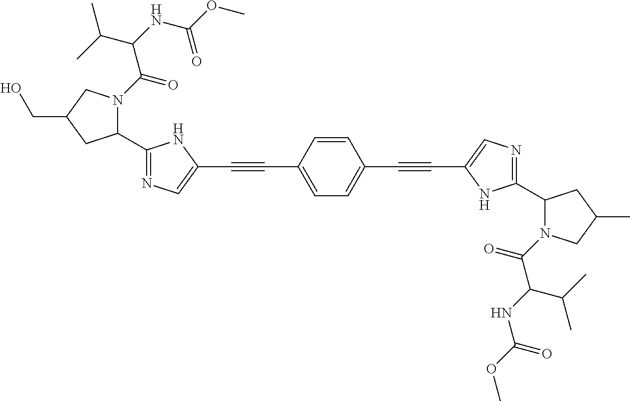 [SMILES: COC(=O)NC(C(=O)N1CC(C)CC1c1ncc(C#Cc2ccc(C#Cc3cnc(C4CC(CO)CN4C(=O)C(NC(=O)OC)C(C)C)[nH]3)cc2)[nH]1)C(C)C] |
| 51 | 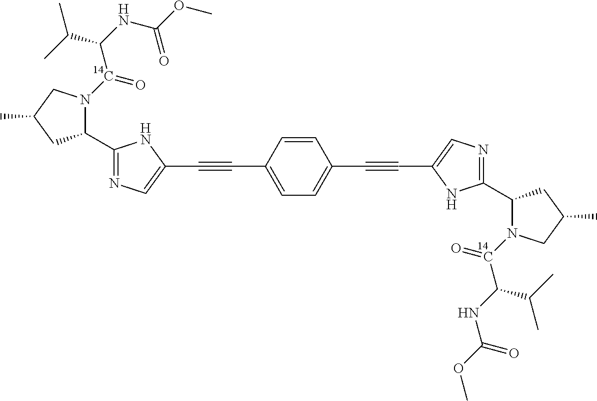 |
| 52 | 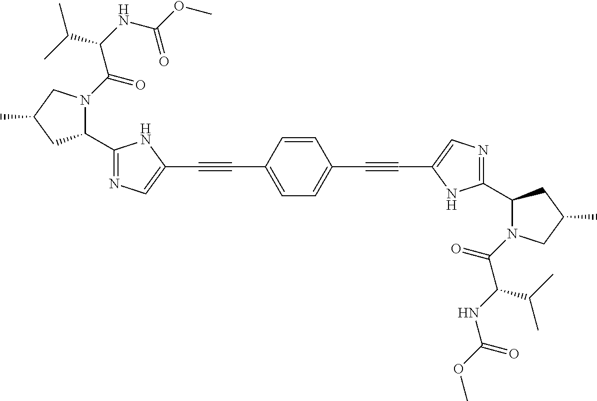 |
| 53 | 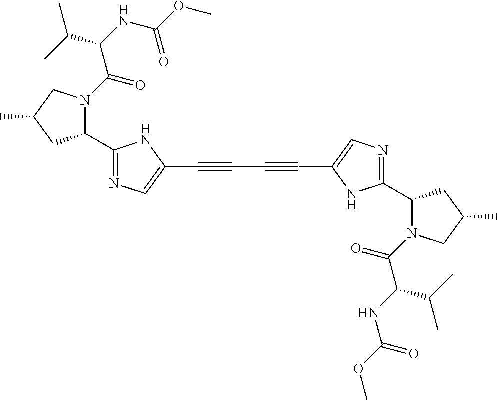 |
| 54 | 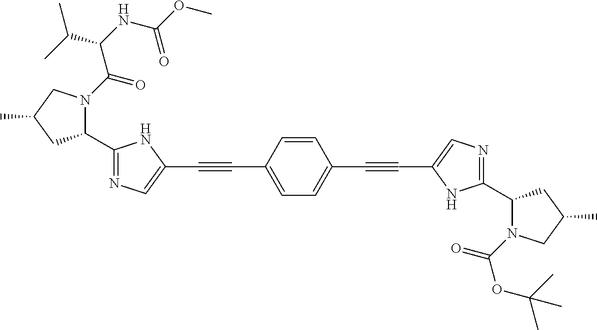 |
| 55 | 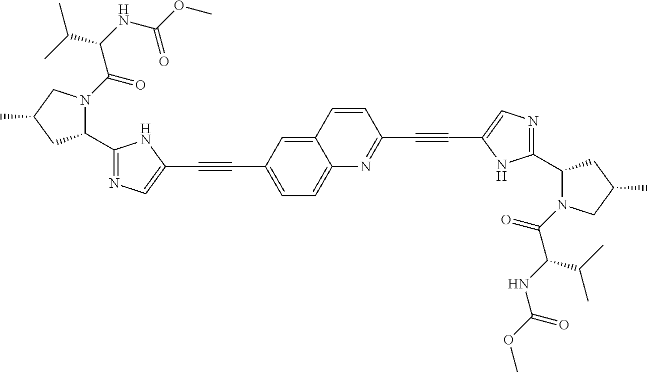 |
| 56 | 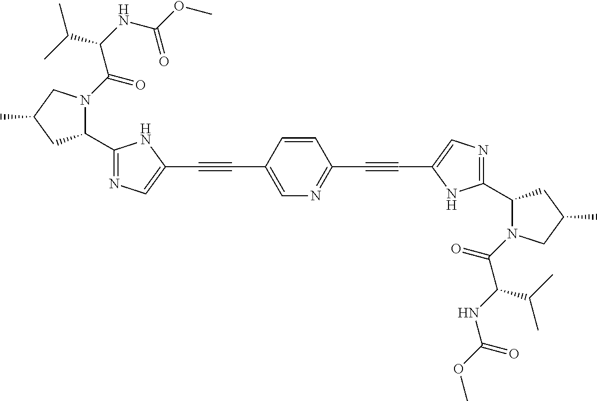 |
| 57 | 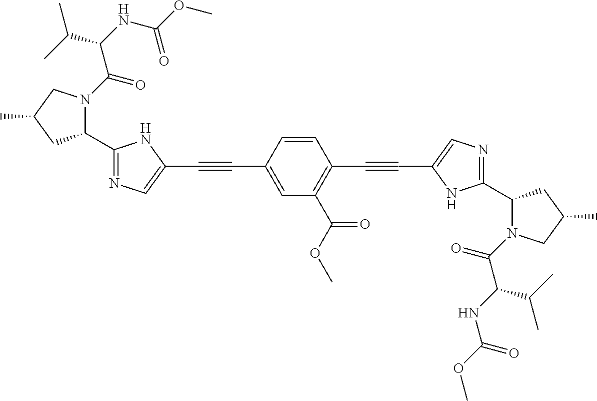 |
| 58 | 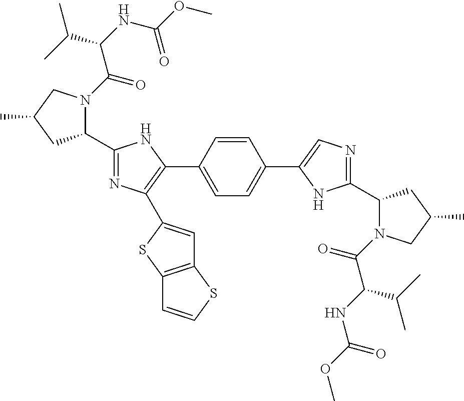 |
| 59 | 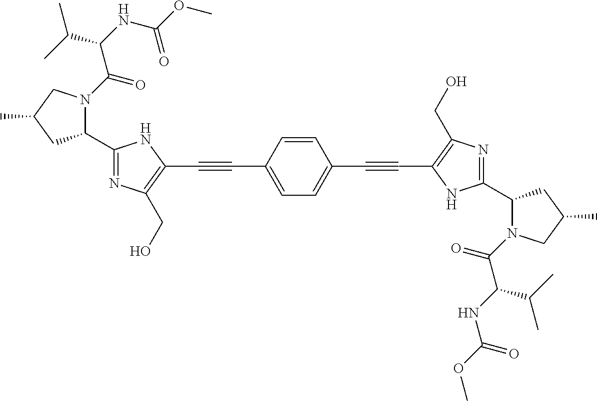 |
| 60 | 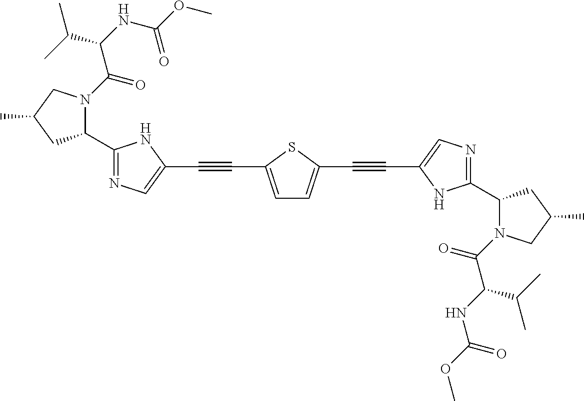 |
| 61 | 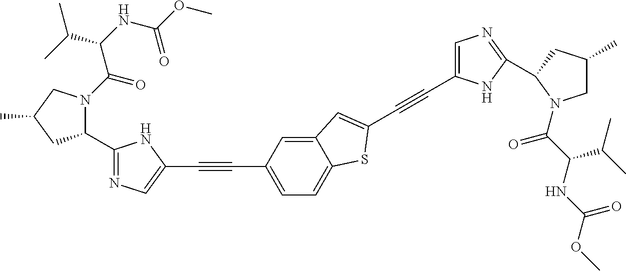 [SMILES: COC(=O)N[C@H](C(=O)N1C[C@@H](C)C[C@H]1c1ncc(C#Cc2ccc3sc(C#Cc4cnc([C@@H]5C[C@H](C)CN5C(=O)[C@@H](NC(=O)OC)C(C)C)[nH]4)cc3c2)[nH]1)C(C)C] |
| 62 | 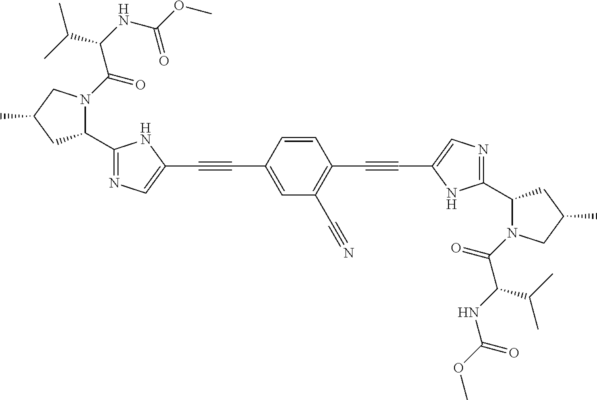 |
| 63 | 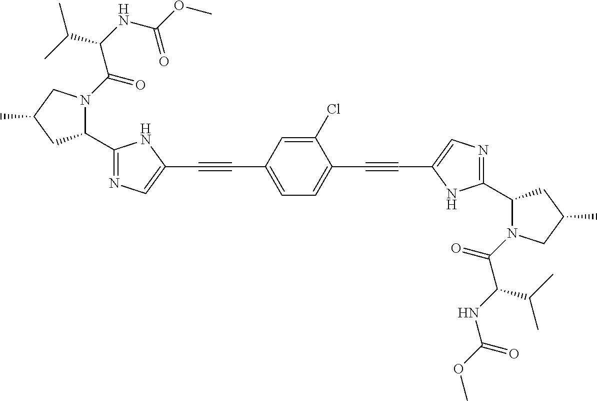 |
| 64 | 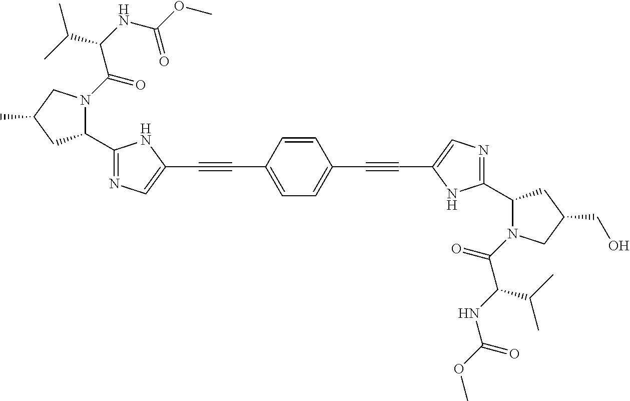 [SMILES: COC(=O)N[C@H](C(=O)N1C[C@@H](C)C[C@H]1c1ncc(C#Cc2ccc(C#Cc3cnc([C@@H]4C[C@H](CO)CN4C(=O)[C@@H](NC(=O)OC)C(C)C)[nH]3)cc2)[nH]1)C(C)C] |
| 65 | 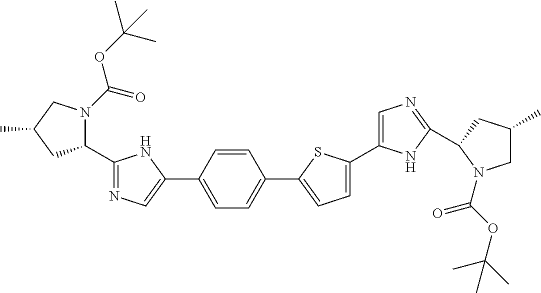 |
| 66 | 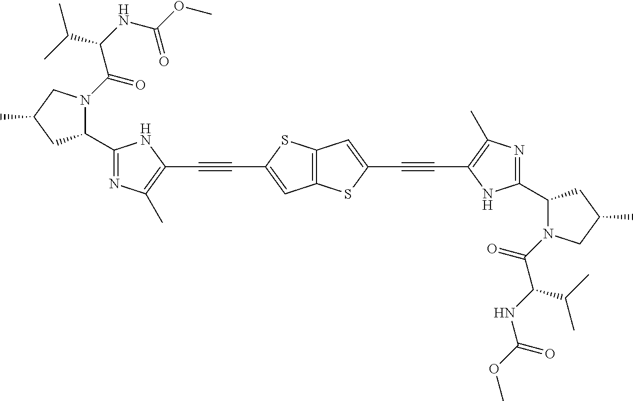 |
| 67 | 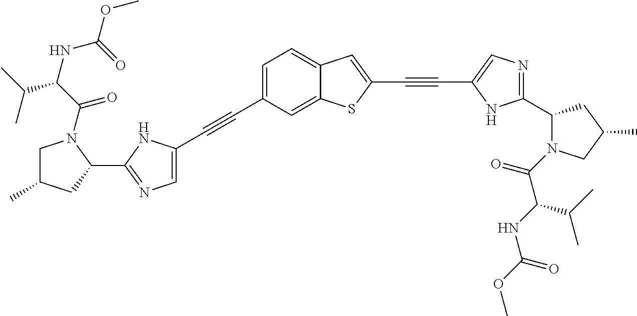 [SMILES: COC(=O)N[C@H](C(=O)N1C[C@@H](C)C[C@H]1c1ncc(C#Cc2ccc3cc(C#Cc4cnc([C@@H]5C[C@H](C)CN5C(=O)[C@@H](NC(=O)OC)C(C)C)[nH]4)sc3c2)[nH]1)C(C)C] |
| 68 | 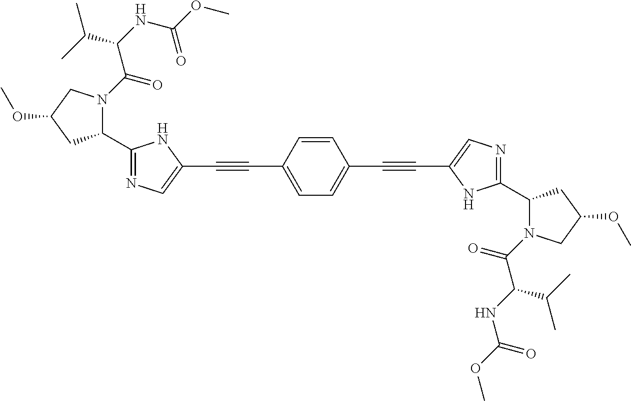 |
| 69 | 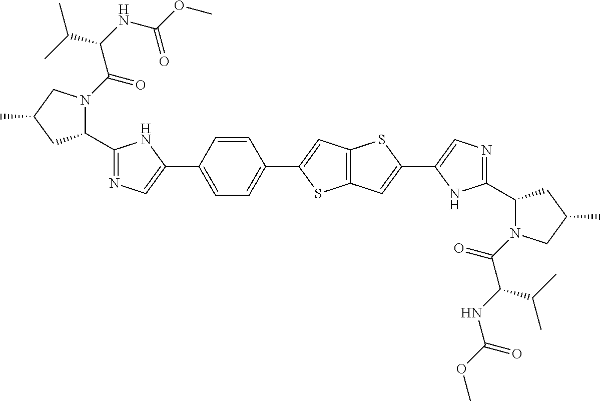 |

or a pharmaceutically acceptable salt thereof.

According to another aspect of the invention, the compounds of the invention are selected from Table 1B:

TABLE 1B

| No. | Structure |
|---|---|
| 2 | 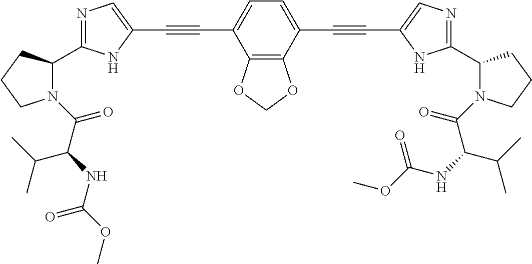 [SMILES: COC(=O)N[C@H](C(=O)N1CCC[C@H]1c1ncc(C#Cc2ccc(C#Cc3cnc([C@@H]4CCCN4C(=O)[C@@H](NC(=O)OC)C(C)C)[nH]3)c3c2OCO3)[nH]1)C(C)C] |
| 3 | 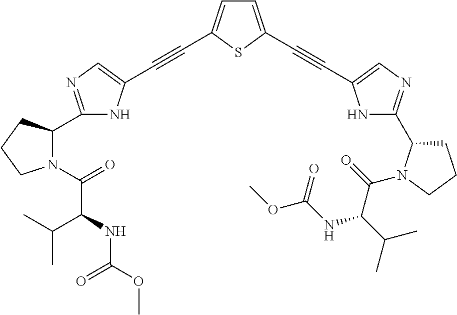 [SMILES: COC(=O)N[C@H](C(=O)N1CCC[C@H]1c1ncc(C#Cc2ccc(C#Cc3cnc([C@@H]4CCCN4C(=O)[C@@H](NC(=O)OC)C(C)C)[nH]3)s2)[nH]1)C(C)C] |
| 13 | 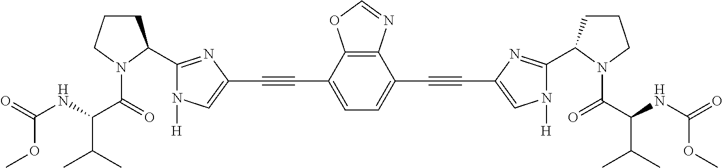 [SMILES: COC(=O)N[C@H](C(=O)N1CCC[C@H]1c1nc(C#Cc2ccc(C#Cc3c[nH]c([C@@H]4CCCN4C(=O)[C@@H](NC(=O)OC)C(C)C)n3)c3ocnc23)c[nH]1)C(C)C] |
| 14 | 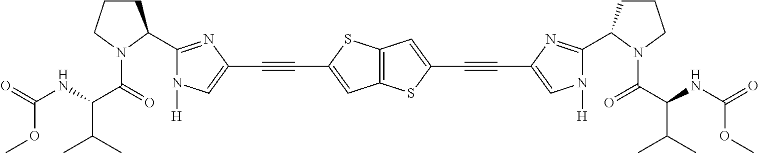 [SMILES: COC(=O)N[C@H](C(=O)N1CCC[C@H]1c1nc(C#Cc2cc3sc(C#Cc4c[nH]c([C@@H]5CCCN5C(=O)[C@@H](NC(=O)OC)C(C)C)n4)cc3s2)c[nH]1)C(C)C] |
| 15 | 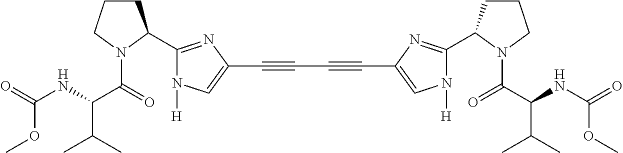 [SMILES: COC(=O)N[C@H](C(=O)N1CCC[C@H]1c1nc(C#CC#Cc2c[nH]c([C@@H]3CCCN3C(=O)[C@@H](NC(=O)OC)C(C)C)n2)c[nH]1)C(C)C] |
| 19 | 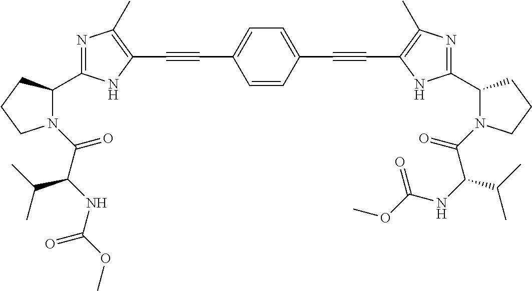 [SMILES: COC(=O)N[C@H](C(=O)N1CCC[C@H]1c1nc(C)c(C#Cc2ccc(C#Cc3[nH]c([C@@H]4CCCN4C(=O)[C@@H](NC(=O)OC)C(C)C)nc3C)cc2)[nH]1)C(C)C] |

In one embodiment, the present invention provides a method for preparing a compound of formula (IV):

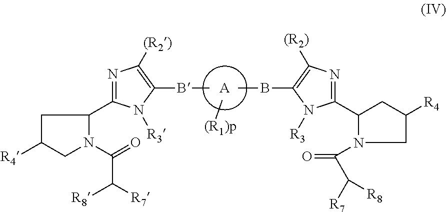

or a pharmaceutically acceptable salt thereof, wherein each of A, B, B′, R¹, p, R₂, R_{2′}, R₃, R_{3′}, R₄, R_{4′}, R₇, R_{7′}, R₈, and R_{8′}, are as defined herein,
wherein said method comprises the steps of:
a) contacting a compound of formula (XXX):

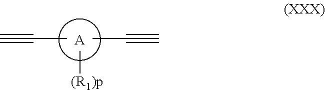

under coupling conditions with a compound of formula (XXXI) and a compound of formula (XXXII); and

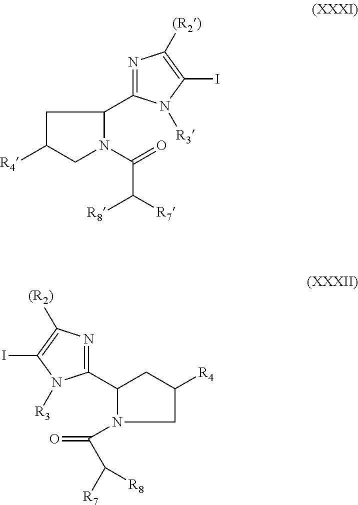

b) optionally hydrogenating the alkyne groups to provide a compound of formula (IV).

In one embodiment, the present invention provides a method for preparing a compound of formula (IV):

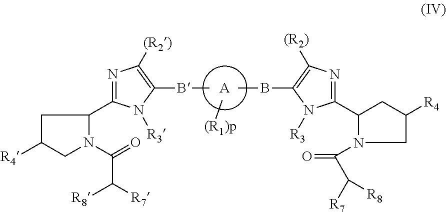

or a pharmaceutically acceptable salt thereof, wherein each of A, B, B′, R¹, p, R₂, R_{2′}, R₃, R_{3′}, R₄, R_{4′}, R₇, R_{7′}, R₈, and R_{8′}, are as defined herein,
wherein said method comprises the steps of:
a) contacting a compound of formula (XXXA) where LG is a leaving group, such as halo:

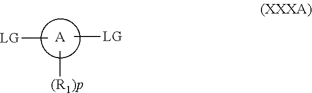

under coupling conditions with a compound of formula (XXXIA) and a compound of formula (XXXIIA); and

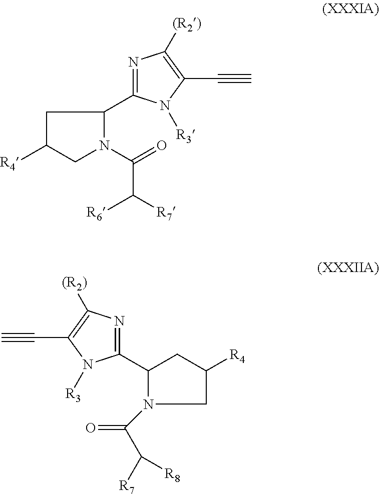

b) optionally hydrogenating the alkyne groups to provide a compound of formula (IV).

In one embodiment, the coupling conditions comprise bis(triphenylphosphine) palladiumchloride, copper iodide, and triethylamine.

In one embodiment, the present invention provides a method for preparing a compound of formula (XXXII):

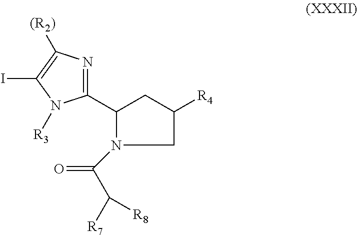

wherein each of R₂, R₃, R₄, R₇, and R₈, are as defined herein,
wherein said method comprises the steps of:
a) reducing a compound of formula (XXVI) to provide a compound of formula (XXVII), where each R is each independently an alkyl group:

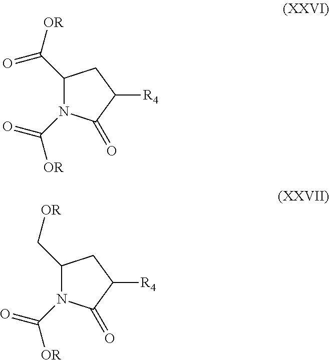

g) oxidizing a compound of formula (XXVII) to provide a compound of formula (XVI):

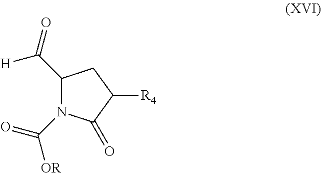

h) contacting a compound of formula (XVI) with a compound of formula (XVII) and optionally a compound of formula R₃-LG, where LG is a leaving group, under reaction conditions sufficient to provide a compound of formula (XV):

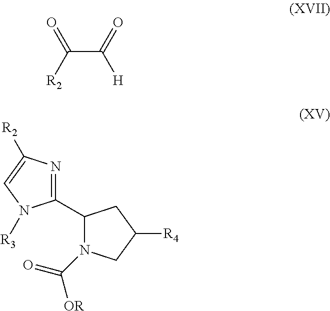

e) halogenating a compound of formula (XV) under reaction conditions sufficient to provide a compound of formula (XIII):

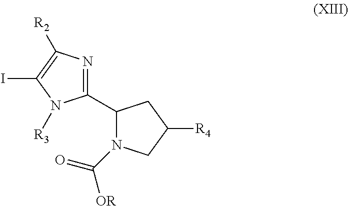

f) reacting a compound of formula (XIII) under deprotection conditions to provide a compound of formula (XI); and:

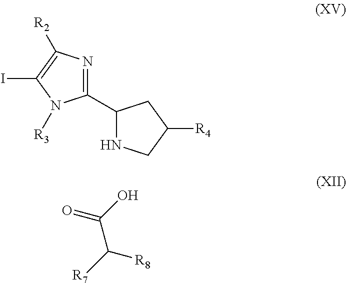

g) contacting a compound of formula (XI) under coupling conditions with a compound of formula (XII) to provide a compound of formula (XXXII):
to provide a compound of formula (XXXII).

In one embodiment, the oxidizing of step g) comprises 2,2,6,6-tetramethyl-piperidin-1-oxyl (TEMPO).

In one embodiment, the present invention provides a method for preparing a compound of formula (XXVI):

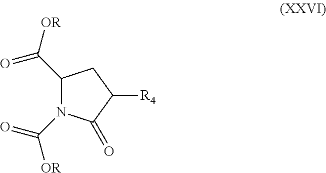

wherein R₄ is as defined herein, and each R is each independently an alkyl group,
wherein said method comprises the steps of:
a) contacting a compound of formula (XXI) under reaction conditions sufficient to provide a compound of formula (XXII):

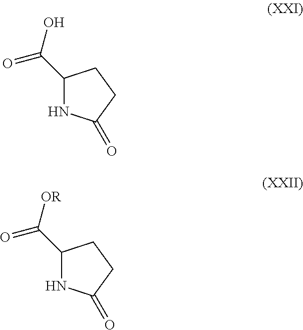

b) contacting a compound of formula (XXII) under reaction conditions sufficient to provide a compound of formula (XXIII), and:

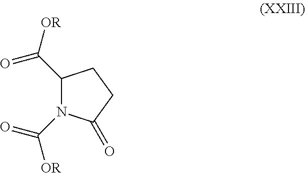

c) contacting a compound of formula (XXIII) under reaction conditions sufficient to provide a compound of formula (XXVI):

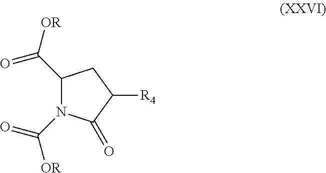

to provide a compound of formula (XXVI).

In one embodiment, the present invention provides a method for preparing a compound of formula (XXXI):

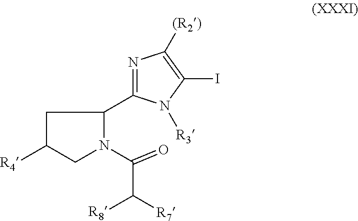

each of R_{2′}, R_{3′}, R_{4′}, R_{7′}, and R_{8′}, are as defined herein,
wherein said method comprises the steps as disclosed for preparing a compound of formula (XXXII), wherein each of R_{2′}, R_{3′}, R_{4′}, R_{7′}, and R_{8′}, are as defined for R₂, R₃, R₄, R₇, and R₈, respectively

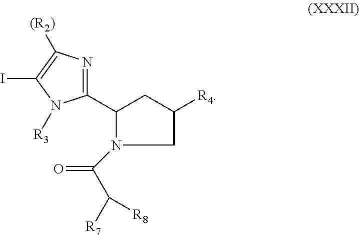

In one embodiment, the present invention provides a method for preparing ((S)-1-{(2S,4S)-2-[5-(4-{2-[(2S,4S)-1-((S)-2-Methoxycarbonylamino-3-methyl-butyryl)-4-methyl-pyrrolidin-2-yl]-3H-imidazol-4-ylethynyl}-phenylethynyl)-1H-imidazol-2-yl]-4-methyl-pyrrolidine-1-carbonyl}-2-methylpropyl)-carbamic acid methyl ester:

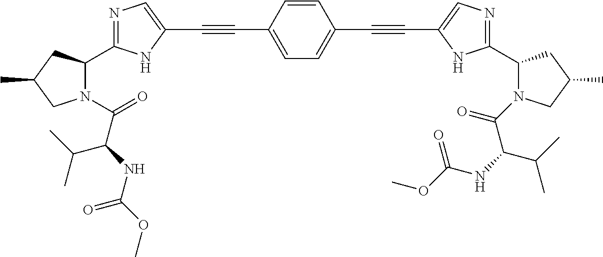

wherein said method comprises:
contacting a compound of formula (X) under coupling conditions with a compound of formula (XXX):

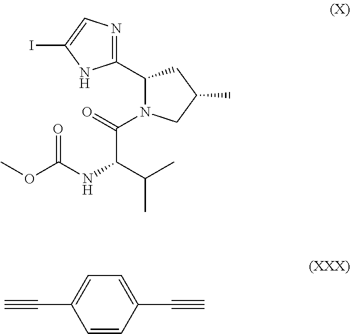

to provide ((S)-1-{(2S,4S)-2-[5-(4-{2-[(2S,4S)-1-((S)-2-Methoxycarbonylamino-3-methyl-butyryl)-4-methyl-pyrrolidin-2-yl]-3H-imidazol-4-ylethynyl}-phenylethynyl)-1H-imidazol-2-yl]-4-methyl-pyrrolidine-1-carbonyl}-2-methylpropyl)-carbamic acid methyl ester.

In one embodiment, the coupling conditions comprise bis(triphenylphosphine) palladiumchloride, copper iodide, and triethylamine.

In one embodiment, the present invention provides a method for preparing a compound of formula (X):

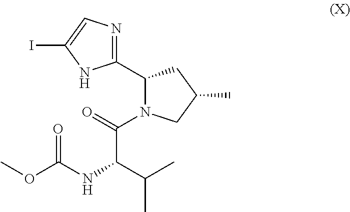

wherein said method comprises
a) reacting a compound of formula (XIII) under deprotection conditions to provide a compound of formula (XI); and:

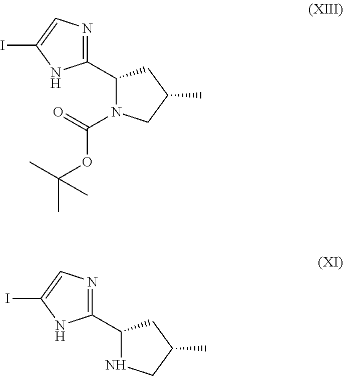 [SMILES: C[C@H]1C[C@@H](c2ncc(I)[nH]2)N(C(=O)OC(C)(C)C)C1 ; C[C@@H]1CN[C@H](c2ncc(I)[nH]2)C1]

b) contacting a compound of formula (XI) under coupling conditions with a compound of formula (XII):

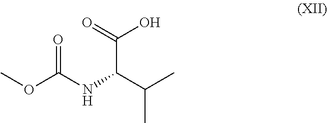

to provide a compound of formula (X).

In one embodiment, the coupling conditions of step b) comprise first contacting a compound of formula (XII) with O-(7-azabenzotriazol-1-yl)-N,N,N′,N′-tetramethyluronium hexafluorophosphate (HATU) and diisopropypyethylamine (DIPEA).

In one embodiment, the deprotection conditions of step a) comprise a mineral acid.

In one embodiment, the present invention provides a method for preparing a compound of formula (XIII):

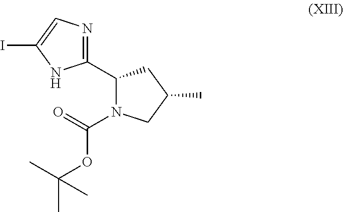 [SMILES: C[C@H]1C[C@@H](c2ncc(I)[nH]2)N(C(=O)OC(C)(C)C)C1]

wherein said method comprises reacting a compound of formula (XIV):

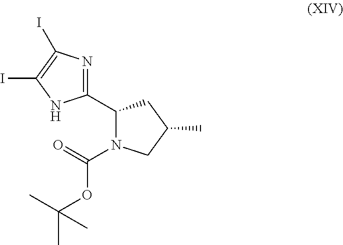 [SMILES: C[C@H]1C[C@@H](c2nc(I)c(I)[nH]2)N(C(=O)OC(C)(C)C)C1]

under reaction conditions sufficient to provide a compound of formula (XIII).

In one embodiment, the reaction conditions comprise methyl magnesium bromide.

In one embodiment, the present invention provides a method for preparing a compound of formula (XIV) having a (2S,4S) configuration:

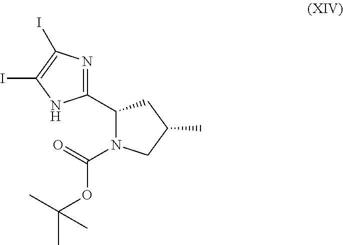 [SMILES: C[C@H]1C[C@@H](c2nc(I)c(I)[nH]2)N(C(=O)OC(C)(C)C)C1]

wherein said method comprises the steps of:
a) hydrogenating a compound of formula (XX) to provide a compound of formula (XIX):

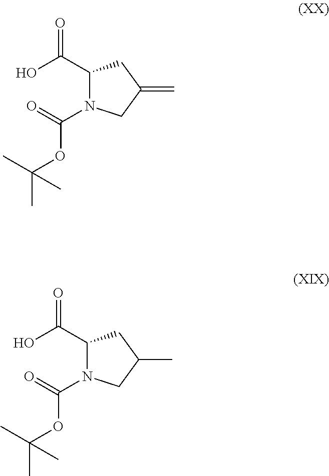

b) reducing a compound of formula (XIX) to provide a compound of formula (XIII):

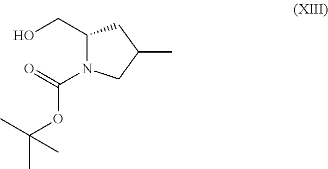

c) oxidizing a compound of formula (XIII) to provide a compound of formula (XVI):

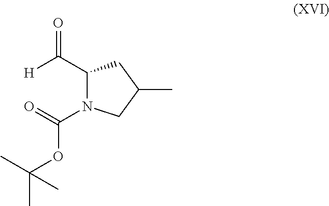

d) contacting a compound of formula (XVI) with a compound of formula (XVII) under reaction conditions sufficient to provide a compound of formula (XV):

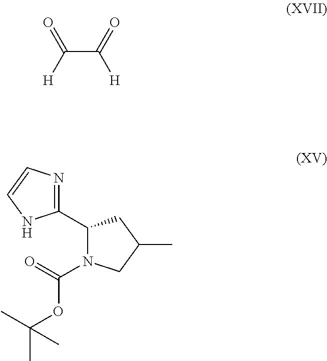

e) halogenating a compound of formula (XV) under reaction conditions sufficient to provide a compound of formula (XV), and:

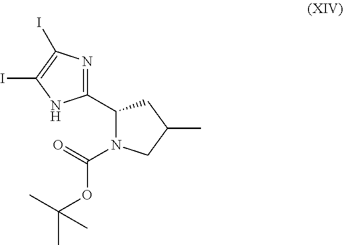

f) separating the mixture of (2S,4S) and (2S,4R) diastereomers to provide a compound of formula (XIV) having a (2S,4S) configuration.

In one embodiment, the separating of step f) comprises silica gel chromatography.

In one embodiment, the reaction conditions of step e) comprise 1-iodopyrrolidine-2,5-dione.

In one embodiment, the oxidizing of step c) comprises 2,2,6,6-tetramethyl-piperidin-1-oxyl (TEMPO).

In one embodiment, the reducing of step b) comprises borane.

In one embodiment, the hydrogenating of step a) comprises platinum oxide and hydrogen gas.

In one embodiment the present invention provides a method for preparing a compound of formula (XIV) having a (2S,4S) configuration:

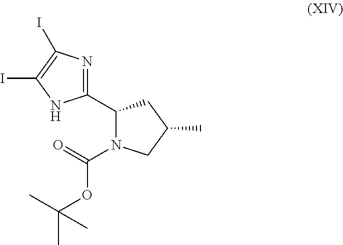 [SMILES: C[C@H]1C[C@@H](c2nc(I)c(I)[nH]2)N(C(=O)OC(C)(C)C)C1]

wherein said method comprises the steps of:
a) contacting a compound of formula (XXI) under reaction conditions sufficient to provide a compound of formula (XXII):

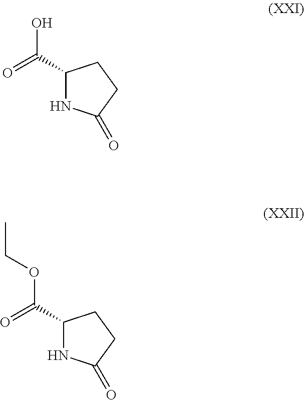

b) contacting a compound of formula (XXII) under reaction conditions sufficient to provide a compound of formula (XXIII):

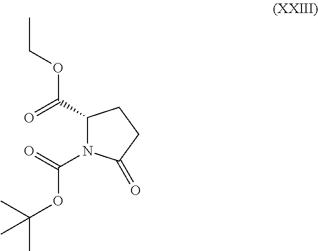 [SMILES: CCOC(=O)[C@@H]1CCC(=O)N1C(=O)OC(C)(C)C]

c) contacting a compound of formula (XXIII) with 1-t-butoxy-N,N,N′,N′-tetramethylmethanediamine under reaction conditions sufficient to provide a compound of formula (XXIV):

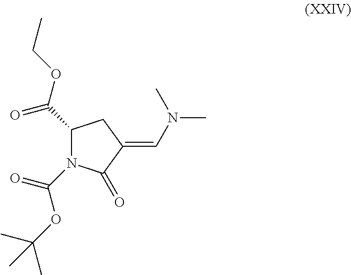 [SMILES: CCOC(=O)[C@@H]1C/C(=C\N(C)C)C(=O)N1C(=O)OC(C)(C)C]

d) reacting a compound of formula (XXIV) under reaction conditions sufficient to provide a compound of formula (XXVI):

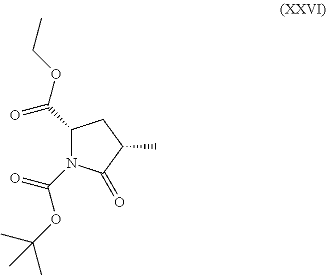 [SMILES: CCOC(=O)[C@@H]1C[C@H](C)C(=O)N1C(=O)OC(C)(C)C]

e) reducing a compound of formula (XXVI) to provide a compound of formula (XXVII):

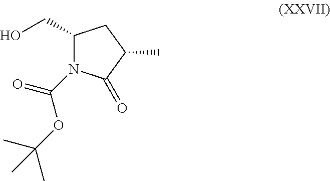 [SMILES: C[C@H]1C[C@@H](CO)N(C(=O)OC(C)(C)C)C1=O]

f) oxidizing a compound of formula (XXVII) to provide a compound of formula (XVI):

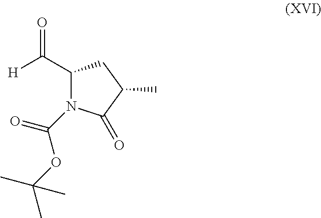 [SMILES: C[C@H]1C[C@@H](C=O)N(C(=O)OC(C)(C)C)C1=O]

g) contacting a compound of formula (XVI) with a compound of formula (XVII) under reaction conditions sufficient to provide a compound of formula (XV); and:

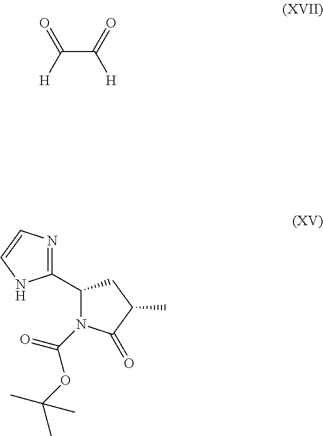

h) halogenating a compound of formula (XV) under reaction conditions sufficient to provide a compound of formula (XIV) having a (2S,4S) configuration.

In one embodiment, the reaction conditions of step d) comprise the steps of:
a) reacting a compound of formula (XXIV) under reaction conditions sufficient to provide a compound of formula (XXV); and:

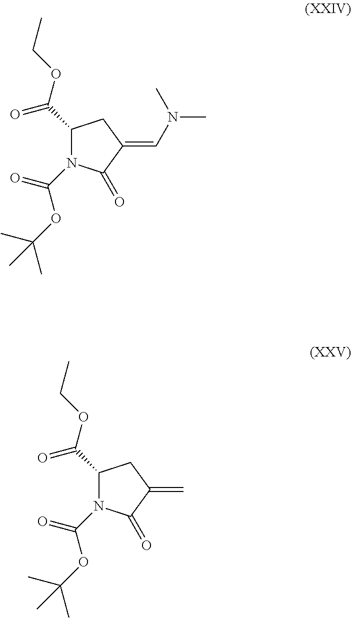

b) hydrogenating a compound of formula (XXV) to provide a compound of formula (XXVI):

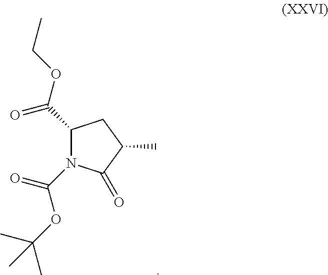 [SMILES: CCOC(=O)[C@@H]1C[C@H](C)C(=O)N1C(=O)OC(C)(C)C]

In one embodiment the present invention provides a method for preparing a compound of formula (III)

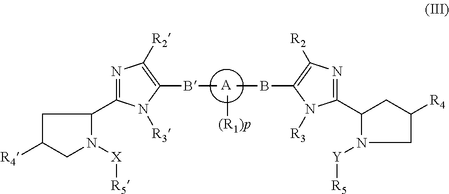

or a pharmaceutically acceptable salt thereof, wherein each of A, B, B′, R¹, p, R₂, R_{2′}, R₃, R_{3′}, R₄, R_{4′}, R₇, R_{7′}, R₈, and R_{8′}, are as defined herein,
wherein said method comprises the steps of:
a) contacting the compound of formula (XXXIV) with a compound of formula R⁵—X—OH and/or R⁵—Y—OH:

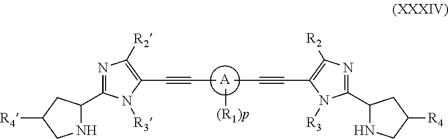

under reaction conditions to provide a compound of formula (III).

In one embodiment the present invention provides a method for preparing a compound of formula (XXXIV)

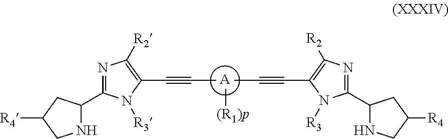

wherein each of A, B, B′, R¹, p, R₂, R_{2′}, R₃, R_{3′}, R₄, R_{4′}, R₇, R_{7′}, R₈, and R_{8′}, are as defined herein and each of B and B′, R₂ and R_{2′}, R₃ and R_{3′}, R₄ and R_{4′}, R₇ and R_{7′}, and R₈, and R_{8′} are the same respectively,
wherein said method comprises the steps of:
a) contacting a compound of formula (XXX) with a compound of formula (XIII) where R is an alkyl group:

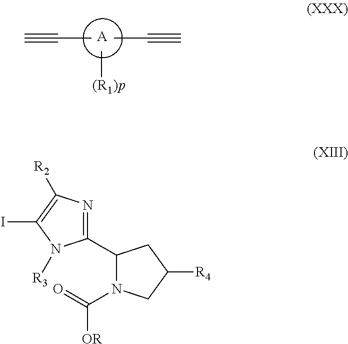

under coupling conditions to provide a compound of formula (XXXIII):

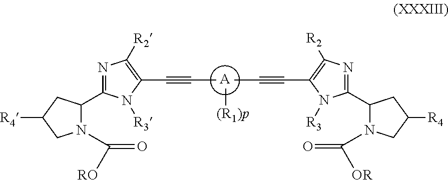

b) contacting the compound of formula (XXXIII) under conditions to provide a compound of formula (XXXIV).

In one embodiment, the coupling conditions comprise bis(triphenylphosphine) palladiumchloride, copper iodide, and triethylamine.

In one embodiment the present invention provides a method for preparing ((S)-1-{(2S,4S)-2-[5-(4-{2-[(2S,4S)-1-((S)-2-Methoxycarbonylamino-3-methyl-butyryl)-4-methyl-pyrrolidin-2-yl]-3H-imidazol-4-ylethynyl}-phenylethynyl)-1H-imidazol-2-yl]-4-methyl-pyrrolidine-1-carbonyl}-2-methylpropyl)-carbamic acid methyl ester:

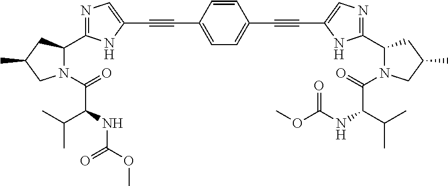

wherein said method comprises the steps of:
a) contacting a compound of formula (X) under coupling conditions with a compound of formula (XXX):

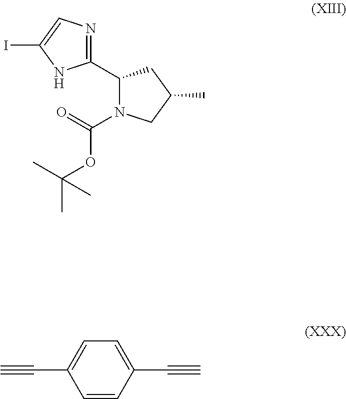

to provide a compound of formula (XXXV):

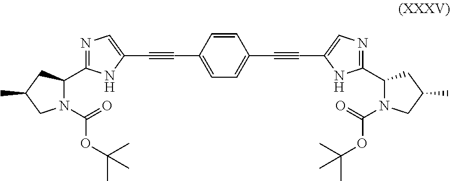

b) contacting the compound of formula (XXXV) under conditions to provide a compound of formula (XXXVI); and

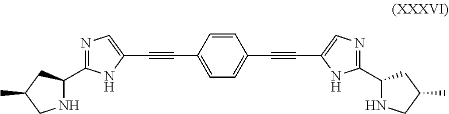

c) contacting the compound of formula (XXXVI) with N-methoxycarbonyl valine under reaction conditions to provide ((S)-1-{(2S,4S)-2-[5-(4-{2-[(2S,4S)-1-((S)-2-Methoxycarbonylamino-3-methyl-butyryl)-4-methyl-pyrrolidin-2-yl]-3H-imidazol-4-ylethynyl}-phenylethynyl)-1H-imidazol-2-yl]-4-methyl-pyrrolidine-1-carbonyl}-2-methylpropyl)-carbamic acid methyl ester.

In one embodiment the coupling conditions of step a) comprise bis(triphenylphosphine) palladiumchloride, copper iodide, and triethylamine.

In one embodiment, the present invention provides a compound according to the invention described herein for treating or preventing a Flaviviridae viral infection in a host.

In one embodiment, the present invention provides a pharmaceutical composition comprising at least one compound according to the invention described herein and at least one pharmaceutically acceptable carrier or excipient.

In one embodiment, the present invention provides a pharmaceutical composition comprising at least one compound according to the invention described herein and at least one pharmaceutically acceptable carrier or excipient, for treating or preventing a Flaviviridae viral infection in a host.

In one embodiment, the present invention provides a pharmaceutical composition comprising at least one compound according to the invention described herein, and further comprising administering at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES).

In another embodiment, there is provided a combination comprising a least one compound according to the invention described herein and one or more additional agents.

In another embodiment, there is provided a combination comprising a least one compound according to the invention described herein and one or more additional agents chosen from viral serine protease inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

In one combination embodiment, the compound and additional agent are administered sequentially.

In another combination embodiment, the compound and additional agent are administered simultaneously.

The combinations referred to above may conveniently be presented for use in the form of a pharmaceutical formulation and thus pharmaceutical formulations comprising a combination as defined above together with a pharmaceutically acceptable carrier therefore comprise a further aspect of the invention.

The additional agents for the compositions and combinations include, for example, ribavirin, amantadine, merimepodib, Levovirin, Viramidine, and maxamine.

The term “viral serine protease inhibitor” as used herein means an agent that is effective to inhibit the function of the viral serine protease including HCV serine protease in a mammal. Inhibitors of HCV serine protease include, for example, those compounds described in WO 99/07733 (Boehringer Ingelheim), WO 99/07734 (Boehringer Ingelheim), WO 00/09558 (Boehringer Ingelheim), WO 00/09543 (Boehringer Ingelheim), WO 00/59929 (Boehringer Ingelheim), WO 02/060926 (BMS), WO 2006039488 (Vertex), WO 2005077969 (Vertex), WO 2005035525 (Vertex), WO 2005028502 (Vertex) WO 2005007681 (Vertex), WO 2004092162 (Vertex), WO 2004092161 (Vertex), WO 2003035060 (Vertex), of WO 03/087092 (Vertex), WO 02/18369 (Vertex), or WO98/17679 (Vertex).

Specific examples of viral serine protease inhibitors include Telaprevir (VX-950, Vertex), VX-500 (Vertex), TMC435350 (Tibotec/Medivir), MK-7009 (Merck), ITMN-191 (R7227, InterMune/Roche) and Boceprevir (SCHSO₃₀₃₄, Schering).

The term “viral polymerase inhibitors” as used herein means an agent that is effective to inhibit the function of a viral polymerase including an HCV polymerase in a mammal. Inhibitors of HCV polymerase include non-nucleosides, for example, those compounds described in:

WO 03/010140 (Boehringer Ingelheim), WO 03/026587 (Bristol Myers Squibb); WO 02/100846 A1, WO 02/100851 A2, WO 01/85172 A1 (GSK), WO 02/098424 A1 (GSK), WO 00/06529 (Merck), WO 02/06246 A1 (Merck), WO 01/47883 (Japan Tobacco), WO 03/000254 (Japan Tobacco) and EP 1 256 628 A2 (Agouron).

Furthermore other inhibitors of HCV polymerase also include nucleoside analogs, for example, those compounds described in: WO 01/90121 A2 (Idenix), WO 02/069903 A2 (Biocryst Pharmaceuticals Inc.), and WO 02/057287 A2(Merck/Isis) and WO 02/057425 A2 (Merck/Isis).

Specific examples of inhibitors of an HCV polymerase, include VCH-759 (ViroChem Pharma), VCH-916 (ViroChem Pharma), VCH-222 (ViroChem Pharma), R1626 (Roche), R7128 (Roche/Pharmasset), PF-868554 (Pfizer), MK-0608 (Merck/Isis), MK-3281 (Merck), A-837093 (Abbott), GS 9190 (Gilead), ana598 (Anadys), HCV-796 (Viropharma) and GSK625433 (GlaxoSmithKline).

The term “viral helicase inhibitors” as used herein means an agent that is effective to inhibit the function of a viral helicase including a Flaviviridae helicase in a mammal.

“Immunomodulatory agent” as used herein means those agents that are effective to enhance or potentiate the immune system response in a mammal. Immunomodulatory agents include, for example, class I interferons (such as α-, β-, δ- and Ω-interferons, τ-interferons, consensus interferons and asialo-interferons), class II interferons (such as γ-interferons) and pegylated interferons.

Specific examples of Immunomodulatory agent as used herein include IL-29 (PEG-Interferon Lambda, ZymoGenetics), Belerofon (Nautilus Biotech) injectable or oral, Oral Interferon alpha (Amarillo Biosciences), BLX-883 (Locteron, Biolex Therapeutics/Octoplus), Omega Interferon (Intarcia Therapeutics), multiferon (Viragen), Albuferon (Human Genome Sciences), consensus Interferon (Infergen, Three Rivers Pharmaceuticals), Medusa Interferon (Flamel Technologies), NOV-205 (Novelos Therapeutics), Oglufanide disodium (Implicit Bioscience), SCV-07 (SciClone), Zadaxin® (thymalfasin, SciClone/Sigma-Tau), AB68 (XTL bio) and Civacir (NABI).

The term “class I interferon” as used herein means an interferon selected from a group of interferons that all bind to receptor type 1. This includes both naturally and synthetically produced class I interferons. Examples of class I interferons include α-, β-, δ- and Ω-interferons, π-interferons, consensus interferons and asialo-interferons. The term “class II interferon” as used herein means an interferon selected from a group of interferons that all bind to receptor type II. Examples of class II interferons include γ-interferons.

Antisense agents include, for example, ISIS-14803.

Inhibitors of internal ribosome entry site (IRES) include ISIS-14803 (ISIS Pharmaceuticals) and those compounds described in WO 2006019831 (PTC therapeutics).

In one embodiment, the additional agent is interferon α, ribavirin, silybum marianum, interleukine-12, amantadine, ribozyme, thymosin, N-acetyl cysteine or cyclosporin.

In one embodiment, the additional agent is interferon α, or ribavirin.

In one embodiment, the additional agent is interferon α 1A, interferon α 1B, interferon α 2A, or interferon α 2B.

Interferon is available in pegylated and non pegylated forms. Pegylated interferons include PEGASYS™ and Peg-intron™.

In one embodiment, the additional agent is interferon α1A, interferon α1B, interferon α 2A (Roferon), PEG-interferon α 2A (Pegasys), interferon α 2B (Intron A) or PEG-interferon α 2B (Peg-Intron).

In one embodiment, the additional agent is standard or pegylated interferon α (Roferon, Pegasys, Intron A, Peg-Intron) in combination with ribavirin.

In one embodiment, the additional agent is chosen from A-831 (AZD0530, Arrow Therapeutics acquired by AstraZeneca), TLR9 agonist: IMO-2125 (Idera Pharmaceuticals), PYN17 (Phynova), Vavituximab (Tarvacin, Peregrine), DEB10-025 (DEBIO), NIM-811 (Novartis), SCY635 (Scynexis), PF-03491390 (IDN-6556, Pfizer), Suvus (formerly BIVN-401, Virostat, Bioenvision), MX-3253 (Celgosivir, Migenix), Viramidine (Taribavirin, Valeant Pharmaceuticals), Hepaconda (Giaconda), TT033 (Benitec/Tacere Bio/Pfizer), SIRNA-034 (Sirna Therapeutics aquired by Merck) and EHC-18 (Enzo Biochem), ACH-1095 (Achillion/Gilead), JKB-022 (Jenkin), CTS-1027 (Conatus), MitoQ (mitoquinone, Antipodean Pharmaceuticals), Alinia (nitazoxanide, Romark Laboratories) and Bavituximab (Peregrine Pharm).

In one embodiment, the additional agent is a therapeutic vaccine chosen from CSL123 (Chiron/CSL), IC41 (Intercell Novartis), GI 5005 (Globeimmune), TG4040 (Transgene), Chronvac C (Tripep/Inovio), GNI-103 (GENimmune), HCV/MF59 (Chiron/Novartis), PeviPRO™ (Pevion biotect).

The recommended dose of PEGASYS™ monotherapy for chronic hepatitis C is 180 mg (1.0 mL vial or 0.5 mL prefilled syringe) once weekly for 48 weeks by subcutaneous administration in the abdomen or thigh.

The recommended dose of PEGASYS™ when used in combination with ribavirin for chronic hepatitis C is 180 mg (1.0 mL vial or 0.5 mL prefilled syringe) once weekly.

The daily dose of Ribavirin is 800 mg to 1200 mg administered orally in two divided doses. The dose should be individualized to the patient depending on baseline disease characteristics (e.g., genotype), response to therapy, and tolerability of the regimen.

The recommended dose of PEG-Intron™ regimen is 1.0 mg/kg/week subcutaneously for one year. The dose should be administered on the same day of the week.

When administered in combination with ribavirin, the recommended dose of PEG-Intron is 1.5 micrograms/kg/week.

In one embodiment, viral serine protease inhibitor is a flaviviridae serine protease inhibitor.

In one embodiment, viral polymerase inhibitor is a flaviviridae polymerase inhibitor.

In one embodiment, viral helicase inhibitor is a flaviviridae helicase inhibitor.

In further embodiments:

viral serine protease inhibitor is HCV serine protease inhibitor;

viral polymerase inhibitor is HCV polymerase inhibitor;

viral helicase inhibitor is HCV helicase inhibitor.

In one embodiment, the present invention provides a method for treating or preventing a Flaviviridae viral infection in a host comprising administering to the host a therapeutically effective amount of at least one compound according to formula (I), (II), (III), or (IV).

In one embodiment, the viral infection is chosen from Flavivirus infections.

In one embodiment, the Flavivirus infection is Hepatitis C virus (HCV), bovine viral diarrhea virus (BVDV), hog cholera virus, dengue fever virus, Japanese encephalitis virus or yellow fever virus.

In one embodiment, the Flaviviridea viral infection is hepatitis C viral infection (HCV).

In one embodiment, the host is human.

In one embodiment, the present invention provides a method for treating or preventing a Flaviviridae viral infection in a host comprising administering to the host a therapeutically effective amount of at least one compound according to the invention described herein, and further comprising administering at least one additional agent.

In one embodiment, the present invention provides a method for treating or preventing a Flaviviridae viral infection in a host comprising administering to the host a therapeutically effective amount of at least one compound according to the invention described herein, and further comprising administering at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES).

The combinations referred to above may conveniently be presented for use in the form of a pharmaceutical formulation and thus pharmaceutical formulations comprising a combination as defined above together with a pharmaceutically acceptable carrier therefore comprise a further aspect of the invention.

The individual components for use in the method of the present invention or combinations of the present invention may be administered either sequentially or simultaneously in separate or combined pharmaceutical formulations.

In one embodiment, the present invention provides the use of a compound according to the invention described herein for treating or preventing Flaviviridae viral infection in a host.

In one embodiment, the present invention provides the use of a compound according to the invention described herein and further comprising at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES). for treating or preventing Flaviviridae viral infection in a host.

In one embodiment, the present invention provides the use of a compound according to the invention described herein for the manufacture of a medicament.

In one embodiment, the present invention provides the use of a compound according to the invention described herein for the manufacture of a medicament for treating or preventing a viral Flaviviridae infection in a host.

In one embodiment, the present invention provides the use of a compound according to the invention described herein and further comprising at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES). for the manufacture of a medicament for treating or preventing a viral Flaviviridae infection in a host.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. The single optical isomer or enantiomer can be obtained by method well known in the art, such as chiral HPLC, enzymatic resolution and chiral auxiliary.

Unless otherwise stated, all tautomeric forms of the compounds of the invention are within the scope of the invention.

In one embodiment, the compounds of the present invention are provided in the form of a single stereoisomer at least 95%, at least 97% and at least 99% free of the corresponding stereoisomers.

In a further embodiment the compound of the present invention are in the form of a single stereoisomer at least 95% free of the corresponding stereoisomers.

In a further embodiment the compound of the present invention are in the form of a single stereoisomer at least 97% free of the corresponding stereoisomers.

In a further embodiment the compound of the present invention are in the form of a single stereoisomer at least 99% free of the corresponding stereoisomers.

There is also provided pharmaceutically acceptable salts of the compounds of the present invention. By the term pharmaceutically acceptable salts of compounds are meant those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toleune-p-sulphonic, tartaric, acetic, trifluoroacetic, citric, methanesulphonic, formic, benzoic, malonic, naphthalene-2-sulphonic and benzenesulphonic acids. Other acids such as oxalic, while not themselves pharmaceutically acceptable, may be useful as intermediates in obtaining the compounds of the invention and their pharmaceutically acceptable acid addition salts.

Salts derived from amino acids are also included (e.g. L-arginine, L-Lysine).

Salts derived from appropriate bases include alkali metals (e.g. sodium, lithium, potassium) and alkaline earth metals (e.g. calcium, magnesium).

A reference hereinafter to a compound according to the invention includes that compound and its pharmaceutically acceptable salts.

With regards to pharmaceutically acceptable salts, see also the list of FDA approved commercially marketed salts listed in Table I of Berge et al., Pharmaceutical Salts, J. of Phar. Sci., vol. 66, no. 1, January 1977, pp. 1-19, the disclosure of which is incorporated herein by reference.

It will be appreciated by those skilled in the art that the compounds in accordance with the present invention can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

In addition to the compounds of this invention, pharmaceutically acceptable derivatives or prodrugs, and esters, of the compounds of this invention may also be employed in compositions to treat or prevent the herein identified disorders.

It will further be appreciated by those skilled in the art that the compounds in accordance with the present invention can exist in different solvate forms, for example hydrates. Solvates of the compounds of the invention may also form when solvent molecules are incorporated into the crystalline lattice structure of the compound molecule during the crystallization process.

### Pro-Drugs

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5th E a Ed.: Smith, M. B. and March, J., John Wiley a Sons, New York: 2001, the entire contents of which are hereby incorporated by reference.

In the formulas and drawings, a line transversing a ring and bonded to a group such as B, B′, R₁, R₄ or R₄′ in formula (I)

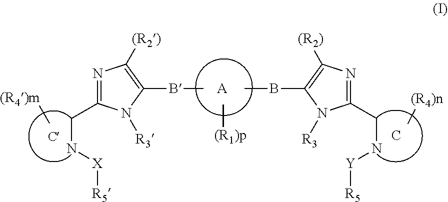

means that the group can be bonded to any carbon, or if applicable, heteroatom such as N, of that ring as valency allows.

The term “alkyl” represents a linear, branched or cyclic hydrocarbon moiety. The terms “alkenyl” and “alkynyl” represent a linear, branched or cyclic hydrocarbon moiety which has one or more double bonds or triple bonds in the chain. Examples of alkyl, alkenyl, and alkynyl groups include but are not limited to methyl, ethyl, propyl, isopropyl, butyl, isobutyl, sec-butyl, tert-butyl, pentyl, isopentyl, neopentyl, tert-pentyl, hexyl, isohexyl, neohexyl, allyl, vinyl, acetylenyl, ethylenyl, propenyl, isopropenyl, butenyl, isobutenyl, hexenyl, butadienyl, pentenyl, pentadienyl, hexenyl, heptenyl, heptadienyl, heptatrienyl, octenyl, propynyl, butynyl, pentynyl, hexynyl, cyclopropyl, cyclobutyl, cyclohexenyl, cyclohexdienyl and cyclohexyl. The terms alkyl, alkenyl, and alkynyl, also include combinations of linear and branched groups, e.g., cyclopropylmethyl, cyclohexylethyl, etc. The term alkenyl also includes C1 alkenyl where the one carbon atom is attached to the remainder of the molecule via a double bond. Where indicated the “alkyl,” “alkenyl,” and “alkynyl” can be optionally substituted such as in the case of haloalkyls in which one or more hydrogen atom is replaced by a halogen, e.g., an alkylhalide. Examples of haloalkyls include but are not limited to trifluoromethyl, difluoromethyl, fluoromethyl, trichloromethyl, dichloromethyl, chloromethyl, trifluoroethyl, difluoroethyl, fluoroethyl, trichloroethyl, dichloroethyl, chloroethyl, chlorofluoromethyl, chlorodifluoromethyl, dichlorofluoroethyl. Aside from halogens, where indicated, the alkyl, alkenyl or alkynyl groups can also be optionally substituted by, for example, halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

The terms “cycloalkyl”, and “cycloalkenyl” represent a cyclic hydrocarbon alkyl or alkenyl, respectively, and are meant to include monocyclic (e.g., cyclopropyl, cyclobutyl, cyclohexyl), spiro (e.g., spiro[2.3]hexanyl), fused (e.g., bicyclo[4.4.0]decanyl), and bridged (e.g., bicyclo[2.2.1]heptanyl)hydrocarbon moieties.

The terms “alkoxy,” “alkenyloxy,” and “alkynyloxy” represent an alkyl, alkenyl or alkynyl moiety, respectively, which is covalently bonded to the adjacent atom ??? through an oxygen atom. Examples include but are not limited to methoxy, ethoxy, propoxy, isopropoxy, butoxy, isobutoxy, sec-butoxy, tert-butoxy, pentyloxy, isopentyloxy, neopentyloxy, tert-pentyloxy, hexyloxy, isohexyloxy, trifluoromethoxy and neohexyloxy. Like the alkyl, alkenyl and alkynyl groups, where indicated the alkoxy, alkenyloxy, and alkynyloxy groups can be optionally substituted by, for example, halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

The term “aryl” represents a carbocyclic moiety containing at least one benzenoid-type ring (i.e., may be monocyclic or polycyclic), and which where indicated may be optionally substituted with one or more substituents. Examples include but are not limited to phenyl, tolyl, dimethylphenyl, aminophenyl, anilinyl, naphthyl, anthryl, phenanthryl or biphenyl. The aryl groups can be optionally substituted where indicated by, for example, halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

The term “aralkyl” represents an aryl group attached to the adjacent atom by an alkyl, alkenyl or alkynyl. Like the aryl groups, where indicated the aralkyl groups can also be optionally substituted. Examples include but are not limited to benzyl, benzhydryl, trityl, phenethyl, 3-phenylpropyl, 2-phenylpropyl, 4-phenylbutyl and naphthylmethyl. Where indicated, the aralkyl groups can be optionally substituted one or more times by, for example, halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

The term “heterocycle” represents a non aromatic, saturated or partially saturated cyclic moiety wherein said cyclic moiety is interrupted by at least one heteroatom selected from oxygen (O), sulfur (S) or nitrogen (N). Heterocycles may be monocyclic or polycyclic rings. Examples include but are not limited to azetidinyl, dioxolanyl, morpholinyl, morpholino, oxetanyl, piperazinyl, piperidyl, piperidinyl, cyclopentapyrazolyl, cyclopentaoxazinyl, cyclopentafuranyl, tetrahydrofuranyl, thiazolinyl, oxazolinyl, pyranyl, aziridinyl, azepinyl, dioxazepinyl, diazepinyl, oxyranyl, oxazinyl, pyrrolidinyl, thiopyranyl, thiolanyl, pyrazolidinyl, dioxanyl, and imidazolidinyl. Where indicated, the heterocyclic groups can be optionally substituted one or more times by, for example, halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

The term “heterocycle-alkyl” represents a heterocycle group attached to the adjacent atom by an alkyl, alkenyl or alkynyl group. It is understood that in, for example, a 4-18 member heterocycle-alkyl moiety, the 4-18 member represent the total of the ring atoms present in the heterocycle moiety and the carbon atoms present in the alkyl, alkenyl or alkynyl group. For example, the following groups are encompassed by a 7 member heterocycle-alkyl (* represents the attachment point):

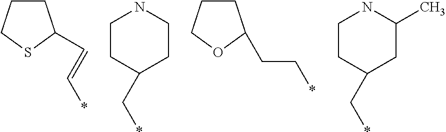

Where indicated the heterocycle-alkyl groups can be optionally substituted one or more times by, for example, halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

The term “heteroaryl” represents an aromatic cyclic moiety wherein said cyclic moiety is interrupted by at least one heteroatom selected from oxygen (O), sulfur (S) or nitrogen (N). Heteroaryls may be monocyclic or polycyclic rings wherein at least one ring in the polycyclic ring system is aromatic and at least one ring (not necessarily the same ring contains a heteroatom. Examples include but are not limited to dithiadiazinyl, furanyl, isooxazolyl, isothiazolyl, imidazolyl, oxadiazolyl, oxazolyl, pyrazinyl, pyridazinyl, pyrimidinyl, pyridyl, pyrazolyl, pyrrolyl, thiatriazolyl, tetrazolyl, thiadiazolyl, triazolyl, thiazolyl, thienyl, tetrazinyl, thiadiazinyl, triazinyl, thiazinyl, furoisoxazolyl, imidazothiazolyl, thienoisothiazolyl, thienothiazolyl, imidazopyrazolyl, pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, benzodioxolyl, dihydrobenzodioxinyl, benzothiadiazolyl, thienofuranyl, imidazopyrazinyl, purinyl, pyrazolopyrimidinyl, imidazopyridinyl, benzimidazolyl, indazolyl, benzoxathiolyl, benzodioxolyl, benzodithiolyl, indolizinyl, indolinyl, isoindolinyl, furopyrimidinyl, furopyridyl, benzofuranyl, isobenzofuranyl, thienopyrimidinyl, thienopyridyl, benzothienyl, benzoxazinyl, benzothiazinyl, quinazolinyl, naphthyridinyl, quinolinyl, isoquinolinyl, benzopyranyl, pyridopyridazinyl, chromen, benzodiazinyl. Where indicated the heteroaryl groups can be optionally substituted one or more times by, for example, halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c}) NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl. It is to be understood that terms such as “thienothienyl” encompasses all connectivites, including but not limited to, thieno[3,2-b]thiophene.

The term “heteroaralkyl” represents an optionally substituted heteroaryl group attached to the adjacent atom by an alkyl, alkenyl or alkynyl group. Where indicated the heteroaralkyl groups can be optionally substituted one or more times by, for example, halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl. It is understood that in, for example, a 6-18 member heteroaralkyl moiety, the 6-18 member represents the total of the ring atoms present in the heterocycle moiety and the carbon atoms in the alkyl, alkenyl or alkynyl groups. For example, the following groups are encompassed by a 7 member heteroaralkyl (* represents the attachment point):

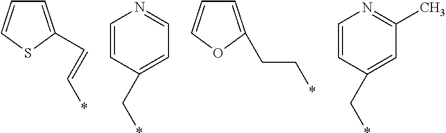

“Halogen atom or halo” is specifically a fluorine atom, chlorine atom, bromine atom or iodine atom.

The term “oxo” represents ═O.

A dash (“-”) that is not between two letters or symbols is used to indicate a point of attachment for a substitutent. For example, —CONR_{d}Rₑ is attached through the carbon of the amide.

A dash line (“-----”) is used to indicate the point of attachment for the group. For example, A is attached through the carbon at position 1 and 4 in the following representation:

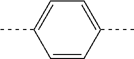

When there is a sulfur atom present, the sulfur atom can be at different oxidation levels, i.e., S, SO, or SO₂. All such oxidation levels are within the scope of the present invention.

The term “independently” means that a substituent can be the same or a different definition for each item.

In general, the term “substituted,” whether preceded by the term “optionally” or not, refers to the replacement of hydrogen radicals on a carbon or nitrogen atom in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. For example, the language, “which is unsubstituted or substituted one or more times by R¹⁰” means that when the group is substituted with more than one R¹⁰ group, the R¹⁰ groups can be different from each other. A ring substituent, such as a heterocycle, can be bound to another ring, such as a cycloalkyl, to form a spiro-bicyclic ring system, e.g., both rings share one common atom.

As one of ordinary skill in the art will recognize, combinations of substituents envisioned by this invention are those combinations that result in the formation of stable or chemically feasible compounds. The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week. When two alkoxy groups are bound to the same atom or adjacent atoms, the two alkoxy groups can form a ring together with the atom(s) to which they are bound.

In certain embodiment, a compound represented by:

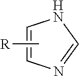

also includes where the R group replaces the H on the nitrogen atom.

Additionally, unless otherwise stated, structures depicted herein are also meant to include compounds that differ only in the presence of one or more isotopically enriched atoms. For example, compounds of this invention, wherein one or more hydrogen atoms are replaced deuterium or tritium, or one or more carbon atoms are replaced by a ¹³C- or ¹⁴C-enriched carbon are within the scope of this invention. Such compounds are useful, for example, as analytical tools, probes in biological assays, or antiviral compounds with improved therapeutic profile.

The terms “host” or “patient” mean human male or female, for example child, adolescent or adult.

It will be appreciated that the amount of a compound of the invention required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 750 mg/kg of body weight per day, for example, in the range of 0.5 to 60 mg/kg/day, or, for example, in the range of 1 to 20 mg/kg/day.

The desired dose may conveniently be presented in a single dose or as divided dose administered at appropriate intervals, for example as two, three, four or more doses per day.

The compound is conveniently administered in unit dosage form; for example containing 10 to 1500 mg, conveniently 20 to 1000 mg, most conveniently 50 to 700 mg of active ingredient per unit dosage form.

Ideally the active ingredient should be administered to achieve peak plasma concentrations of the active compound of from about 1 to about 75 μM, about 2 to 50 μM, about 3 to about 30 μM. This may be achieved, for example, by the intravenous injection of a 0.1 to 5% solution of the active ingredient, optionally in saline, or orally administered as a bolus containing about 1 to about 500 mg of the active ingredient. Desirable blood levels may be maintained by a continuous infusion to provide about 0.01 to about 5.0 mg/kg/hour or by intermittent infusions containing about 0.4 to about 15 mg/kg of the active ingredient.

When the compounds of the present invention or a pharmaceutically acceptable salts thereof is used in combination with a second therapeutic agent active against the same virus the dose of each compound may be either the same as or differ from that when the compound is used alone. Appropriate doses will be readily appreciated by those skilled in the art.

While it is possible that, for use in therapy, a compound of the invention may be administered as the raw chemical it is preferable to present the active ingredient as a pharmaceutical composition. The invention thus further provides a pharmaceutical composition comprising compounds of the present invention or a pharmaceutically acceptable derivative thereof together with one or more pharmaceutically acceptable carriers therefore and, optionally, other therapeutic and/or prophylactic ingredients. The carrier(s) must be “acceptable” in the sense of being compatible with the other ingredients of the formulation and not deleterious to the recipient thereof.

Pharmaceutical compositions include those suitable for oral, rectal, nasal, topical (including buccal and sub-lingual), transdermal, vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Pharmaceutical compositions suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

For topical administration to the epidermis, the compounds according to the invention may be formulated as ointments, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Compositions suitable for topical administration in the mouth include lozenges comprising active ingredient in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical compositions suitable for rectal administration wherein the carrier is a solid are for example presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

Compositions suitable for vaginal administration may be presented as pessaries, tampons, creams, gels, pastes, foams or sprays containing in addition to the active ingredient such carriers as are known in the art to be appropriate.

For intra-nasal administration the compounds of the invention may be used as a liquid spray or dispersible powder or in the form of drops. Drops may be formulated with an aqueous or non-aqueous base also comprising one more dispersing agents, solubilizing agents or suspending agents. Liquid sprays are conveniently delivered from pressurized packs.

For administration by inhalation the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the compounds according to the invention may take the form of a dry powder composition, for example a powder mix of the compound and a suitable powder base such as lactose or starch. The powder composition may be presented in unit dosage form in, for example, capsules or cartridges or e.g. gelatin or blister packs from which the powder may be administered with the aid of an inhalator or insufflator.

When desired the above described formulations adapted to give sustained release of the active ingredient may be employed.

The following general schemes and examples are provided to illustrate various embodiments of the present invention and shall not be considered as limiting in scope. It will be appreciated by those of skill in the art that other compounds of the present invention can be obtained by substituting the generically or specifically described reactants and/or operating conditions used in the following examples.

In the foregoing and in the following examples, all temperatures are set forth uncorrected in degrees Celsius; and, unless otherwise indicated, all parts and percentages are by weight.

The following abbreviations may be used as follows:
aq aqueous
conc concentrate
DCM methylene chloride
DIPEA Diisopropylethylamine
DMF dimethylformamide
DMSO Dimethylsulfoxide
EtOAc Ethyl acetate
HATU O-(7-Azabenzotriazol-1-yl)-N,N,N′,N′-tetramethyluronium hexafluorophosphate
M molar
MeOH Methanol
MTBE methyl ter-butyl ether
n-BuLi n-butyl lithium
PdCl₂dppf (1,1′-Bis-(diphenylphosphino)-ferrocene)palladium (II) dichloride
Pd(PPh₃)₂Cl₂ trans-dichlorobis(triphenyl phosphine) Palladium (II)
RT room temperature
TEA Triethylamine
THF Tetrahydrofuran

The compounds of this invention may be prepared in light of the specification using steps generally known to those of ordinary skill in the art. Those compounds may be analyzed by known methods, including but not limited to LCMS (liquid chromatography mass spectrometry) HPLC (high performance liquid chromatography) and NMR (nuclear magnetic resonance). It should be understood that the specific conditions shown below are only examples, and are not meant to limit the scope of the conditions that can be used for making compounds of this invention. Instead, this invention also includes conditions that would be apparent to those skilled in that art in light of this specification for making the compounds of this invention. Unless otherwise indicated, all variables in the following schemes are as defined herein. General Schemes:

Mass spec. samples were analyzed on a MicroMass Quattro Micro of MicroMass LCZ mass spectrometer operated in single MS mode with electrospray ionization. Samples were introduced into the mass spectrometer using chromatography. Mobile phase for all mass spec. analyses consisted of 10 mM pH 7 ammonium acetate and a 1:1 acetonitrile-methanol mixture. Method A: Column gradient conditions were 5%-100% acetonitrile-methanol over 3.5 mins gradient time and 4.8 mins run time on an ACE5C8 3.0×75 mm column. Flow rate was 1.2 ml/min. Method B: Column gradient were 5%-100% acetonitrile-methanol over 10 mins gradient time and 12 mins run time on a ACE5C8 4.6×150 mm column. Flow rate was 1.5 mL/min. As used herein, the term “Rt(min)” refers to the LCMS retention time, in minutes, associated with the compound. Unless otherwise indicated, the LCMS method utilized to obtain the reported retention time is as detailed above. If the Rt(min) is <5 min method A was used, if the Rt(min) is >5 min then method B was used.

1H-NMR spectra were recorded at 400 MHz using a Bruker DPX 400 or Varian instrument.

Purification by reverse phase HPLC is carried out under standard conditions using a Phenomenex Gemini C18 column, 21.2 mmID×250 mm, 5 μm, 110 Å. Elution is performed using a linear gradient 20 to 90% (CH₃CN in water or CH₃CN in water with 0.02% HCl) with a flow rate of 5.0 mL/minute.

### General Procedure 1:

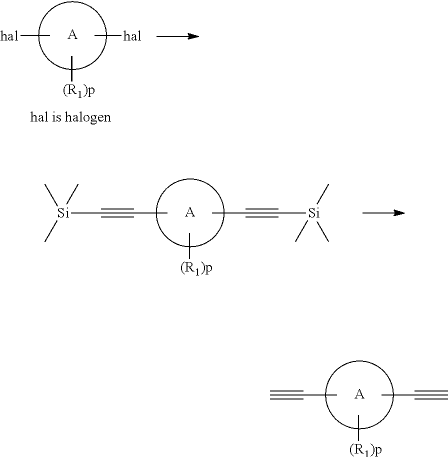

The diethynyl intermediate is prepared in 2 steps from the commercially available bis-halogenated aryl or heterocycle. A Sonogashira coupling with bis(trimethylsilyl)acetylene using CuI and palladium catalysts in solvent such as DMF in presence of base such as TEA or DIPEA give the bis-trimethyl ethynyl silane intermediate. The silyl groups are hydrolyzed in presence of a base such as K₂CO₃ in MeOH to give the expected diethynyl intermediate.

The Sonogashira coupling reaction is a well established method for producing acetylene containing compounds. Conditions for such coupling are well known in the art and can be found for example in the examples of the present application, in Yamagushi et al. (Synlett 1999, No. 5, 549-550), or in Tykwinski et al. Angew. Chem. Inte. Ed. 2003, 42, 1566-1568.

### General Procedure 2:

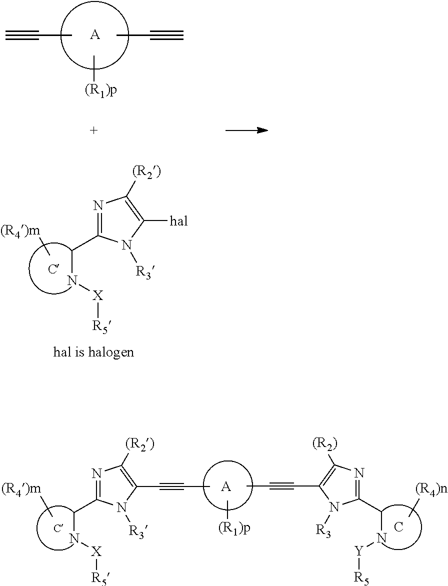

The compound is formed from the diethynyl intermediate and the iodo or bromo imidazole intermediate by a Sonogoshira coupling using CuI and palladium catalysts in solvents such as DMF in presence of base such as TEA or DIPEA.

In one embodiment, the halogen is iodo or bromo.

In one embodiment, the halogen is iodo.

### General Procedure 3:

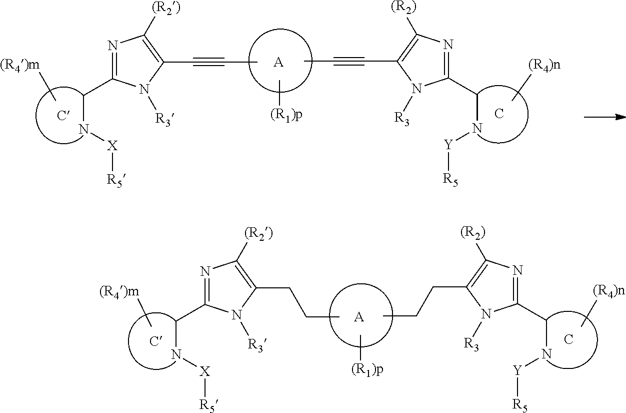

Reduction of the triple bond is done under standard hydrogenation procedure as known by those of ordinary skill in the art. The compound having triple bonds is dissolved in a suitable solvent such as methanol and a catalytic amount of a 1M solution of HCl is added followed by a catalytic amount of 10% Pd/C. The reaction mixture is stirred at RT under 1 atmosphere of hydrogen until completion of the reaction, filtered and the filtrate concentrated to dryness to afford the alkyl derivatives.

### General Procedure 4:

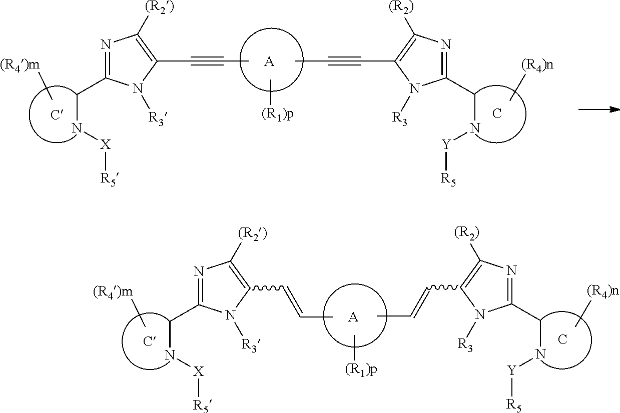

Reduction of the triple bonds to double bonds could be done under standard hydrogenation procedure as known in the art. Such conditions are described for example in the following references:
Tri H. V. Huynh et al. Organic Letters, 2009, vol. 11, 999-1002.
Sellarajah, Shan et al. J. Med. Chem. 2004, 47 (22) 5515
Chandrasehkar, S et al. Tetrahedron Letters 2004, 45, 2421-2423.

### General Procedure 5:

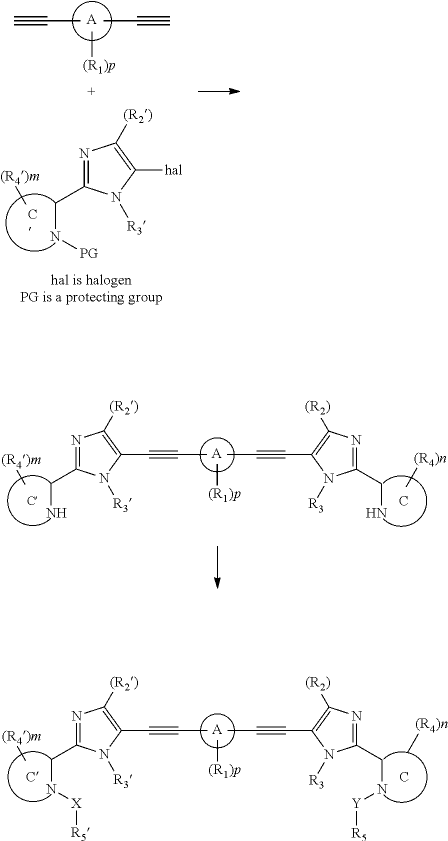

Alternatively, the compound is formed from the diethynyl intermediate and the protected iodo or bromo imidazole intermediate by a Sonogoshira coupling using CuI and palladium catalysts in solvents such as DMF in presence of base such as TEA or DIPEA. Removal of the protecting group optionally followed by coupling with the complimentary functionalized X-R5′ or Y-R5 group can be accomplished under standard reaction conditions known in the art.

In one embodiment, the halogen is iodo or bromo.

In one embodiment, the halogen is iodo.

In one embodiment, the protecting group is tert-butoxycarbonyl.

Mass spec. samples were analyzed on a Micromass Platform LCZ mass spectrometer operated in single MS mode with electrospray ionization. Samples were introduced into the mass spectrometer using chromatography. Mobile phase for all mass spec. analyses consisted of H₂O+0.01% TFA and CH₃CN+0.01% TFA. Method: Column gradient conditions were 5%-85% CH₃CN+0.01% TFA in 20 minutes gradient time on a SymmetryShield RP18 3.5 um, 2.1×50 mm column. Flow rate was 0.5 mL/minute. As used herein, the term “Rt(minute)” refers to the LCMS retention time, in minutes, associated with the compound. Unless otherwise indicated, the LCMS method utilized to obtain the reported retention time is detailed above.

### Intermediate 1

### (2S)-2-(methoxycarbonylamino)-3-methyl-butanoic acid

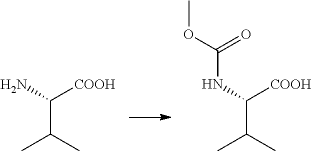

L-Valine (140 g, 1.195 mol) is added to a stirred solution of 1 M sodium hydroxide (1.183 L, 1.183 mol). After complete dissolution, sodium carbonate (65.8 g, 621.4 mmol) is added followed by methyl chloroformate (122 g, 99.75 mL, 1.291 mol) at 0° C. over 40 minutes. The reaction mixture is stirred at RT for 3.5 hours, then washed with diethyl ether (3×200 ml). The aqueous layer is cooled to 0° C., and acidified to pH 1-2. The white solid formed is filtered on a Buchner, washed with cold water and dried to afford the title compound (2S)-2-(methoxycarbonylamino)-3-methyl-butanoic acid (140 g, 67%).

### Intermediate 2

### (2S)-2-[Methoxycarbonyl(methyl)amino]-3-methyl-butanoic acid

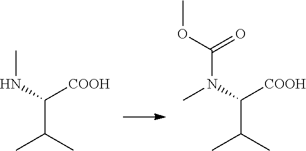

(2S)-3-Methyl-2-methylamino-butanoic acid (5 g, 38.12 mmol) is added to a stirring solution of sodium hydroxide (76.2 mL of 1 M, 76.24 mmol). After complete dissolution, disodium carbonate (2.1 g, 19.82 mmol) is added followed by methyl chloroformate (3.18 mL, 41.17 mmol) at 0° C. over 40 minutes. The reaction mixture is stirred at RT for 4 hours, then washed with diethyl ether (2×75 ml). The aqueous layer is cooled to 0° C., acidified to pH 1-2 and extracted with CH₂Cl₂. The organic phase is dried over MgSO₄, filtered and concentrated to dryness to give the title compound (2S)-2-[methoxycarbonyl(methyl)amino]-3-methyl-butanoic acid (5.12 g, 71%) as a clear oil.

### Intermediate 3

### Methyl N-[(1S)-1-[(2S)-2-(5-iodo-1H-imidazol-2-yl)pyrrolidine-1-carbonyl]-2-methyl-propyl]carbamate

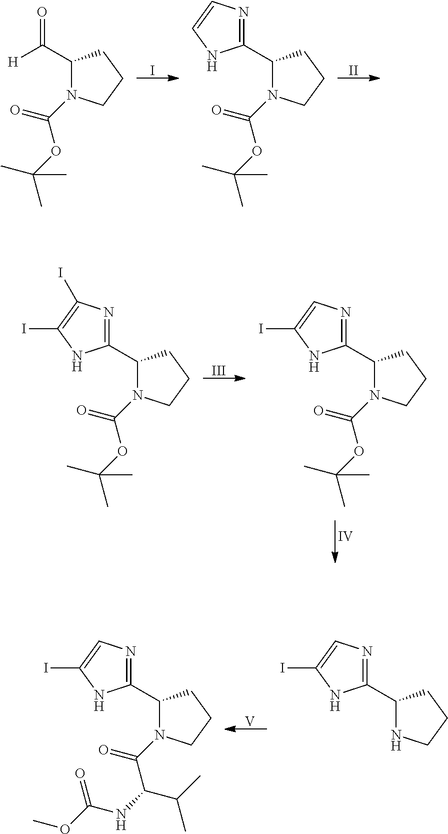

### Step I:

### tert-Butyl(2S)-2-(1H-imidazol-2-yl)pyrrolidine-1-carboxylate

To a stirred solution of commercially available tert-butyl (2S)-2-formylpyrrolidine-1-carboxylate (15 g, 75.3 mmol) in a 1/1 mixture MeOH (30 mL)/NH₄OH (30 mL) is added oxaldehyde (3.4 mL, 75.3 mmol) over 10 minutes. The reaction is stirred at RT for 72 hours and evaporated to dryness. The residue is purified by flash column chromatography on silica gel (EtOAc/Hexanes 50% to 75%) to give tert-butyl(2S)-2-(1H-imidazol-2-yl)pyrrolidine-1-carboxylate (5.2 g, 29%).

### Step II:

### tert-Butyl(2S)-2-(4,5-diiodo-1H-imidazol-2-yl)pyrrolidine-1-carboxylate

To a stirred solution of tert-butyl (2S)-2-(1H-imidazol-2-yl)pyrrolidine-1-carboxylate (5 g, 21 mmol) in CH₂Cl₂ (120 mL) is added 1-iodopyrrolidine-2,5-dione (10.4 g, 46.3 mmol) at 0° C. over 15 minutes. The reaction mixture is stirred 2 hours at 0° C., concentrated to dryness, and the residue is purified by flash column chromatography on silica gel (25% EtOAc in Hexanes) to give tert-butyl(2S)-2-(4,5 diiodo-1H-imidazol-2-yl)pyrrolidine-1-carboxylate (8.6 g, 83%).

### Step III:

### tert-Butyl(2S)-2-(5-iodo-1H-imidazol-2-yl)pyrrolidine-1-carboxylate

To a cooled (−78° C.) solution of tert-butyl (2S)-2-(4,5-diiodo-1H-imidazol-2-yl)pyrrolidine-1-carboxylate (6.5 g, 13.29 mmol) in THF (65 mL) is added dropwise 1.5M n-BuLi in hexanes (22.15 mL, 33.22 mmol) over 30 minutes. The reaction mixture is stirred 30 minutes and an additional 1.5 eq of 1.5 M n-BuLi in hexanes is added over 30 minutes. After addition of 20 mL 1N HCl the reaction mixture is allowed to stir at RT. The reaction mixture is extracted by EtOAc, washed with H₂O, dried over Na₂SO₄, filtered, concentrated to dryness, and purified by flash column chromatography on silica gel (0 to 50% EtOAc in Hexanes) to give tert-butyl (2S)-2-(5-iodo-1H-imidazol-2-yl)pyrrolidine-1-carboxylate (2.93 g, 60%).

### Step IV:

### 5-Iodo-2-[(2S)-pyrrolidin-2-yl]-1H-imidazole (HCl salt)

To tert-butyl (2S)-2-(5-iodo-1H-imidazol-2-yl)pyrrolidine-1-carboxylate (2.9 g, 7.9 mmol) is added a 4 M solution of HCl in dioxane (29 mL, 795.4 mmol) at 0° C. The reaction mixture is stirred for 4 hours at RT, evaporated to dryness to afford 5-iodo-2-[(2S)-pyrrolidin-2-yl]-1H-imidazole (HCl salt) (2.4 g, 100%) as a white solid.

### Step V:

### Methyl N-[(1S)-1-[(2S)-2-(5-iodo-1H-imidazol-2-yl)pyrrolidine-1-carbonyl]-2-methyl-propyl]carbamate

To a chilled solution (0° C.) of (2S)-2-(methoxycarbonylamino)-3-methyl-butanoic acid (2.2 g, 12.7 mmol) and HATU (5.4 g, 14.3 mmol) in DMF (48 mL) is added DIPEA (10.3 g, 13.9 mL, 79.8 mmol) followed by 5-iodo-2-[(2S)-pyrrolidin-2-yl]-1H-imidazole HCL salt (2.4 g, 7.9 mmol). The reaction mixture is stirred overnight at RT, diluted with H₂O and EtOAc. The organic phase is separated, dried over Na₂SO₄, filtered and concentrated to dryness. The residue is purified by flash column chromatography on silica gel (0 to 100% EtOAc in Hexanes) to give Methyl N-[(1S)-1-[(2S)-2-(5-iodo-1H-imidazol-2-yl)pyrrolidine-1-carbonyl]-2-methyl-propyl]carbamate (1.3 g, 35%).

### Intermediate 4

### Methyl N-[(1S)-1-[(2S,4S)-2-(5-iodo-1H-imidazol-2-yl)-4-methyl-pyrrolidine-1-carbonyl]-2-methyl-propyl]carbamate

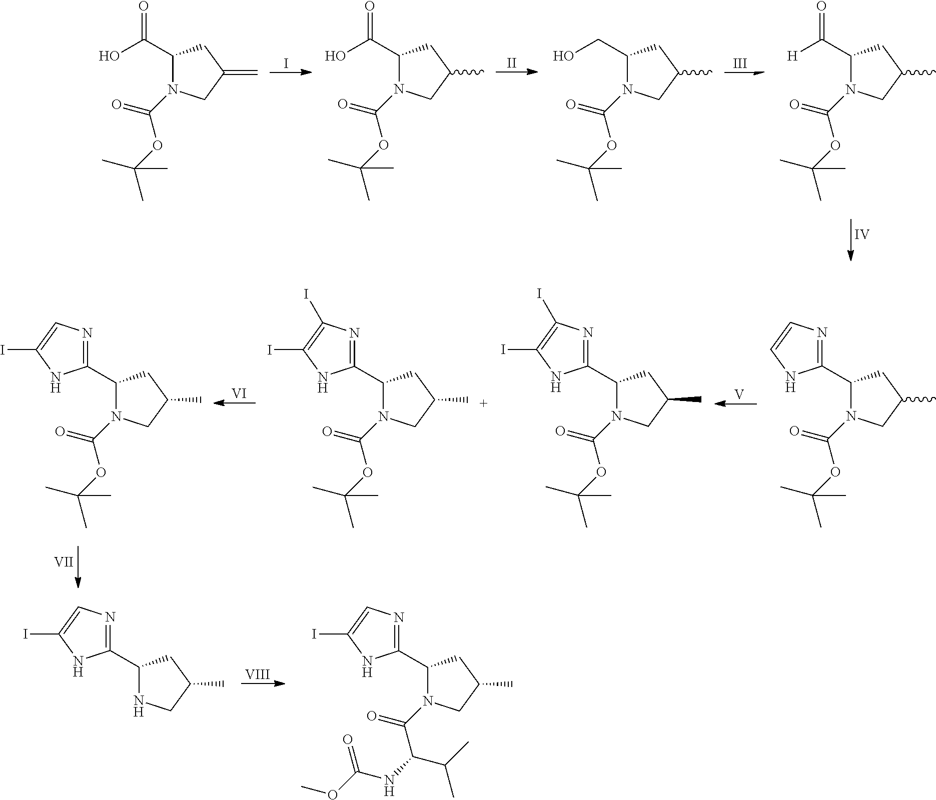

### Step I:

### (2S)-1-tert-Butoxycarbonyl-4-methyl-pyrrolidine-2-carboxylic acid

A solution of (2S)-1-tert-butoxycarbonyl-4-methylene-pyrrolidine-2-carboxylic acid (25 g, 110 mmol) in methanol or ethanol (250 mL) is purged 3 times under N_{2(g)} before the addition of PtO₂ (2.5 g, 11 mmol). The solution is purged again with vacuum and H_{2(g)}, and this purge is repeated three times. Then the reaction mixture is stirred for 20 hours under one atmosphere of hydrogen. The reaction mixture is filtered on celite to remove the catalyst, and the filtrate is concentrated to dryness to give (2S)-1-tert-butoxycarbonyl-4-methyl-pyrrolidine-2-carboxylic acid (24.9 g, 98.7%) as a white solid (mixture of cis/trans approx. 80/20 ratio).

### Step II:

### tert-Butyl (2S)-2-(hydroxymethyl)-4-methyl-pyrrolidine-1-carboxylate

To a solution of (2S)-1-tert-butoxycarbonyl-4-methyl-pyrrolidine-2-carboxylic acid (26.6 g, 116.0 mmol) in THF (160 mL) is added 1 M Borane in THF (243.6 mL, 243.6 mmol) at 0° C. The reaction mixture is stirred at RT overnight. Then 50 mL of a saturated aqueous solution of NH₄Cl is carefully added (dropwise) at 4° C., followed by 100 mL of H₂O. The mixture is extracted with EtOAc and the organic phase is washed with H₂O, dried over Na₂SO₄, filtered and concentrated to dryness. The residue is purified by flash column chromatography on silica gel (0 to 20% EtOAc in Hexanes) to give tert-butyl (2S)-2-(hydroxymethyl)-4-methyl-pyrrolidine-1-carboxylate (23.5 g, 94%).

### Step III:

### tert-Butyl (2S)-2-formyl-4-methyl-pyrrolidine-1-carboxylate

To a solution of oxalyl chloride (319.4 mL of 2 M, 638.8 mmol) in CH₂Cl₂ (460 mL) is added DMSO (90.69 mL, 1.28 mol) over 30 minutes, keeping the internal temperature around −60° C. tert-Butyl (2S)-2-(hydroxymethyl)-4-methyl-pyrrolidine-1-carboxylate (55 g, 255.5 mmol) in CH₂Cl₂ (460 mL) is then added over 50 minutes at −78° C. The reaction mixture is stirred for 20 minutes before dropwise addition of DIPEA (445 mL, 2.55 mol). The reaction mixture is stirred at −78° C. for 2 hours and is allowed to warm to RT over 2 hours. To this mixture is added slowly 800 mL of 1N HCl. After stirring, the organic phase is separated, dried over Na₂SO₄, filtered and concentrated to dryness. The residue is purified by flash column chromatography on silica gel (0 to 20% EtOAc in Hexanes) to give tert-butyl (2S)-2-formyl-4-methyl-pyrrolidine-1-carboxylate (48.5 g, 227.4 mmol, 85%) as a brown oil (mixture cis/trans 77/23).

### Step IV:

### tert-Butyl (2S)-2-(1H-imidazol-2-yl)-4-methyl-pyrrolidine-1-carboxylate

To a stirred solution of tert-butyl (2S)-2-formyl-4-methyl-pyrrolidine-1-carboxylate (45 g, 211 mmol) in MeOH (90 mL) is added NH₄OH (90 mL). Oxaldehyde (85.6 g, 67.7 mL of 40% w/v, 466.7 mmol) is added by portions (exothermic reaction). The reaction mixture is stirred at RT overnight, diluted with H₂O (300 ml) and is extracted with CH₂Cl₂ (2×300 ml). The aqueous phase is extracted a second time with CH₂Cl₂ and the combined organic layers are washed with H₂O, dried over Na₂SO₄, filtered and evaporated to dryness. The residue is purified by recrystallization in EtOAc, to give 24 g of the title compound. The filtrate is evaporated to dryness and the residue is purified by flash column chromatography on silica gel (25 to 100% EtOAc in Hexanes) to give 9.67 g of title compound. The two isolated solids are combined to give tert-butyl (2S)-2-(1H-imidazol-2-yl)-4-methyl-pyrrolidine-1-carboxylate (33.67 g, 63.5%).

¹H NMR (400 MHz, dmso, mixture of Cis and Trans isomers and its rotamers) δ 11.71 (s, 1H), 6.85 (s, 2H), 4.86-4.58 (m, 2H), 3.75-3.5 (m, 2H), 3.03-2.82 (m, 2H), 2.36-2.25 (m, 1H), 2.25-2.11 (m, 1H), 1.6-1.45 (m, 1H), 1.39 (s, minor rotamer of minor isomer), 1.37 (s, minor rotamer of major isomer), 1.15 (s, major rotamer of minor isomer), 1.09 (s, major rotamer of major isomer) 1.005 (d, minor isomer) 0.99 (d, major isomer).

### Step V:

### tert-Butyl (2S,4S)-2-(4,5-diiodo-1H-imidazol-2-yl)-4-methyl-pyrrolidine-1-carboxylate

To a stirred solution of tert-butyl (2S)-2-(1H-imidazol-2-yl)-4-methyl-pyrrolidine-1-carboxylate (36.6 g, 145.6 mmol) in CH₂Cl₂ (366.0 mL) at 5° C. is added 1-iodopyrrolidine-2,5-dione (68.80 g, 305.8 mmol) over 15 minutes. After 1 hour, a 10% sodium thiosulfate solution (800 ml) is added. After stirring for 10 minutes, the organic phase is separated, washed with water, dried over Na₂SO₄, filtered and evaporated to dryness. The crude is purified by flash column chromatography on silica gel (0 to 50% EtOAc in Hexanes) to give tert-butyl (2S,4S)-2-(4,5-diiodo-1H-imidazol-2-yl)-4-methyl-pyrrolidine-1-carboxylate (52.3 g, 65.7%).

¹H NMR (400 MHz, dmso, 2.5:1 mixture of rotamers), peaks for the major rotamer δ 12.70 (s, 1H), 4.57 (dd, 1H), 3.62-3.52 (m, 1H), 2.95 (t, 1H), 2.35-2.0 (m, 2H), 1.50 (dd, 1H), 1.10 (s, 9H), 1.01 (d, 3H). tert-Butyl (2S,4R)-2-(4,5-diiodo-1H-imidazol-2-yl)-4-methyl-pyrrolidine-1-carboxylate (10.5 g, 13%) is also isolated.

¹H NMR (400 MHz, DMSO, 1.2:1 mixture of rotamers), peaks for the major rotamer, δ 12.65 (br s, 1H), 4.69 (dd, 1H), 3.69-3.50 (m, 1H), 2.82 (t, 1H), 2.45-2.3 (m, 1H), 1.91-1.68 (m, 2H), 1.15 (s, 9H), 0.97 (d, J=6.6 Hz, 3H). Selected peaks for the minor rotamer: 4.77 (d), 1.38 (s).

### Step VI:

### tert-Butyl (2S,4S)-2-(5-iodo-1H-imidazol-2-yl)-4-methyl-pyrrolidine-1-carboxylate

A solution of LiCl in THF (3.9 mL of a 0.5 M solution, 1.99 mmol) is added to tert-butyl (2S,4S)-2-(4,5-diiodo-1H-imidazol-2-yl)-4-methyl-pyrrolidine-1-carboxylate (1 g, 1.99 mmol). After stirring for 5 minutes at RT the reaction mixture is cooled down to −20° C. and a solution of methyl magnesium chloride in THF (946.7 μL of 2.1 M, 1.99 mmol) is added dropwise. After stirring for 20 minutes at −20° C., a solution of isopropyl magnesium chloride in THF (3.2 mL of 1.24 M, 3.97 mmol) is added dropwise. The reaction mixture is slowly warmed up to RT and stirred for 2 hours. The reaction mixture is cooled down to 0° C. and a saturated aqueous NH₄Cl solution is slowly added followed by water. This mixture is then extracted with EtOAc (3×20 mL), and the combined organic layers are washed with brine, dried over Na₂SO₄, filtered and concentrated to dryness. The residue is purified by flash column chromatography on silica gel (0 to 25% EtOAC/Hexane) to afford tert-butyl (2S,4S)-2-(5-iodo-1H-imidazol-2-yl)-4-methyl-pyrrolidine-1-carboxylate (636 mg, 83%) as a white solid.

¹H NMR (400 MHz, dmso, 2:1 mixture of rotamers), peaks for the major rotamer, δ 12.15 (s, 1H), 7.19 (s, 1H), 4.65-4.57 (m, 1H), 3.65-3.55 (m, 1H), 2.95 (t, 1H), 2.4-2.1 (m, 2H), 1.52 (dd, 1H), 1.10 (s, 9H), 1.00 (d, 3H). Selected peaks for minor rotamer, 12.09 (s), 7.15 (s), 1.36 (s).

### Step VII:

### 5-Iodo-2-[(2S,4S)-4-methylpyrrolidin-2-yl]-1H-imidazole as a HCl salt

To a solution of tert-butyl (2S,4S)-2-(5-iodo-1H-imidazol-2-yl)-4-methyl-pyrrolidine-1-carboxylate (1.6 g, 4.242 mmol) in MeOH (16 mL) is added a 4M HCl in dioxane solution (16 ml) at 0° C. The reaction mixture is stirred at RT overnight and evaporated to dryness to afford 5-iodo-2-[(2S,4S)-4-methylpyrrolidin-2-yl]-1H-imidazole (1.37 g, 92.5%) as a yellow solid.

¹H NMR (400 MHz, dmso) δ 9.98 (br s, 1H), 9.17 (br s, 1H), 7.46 (s, 1H), 4.8-4.6 (m, 1H), 3.45-3.35 (m, 1H), 2.9-2.75 (m, 1H), 2.5-2.3 (m, 2H), 1.88-1.78 (m, 1H), 1.09 (d, 3H).

### Step VIII:

### Methyl N-[(1S)-1-[(2S,4S)-2-(5-iodo-1H-imidazol-2-yl)-4-methyl-pyrrolidine-1-carbonyl]-2-methyl-propyl]carbamate

To a solution of (2S)-2-(methoxycarbonylamino)-3-methyl-butanoic acid (644.5 mg, 3.68 mmol) in DMF (25 mL) at 0° C. is added HATU (1.4 g, 3.68 mmol), DIPEA (2.5 mL, 14.57 mmol) followed by 5-iodo-2-[(2S,4S)-4-methylpyrrolidin-2-yl]-1H-imidazole as HCl salt (1.28 g, 3.64 mmol). The reaction mixture is stirred at RT for 20 hours, diluted with EtOAc and H₂O. The organic phase is separated, washed with H₂O, dried over Na₂SO₄, filtered and concentrated to dryness. The residue is purified by flash column chromatography on silica gel (0 to 100% EtOAC/Hexane) to afford methyl N-[(1S)-1-[(2S,4S)-2-(5-iodo-1H-imidazol-2-yl)-4-methyl-pyrrolidine-1-carbonyl]-2-methyl-propyl]carbamate (1.3 g, 87.3%) as a white solid.

¹H NMR (400 MHz, dmso) δ 12.03 (s, 1H), 7.19 (d, 1H), 7.18 (s, 1H), 4.83 (dd, 1H), 4.16-3.91 (m, 2H), 3.52 (s, 3H), 3.16 (t, 1H), 2.38-2.08 (m, 2H), 1.9-1.72 (m, 1H), 1.72-1.61 (m, 1H), 1.06 (d, 3H), 0.76 (d, 3H), 0.755 (m, 3H).

### Intermediate 5

### Methyl (S)-1-((2S,4R)-2-(4-iodo-1H-imidazol-2-yl)-4-methylpyrrolidin-1-yl)-3-methyl-1-oxobutan-2-ylcarbamate

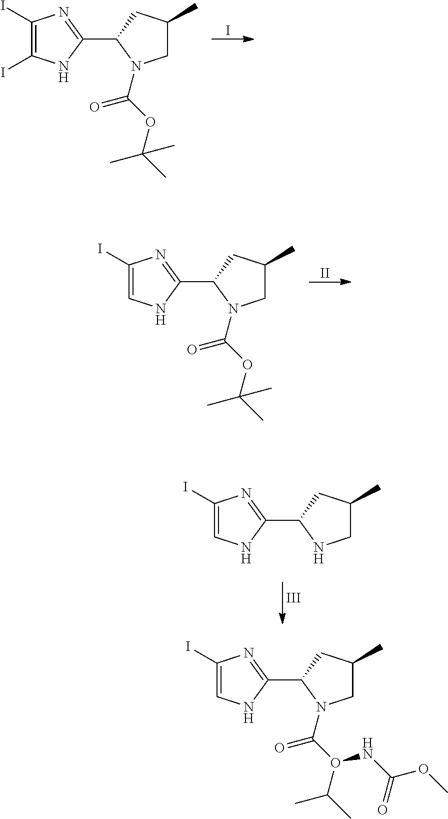

### Step I:

### (2S,4R)-tert-Butyl 2-(4-iodo-1H-imidazol-2-yl)-4-methylpyrrolidine-1-carboxylate

A solution of LiCl in THF (2.000 mL of 0.5 M, 1.000 mmol) is added to tert-butyl (2S,4R)-2-(4,5-diiodo-1H-imidazol-2-yl)-4-methyl-pyrrolidine-1-carboxylate (503.12 mg, 1.000 mmol) in 25 mL flask. The mixture is stirred at RT for 5 minutes, cooled to −20° C., and a solution of 3 M methyl magnesium chloride in THF (333.3 μL, 1.000 mmol) is added drop wise. The reaction mixture is stirred at −20° C. for 20 minutes, and a solution of isopropyl magnesium chloride in THF (846.2 μL of 1.3 M, 1.100 mmol) is added drop wise. The reaction mixture is slowly warmed up to RT over 1 hour, and left overnight. An additional amount of isopropyl magnesium chloride in THF (423.1 μL of 1.3 M, 0.5500 mmol) is added at RT, and the mixture is stirred for 1 hour. Another additional amount of isopropyl magnesium chloride in THF (423.1 μL of 1.3 M, 0.5500 mmol) is added, and the mixture is stirred at RT for 2 hours. An additional amount of isopropyl magnesium chloride in THF (423.1 μL of 1.3 M, 0.5500 mmol) is added, and the mixture is stirred for 30 minutes. The reaction mixture is poured into cold saturated aq. NH₄Cl solution, extracted with EtOAc (3×20 mL), and the combined extracts are washed with brine, dried over Na₂SO₄, filtered and concentrated to dryness. The residue is purified by flash column chromatography on silica gel (0 to 20% EtOAC/CH₂Cl₂) to afford (2S,4R)-tert-butyl 2-(4-iodo-1H-imidazol-2-yl)-4-methylpyrrolidine-1-carboxylate (240 mg, 0.6133 mmol, 61.34%) as white solid.

Rf=0.5 (EA:Hex, 1:1).

¹H NMR (400 MHz, CDCl₃) δ 10.59 (s, 1H), 7.02 (s, 1H), 4.90 (d, J=8.1 Hz, 1H), 3.55-3.39 (m, 1H), 3.05-2.82 (m, 2H), 2.6-2.45 (m, 1H), 1.80-1.65 (m, 1H), 1.46 (s, 9H), 1.06 (dd, J=6.7 Hz, 3H).

### Step II:

### 4-Iodo-2-((2S,4R)-4-methylpyrrolidinium-2-yl)-1H-imidazol-3-ium chloride

To a stirred solution of tert-butyl (2S,4R)-2-(4-iodo-1H-imidazol-2-yl)-4-methyl-pyrrolidine-1-carboxylate (229 mg, 0.6071 mmol) in MeOH (1 mL) is added HCl in dioxane (1.518 mL of 4 M, 6.071 mmol). The resulting solution is stirred at RT and a thick precipitate is formed within 20 min The precipitate is diluted with diethyl ether, filtered, and dried under high vacuum to afford 4-iodo-2-((2S,4R)-4-methylpyrrolidinium-2-yl)-1H-imidazol-3-ium chloride (185 mg, 0.5250 mmol, 86.48%) as a white solid. ¹H NMR (400 MHz, DMSO-d₆) δ 10.30 (br s, 1H), 9.24 (br s, 1H), 7.46 (s, 1H), 4.85-4.7 (m, 1H), 3.5-3.35 (m, 1H), 2.85-2.72 (m, 1H), 2.61-2.5 (m, 1H), 2.38-2.24 (m, 1H), 1.98-1.85 (m, 1H), 1.05 (d, J=6.8 Hz, 3H).

### Step III:

### Methyl (S)-1-((2S,4R)-2-(4-iodo-1H-imidazol-2-yl)-4-methylpyrrolidin-1-yl)-3-methyl-1-oxobutan-2-ylcarbamate

To a cold (0-4° C.) stirred solution of (2S)-2-(methoxycarbonylamino)-3-methyl-butanoic acid (50.10 mg, 0.2860 mmol), 4-iodo-2-((2S,4R)-4-methyl-pyrrolidinium-2-yl)-1H-imidazol-3-ium chloride (96 mg, 0.2724 mmol) and HATU (108.7 mg, 0.2860 mmol) in DMF (1.918 mL) is added DIPEA (140.9 mg, 189.9 μL, 1.090 mmol). The reaction mixture is slowly warmed up to RT, stirred overnight, diluted with water (5 mL), and extracted with ethyl acetate (3×10 mL). The combined extracts are washed with a saturated aqueous sodium bicarbonate solution, brine, dried over Na₂SO₄, and concentrated to dryness. The residue is purified by flash column chromatography on silica gel (50% to 100% EtOAC/hexanes) to afford methyl (S)-1-((2S,4R)-2-(4-iodo-1H-imidazol-2-yl)-4-methylpyrrolidin-1-yl)-3-methyl-1-oxobutan-2-ylcarbamate (119 mg, 0.2595 mmol, 95.26%) as white solid.

Rf=0.47 (EtOAc).

¹H NMR (400 MHz, CD₃OD) δ 7.06 (s, 1H), 5.13 (dd, J=8.4, 3.7 Hz, 1H), 4.14 (d, J=7.5 Hz, 1H), 3.92 (dd, J=9.7, 7.1 Hz, 1H), 3.62 (s, 3H), 3.43 (dd, J=9.6, 8.0 Hz, 1H), 2.71-2.6 (m, 1H), 2.30-1.81 (m, 3H), 1.09 (d, J=6.7 Hz, 3H), 0.88 (d, J=6.7 Hz, 3H), 0.861 (d, J=6.7 Hz, 3H).

LC/MS: m/z 434.9 (M+H⁺).

### Intermediate 6

### Methyl N-[(1S)-1-[(2S,4S)-2-(4-iodo-1H-imidazol-2-yl)-4-methyl-pyrrolidine-1-carbonyl]-2-methyl-propyl]-N-methyl-carbamate

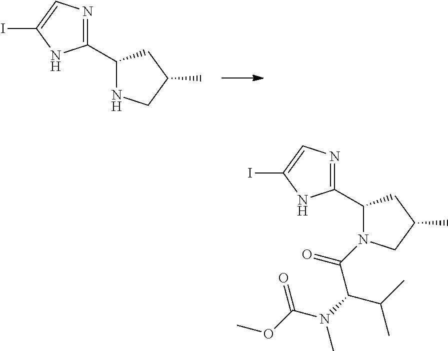

To a stirring solution of 4-iodo-2-[(2S,4S)-4-methylpyrrolidin-2-yl]-1H-imidazole (250 mg, 0.90 mmol), (2S)-2-[methoxycarbonyl(methyl)amino]-3-methyl-butanoic acid (179.2 mg, 0.94 mmol) and 2,4,6-collidine (357.7 μL, 2.7 mmol) in DMF (4 mL) is added HATU (581.2 mg, 0.94 mmol) at 0° C. The reaction mixture is stirred at RT overnight and diluted with EtOAc. The organic layer is washed with H₂O, brine, dried over Na₂SO₄, filtered and concentrated to dryness. The residue is purified by flash column chromatography on silica gel (10 to 100% EtOAc/Hexane) to afford Methyl N-[(1S)-1-[(2S,4S)-2-(4-iodo-1H-imidazol-2-yl)-4-methyl-pyrrolidine-1-carbonyl]-2-methyl-propyl]-N-methyl-carbamate (180 mg, 44.5%).

¹H NMR (400 MHz, CD₃OD): δ [ppm] 7.04 (s, 1H), 4.89 (m, 1H), 4.48 (d, 1H), 4.33 (d, 0.5H), 4.19 (m, 1H), 4.9 (m, 0.5H), 3.7 (s, 3H), 2.79 (s, 3H), 2.4 (m, 1H), 2.25-2.2 (m, 1H), 2.11-2.05 (m, 1H), 1.79-1.7 (m, 1H), 1.12 (s, 3H), 0.8 (dd, 3H), 0.76 (dd, 3H).

LC/MS: m/z=448.95 (M+H⁺).

### Intermediate 7

### Methyl N-[(1S)-1-[(2S)-2-(4-ethynyl-1H-imidazol-2-yl)pyrrolidine-1-carbonyl]-2-methyl-propyl]carbamate

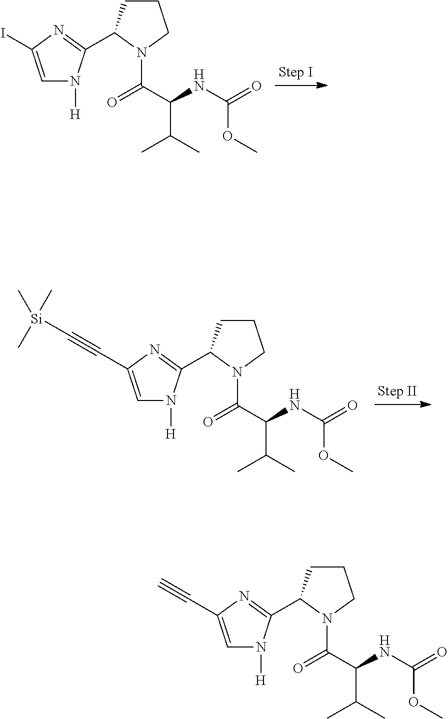

### Step I

### Methyl N-[(1S)-2-methyl-1-[(2S)-2-[4-(2-trimethylsilylethynyl)-1H-imidazol-2-yl]pyrrolidine-1-carbonyl]propyl]carbamate

Ethynyl(trimethyl)silane (91.1 mg, 131.1 μL, 0.93 mmol), methyl N-[(1S)-1-[(2S)-2-(4-iodo-1H-imidazol-2-yl)pyrrolidine-1-carbonyl]-2-methyl-propyl]carbamate (130 mg, 0.31 mmol), Pd(dppf)Cl₂-DCM (25.2 mg, 0.031 mmol), TEA (62.60 mg, 86.2 μL, 0.62 mmol), and CuI (11.78 mg, 0.062 mmol) are dissolved in 10 mL of DMF. The system is purged with nitrogen and the mixture is heated to 70° C. overnight under nitrogen. After evaporation of the solvent under vacuum, the residue is purified by flash column chromatography on silica gel (0 to 5% methanol/CH₂Cl₂) to afford methyl N-[(1S)-2-methyl-1-[(2S)-2-[4-(2-trimethylsilylethynyl)-1H-imidazol-2-yl]pyrrolidine-1-carbonyl]propyl]carbamate (100 mg).

¹H NMR (400 MHz, CDCl₃): δ [ppm] 10.35 (s, 1H), 6.93 (s, 1H), 5.50 (d, 1H), 4.95 (m, 1H), 4.07 (m, 1H), 3.37-3.69 (m, 5H), 1.69-2.10 (m, 5H), 0.60-0.83 (m, 6H), 0.00 (m, 9H).

LC/MS: m/z 391.00 (M+H⁺).

### Step II:

### Methyl N-[(1S)-1-[(2S)-2-(4-ethynyl-1H-imidazol-2-yl)pyrrolidine-1-carbonyl]-2-methyl-propyl]carbamate

To a solution of methyl N-[(1S)-2-methyl-1-[(2S)-2-[4-(2-trimethylsilylethynyl)-1H-imidazol-2-yl]pyrrolidine-1-carbonyl]propyl]carbamate (100 mg, 0.256 mmol) in 3 mL of THF is added 1M TBAF/THF (384 L, 0.38 mmol). The mixture is stirred at RT for 20 minutes and the solvent is removed under reduced pressure. The residue is purified by flash column chromatography on silica gel (0 to 5% methanol/CH₂Cl₂) to afford methyl N-[(1S)-1-[(2S)-2-(4-ethynyl-1H-imidazol-2-yl)pyrrolidine-1-carbonyl]-2-methyl-propyl]carbamate (56 mg).

### Intermediate 8

### Methyl (2S,3R)-1-((2S,4S)-2-(4-iodo-1H-imidazol-2-yl)-4-methylpyrrolidin-1-yl)-3-methoxy-1-oxobutan-2-ylcarbamate

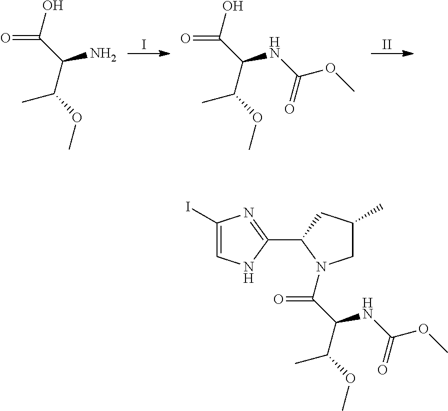

### Step I:

### (2S,3R)-3-methoxy-2-(methoxycarbonylamino)butanoic acid

To a solution of (2S,3R)-2-amino-3-methoxy-butanoic acid (2 g, 15.02 mmol) in aq. NaOH (14.87 mL of 1 M, 14.87 mmol) is added Na₂CO₃ (827.8 mg, 7.810 mmol). The reaction mixture is stirred for few minutes until solution become clear, and cooled to ice-bath temperature. Methyl chloroformate (1.533 g, 1.253 mL, 16.22 mmol) is added drop wise over a period of 10 minutes and the reaction mixture is stirred for 4.5 hours at RT. The aqueous solution is washed with diethyl ether (3×30 mL), cooled to ice-bath temperature, acidified with HCl_{conc}, extracted with CH₂Cl₂ (3×30 mL). The combined organic extracts are washed with brine, dried over Na₂SO₄, filtered, and concentrated to afford (2S,3R)-3-methoxy-2-(methoxycarbonylamino)butanoic acid (1.96 g, 10.25 mmol, 68.25%) as white solid.

¹H NMR (400 MHz, CD₃OD) δ 4.92 (br s, 1H), 4.18 (d, 1H), 3.93 (td, 1H), 3.65 (s, 3H), 3.29 (s, 3H), 1.16 (d, 3H).

### Step II:

Methyl (2S,3R)-1-((2S,4S)-2-(4-iodo-1H-imidazol-2-yl)-4-methylpyrrolidin-1-yl)-3-methoxy-1-oxobutan-2-ylcarbamate

To a cold (0-4° C.) stirred solution of 5-iodo-2-[(2S,4S)-4-methylpyrrolidin-1-ium-2-yl]-1H-imidazole hydrochloride (90 mg, 0.2564 mmol) and (2S,3R)-3-methoxy-2-(methoxycarbonylamino)butanoic acid (51.47 mg, 0.2692 mmol) in DMF (1.800 mL) is sequentially added HATU (102.4 mg, 0.2692 mmol) and DIPEA (132.6 mg, 178.7 μL, 1.026 mmol). The reaction mixture is slowly warmed up to RT, stirred overnight, diluted with water (5 mL), and extracted with EtOAc (3×10 mL). The combined extracts are washed with saturated aqueous sodium bicarbonate solution, brine, dried over Na₂SO₄, and concentrated to dryness. The residue is purified by flash column chromatography on silica gel (50 to 100% EtOAc/hexanes). The residue is dissolved in toluene and concentrated to dryness to remove azeotropically trace amounts of DMF to afford methyl (2S,3R)-1-((2S,4S)-2-(4-iodo-1H-imidazol-2-yl)-4-methylpyrrolidin-1-yl)-3-methoxy-1-oxobutan-2-ylcarbamate (107 mg, 0.2376 mmol, 92.72%) as solid.

Rf=0.15 (EtOAc-Hexanes, 7:3).

¹H NMR (400 MHz, CDCl₃) δ 10.48 (s, 1H), 6.99 (s, 1H), 5.63 (d, 1H), 5.15 (t, 1H), 4.51 (br s, 1H), 3.94-3.84 (m, 1H), 3.68 (s, 3H), 3.6-3.5 (m, 1H), 3.15 (s, 3H), 3.07 (t, 1H), 2.47 (t, 2H), 2.35-2.2 (m, 1H), 1.7-1.55 (m, 1H), 1.13 (d, 3H), 1.08 (d, 3H).

### Intermediate 9

### Methyl N-[(1S)-1-[(2S,4S)-2-(5-iodo-1H-imidazol-2-yl)-4-methyl-pyrrolidine-1-carbonyl]-2-methyl-propyl]carbamate

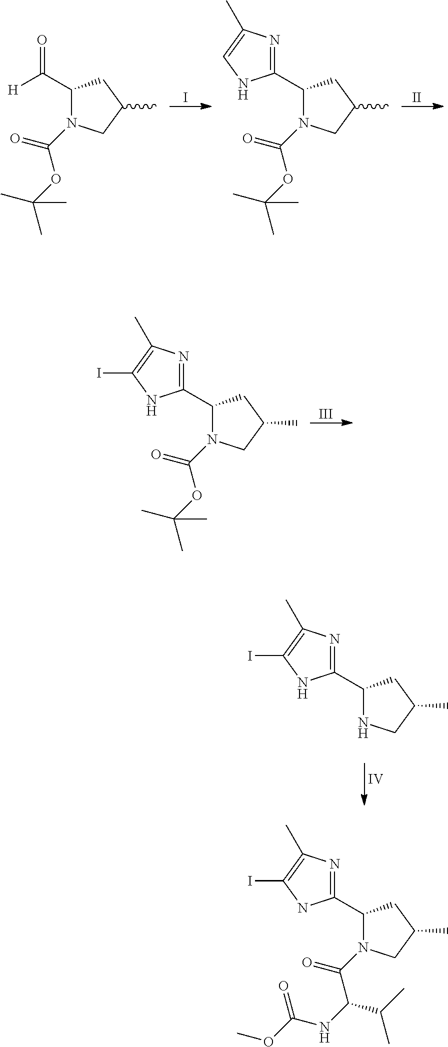

### Step I

### tert-Butyl (2S)-4-methyl-2-(4-methyl-1H-imidazol-2-yl)pyrrolidine-1-carboxylate

A stirred solution of tert-butyl (2S)-2-formyl-4-methyl-pyrrolidine-1-carboxylate (282 mg, 1.322 mmol) in MeOH (5.6 mL) is cooled to −20° C. and gaseous ammonia is bubbled for 10 minutes. 2-oxopropanal (35% w/w in water, 1.905 g, 9.254 mmol) is added and the reaction mixture is warmed to room temperature over one hour. The mixture is then heated to 65° C. for 1 hour, concentrated and 5 mL of water is added to the residue. The aqueous layer is extracted with CH₂Cl₂ (3×10 mL). The combined organic layers are dried over Na₂SO₄, filtered, and evaporated to dryness. The residue is purified by flash column chromatography on silica gel (0 to 20% MeOH in CH₂Cl₂) to afford tert-Butyl (2S)-4-methyl-2-(4-methyl-1H-imidazol-2-yl)pyrrolidine-1-carbo-xylate 307 mg (88%).

### Step II

### tert-butyl (2S,4S)-2-(5-iodo-4-methyl-1H-imidazol-2-yl)-4-methyl-pyrrolidine-1-carboxylate

To a stirred solution of tert-butyl (2S)-4-methyl-2-(4-methyl-1H-imidazol-2-yl)pyrrolidine-1-carboxylate (307 mg, 1.013 mmol) in CH₂Cl₂ (15 mL) is added N-iodosuccinimide (240 mg, 1.013 mmol) at 5° C. The reaction mixture is stirred for one hour and 2 mL of water is added. The organic layer is separated, dried over Na₂SO₄, and evaporated to dryness. The residue is purified by flash column chromatography on silica gel (12 to 100% EtOAc in Hexanes) to give tert-butyl (2S,4S)-2-(5-iodo-4-methyl-1H-imidazol-2-yl)-4-methyl-pyrrolidine-1-carboxylate (246 mg, 62%).

### Step III:

### 4-Iodo-5-methyl-2-[(2S,4S)-4-methylpyrrolidin-2-yl]-1H-imidazole hydrochloride

tert-Butyl (2S,4S)-2-(5-iodo-4-methyl-1H-imidazol-2-yl)-4-methyl-pyrrolidine-1-carboxylate (125 mg, 0.320 mmol) is stirred with HCl (4M in dioxane, 2 mL) overnight. The reaction mixture is then concentrated to afford 5-iodo-4-methyl-2-[(2S,4S)-4-methylpyrrolidin-2-yl]-1H-imidazole hydrochloride used as is for the next step.

### Step IV:

### Methyl N-[(1S)-1-[(2S,4S)-2-(5-iodo-4-methyl-1H-imidazol-2-yl)-4-methyl-pyrrolidine-1-carbonyl]-2-methyl-propyl]carbamate

5-iodo-4-methyl-2-[(2S,4S)-4-methylpyrrolidin-2-yl]-1H-imidazole hydrochloride (0.320 mmol) and (2S)-2-(methoxycarbonylamino)-3-methyl-butanoic acid (56 mg, 0.320 mmol) are dissolved in DMF (2 mL) and the mixture is cooled in an ice bath. DIPEA (167 μL, 0.960 mmol) is added followed by HATU (134 g, 0.352 mmol). The reaction mixture is warmed to room temperature, stirred for 20 hours, diluted with 5 mL of saturated NaHCO₃ (aq.) and extracted with EtOAc. The combined organic layers are washed with water, dried over Na₂SO₄, and evaporated to dryness. The residue is purified by flash column chromatography on silica gel (0 to 20% MeOH/CH₂Cl₂) to afford methyl N-[(1S)-1-[(2S,4S)-2-(5-iodo-4-methyl-1H-imidazol-2-yl)-4-methyl-pyrrolidine-1-carbonyl]-2-methyl-propyl]carbamate (125 mg, 88% for the last two steps).

### Intermediate 10

### Methyl N-[(1S)-1-[(2S)-2-(5-iodo-4-methyl-1H-imidazol-2-yl)-pyrrolidine-1-carbonyl]-2-methyl-propyl]carbamate

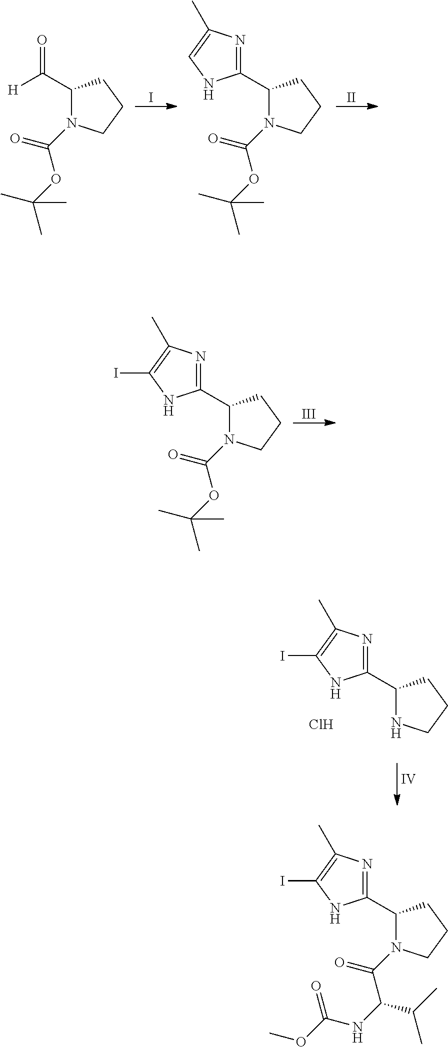

### Step I:

### tert-butyl (2S)-2-(4-methyl-1H-imidazol-2-yl)pyrrolidine-1-carboxylate

A stirred solution of tert-butyl (2S)-2-formyl-pyrrolidine-1-carboxylate (540 mg, 2.710 mmol) in MeOH (10.8 mL) is cooled to −20° C. and gaseous ammonia is bubbled for 10 minutes. 2-oxopropanal (35% w/w in water, 3.9 g, 19.0 mmol) is then added and the reaction mixture is warmed to RT over one hour. The mixture is then heated to 65° C. for 1 hour. The solvent is evaporated and 5 mL of water is added to the residue. The product is then extracted with EtOAc (3×10 mL). The combined organic layers are dried over Na₂SO₄, filtered, and evaporated to dryness. The residue is purified by flash column chromatography on silica gel (20 to 100% EtOAc in hexanes) to give tert-butyl (2S)-2-(4-methyl-1H-imidazol-2-yl)pyrrolidine-1-carboxylate (644 mg, 88%).

### Step II:

### tert-Butyl (2S)-2-(5-iodo-4-methyl-1H midazol-2-yl)-pyrrolidine-1-carboxylate

To a stirred solution of tert-butyl (2S)-2-(4-methyl-1H-imidazol-2-yl)pyrrolidine-1-carboxylate (625 mg, 2.178 mmol) in CH₂Cl₂ (5.5 mL) at 5° C. is added N-iodosuccinimide (516 mg, 2.178 mmol). The reaction is stirred for one hour and 5 mL of water is added. The organic layer is separated, dried over Na₂SO₄, and evaporated to dryness. The residue is purified by flash column chromatography on silica gel (0 to 20% MeOH in CH₂Cl₂) to give tert-butyl (2S)-2-(5-iodo-4-methyl-1H-imidazol-2-yl)-pyrrolidine-1-carboxylate.

### Step III:

### 5-iodo-4-methyl-2-[(2S)-pyrrolidin-2-yl]-1H-imidazole hydrochloride

tert-butyl (2S)-2-(5-iodo-4-methyl-1H-imidazol-2-yl)-pyrrolidine-1-carboxylate (2.178 mmol from previous step) is stirred with HCl (4M in dioxane, 5.4 mL) for one hour. Diethyl ether is added (5 ml) and a precipitate is formed. The precipitate is collected by filtration and dried under vacuum to afford 5-iodo-4-methyl-2-[(2S)-pyrrolidin-2-yl]-1H-imidazole hydrochloride (505 mg, 74% for the last two steps).

### Step IV:

### Methyl N-[(1S)-1-[(2S)-2-(5-iodo-4-methyl-1H-imidazol-2-yl)-pyrrolidine-1-carbonyl]-2-methyl-propyl]carbamate

5-iodo-4-methyl-2-[(2S,4S)-pyrrolidin-2-yl]-1H-imidazole hydrochloride (492 mg, 1.569 mmol) and (2S)-2-(methoxycarbonylamino)-3-methyl-butanoic acid (275 mg, 1.569 mmol) are dissolved in DMF (4.9 mL) and the mixture is cooled in an ice bath. DIPEA (820 μL, 4.707 mmol) is added followed by HATU (656 g, 1.726 mmol). The reaction mixture is warmed to room temperature and stirred for 20 hours. The reaction mixture is then diluted with 10 mL of saturated NaHCO₃ (aq.) and extracted with EtOAc. The combined organic layers are dried over Na₂SO₄, filtered, and evaporated to dryness. The residue is diluted in 5 mL of xylene and evaporated again. The residue is purified by flash column chromatography on silica gel (0 to 20% MeOH/CH₂Cl₂) to afford methyl N-[(1S)-1-[(2S)-2-(5-iodo-4-methyl-1H-imidazol-2-yl)-pyrrolidine-1-carbonyl]-2-methyl-propyl]carbamate (507 mg, 74%).

### Intermediate 11

### 4,7-diethynyl-1,3-benzodioxole

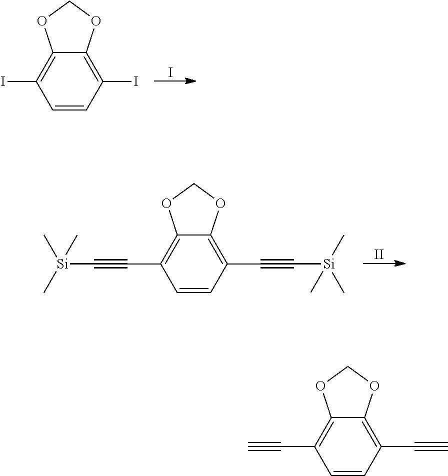

### Step I:

### Trimethyl-[2-[7-(2-trimethylsilylethynyl)-1,3-benzodioxol-4-yl]ethynyl]silane

To a stirred solution of 4,7-diiodo-1,3-benzodioxole (500 mg, 1.3 mmol), Pd(PPh₃)₂Cl₂ (93.8 mg, 0.13 mmol), CuI (50.9 mg, 0.26 mmol) in DMF (5 mL) is added ethynyl(trimethyl)silane (472 μL, 3.3 mmol) and TEA (931 μL, 6.6 mmol). The reaction mixture is stirred at 80° C. for 16 hours, diluted with EtOAc, H₂O, and filtered on celite. The organic phase is separated, dried over Na₂SO₄, filtered and concentrated to dryness. The residue is purified by flash column chromatography on silica gel (0 to 10% EtOAc in Hexanes) to give trimethyl-[2-[7-(2-trimethylsilylethynyl)-1,3-benzodioxol-4-yl]ethynyl]silane (344 mg, 81%).

### Step II:

### 4,7-diethynyl-1,3-benzodioxole

To a solution of trimethyl-[2-[7-(2-trimethylsilyl-ethynyl)-1,3-benzodioxol-4-yl]ethynyl]silane (340 mg, 1.08 mmol) in MeOH (6.8 mL) is added K₂CO₃ (328.7 mg, 2.37 mmol). The reaction mixture is stirred at RT for 2 hours and evaporated to dryness. The residue is diluted with EtOAc and H₂O. The organic phase is separated, dried over Na₂SO₄, filtered and concentrated to give 4,7-diethynyl-1,3-benzodioxole (180 mg, 98%) as a brown solid.

### Intermediate 12

### 2,6-Diethynylnaphthalene

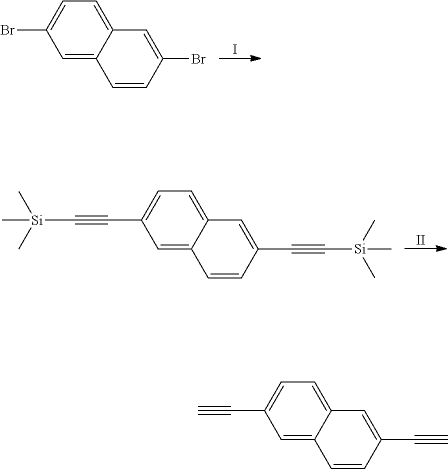

### Step I:

### 2,6-Bis((trimethylsilyl)ethynyl)naphthalene

To a suspension of 2,6-dibromonaphthalene (500 mg, 1.748 mmol), CuI (66.58 mg, 0.3496 mmol) and Pd(dppf)Cl₂-DCM (214.1 mg, 0.2622 mmol) in DMF (8 mL) is sequentially added TEA (707.5 mg, 974.5 μL, 6.992 mmol) and cold ethynyl(trimethyl)silane (377.7 mg, 543.5 μL, 3.846 mmol) under nitrogen atmosphere. The reaction mixture is heated at 80° C. for 7.5 hrs in a sealed tube and a precipitate is formed in the reaction tube after few hours of heating. The reaction mixture is cooled to RT, and filtered off. The white precipitate is washed with DMF (3.0 mL), dried under high vacuum to afford 2,6-bis((trimethylsilyl)ethynyl)naphthalene (620 mg) as white solid.

¹H NMR spectra showed 2.6:1 mixture of the desired compound and triethyl ammonium bromide, this mixture is used as such in the next step without further purification.

¹H NMR (400 MHz, CDCl₃) δ 7.91 (s, 2H), 7.67 (d, 2H), 7.47 (d, 2H), 0.27 (s, 18H).

### Step II:

### 2,6-Diethynylnaphthalene

To a stirred suspension of trimethyl-[2-[6-(2-trimethylsilylethynyl)-2-naphthyl]ethynyl]silane (590 mg, 1.472 mmol) in MeOH (10 mL) is added K₂CO₃ (447.5 mg, 3.238 mmol) in one portion, and the heterogeneous mixture is stirred at RT for 2.5 hours. To this reaction mixture is added CH₂Cl₂ (3.0 mL) and the mixture is stirred for an additional 30 minutes, concentrated, diluted with water (6 mL), and extracted with CH₂Cl₂ (3×10 mL). The combined organic extracts are washed with brine, dried over Na₂SO₄, filtered, and concentrated to dryness. The residue is purified by flash column chromatography on silica gel (0 to 5% EtOAc in Hexanes) to give 2,6-diethynylnaphthalene (157 mg, 0.8901 mmol, 60.46%) as white solid.

Rf=0.37 (EA:hex, 1:20).

¹H NMR (400 MHz, CDCl₃) δ 8.00-7.95 (m, 2H), 7.73 (d, 2H), 7.53 (dd, 2H), 3.17 (s, 2H).

### Intermediate 13

### 1,4-Diethynyl-2,5-dimethyl-benzene

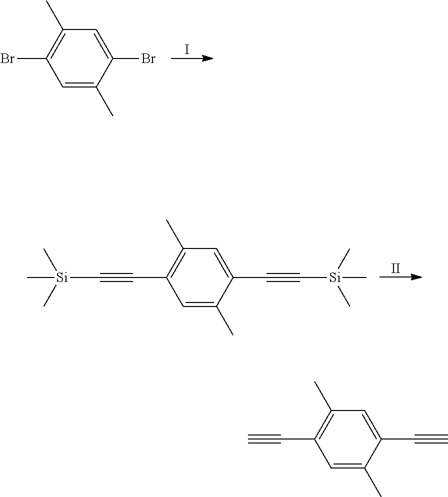

### Step I:

### 1,4-Dimethyl-2,5-bis-trimethylsilyl)ethynyl-benzene

1,4-Dimethyl-2,5-bis-trimethylsilyl)ethynyl-benzene is prepared from 1,4-dibromo-2,5-dimethyl-benzene according to the procedure reported for 2,6-bis((trimethylsilyl)ethynyl)naphthalene.

### Step II:

### 1,4-Diethynyl-2,5-dimethyl-benzene

To a stirred suspension of 1,4-dimethyl-2,5-bis-trimethylsilyl)ethynyl-benzene (700 mg, 2.345 mmol) in MeOH (14.70 mL) is added dipotassium carbonate (713.0 mg, 5.159 mmol) in one portion and the heterogeneous mixture is stirred at RT for 2.5 hours. To this mixture is added CH₂Cl₂ (3.0 mL), and the mixture is stirred for an additional 30 minutes, concentrated, diluted with water (6 mL), and extracted with CH₂Cl₂ (3×15 mL). The combined organic extracts are washed with brine, dried over Na₂SO₄, filtered and concentrated to dryness. The residue is purified by flash column chromatography on silica gel (0 to 5% MeOH in CH₂Cl₂) to give 1,4-diethynyl-2,5-dimethyl-benzene (248 mg, 1.608 mmol, 68.58%) as light yellow solid.

Rf=0.43 (Hexanes).

¹H NMR (400 MHz, CDCl₃) δ 7.29 (s, 2H), 3.31 (s, 2H), 2.37 (s, 6H).

### Intermediate 14

### 1,4-Diethynyl-2-methyl-benzene

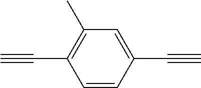

The title compound is synthesized from 1,4-dibromo-2-methyl benzene as described in Intermediate 13.

### Intermediate 15

### 2,5-Diethynyl-thieno[3,2-b]thiophene

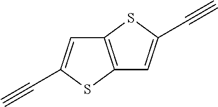

The title compound is synthesized from 2,5-Dibromo-thieno[3,2-b]thiopheneas described in Intermediate 13.

### Intermediate 16

### 2,5-Diethynyl-thieno[3,2-b]thiophene

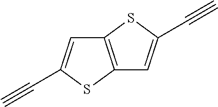

The title compound is synthesized from 2,5-Dibromo-thieno[3,2-b]thiopheneas described in Intermediate 13.

### Intermediate 17

(2S,4S)-4-methyl-N-(t-butoxycarbonyl)prolinol

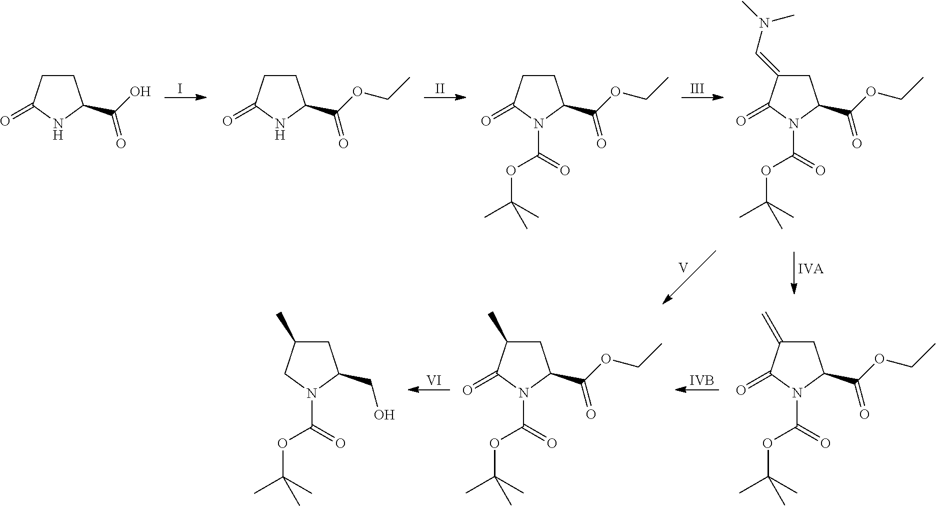

### Step I:

### Ethyl-(2S)-pyroglutamate

To a solution of L-pyroglutamic acid (445 g, 3.45 mol) in ethanol (3.6 L) was added 98% sulfuric acid (18 mL). The resulting reaction mixture was stirred at room temperature overnight. Sodium carbonate (140 g) was added and the stirring was continued for an additional 1.5 hr. The suspension was filtered and the filtrate was evaporated under reduced pressure. The residue was azeotroped with dichloromethane to afford 541 g (100%) of ethyl-(2S)-pyroglutamate as a viscous oil. This material was used in the next step without any further purification.

### Step II:

### Ethyl-(2S)-N-(t-butoxycarbonyl)pyroglutamate

To a solution of ethyl L-pyroglutamate (542 g, 3.45 mol), dimethylaminopyridine (42.14 g, 3.79 mol), and triethylamine (577 mL, 4.139 mol) in dichloromethane (4 L) was added in portions BOC anhydride (827.8 g, 3.79 mol). The resulting mixture was stirred at room temperature overnight. The reaction mixture was washed with saturated aqueous ammonium chloride solution. The dichloromethane layer was separated, dried with magnesium sulfate, filtered, and evaporated in vacuo to afford 887.4 g (100%) of (ethyl-(2S)-N-(t-butoxycarbonyl)pyroglutamate as a light red oil that solidified upon standing.

The crude product contained traces of dimethylaminopyridine and dichloromethane but was clean enough to use in the next step without further purification.

### Step III:

### Ethyl-(2S)-4-(N,N-dimethylaminomethylidene-N-(t-butoxy]carbonyl)pyroglutamate

To a solution of ethyl-(2S)-N-(t-butoxycarbonyl)pyroglutamate (150 g, 583 mmol) in 1,2-dimethoxy]ethane (1.5 L) was added 1-t-butoxy-N,N,N′,N′-tetramethylmethanediamine (162 g, 932.8 mmol). The reaction was heated to reflux overnight, cooled, and evaporated in vacuo. The residue was triturated with hexanes and filtered to afford 170 g (93%) of ethyl-(2S)-4-(N,N-dimethylaminomethylidene-N-(t-butoxycarbonyl)pyroglutamate as a red solid.

### Step IVA:

### Ethyl-(2S)-4-methylene-N-(t-butoxycarbonyl)pyroglutamate

To a solution of ethyl-(2S)-4-(N,N-dimethylaminomethylidene-N-(t-butoxycarbonyl)pyroglutamate (23.6 g, 75.4 mmol) in tetrahydrofuran (120 mL) was added 1N HCl (70 mL). The reaction stirred at room temperature for two hours. The layers were separated and the aqueous layer was extracted with an additional 50 mL of tetrahydrofuran. The tetrahydrofuran layers were combined and potassium carbonate (14.9 g, 107.5 mmol) and 37% formaldehyde in water 60 mL) were added. The mixture was stirred for 45 minutes. The layers were separated and the tetrahydrofuran layer was dried with magnesium sulfate. The solvent was evaporated in vacuo and the oil that remained was dissolved in dichloromethane and filtered over a plug of silica gel. The plug was eluted with 25% ethyl acetate/dichloromethane and the filtrate was evaporated in vacuo to afford 16.4 g (81%) of ethyl-(2S)-4-methylene-N-(t-butoxycarbonyl)pyroglutamate was a yellow solid.

### Step IVB:

### Ethyl-(2S,4S)-4-methyl-N-(t-butoxycarbonyl)pyroglutamate

To a solution of ethyl-(2S)-4-methylene-N-(t-butoxycarbonyl)pyroglutamate (9.6 g, 35.7 mmol) in methanol (96 mL) was platinum (IV) oxide (404 mg, 1.782 mmol). The reaction degassed and stirred under an atmosphere of hydrogen overnight. The reaction was filtered through Celite and the filtercake was rinsed with methanol. The filtrate was evaporated in vacuo to afford 8.6 g (89%) of ethyl-(2S,4S)-4-methyl-N-(t-butoxycarbonyl)pyroglutamate as a clear, colorless oil.

### Step V:

### Ethyl-(2S,4S)-4-methyl-N-(t-butoxycarbonyl)pyroglutamate

A Parr vessel was charged with a solution of ethyl-(2S)-4-(N,N-dimethylaminomethylidene-N-(t-butoxy]carbonyl)pyroglutamate (30 g, 96.04 mmol) in ethanol (300 mL) and 10.22 g (9.60 mmol) of 10% wet, Degussa-type Pd/C. The atmosphere above the solution was evacuated and replaced with hydrogen at 50 psi. The reaction shaken for a day replacing the pressure of hydrogen as necessary. The filtrate was filtered over Celite and the filterbed was washed with ethyl acetate. The combined filtrates were evaporated in vacuo to afford an oil. The oil was purified on a silica gel column using 10-30% ethyl acetate/hexanes to afford 18.6 g (71%) of ethyl-(2S,4S)-4-methyl-N-(t-butoxycarbonyl)pyroglutamate as a clear, viscous oil.

### Step VI:

### (2S,4S)-4-methyl-N-(t-butoxycarbonyl)prolinol

To a solution of ethyl-(2S,4S)-4-methyl-N-(t-butoxycarbonyl)pyroglutamate (11 g, 40.5 mmol) in tetrahydrofuran (66 mL) cooled to 0° C. was added sodium borohydride (3.84 g, 101.4 mmol). The reaction stirred for 15 minutes and boron trifluoride etherate (14.39 g, 101.4 mmol) was added dropwise. The reaction was stirred at 0° C. for three hours and then warmed to ambient temperature and stirred for twelve hours. The reaction was poured into ice cold water, stirred for thirty minutes, and evaporated in vacuo. The residue was dissolved in ethyl acetate and washed successively with 1N hydrochloric acid, water and brine. The organic solution was dried with sodium sulfate, filtered, and evaporated in vacuo. The crude product was purified through column chromatography on a silica gel column using 10-40% ethyl acetate in hexanes to afford 8.73 g (69%) of (2S,4S)-4-methyl-N-(t-butoxycarbonyl)prolinol as a clear, colorless oil.

### Intermediate 18: Method A

### (3S,5S)-tert-butyl 5-(5-iodo-1H-imidazol-2-yl)-3-methyl-2-oxopyrrolidine-1-carboxylate

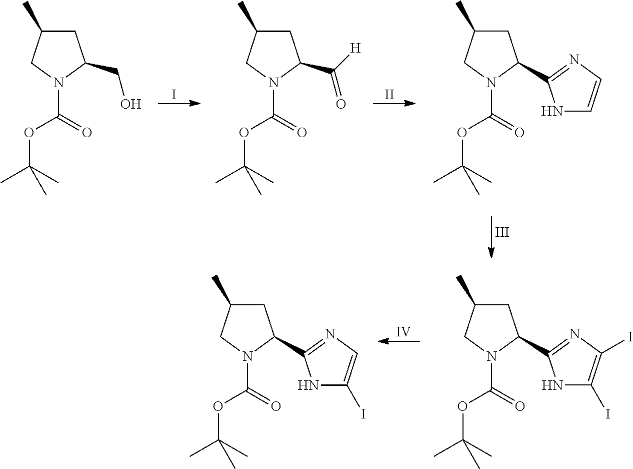

### Step I:

### (2S,4S)-tert-butyl 2-formyl-4-methylpyrrolidine-1-carboxylate

4-Methyl-N-(t-butoxycarbonyl)-(2S)-prolinol is oxidized to (2S,4S)-tert-butyl 2-formyl-4-methylpyrrolidine-1-carboxylate under standard oxidizing conditions known in the art for oxidizing an alcohol to an aldehyde (e.g. TEMPO/bleach).

### Step II:

### (2S,4S)-tert-butyl 2-(1H-imidazol-2-yl)-4-methylpyrrolidine-1-carboxylate

The (2S,4S)-tert-butyl 2-formyl-4-methylpyrrolidine-1-carboxylate is converted to the (2S,4S)-tert-butyl 2-(1H-imidazol-2-yl)-4-methylpyrrolidine-1-carboxylate using ammonium hydroxide and glyoxal in methanol (see Intermediate 3, Step I).

### Step III:

### (2S,4S)-tert-butyl 2-(4,5-diiodo-1H-imidazol-2-yl)-4-methylpyrrolidine-1-carboxylate

The (2S,4S)-tert-butyl 2-(1H-imidazol-2-yl)-4-methylpyrrolidine-1-carboxylate is reacted with N-iodosuccinimide to afford the bis-iodoimidazole (2S,4S)-tert-butyl 2-(4,5-diiodo-1H-imidazol-2-yl)-4-methylpyrrolidine-1-carboxylate (see Intermediate 3, Step 11).

### Step IV:

### (2S,4S)-tert-butyl 2-(5-iodo-1H-imidazol-2-yl)-4-methylpyrrolidine-1-carboxylate

The 4-iodo substituent of (2S,4S)-tert-butyl 2-(4,5-diiodo-1H-imidazol-2-yl)-4-methylpyrrolidine-1-carboxylate is selectively removed by metallation, preferably with isopropyl magnesium chloride, followed by a quench with a proton source such as water to afforde the mono-iodoimidazole (2S,4S)-tert-butyl 2-(5-iodo-1H-imidazol-2-yl)-4-methylpyrrolidine-1-carboxylate (see Intermediate 3, Step III).

### Intermediate 18: Method B

### Methyl N-[(1S)-1-[(2S,4S)-2-(5-iodo-1H-imidazol-2-yl)-4-methyl-pyrrolidine-1-carbonyl]-2-methyl-propyl]carbamate

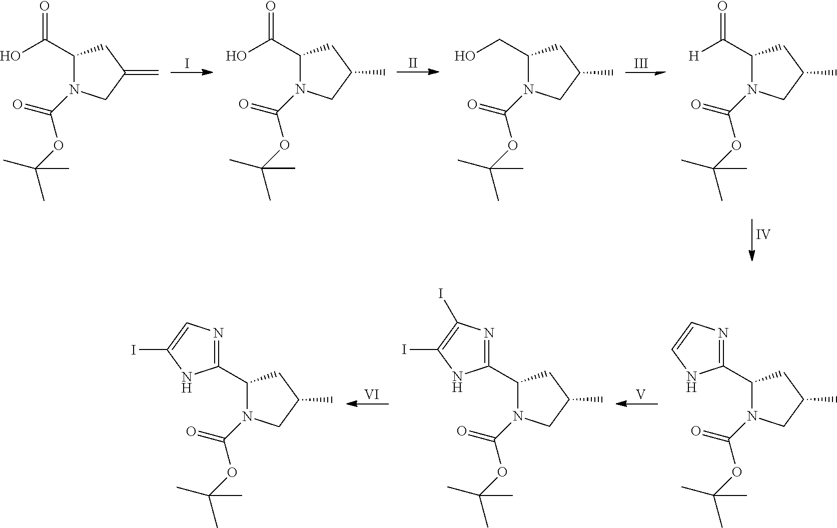

### Step I:

In a 2 L Parr flask under nitrogen, a solution of (2S)-1-tert-butoxycarbonyl-4-methylene-pyrrolidine-2-carboxylic acid (175 g, 770.1 mmol) in ethanol (700 mL) is added PtO₂ (5.250 g, 23.12 mmol). The suspension is purged 3 times, (vacuum/hydrogen) and stirred at rt on a Parr apparatus under 30-40 psi of hydrogen. The Initial H₂ consumption is rapid and then the reaction is completed after 60 minutes. The catalyst is decanted and the supernatant is cannulated in 1 L Erlenmeyer, then filtered through Celite and washed with ethanol. The mother liquor is put aside. The reaction is repeated 7 times using a total of 8.8 g of PtO₂. The reaction mixtures of the 7 runs are combined (ratio of cis/trans 3.6/1) and evaporated to a volume of 3 L. Isopropyl acetate (1.5 L) is added and 2 L of the solution is evaporated. Isopropyl acetate (1.5 L) is added and 1 L of the solution is evaporated. The compound is started to crystallize in the solution, heptane (1.5 Liters) and isopropyl acetate (1 L) are added and the suspension is stirred at rt for 1 hour and then at 0° C. for 2 hours. The mixture is filtered and washed with a cold solution of 2:1 heptane/isopropyl acetate (10. The cake is dried for 2 hours, then put in the vacuum oven yielding (2S)-1-tert-butoxycarbonyl-4-methyl-pyrrolidine-2-carboxylic acid; 883.44 g (71% yield) as a pale gray crystalline powder, 9:1 (S:R).

The crude product (850 g) is recrystallized in isopropyl acetate (2 volume) and heptane (1 volume). The recrystallization process is repeated 2 times to reach a ratio of 23/1 (S:R).

### Step II:

In a 22 L, 3 neck round bottom flask under nitrogen equipped with a thermocouple, an addition funnel and a mechanical stirrer (2S,4S)-1-tert-butoxycarbonyl-4-methyl-pyrrolidine-2-carboxylic acid (672 g, 2.931 mol) and THF (4 L) are added and cooled in an ice bath until the internal temperature is about 3° C. The Borane/THF solution (5.527 kg, 6.155 L of 1 M, 6.155 mol) is added dropwise over 3.5 hours using the addition funnel. An exotherm of 10° C. and a very strong gas formation are occurred during the addition of the first 1800 mL. The reaction mixture is stirred overnight at room temp. TLC: (25% EtOAC/Hexane) shows no starting material. Then 1 L of a saturated aqueous solution of NH₄Cl is carefully added (dropwise) at 4° C. An exotherm of 20° C. and a very strong gas formation are observed. H₂O (5000 mL) is added and the mixture is extracted with EtOAc (2 L), the phases are separated and the aqueous phase is re-extracted with EtOAc (2 L). The organic phases are merged and the solvent is evaporated. To the residual mixture is added EtOAc (3 L) and the phases are separated and dried over Na₂SO₄, filtered and concentrated to a small volume. The residual solution contains water and a white solid. The mixture is transferred to a 5 L extraction funnel and added EtOAc (1 L) and water (500 mL). The organic phase is dried over sodium sulfate and the mixture is stirred for 30 minutes, filtered and evaporated to dryness. The residue is purified by a plug of silica gel using 0 to 30% EtOAc/Hexanes as eluant to give tert-butyl (2S,4S)-2-(hydroxymethyl)-4-methyl-pyrrolidine-1-carboxylate (604 g, 2.806 mol, 95% yield).

### Step III:

In a 12 L of three-neck RBF under nitrogen equipped with a mechanical stirrer, a thermocouple and a 2 L addition funnel, oxalyl chloride (2.2 kg, 1.52 L of 2 M, 3.05 mol) and CH₂Cl₂ (2.1 L) are added. The solution is cooled to −78° C. in an acetone/dry ice bath while stirring. The DMSO (476.6 g, 432.9 mL, 6.1 mol) is added dropwise using the addition funnel, while keeping the internal temp around −67±5° C. A solution of tert-butyl (2S,4S)-2-(hydroxymethyl)-4-methyl-pyrrolidine-1-carboxylate (262.65 g, 1.22 mol) in CH₂Cl₂ (1.8 L) is added and the mixture is stirred for 20 minutes at −75±5° C. Finally DIPEA (1.57 kg, 2.13 L, 12.2 mol) is added over 90 minutes keeping the internal temp at −73±2° C. The reaction mixture is stirred for 2 hours at −76±2° C. and then the temperature is raised to room temp over 2 hours. A solution of HCl 1N (3400 ml) is slowly added using an addition funnel, an exotherm of 6° C. is observed, the phases are separated (aqueous pH=8) and the HCl washing procedure is repeated until the pH of the aqueous phase is below 2. The organic phase is dried using Na₂SO₄, filtered and concentrated to give a yellow oil (300 g). The Swern reaction was repeated on the same scale and the two crude reactions are combined and purified together. The residue is purified by a plug of silica gel using 0 to 15% EtOAc in Hexanes as eluant to give tert-butyl (2S,4S)-2-formyl-4-methyl-pyrrolidine-1-carboxylate (480 g, 2.25 mol, 92.2%) as a golden oil.

### Step IV:

In a 5 L 3 neck RBF under nitrogen equipped with a thermocouple, a refrigerant and an addition funnel, tert-butyl (2S,4S)-2-formyl-4-methyl-pyrrolidine-1-carboxylate (480 g, 2.25 mol) and MeOH (960 ml) are added while stirring an exotherm of +20° C. is observed. NH₄OH (960.0 mL) is added, an exotherm of 12° C. is noted. Oxaldehyde (913.7 g, 722.3 mL of 40% w/v, 4.98 mol) is added over 39 minutes using the addition funnel. A very big exotherm up to 70° C. is observed. The reaction mixture is stirred overnight at room temperature. In the morning the reaction mixture is all solidified with no apparent liquid. Water (1.5 L) is added and stirred for 1 hour. The suspension is filtered on a Buchner funnel and washed with water (˜2 L) until the mother liquor is pale brown. The wet solid is transferred in a 12 L RBF, then water (3 L) is added and the suspension is stirred for 2 hours, filtered and washed with water (1.5 L) and heptane (1.5 L) and then dried for 2 days in a vacuum oven at 45° C. The crude gray solid contains 8% of 2-(1H-imidazol-2-yl)-1H-imidazole as a side product impurity. The solid is transferred into a 12 L 5 neck RBF, ethyl acetate (4 L) is added and the mixture is refluxed. The water is azeotroped with ethyl acetate (1.5 L). The hot solution is filtered to remove the insoluble 2-(1H-imidazol-2-yl)-1H-imidazole. The remaining solution is evaporated to 1 L and then the suspension is stirred in an ice/bath, filtered and washed with cold ethyl acetate to give tert-butyl (2S,4S)-2-(1H-imidazol-2-yl)-4-methyl-pyrrolidine-1-carboxylate (330 g, 1.31 mol, 58% yield) as a pale gray solid, 99.4% pure.

### Step V:

In a 12 L 4 neck RBF under nitrogen equipped with mechanical stirrer, a thermocouple a refrigerant and an addition funnel, tert-butyl (2S,4S)-2-(1H-imidazol-2-yl)-4-methyl-pyrrolidine-1-carboxylate (334.4 g, 1.331 mol) and CH₂Cl₂ (3.344 L) are added while stirring. The solution is cooled to below 5° C. using an ice bath. NIS (628.8 g, 2.795 mol) is added in 6 batches of 104.8 g each over 30 minutes while keeping the internal temperature below 5° C. The reaction mixture is stirred for 2.5 hours and then HPLC shows the completion of the reaction. A solution of 10% sodium thiosulfate solution (4 L) is added and the reaction mixture is stirred for 15 minutes and the phases are separated. The organic phase is transferred back in the 12 L reactor, water (2 L) and CH₂Cl₂ (1 L) are added, the mixture is stirred for 15 minutes. The organic phase is dried over sodium sulfate, filtered and evaporated to dryness to afford tert-butyl (2S,4S)-2-(4,5-diiodo-1H-imidazol-2-yl)-4-methyl-pyrrolidine-1-carboxylate, (646.3 g, 93% yield, 96% purity). ¹H NMR (400 MHz, dmso, 2.5:1 mixture of rotamers), peaks for the major rotamer: δ 12.70 (s, 1H), 4.57 (dd, 1H), 3.62-3.52 (m, 1H), 2.95 (t, 1H), 2.35-2.0 (m, 2H), 1.50 (dd, 1H), 1.10 (s, 9H), 1.01 (d, 3H).

### Step VI:

In a 5 L 3 neck round bottom flask under nitrogen equipped with a mechanical stirrer, LiCl (54.09 g, 1.276 mol) and THF (2.504 L) are added and stirred at rt overnight. In a 12 L 3 neck round bottom flask under nitrogen equipped with a temperature reader, a mechanical stirrer and an 1 L addition funnel, tert-butyl (2S,4S)-2-(4,5-diiodo-1H-imidazol-2-yl)-4-methyl-pyrrolidine-1-carboxylate (642 g, 1.276 mol) and LiCl solution in THF are added while stirring. The reaction mixture is cooled down to −20° C. To the beige suspension is added dropwise a solution of methyl magnesium chloride in THF (425.3 mL of 3 M, 1.276 mol) over 60 minutes keeping the internal temperature between −17 and −20° C. The reaction mixture is stirred at the same temperature for 20 minutes. A solution of isopropyl magnesium chloride in THF (1.276 L of 2 M, 2.552 mol) is added dropwise over 70 minutes while keeping the internal temperature between −17 and −23° C., then the mixture is slowly warmed up to rt. TLC 50% ETOAC/Hexane shows that the reaction is completed. The reaction mixture is cooled down in an ice/water bath to −10° C. and a 10% aq. NH₄Cl solution (2 L) is added dropwise. An exotherm of 35° C. and gas formation are observed. Water (1 L) and ethyl acetate (2 L) are added and the mixture is stirred for 30 minutes. The phases are separated and the aqueous phase is re-extracted with EtOAc (2 L). The organic phases are combined and 2 L of water is added, the mixture is stirred for 10 minutes. The phases are separated and the organic phase is dried over Na₂SO₄ and filtered. The solvent is concentrated to a small volume and heptane (1000 mL) is added and evaporated slowly. The suspension is stirred for 30 minutes at rt and then in an ice bath for 1 hour, filtered and washed with cold heptane/ethyl acetate (95/5). The solid is dried in the vacuum oven at 35° C. to afford tert-butyl (2S,4S)-2-(5-iodo-1H-imidazol-2-yl)-4-methyl-pyrrolidine-1-carboxylate (420.24 g; 91% pure, 87.3% yield)) as a beige solid, used as it is for the next step.

### Intermediate 19

### Trimethyl-[2-[5-(2-trimethylsilylethynyl)-2-pyridyl]ethynyl]silane

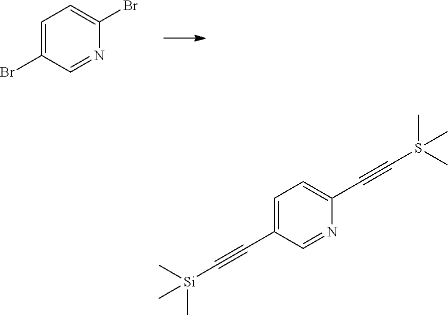

A stirred suspension of 2,5-dibromopyridine (1.5 g, 6.332 mmol), copper Iodide (120.6 mg, 0.6332 mmol) and PdCl₂(dppf)-CH₂Cl₂ (517.1 mg, 0.6332 mmol) in DMF (24.00 mL) is degassed and added triethyl amine (2.563 g, 3.530 mL, 25.33 mmol). Then ethynyl(trimethyl)silane (1.368 g, 1.968 mL, 13.93 mmol) is added under nitrogen atmosphere and the resultant reaction mixture is heated at 80° C. for 8 hours in a sealed tube. The reaction mixture is concentrated, passed through a small plug of silica and eluted with 10% ethyl acetate-hexanes. The solvents are concentrated and the residue is purified by silica gel column chromatography using EtOAc-Hexanes (0:100 to 1:90) as eluent to obtain trimethyl-[2-[6-(2-trimethylsilylethynyl)-3-pyridyl]ethynyl]silane (780 mg, 2.811 mmol, 43.43%) as light brown solid. Rf=0.61 (10% EtOAc-Hexanes). ¹H NMR (400 MHz, CDCl₃): δ 8.61 (dd, 1H), 7.67 (dd, 1H), 7.37 (dd, 1H), 0.254 (s, 9H), 0.246 (s, 9H).

LC/MS: m/z=271.86 (M+H⁹⁺). Rt=18.7 minutes.

### Intermediate 20

### tert-Butyl (1S,3S,5S)-3-(4-iodo-1H-imidazol-2-yl)-4-azabicyclo[3.1.0]hexane-4-carboxylate

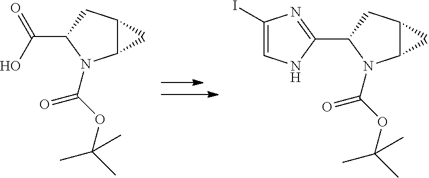 [SMILES: CC(C)(C)OC(=O)N1[C@H](C(=O)O)C[C@@H]2C[C@@H]21 ; CC(C)(C)OC(=O)N1[C@H](c2nc(I)c[nH]2)C[C@@H]2C[C@@H]21]

The title compound is prepared from (1S,3S,5S)-2-tert-butoxycarbonyl-2-azabicyclo[3.1.0]hexane-3-carboxylic using the same sequence of reactions (Step II to VI) as described for intermediate 4.

LC/MS: m/z=375.78 (M+H⁺). Rₜ=6.33 minutes.

### Intermediate 21

### tert-Butyl (2S,4S)-2-(4-iodo-1H-imidazol-2-yl)-4-methoxy-pyrrolidine-1-carboxylate

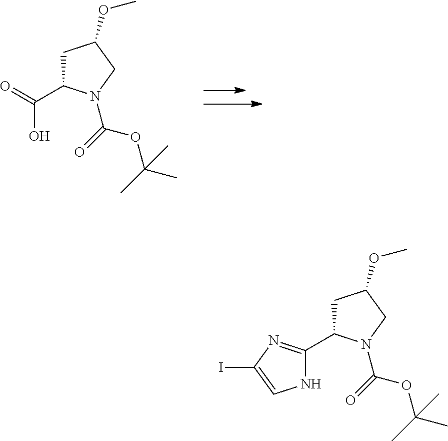 [SMILES: CO[C@H]1C[C@@H](C(=O)O)N(C(=O)OC(C)(C)C)C1 ; CO[C@H]1C[C@@H](c2nc(I)c[nH]2)N(C(=O)OC(C)(C)C)C1]

Title compound is prepared using the same sequence of reactions (step 11 to VI) as described for intermediate 4 starting from tert-butyl (2S,4S)-2-(hydroxymethyl)-4-methoxy-pyrrolidine-1-carboxylate. Rf=0.18 (70% EtOAc-Hexanes).

¹H NMR (400 MHz, CDCl₃, peaks for the major rotamer): δ 9.85 (s, 1H), 6.99 (s, 1H), 4.96 (br s, 1H), 4.1-3.3 (m, 3H), 3.35 (s, 3H), 2.4-2.2 (m, 2H), 1.28 (s, 9H).

LC/MS: m/z=393.78 (M+H⁺). Rt=8.46 minutes.

HPLC (Rt)=22.55 minutes, Method: 0-40% acetonitrile-water in 40 min, Gemini C18 3 μm, 4.6 mm×250 mm.

### Intermediate 22:

### 4,4,5,5-Tetramethyl-2-[6-(4,4,5,5-tetramethyl-1,3,2-dioxaborolan-2-yl)-2-naphthyl]-1,3,2-dioxaborolane

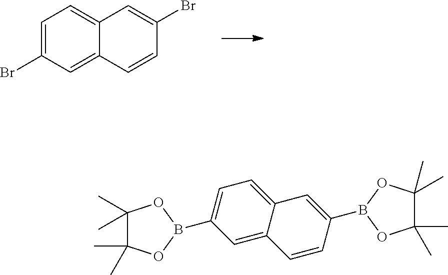

To a stirred suspension of 2,6-dibromonaphthalene (1 g, 3.497 mmol), 4,4,5,5-tetramethyl-2-(4,4,5,5-tetramethyl-1,3,2-dioxaborolan-2-yl)-1,3,2-dioxaborolane (2.131 g, 8.393 mmol) and KOAc (2.059 g, 20.98 mmol) in dioxnae (30.00 mL) is added Pd(dppf)Cl₂-CH₂Cl₂ (571.2 mg, 0.6994 mmol) in one portion, degassed and filled with nitrogen. The flask is sealed, heated at 100° C. overnight (crude LC-MS shows the desired compound) and cooled to rt. The reaction mixture is filtered through celite, washed with CH₂Cl₂ and concentrated. The residue is purified on a small plug of silica gel using CH₂Cl₂. The organic layer is diluted with heptanes (20 mL), concentrated on rotary evaporator until CH₂Cl₂ is removed. The resultant product is collected by filtration, washed with heptanes, dried under high vacuum to afford 4,4,5,5-tetramethyl-2,6-(4,4,5,5-tetramethyl-1,3,2-dioxaborolan-2-yl)-2-naphthyl]-1,3,2-dioxaborolane (760 mg, 1.990 mmol, 56.90%) as light brown solid. Rf=0.39 (1:9 EtOAc-Hexanes).

LC/MS, m/z=381 (M+H⁺); Rt: 18.94 minutes.

¹H NMR (400 MHz, CDCl₃): δ 8.357 (s, 2H), 7.86 (d, 2H), 7.827 (d, 2H), 1.39 (s, 24H).

### Intermediate 23

### 4,4,5,5-Tetramethyl-2-[5-(4,4,5,5-tetramethyl-1,3,2-dioxaborolan-2-yl)thieno[3,2-b]thiophen-2-yl]-1,3,2-dioxaborolane

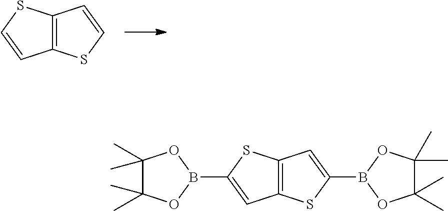

To a solution of thieno[3,2-b]thiophene (1.5 g, 10.70 mmol) in THF (25.5 mL) at −78° C. under N₂ is added dropwise a solution of BuLi in hexanes (8.988 mL of 2.5 M, 22.47 mmol), stirred for 20 minutes, cooling bath is replaced with ice bath and stirred for 50 minutes. The resultant thick suspension is quenched with 2-isopropoxy-4,4,5,5-tetramethyl-1,3,2-dioxaborolane (4.181 g, 4.584 mL, 22.47 mmol). The reaction mixture is kept overnight and then quenched with saturated aq. NH₄Cl solution. After extraction with CH₂Cl₂ (2×100 mL), the combined extracts are washed with brine and dried (Na₂SO₄). Organic solution is diluted with −20 mL of ethyl acetate, concentrated slowly on rotary evaporator until CH₂Cl₂ is removed. The resultant white fine crystals are collected by filtration. The solid is washed with heptanes and dried under high vacuum to afford 4,4,5,5-tetramethyl-2-[5-(4,4,5,5-tetramethyl-1,3,2-dioxaborolan-2-yl)thieno[3,2-b]thiophen-2-yl]-1,3,2-dioxaborolane (2.57 g, 6.554 mmol, 61.25%) as half-white solid. ¹H NMR (400 MHz, CDCl₃): δ 7.75 (s, 4H), 1.343 (s, 24H).

### EXAMPLE 1

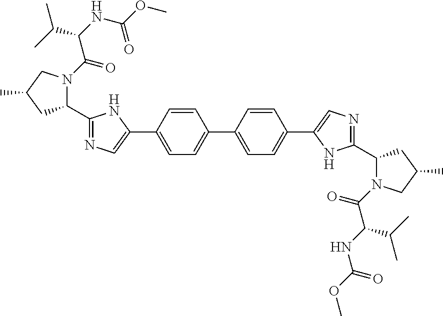

The above compound was prepared according to the procedures disclosed herein.

### EXAMPLE 2

((S)-1-{(S)-2-[5-(7-{2-[(S)-1-((S)-2-(methoxycarbonylamino)-3-methyl-butanoyl]pyrrolidin-2-yl]-3H-imidazol-4-ylethynyl}-benzo[1,3]dioxol-4-ylethynyl)1H-1H-imidazol-2-yl]pyrrolidine-1-carbonyl}-2-methyl-propyl]-carbamic acid methyl ester

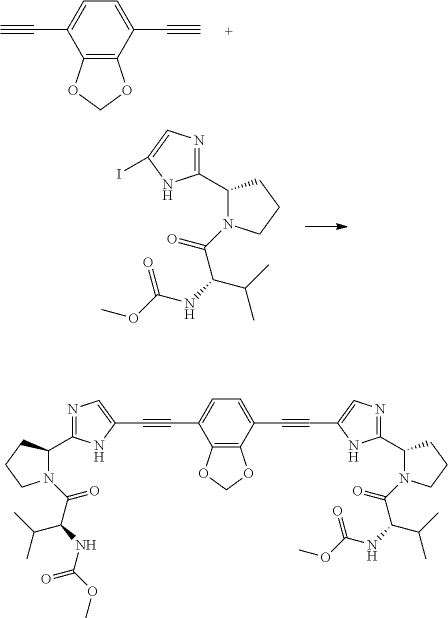 [SMILES: C#Cc1ccc(C#C)c2c1OCO2 ; COC(=O)N[C@H](C(=O)N1CCC[C@H]1c1ncc(I)[nH]1)C(C)C ; COC(=O)N[C@H](C(=O)N1CCC[C@H]1c1ncc(C#Cc2ccc(C#Cc3cnc([C@@H]4CCCN4C(=O)[C@@H](NC(=O)OC)C(C)C)[nH]3)c3c2OCO3)[nH]1)C(C)C]

To a solution of 4,7-diethynyl-1,3-benzodioxole (40.50 mg, 0.24 mmol), methyl N-[(1S)-1-[(2S)-2-(5-iodo-1H-imidazol-2-yl)pyrrolidine-1-carbonyl]-2-methyl-propyl]carbamate (200 mg, 0.47 mmol), Pd(PPh₃)₂Cl₂ (16.7 mg, 0.024 mmol), CuI (4.53 mg, 0.024 mmol) in DMF is added TEA (165.8 μL, 1.19 mmol). The reaction mixture is stirred at 80° C. for 16 hours, diluted with EtOAc and H₂O, and filtered on celite. The organic phase is dried over Na₂SO₄, filtered and concentrated to dryness. The residue is purified by reverse phase flash column chromatography on silica gel (25 to 50% CH₃CN in H₂O) and repurified by reverse phase HPLC using a gradient of CH₃CN/water to give ((S)-1-{(S)-2-[5-(7-{2-[(S)-1-((S)-2-(methoxycarbonylamino)-3-methyl-butanoyl]pyrrolidin-2-yl]-3H-imidazol-4-ylethynyl}-benzo[1,3]dioxol-4-ylethynyl)1H-1H-imidazol-2-yl]pyrrolidine-1-carbonyl}-2-methyl-propyl]-carbamic acid methyl ester (19 mg, 9.4%) as a white solid.

LC/MS: m/z=755.5 (M+H⁺).

### EXAMPLE 3

### Methyl N-[(1S)-1-[(2S)-2-[4-[2-[5-[2-[2-[(2S)-1-[2-(Methoxycarbonylamino)-3-methyl-butanoyl]pyrrolidin-2-yl]-1H-imidazol-4-yl]ethynyl]-2-thienyl]ethynyl]-1H-imidazol-2-yl]pyrrolidine-1-carbonyl]-2-methyl-propyl]carbamate

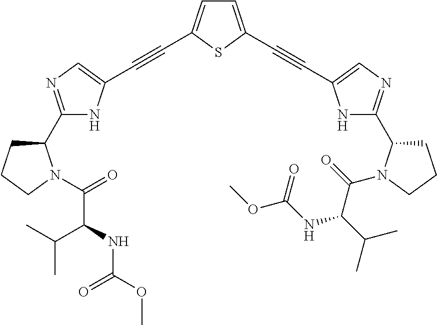 [SMILES: COC(=O)N[C@H](C(=O)N1CCC[C@H]1c1ncc(C#Cc2ccc(C#Cc3cnc([C@@H]4CCCN4C(=O)[C@@H](NC(=O)OC)C(C)C)[nH]3)s2)[nH]1)C(C)C]

The title compound is synthesized from 2,5-diethynyl-thiophene and methyl N-[(1S)-1-[(2S)-2-(5-iodo-1H-imidazol-2-yl)pyrrolidine-1-carbonyl]-2-methyl-propyl]carbamate as described in Example 13.

### EXAMPLE 4A

### ((S)-1-{(2S,4S)-2-[5-(4-{2-[(2S,4S)-1-((S)-2-Methoxycarbonylamino-3-methyl-butyryl)-4-methyl-pyrrolidin-2-yl]-3H-imidazol-4-ylethynyl}-phenylethynyl)-1H-imidazol-2-yl]-4-methyl-pyrrolidine-1-carbonyl}-2-methylpropyl)-carbamic acid methyl ester

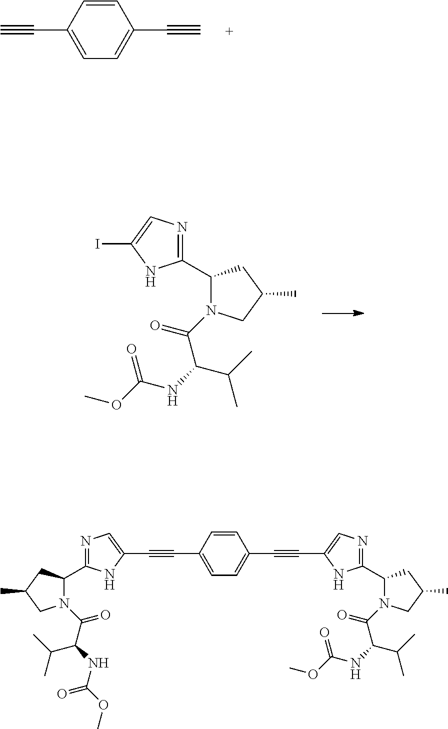

To a stirring solution of 1,4-diethynylbenzene (145.3 mg, 1.152 mmol), methyl N-[(1S)-1-[(2S,4S)-2-(5-iodo-1H-imidazol-2-yl)-4-methyl-pyrrolidine-1-carbonyl]-2-methyl-propyl]carbamate (1000 mg, 2.303 mmol), Pd(dppf)Cl₂-DCM (47.04 mg, 0.05760 mmol), and CuI (21.94 mg, 0.1152 mmol) in DMF (8 mL, degassed) is added triethylamine (582.9 mg, 802.9 μL, 5.760 mmol). The reaction mixture is stirred at 80° C. for 16 hours, diluted with 100 ml of EtOAc and 20 ml of H₂O, and is filtrated on celite. The organic phase is separated, dried over Na₂SO₄, filtered and concentrated to dryness to give 900 mg of brown solid. The residue is purified by flash column chromatography on silica gel (0 to 10% MeOH/Toluene) to afford 400 mg of the title compound with a purity of 85%. The solid is purified by recrystallization in MeOH to afford ((S)-1-{(2S,4S)-2-[5-(4-{2-[(2S,4S)-1-((S)-2-methoxycarbonylamino-3-methyl-butyryl)-4-methyl-pyrrolidin-2-yl]-3H-imidazol-4-ylethynyl}-phenylethynyl)-1H-imidazol-2-yl]-4-methyl-pyrrolidine-1-carbonyl}-2-methylpropyl)-carbamic acid methyl ester (240 mg, 28%) as a white solid.

¹H NMR (400 MHz, cdcl₃, mixture of rotamers), peaks for the major rotamer, δ 10.70 (s, 2H), 7.45-7.35 (m, 6H), 7.215 (d, 2H), 5.45-5.4 (m, 2H), 5.18-5.07 (m, 2H), 4.39-4.29 (m, 2H), 4.05-3.9 (m, 2H), 3.68 (s, 6H), 3.1-3.0 (m, 2H), 2.75-2.2 (m, 4H), 1.95-1.8 (m, 2H), 1.16 (d, 6H), 0.87-0.8 (doublets, 12H).

### EXAMPLE 4B

### ((S)-1-{(2S,4S)-2-[5-(4-{2-[(2S,4S)-1-((S)-2-Methoxycarbonylamino-3-methyl-butyryl)-4-methyl-pyrrolidin-2-yl]-3H-imidazol-4-ylethynyl}-phenylethynyl)-1H-imidazol-2-yl]-4-methyl-pyrrolidine-1-carbonyl}-2-methylpropyl)-carbamic acid methyl ester

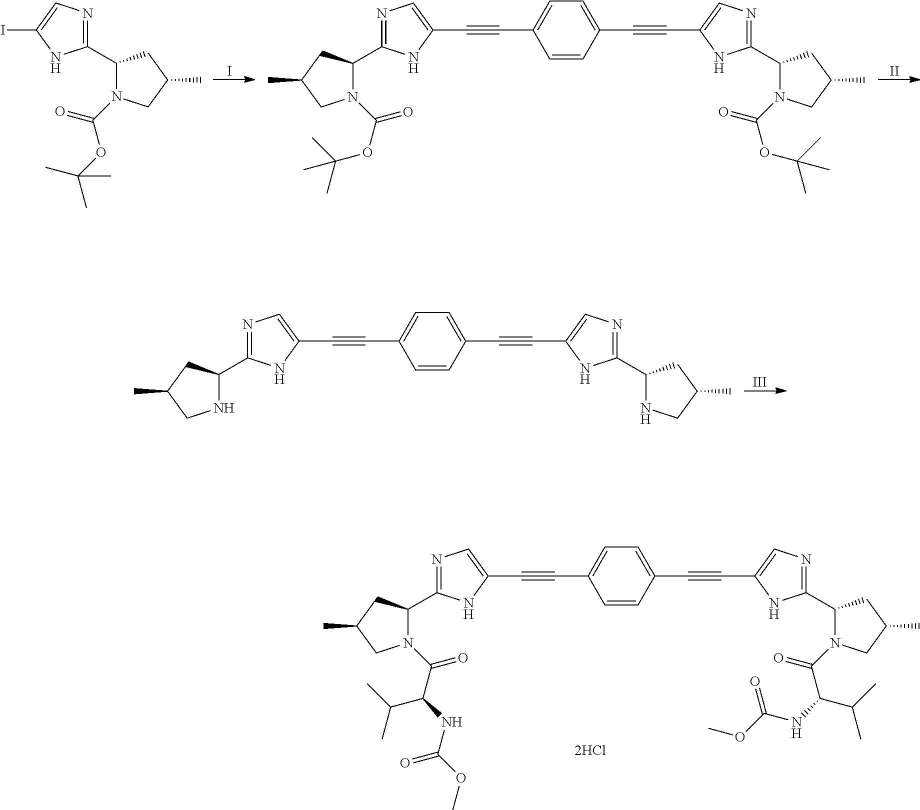

### Step I:

### tert-Butyl (2S,4S)-2-[4-[2-[4-[2-[2-[(2S,4S)-1-tert-butoxycarbonyl-4-methyl-pyrrolidin-2-yl]-1H-imidazol-4-yl]ethynyl]phenyl]ethynyl]-1H-imidazol-2-yl]-4-methyl-pyrrolidine-1-carboxylate

To a stirred suspension of tert-butyl (2S,4S)-2-(5-iodo-1H-imidazol-2-yl)-4-methyl-pyrrolidine-1-carboxylate (1 g, 2.556 mmol), copper Iodide (46.4 mg, 0.24 mmol) and Pd(dppf)Cl₂-DCM (198.8 mg, 0.24 mmol) in DMF (11 mL) is sequentially added TEA (739.2 mg, 1.018 mL, 7.31 mmol) and 1,4-diethynylbenzene (153.5 mg, 1.217 mmol) under nitrogen atmosphere. The reaction mixture is heated at 80° C. for 2 hours, cooled to RT, and diluted with water (10 mL). The reaction mixture is stirred at RT for 30 minutes, and cooled at 0° C. for 15 min. The reaction mixture is filtered to collect a precipitate. The precipitate is purified by flash column chromatography on silica gel (10% MeOH/Toluene) to afford tert-butyl (2S,4S)-2-[4-[2-[4-[2-[2-[(2S,4S)-1-tert-butoxycarbonyl-4-methyl-pyrrolidin-2-yl]-1H-imidazol-4-yl]ethynyl]phenyl]ethynyl]-1H-imidazol-2-yl]-4-methyl-pyrrolidine-1-carboxylate (260 mg, 31.5%, purity, 92%) as solid.

LC/MS, m/z: 625.45 (M+H⁺).

### Step II:

### (S,S)-4,4′-(1,4-phenylenebis(ethyne-2,1-diyl))bis(2-((2S,4S)-4-methylpyrrolidinium-2-yl)-1H-imidazol-3-ium)chloride

To a stirred mixture of tert-butyl (2S,4S)-2-[4-[2-[4-[2-[2-[(2S,4S)-1-tert-butoxycarbonyl-4-methyl-pyrrolidin-2-yl]-1H-imidazol-4-yl]ethynyl]phenyl}ethynyl]-1H-imidazol-2-yl]-4-methyl-pyrrolidine-1-carboxylate (260 mg, 0.4162 mmol) in MeOH (1.3 mL) at 0° C. (using water/ice bath) is added HCl in dioxane (1.457 mL of 4 M, 5.827 mmol) over 2 minutes. The reaction mixture is warmed up to RT and stirred for 19 hours. The reaction mixture is filtered and washed with MTBE to give (S,S)-4,4′-(1,4-phenylenebis(ethyne-2,1-diyl))bis(2-((2S,4S)-4-methylpyrrolidinium-2-yl)-1H-imidazol-3-ium) chloride (192 mg, 95%) as pale brownish powder.

LC/MS, m/z: 425 (M+H⁺).

### Step III:

### Methyl N-[(1S)-1-[(2S,4S)-2-[5-[2-[4-[2-[2-[(2S,4S)-1-[(2S)-2-(methoxycarbonylamino)-3-methyl-butanoyl]-4-methyl-pyrrolidin-2-yl]-1H-imidazol-5-yl]ethynyl]phenyl]ethynyl]-1H-imidazol-2-yl]-4-methyl-pyrrolidine-1-carbonyl]-2-methyl-propyl]carbamate

(2S)-2-(methoxycarbonylamino)-3-methyl-butanoic acid (113.3 mg, 0.6470 mmol), (S,S)-4,4′-(1,4-phenylenebis(ethyne-2,1-diyl))bis(2-((2S,4S)-4-methylpyrrolidinium-2-yl)-1H-imidazol-3-ium)chloride (180 mg, 0.32 mmol), HATU (240 mg, 0.63 mmol), DIPEA (326.3 mg, 439.8 μL, 2.525 mmol) are dissolved in DMF (3.6 mL) at 0° C. The reaction mixture is warmed to RT and stirred overnight. The reaction mixture is diluted with water, stirred for 30 minutes, and filtered to collect a precipitate. The precipitate is purified by flash column chromatography on silica gel (0 to 10% MeOH in toluene) to afford methyl N-[(1S)-1-[(2S,4S)-2-[5-[2-[4-[2-[2-[(2S,4S)-1-[(2S)-2-(methoxycarbonylamino)-3-methyl-butanoyl]-4-methyl-pyrrolidin-2-yl]-1H-imidazol-5-yl]ethynyl]phenyl]ethynyl]-1H-imidazol-2-yl]-4-methyl-pyrrolidine-1-carbonyl]-2-methyl-propyl]carbamate (129 mg, 0.1549 mmol, 49.1%) as a beige solid. LC/MS, m/z: 739.3 (M+H⁺).

### EXAMPLE 4C

### ((S)-1-{(2S,4S)-2-[5-(4-{2-[(2S,4S)-1-((S)-2-Methoxycarbonylamino-3-methyl-butyryl)-4-methyl-pyrrolidin-2-yl]-3H-imidazol-4-ylethynyl}-phenylethynyl)-1H-imidazol-2-yl]-4-methyl-pyrrolidine-1-carbonyl}-2-methylpropyl)-carbamic acid methyl ester

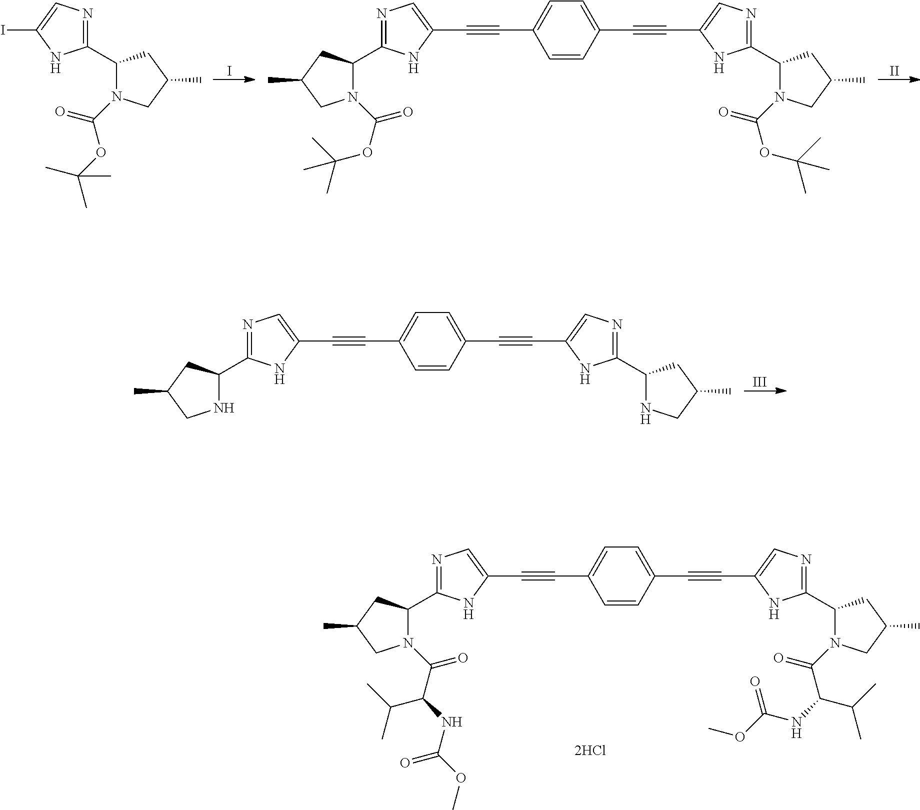

### Step I:

In a 3 neck, 5 L RBF equipped with a thermocouple and a stirring bar, tert-butyl (2S,4S)-2-(5-iodo-1H-imidazol-2-yl)-4-methyl-pyrrolidine-1-carboxylate (400 g, 996.8 mmol), the trimethyl-[2-[4-(2-trimethylsilylethynyl)phenyl]ethynyl]silane (137.5 g, 498.2 mmol), the PdCl₂(dppf)₂-CH₂Cl₂ (20.34 g, 24.91 mmol) and copper Iodide (4.744 g, 24.91 mmol) are added under nitrogen atmosphere. DMF (2 L) is added and the suspension is stirred and then degassed twice (vacuum 5 minutes followed by bubbled stream of nitrogen gas for 5 minutes). DBU (834.3 g, 819.5 mL, 5.480 mol) is added over 1 minute. The solution is degassed twice (vacuum 5 minutes followed by bubbled stream of nitrogen gas for 5 minutes) and degassed water (13.46 g, 13.46 mL, 747.3 mmol) is added. The stirring is switched from magnetic to mechanical under N₂ atmosphere. The reaction mixture is heated to 60° C. and then degassed water (13.46 g, 13.46 mL, 747.3 mmol) is added. The reaction is followed by HPLC. After 4 hours the reaction mixture is transferred in a 22 L reactor with stirring and water (18 L) is added over 60 minutes (exotherm of 10° C. is observed). The reaction mixture is cooled to below 5° C. in an ice bath and stirred for 30 minutes. The solid is filtered and washed with water (5 L) and heptane (4 L). The solid is dried in a vacuum oven at 35° C. overnight. The wet solid is dissolved in CH₂Cl₂/MeOH 3 L; 90/10) and stirred for 20 minutes. The phases are separated and the aqueous phase is re-extracted with CH₂Cl₂/MeOH (500 ml; 90/10). The phases are separated, the organic phases are merged and silica gel (800 mL) is added to the solution and then it is evaporated to dryness. The residue is purified by a plug of silica gel using 1 to 10% MeOH/CH₂Cl₂ as eluant. The selected fractions are concentrated to a thick paste and stirred in an ice bath for 30 minutes and then filtered to afford tert-butyl (2S,4S)-2-[4-[2-[4-[2-[2-[(2S,4S)-1-tert-butoxycarbonyl-4-methyl-pyrrolidin-2-yl]-1H-imidazol-4-yl]ethynyl]phenyl}ethynyl]-1H-imidazol-2-yl]-4-methyl-pyrrolidine-1-carboxylate (234 g, 366.6 mmol, 73.57% yield) as a pale yellow solid; 97.8% pure.

### Step II:

In a 3 neck, 5 L RBF equipped with a mechanical stirrer, a temperature controller J-Kem model 260 and a heating mantel, tert-butyl (2S,4S)-2-[5-[2-[4-[2-[2-[(2S,4S)-1-tert-butoxycarbonyl-4-methyl-pyrrolidin-2-yl]-1H-imidazol-5-yl]ethynyl]phenyl]ethynyl]-1H-imidazol-2-yl]-4-methyl-pyrrolidine-1-carboxylate (234.5 g, 367.1 mmol) and methanol (1.834 L) are added while stirring. Charcoal (233.7 g, 19.46 mol) is added and the suspension is heated at reflux for 1.5 hours. Celite (100 g) is added and the mixture is stirred for 5 minutes and filtered through celite and washed with methanol. The solution is evaporated to 1 L and transferred in a 3 neck, 5 L RBF equipped with a mechanical stirrer, the solution is cooled in an ice bath. HCl in Dioxane (1.285 L of 4 M, 5.139 mol) is added over 35 minutes while keeping the internal temperature below 15° C. At the end of the addition, an off white solid is started to form and it becomes thicker, the reaction is slowly warmed to rt and followed by HPLC. After 5.5 hours, the suspension becomes very thick. MTBE (917.2 mL) is added and the mixture is stirred for 1 hour at rt, filtered and washed with MTBE. The solid is dried in a vacuum oven at 35° C. for 2 days to afford (S,S)-4,4′-(1,4-phenylenebis(ethyne-2,1-diyl))bis(2-((2S,4S)-4-methylpyrrolidinium-2-yl)-1H-imidazol-3-ium)chloride (206.9 g, 360.9 mmol, 98.31%) 99.5% pure as a yellow powder.

### Step III:

In a 12 L 4 neck RBF under nitrogen equipped with mechanical stirrer, a thermocouple and an addition funnel, (S,S)-4,4′-(1,4-phenylenebis(ethyne-2,1-diyl))bis(2-((2S,4S)-4-methylpyrrolidinium-2-yl)-1H-imidazol-3-ium)chloride (196.27 g, 342.4 mmol), the (2S)-2-(methoxycarbonylamino)-3-methyl-butanoic acid (120.0 g, 684.8 mmol) and HATU (260.4 g, 684.8 mmol) are added. The powder is stirred under nitrogen and DMF (1.953 L) is added, the solution is then cooled to 3° C. using an ice/water bath. N,N-diisopropylethylamine (354.0 g, 477.1 mL, 2.739 mol) is added dropwise over 80 minutes while keeping the internal temperature at 4±2° C. The reaction mixture is stirred for 1.5 hours and followed by HPLC/MS that shows that the reaction is completed. Cold water (5.859 L) is added dropwise over 90 minutes while keeping the internal temperature below 8.5° C. The suspension is stirred for 1 hour in an ice bath, filtered and washed with cold water (600 ml) and then with cold heptane (400 ml). The crude product is dried overnight at 45° C. in vacuum oven. The crude powder (253 g) is transferred in a 12 L 5 neck RBF equipped with a mechanical stirrer, a condenser, a temperature controller J-Kem model 260 and a heating mantel. Methanol (6 L) is added and the mixture is heated at reflux until the compound is dissolved. The hot solution is filtered and washed with methanol (500 mL). The solution is transferred in a 12 L 5 neck RBF equipped with a mechanical stirrer, a temperature controller J-Kem model 260, a heating mantel and a distillation head. Methanol (4 L) is distilled off while stirring. The heating is turned off and the solution is slowly brought back to room temperature and slowly crystallized out overnight. The suspension is cooled for 30 minutes in an ice bath, filtered on a large Buchner and washed with cold methanol (200 mL). The cake is dried in a vacuum oven at 35° C. for 3 days to give methyl N-[(1S)-1-[(2S,4S)-2-[5-[2-[4-[2-[2-[(2S,4S)-1-[(2S)-2-(methoxycarbonylamino)-3-methyl-butanoyl]-4-methyl-pyrrolidin-2-yl]-1H-imidazol-5-yl]ethynyl]phenyl]ethynyl]-1H-imidazol-2-yl]-4-methyl-pyrrolidine-1-carbonyl]-2-methyl-propyl]carbamate (172 g, 228.5 mmol, 66.73% yield) as an off white powder 98.16% pure by HPLC.

### EXAMPLE 5

### Methyl N-[(1S)-2-methyl-1-[(2S,4S)-4-methyl-2-[4-[2-[2,3,5,6-tetradeuterio-4-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]phenyl}ethynyl]-1H-imidazol-2-yl]pyrrolidine-1-carbonyl]-2-methyl-propyl]carbamate

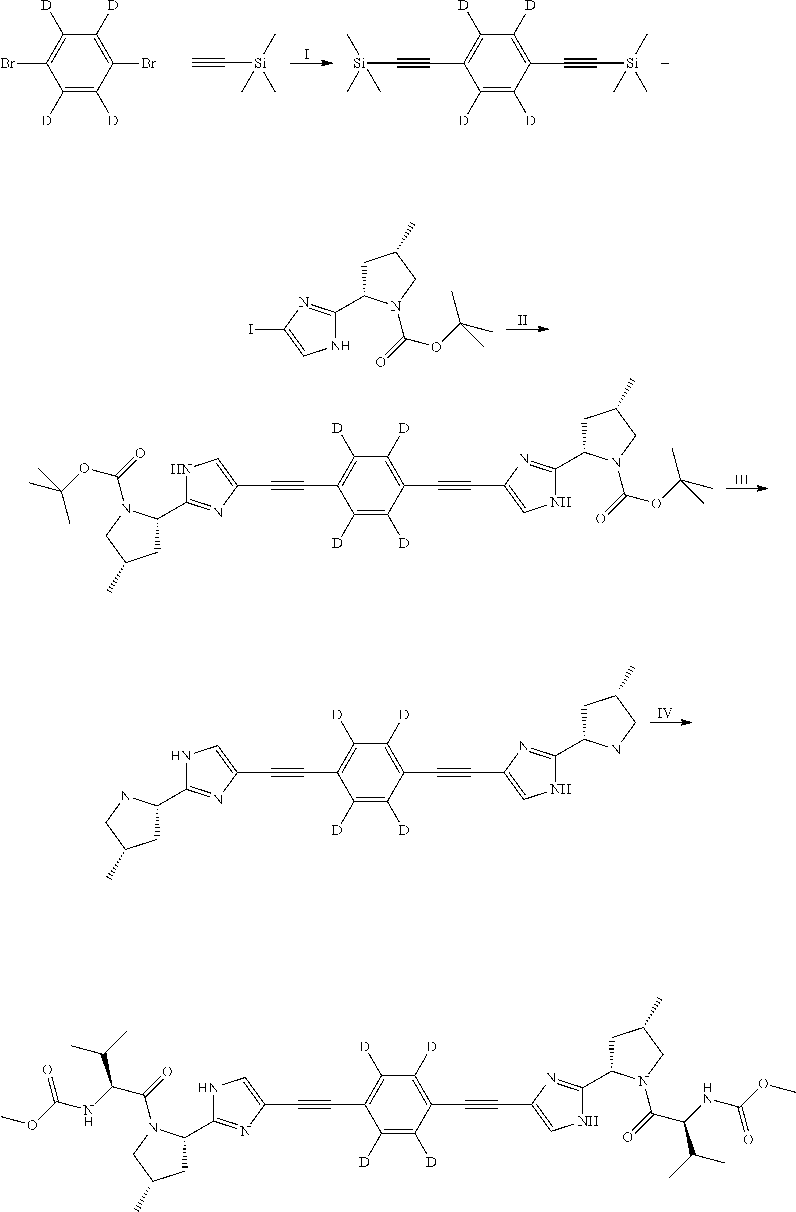

### Step I

To a solution of 1,4-dibromo-2,3,5,6-tetradeuterio-benzene (500 mg, 2.084 mmol) in DMF (10 mL) are added PdCl₂(dppf)-CH₂Cl₂ (85.09 mg, 0.1042 mmol) and CuI (19.84 mg, 0.1042 mmol). After degassing, DIPEA (1.077 g, 1.451 mL, 8.336 mmol) and ethynyl-trimethyl-silane (614.1 mg, 883.6 μL, 6.252 mmol) are added to the reaction mixture. Then the solution is heated at 45° C. under nitrogen overnight. After removal of the solvent under reduced pressure, the residue is purified by silica gel column chromatography using hexane to provide trimethyl-[2-[2,3,5,6-tetradeuterio-4-(2-trimethylsilylethynyl)phenyl]ethynyl]silane (360 mg, 1.311 mmol, 62.93%) as yellow solid. ¹H NMR (CD₃OD, 400 MHz): 0.00 (m, 9H).

### Step II

To a solution of tert-butyl (2S,4S)-2-(4-iodo-1H-imidazol-2-yl)-4-methyl-pyrrolidine-1-carboxylate (288.5 mg, 0.7648 mmol) in DMF (3 mL) are added trimethyl-[2-[2,3,5,6-tetradeuterio-4-(2-trimethylsilylethynyl)phenyl]ethynyl]silane (100 mg, 0.3642 mmol), PdCl₂(dppf)-CH₂Cl₂ (14.87 mg, 0.01821 mmol), CuI (3.468 mg, 0.01821 mmol) and DBU (665.3 mg, 653.5 μL, 4.370 mmol). After degassing, water (19.69 mg, 19.69 μL, 1.093 mmol) is added to the reaction mixture. Then the solution is heated at 60° C. under nitrogen overnight. After removal of the solvent under reduced pressure, the residue is purified by silica gel column chromatography using MeOH (0-5%) in CH₂Cl₂ to provide tert-butyl (2S,4S)-2-[4-[2-[4-[2-[2-[(2S,4S)-1-tert-butoxycarbonyl-4-methyl-pyrrolidin-2-yl]-1H-imidazol-4-yl]ethynyl]-2,3,5,6-tetradeuterio-phenyl]ethynyl]-1H-imidazol-2-yl]-4-methyl-pyrrolidine-1-carboxylate (141 mg, 0.2242 mmol, 61.57%) as a off-white solid. LC/MS: m/z=629.20 (M+H⁺).

### Step III

To a solution of tert-butyl (2S,4S)-2-[4-[2-[4-[2-[2-[(2S,4S)-1-tert-butoxycarbonyl-4-methyl-pyrrolidin-2-yl]-1H-imidazol-4-yl]ethynyl]-2,3,5,6-tetradeuterio-phenyl]ethynyl]-1H-imidazol-2-yl]-4-methyl-pyrrolidine-1-carboxylate (196 mg, 0.3117 mmol) in 3 mL of methanol is added HCl/dioxane (779.2 μL of 4 M, 3.117 mmol). The mixture is stirred at rt for 1 hour and then concentrated to dryness. The residue is used in the next step without any further purification.

### Step IV

To a solution of 2-[(2S,4S)-4-methylpyrrolidin-2-yl]-4-[2-[2,3,5,6-tetradeuterio-4-[2-[2-[(2S,4S)-4-methylpyrrolidin-2-yl]-1H-imidazol-4-yl]ethynyl]phenyl]ethynyl]-1H-imidazole (hydrochloric acid (4)) (179 mg, 0.3116 mmol) HCl salt in DMF (5 mL) are added (2S)-2-(methoxycarbonylamino)-3-methyl-butanoic acid (136.5 mg, 0.7790 mmol), HATU (296.2 mg, 0.7790 mmol) and DIPEA (402.7 mg, 542.7 μL, 3.116 mmol). The mixture is stirred at rt overnight. After removal of the solvent under reduced pressure, the residue is purified twice by silica gel column chromatography using MeOH (0-6%) in CH₂Cl₂ to provide methyl N-[(1S)-2-methyl-1-[(2S,4S)-4-methyl-2-[4-[2-[2,3,5,6-tetradeuterio-4-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]phenyl]ethynyl]-1H-imidazol-2-yl]pyrrolidine-1-carbonyl]-2-methyl-propyl]carbamate (184 mg, 0.2353 mmol, 75.51%) as an off-white solid. LC/MS: m/z=743.58 (M+W). ¹H NMR (CD₃OD, 400 MHz): δ 7.15-7.24 (d, 2H), 4.95 (m, 2H), 4.20 (m, 4H), 3.62 (s, 6H), 2.46 (m, 4H), 1.96 (m, 4H), 1.15-1.30 (m, 8H), 0.84 (m, 12H).

### EXAMPLE 6

### Methyl N-[(1S)-1-[(2S,4S)-2-[4-[2-[6-[2-[2-[(2S,4S)-1-[(2S)-2-(methoxycarbonylamino)-3-methyl-butanoyl]-4-methyl-pyrrolidin-2-yl]-1H-imidazol-3-ium-4-yl]ethynyl]-2-naphthyl]ethynyl]-1H-imidazol-3-ium-2-yl]-4-methyl-pyrrolidine-1-carbonyl]-2-methyl-propyl]carbamate as HCl salt

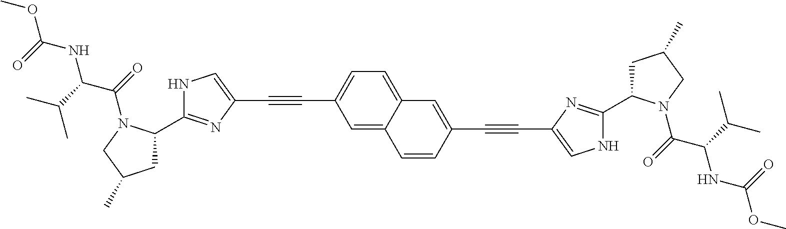

To a stirred suspension of methyl N-[(1S)-1-[(2S,4S)-2-(5-iodo-1H-imidazol-2-yl)-4-methyl-pyrrolidine-1-carbonyl]-2-methyl-propyl]carbamate, CuI and Pd(dppf)Cl₂-DCM in DMF is sequentially added TEA and 2,6-diethynylnaphthalene under nitrogen atmosphere. The reaction mixture is heated at 70° C. for 3 hours in a sealed tube, and is concentrated to dryness. The residue is purified by flash column chromatography on silica gel (0 to 15% MeOH in EtOAc) to give a mixture of diastereomers. The mixture is purified by reverse phase HPLC using a gradient of CH₃CN/water to afford the title compound as HCl salt.

¹H NMR (400 MHz, CD₃OD) δ 8.11 (s, 2H), 7.925 (d, 2H), 7.67 (s, 2H), 7.61 (d, 2H), 5.09 (dd, 2H), 4.29 (t, 2H), 4.18 (d, 2H), 3.64 (s, 6H), 3.4-3.25 (m, 2H), 2.65-2.4 (m, 4H), 2.05-1.75 (m, 4H), 1.208 (d, 6H), 0.88 (d, 6H), 0.86 (d, 6H).

LC/MS: m/z=789.61 (M+H⁺).

### EXAMPLE 7

### Dimethyl (2S,2′S,3R,3′R)-1,1′-((3S,3′S,5S,5′S)-5,5′-(4,4′-(1,4-phenylenebis(ethyne-2,1-diyl))bis(1H-imidazole-4,2-diyl))bis(3-methylpyrrolidine-5,1-diyl))bis(3-methoxy-1-oxobutane-2,1-diyl)dicarbamate

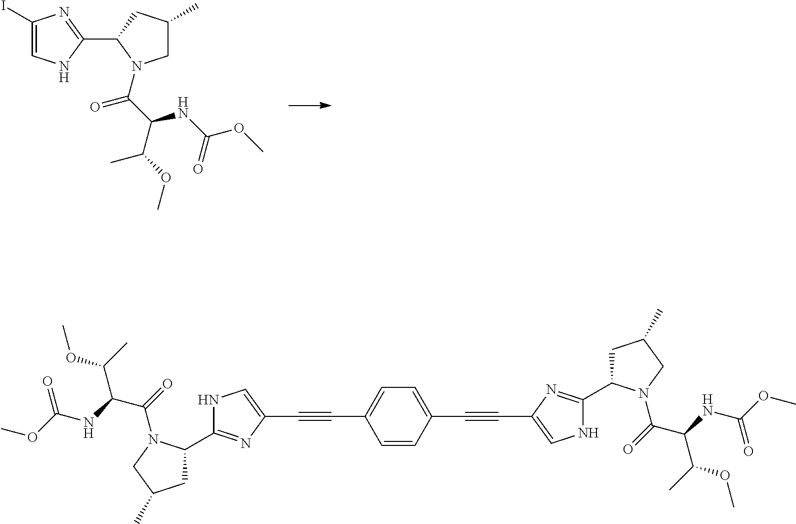

To a stirred suspension of methyl N-[(1S,2R)-1-[(2S,4S)-2-(4-iodo-1H-imidazol-2-yl)-4-methyl-pyrrolidine-1-carbonyl]-2-methoxy-propyl]carbamate (99.96 mg, 0.2220 mmol), CuI (4.228 mg, 0.02220 mmol) and Pd(dppf)Cl₂-DCM (18.13 mg, 0.02220 mmol) in DMF (1.5 mL) is sequentially added TEA (67.39 mg, 92.82 μL, 0.6660 mmol) and 1,4-diethynylbenzene (14 mg, 0.1110 mmol) under nitrogen atmosphere. The reaction mixture is heated at 70° C. for 3 hours, diluted with water (10 mL) and extracted with CH₂Cl₂ (3×10 mL). The combined extracts are washed with water, brine, dried over Na₂SO₄, filtered, and concentrated to dryness. The residue is purified by flash column chromatography on silica gel (5 to 25% MeOH in toluene) and is repurified by reverse phase HPLC using a gradient of CH₃CN/water to afford dimethyl (2S,2′S,3R,3′R)-1,1′-((3S,3′S,5S,5′S)-5,5′-(4,4′-(1,4-phenylenebis(ethyne-2,1-diyl))bis(1H-imidazole-4,2-diyl))bis(3-methylpyrrolidine-5,1-diyl))bis(3-methoxy-1-oxobutane-2,1-diyl)dicarbamate (11.4 mg, 0.01321 mmol, 11.90%) as HCl salt.

Rf=0.42 (MeOH-Toluene, 1:4),

¹H NMR (400 MHz, CD₃OD) δ 7.64 (s, 2H), 7.50 (s, 4H), 5.03 (dd, 2H), 4.35 (d, 2H), 4.26-4.1 (m, 2H), 3.6-3.4 (m, 2H), 3.56 (s, 6H), 3.3-3.1 (m, 1H), 3.2 (s, 6H), 2.6-2.3 (m, 4H), 1.75-1.55 (m, 2H), 1.11 (d, 6H), 0.99 (d, 6H).

LC/MS: m/z=771.58 (M+H⁺).

### EXAMPLE 8

### Dimethyl (2S,2′S)-1,1′-((3S,3′S,5S,5′S)-5,5′-(4,4′-(2,5-dimethyl-1,4-phenylene)bis(ethyne-2,1-diyl)bis(1H-imidazole-4,2-diyl))bis(3-methylpyrrolidine-5,1-diyl))bis(3-methyl-1-oxobutane-2,1-diyl)dicarbamate

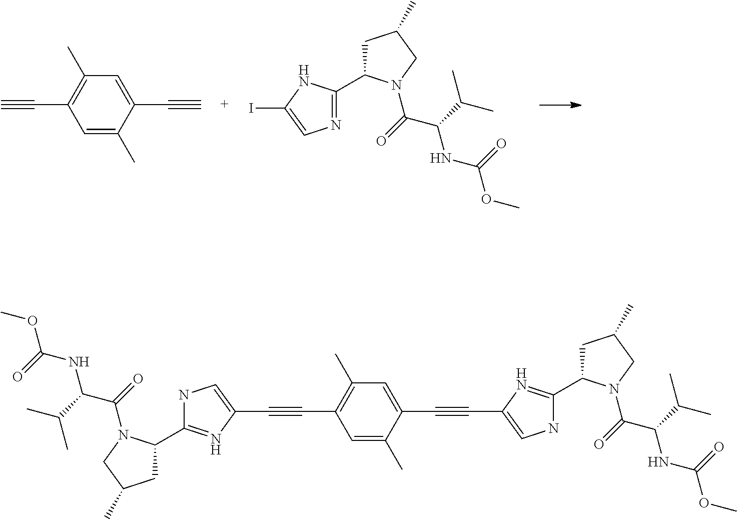

To a stirred suspension of methyl N-[(1S)-1-[(2S,4S)-2-(5-iodo-1H-imidazol-2-yl)-4-methyl-pyrrolidine-1-carbonyl]-2-methyl-propyl]carbamate (73.22 mg, 0.1686 mmol), CuI (3.211 mg, 0.01686 mmol) and Pd(dppf)Cl₂-DCM (13.77 mg, 0.01686 mmol) in DMF (1.144 mL) is sequentially added TEA (70.50 μL, 0.5058 mmol) and 1,4-diethynyl-2,5-dimethyl-benzene (13 mg, 0.08430 mmol) under nitrogen atmosphere. The reaction mixture is heated at 50° C. overnight, diluted with water (10 mL), and a precipitate is formed. The precipitate is filtered, washed with water, and dried under high vacuum to afford the title compound (65 mg) which is converted into HCl salt using 4N HCl in dioxane. The mixture is freeze dried and purified by reverse phase HPLC using a gradient of CH₃CN/water to afford dimethyl (2S,2′S)-1,1′-((3S,3′S,5S,5′S)-5,5′-(4,4′-(2,5-dimethyl-1,4-phenylene)bis(ethyne-2,1-diyl)bis(1H-imidazole-4,2-diyl))bis(3-methylpyrrolidine-5,1-diyl))bis(3-methyl-1-oxobutane-2,1-diyl)dicarbamate as HCl salt (3.9 mg, 0.004276 mmol, 5.072%) as light yellow solid.

Rf=0.29 (MeOH-Toluene, 1:9).

¹H NMR (400 MHz, CD₃OD) δ 7.79 (s, 2H), 7.44 (s, 2H), 5.10 (dd, 2H), 4.30 (t, 2H), 4.17 (d, 2H), 3.63 (s, 6H), 3.39-3.31 (m, 2H), 2.45 (s, 6H), 2.65-2.45 (m, 4H), 2.04-1.90 (m, 2H), 1.8-1.7 (m, 2H), 1.20 (d, 6H), 0.87 (d, 6H), 0.85 (d, 6H).

LC/MS: m/z=767.59 (M+H⁺).

### EXAMPLE 9

### Dimethyl (2S,2′S)-1,1′-((3R,3′R,5S,5′S)-5,5′-(4,4′-(1,4-phenylenebis-(ethyne-2,1-diyl))bis(1H-imidazole-4,2-diyl))bis(3-methylpyrrolidine-5,1-diyl))bis(3-methyl-1-oxobutane-2,1-diyl)dicarbamate

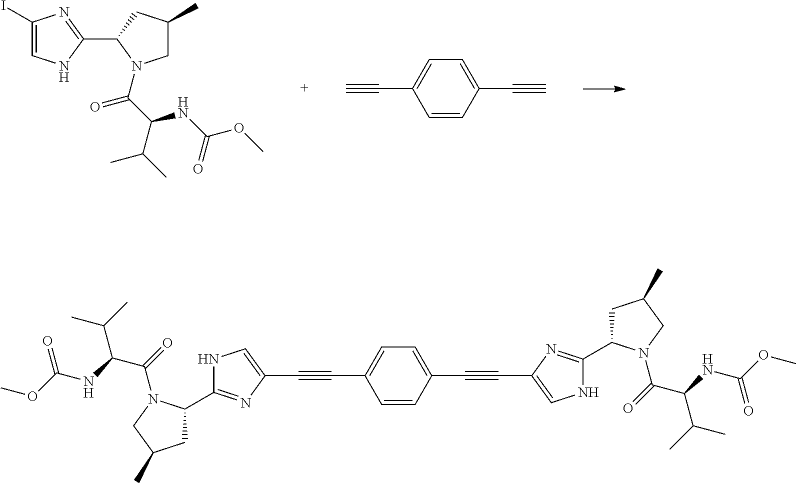

To a stirred suspension of methyl (S)-1-((2S,4R)-2-(4-iodo-1H-imidazol-2-yl)-4-methylpyrrolidin-1-yl)-3-methyl-1-oxobutan-2-ylcarbamate (96.41 mg, 0.21 mmol), CuI (4.23 mg, 0.022 mmol) and Pd(dppf)Cl₂-DCM (18.13 mg, 0.022 mmol) in DMF (1.5 mL) is sequentially added TEA (92.82 μL, 0.67 mmol) and 1,4-diethynylbenzene (14 mg, 0.11 mmol) under nitrogen atmosphere. The reaction mixture is heated at 70° C. for 3 hours, diluted with water (6 mL), and a precipitate is filtered off. The precipitate is dissolved in CH₂Cl₂, filtered through a pad of celite, and concentrated to dryness. The residue is purified by flash column chromatography on silica gel (5 to 10% MeOH in toluene) to afford the title compound (43 mg) which is converted into its HCl salt and purified by reverse phase HPLC using a gradient of CH₃CN/water to afford dimethyl (2S,2′S)-1,1′-((3R,3′R,5S,5′S)-5,5′-(4,4′-(1,4-phenylenebis-ethyne-2,1-diyl))bis(1H-imidazole-4,2-diyl))bis(3-methylpyrrolidine-5,1-diyl))bis(3-methyl-1-oxobutane-2,1-diyl)dicarbamate as HCl salt (17.1 mg, 0.0199 mmol, 17.93%) as white solid.

¹H NMR (400 MHz, CD₃OD) δ 7.76 (s, 2H), 7.60 (s, 4H), 5.25 (dd, 2H), 4.17 (d, 2H), 4.02-3.86 (m, 2H), 3.64 (s, 6H), 3.61-3.48 (m, 2H), 2.7-2.58 (m, 2H), 2.28-1.99 (m, 6H), 1.14 (d, 6H), 0.91 (d, 6H), 0.89 (d, 6H).

LC/MS: m/z=739.5 (M+H⁺).

### EXAMPLE 10

### Methyl N-[(1S)-1-[(2S,4S)-2-[5-[2-[5-[2-[2-[(2S,4S)-1-[(2S)-2-(methoxycar-bonylamino)-3-methyl-butanoyl]-4-methyl-pyrrolidin-2-yl]-1H-imidazol-5 yl]ethynyl]thieno[3,2-b]thiophen-2-yl]ethynyl]-1H-imidazol-2-yl]-4-methyl-pyrrolidine-1-carbonyl]-2-methyl-propylicarbamate

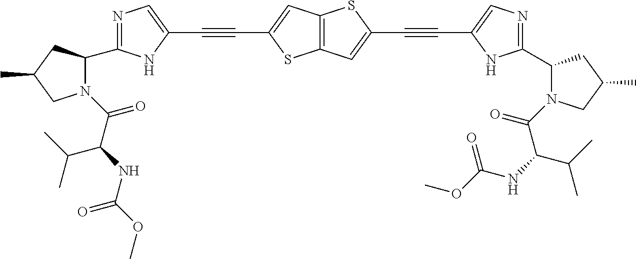

The title compound is synthesized from 2,5-Diethynyl-thieno[3,2-b]thiophene and methyl N-[(1S)-1-[(2S,4S)-2-(5-iodo-1H-imidazol-2-yl)-4-methyl-pyrrolidine-1-carbonyl]-2-methyl-propyl]carbamate as described in Example 4A.

LC/MS: m/z=801.5 (M+W).

### EXAMPLE 11

### Methyl N-[(1S)-1-[(2S,4S)-2-[4-[2-[4-[2-[2-[(2S,4S)-1-[(2S)-2-[methoxycarbonyl(methyl)amino]-3-methyl-butanoyl]-4-methyl-pyrrolidin-2-yl]-1H-imidazol-4-yl]ethynyl]phenyl}ethynyl]-1H-imidazol-2-yl]-4-methyl-pyrrolidine-1-carbonyl]-2-methyl-propyl]-N-methyl-carbamate

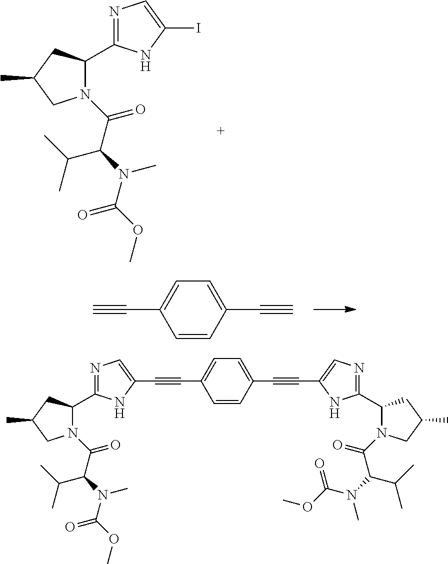

To a stirred suspension of methyl N-[(1S)-1-[(2S,4S)-2-(5-iodo-1H-imidazol-2-yl)-4-methyl-pyrrolidine-1-carbonyl]-2-methyl-propyl]-N-methyl-carbamate (75 mg, 0.17 mmol), CuI (3.18 mg, 0.017 mmol) and Pd(dppf)Cl₂-DCM (13.6 mg, 0.017 mmol) in DMF (1.2 mL) is sequentially added TEA (70 μL, 0.50 mmol) and 1,4-diethynylbenzene (10.55 mg, 0.083 mmol) under nitrogen atmosphere. The reaction mixture is heated at 70° C. for 1 hour, diluted with water and extracted with CH₂Cl₂. The organic phase is washed with water, brine, dried over Na₂SO₄, filtered and concentrated to dryness. The residue is purified by flash column chromatography on silica gel (1 to 30% MeOH in CH₂Cl₂) and purified by reverse phase HPLC using a gradient of CH₃CN/water to give methyl N-[(1S)-1-[(2S,4S)-2-[4-[2-[4-[2-[2-[(2S,4S)-1-[(2S)-2-[methoxycarbonyl(methyl)amino]-3-methyl-butanoyl]-4-methyl-pyrrolidin-2-yl]-1H-imidazol-4-yl]ethynyl]phenyl]ethynyl]-1H-imidazol-2-yl]-4-methyl-pyrrolidine-1-carbonyl]-2-methyl-propyl]-N-methyl-carbamate.

¹H NMR (400 MHz, CD₃OD) δ 7.65 (m, 2H), 7.47 (s, 4H), 4.95 (m, 2H), 4.43 (d, 2H), 4.29 (m, 1H), 4.19-4.14 (m, 2H), 4.0-3.8 (m, 1H), 3.64 (s, 6H), 2.74 (s, 6H), 2.57-2.46 (m, 2H), 2.33-2.27 (m, 2H), 2.07-2.01 (m, 2H), 1.70-1.62 (m, 2H), 1.08 (d, 6H), 0.84 (dd, 6H), 0.70 (dd, 6H).

LC/MS: m/z=767.46 (M+H⁺).

### EXAMPLE 12

### Methyl N-[(1S)-1-[(2S,4S)-2-[4-[2-[4-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]phenyl}ethynyl]-1H-imidazol-2-yl]-4-methyl-pyrrolidine-1-carbonyl]-2-methyl-propyl]-N-methyl-carbamate

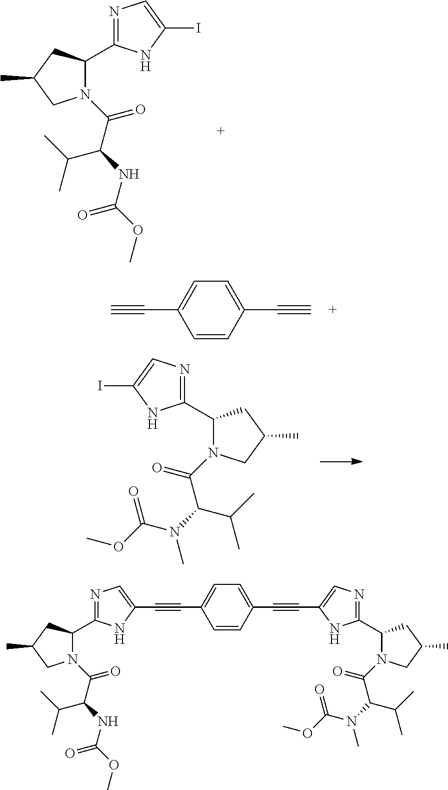

To a stirred suspension of methyl N-[(1S)-1-[(2S,4S)-2-(5-iodo-1H-imidazol-2-yl)-4-methyl-pyrrolidine-1-carbonyl]-2-methyl-propyl]carbamate (100 mg, 0.23 mmol), methyl N-[(1S)-1-[(2S,4S)-2-(5-iodo-1H-imidazol-2-yl)-4-methyl-pyrrolidine-1-carbonyl]-2-methyl-propyl]-N-methyl-carbamate (103.2 mg, 0.23 mmol), CuI (8.7 mg, 0.04 mmol) and Pd(dppf)Cl₂-DCM (37.6 mg, 0.04 mmol) in DMF (3 mL) is sequentially added TEA (192.6 μL, 1.38 mmol) and 1,4-diethynylbenzene (29 mg, 0.23 mmol) under nitrogen atmosphere. The reaction mixture is heated at 70° C. for 1 hour, diluted with water and extracted with CH₂Cl₂. The organic phase is washed with water, brine, dried over Na₂SO₄, filtered and concentrated to dryness. The residue is purified by flash column chromatography on silica gel (1 to 30% MeOH in CH₂Cl₂) and purified by reverse phase HPLC using a gradient of CH₃CN/water to give methyl N-[(1S)-1-[(2S,4S)-2-[4-[2-[4-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]phenyl]ethynyl]-1H-imidazol-2-yl]-4-methyl-pyrrolidine-1-carbonyl]-2-methyl-propyq-N-methyl-carbamate as a white powder.

¹H NMR (400 MHz, CD₃OD) δ 7.55 (m, 2H), 7.47 (s, 4H), 5.00-4.92 (m, 2H), 4.42 (d, 1H), 4.41-4.14 (m, 2H), 4.22 (m, 1H), 4.08 (d, 1H), 3.99 (m, 1H), 3.64 (s, 3H), 3.54 (s, 3H), 2.74 (s, 3H), 2.55-2.44 (m, 2H), 2.34 (m, 2H), 2.03 (m, 1H), 1.87 (m, 1H), 1.67 (m, 2H), 1.10-1.04 (m, 6H), 0.79-0.69 (m, 12H).

LC/MS: m/z=753.4 (M+H⁺).

### EXAMPLE 13

### Methyl N-[(1S)-1-[(2S)-2-[4-[2-[4-[2-[2-[(2S)-1-[(2S)-2-(methoxycarbonylamino)-3-methyl-butanoyl]pyrrolidin-2-yl]-1H-imidazol-4-yl]ethynyl]-1,3-benzoxazol-7-yl]ethynyl]-1H-imidazol-2-yl]pyrrolidine-1-carbonyl]-2-methyl-propyl]carbamate

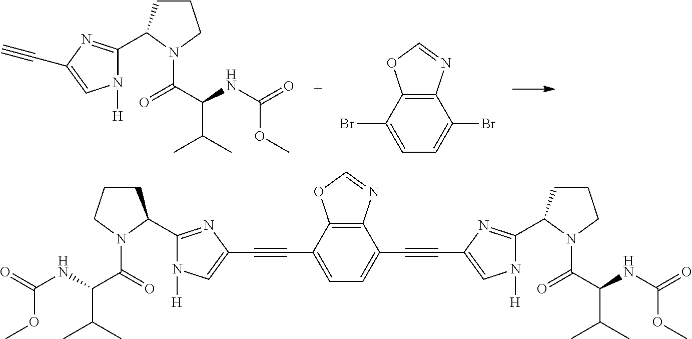 [SMILES: C#Cc1c[nH]c([C@@H]2CCCN2C(=O)[C@@H](NC(=O)OC)C(C)C)n1 ; Brc1ccc(Br)c2ocnc12 ; COC(=O)N[C@H](C(=O)N1CCC[C@H]1c1nc(C#Cc2ccc(C#Cc3c[nH]c([C@@H]4CCCN4C(=O)[C@@H](NC(=O)OC)C(C)C)n3)c3ocnc23)c[nH]1)C(C)C]

4,7-Dibromo-1,3-benzoxazole (19 mg, 0.07 mmol), methyl N-[(1S)-1-[(2S)-2-(4-ethynyl-1H-imidazol-2-yl)pyrrolidine-1-carbonyl]-2-methyl-propyl]carbamate (56 mg, 0.17 mmol), Pd(dppf)Cl₂-DCM (5.7 mg, 0.007 mmol), TEA (19.6 uL, 0.14 mmol) and CuI (2.6 mg, 0.014 mmol) are dissolved in 3 mL of DMF. The system is flushed with nitrogen and the mixture is heated at 70° C. overnight. After evaporation of the solvent under vacuum, the residue is purified by flash column chromatography on silica gel (0 to 7% MeOH in CH₂Cl₂) and is further purified by reverse phase HPLC using a gradient of CH₃CN/water to give methyl N-[(1S)-1-[(2S)-2-[4-[2-[4-[2-[2-[(2S)-1-[(2S)-2-(methoxycarbonylamino)-3-methyl-butanoyl]pyrrolidin-2-yl]-1H-imidazol-4-yl]ethynyl]-1,3-benzoxazol-7-yl]ethynyl]-1H-imidazol-2-yl]pyrrolidine-1-carbonyl]-2-methyl-propyl]carbamate (10.4 mg).

¹H NMR (400 MHz, CD₃OD): δ [ppm] 8.58 (s, 1H), 7.38-7.50 (m, 4H), 5.08 (m, 2H), 4.18 (d, 2H), 3.80-3.94 (m, 4H), 3.55 (s, 6H), 1.99-2.26 (m, 10H), 0.86-0.97 (m, 12H).

LC/MS: m/z 752.58 (M+H⁺).

¹H NMR (400 MHz, CD₃OD) δ 7.29 (m, 2H), 7.12 (m, 4H), 5.05 (m, 2H), 4.17 (d, 2H), 3.98-3.92 (m, 2H), 3.82-3.78 (m, 2H), 3.62 (s, 6H), 2.24 (m, 4H), 2.12 (m, 2H), 2.03-1.97 (m, 4H), 1.3-1.27 (m, 6H), 0.95-0.85 (m, 12H).

LC/MS: m/z=717.5 (M+H⁺).

### EXAMPLES 14 AND 15

### methyl N-[(1S)-1-[(2S)-2-[4-[2-[2-[2-[2-[(2S)-1-[(2S)-2-(methoxycarbonylamino)-3-methyl-butanoyl]pyrrolidin-2-yl]-1H-imidazol-4-yl]ethynyl]thieno[3,2-b]thiophen-5-yl]ethynyl]-1H-imidazol-2-yl]pyrrolidine-1-carbonyl]-2-methyl-propyl]carbamate (14) and methyl N-[(1S)-1-[(2S)-2-[4-[4-[2-[(2S)-1-[(2S)-2-(methoxycarbonylamino)-3-methyl-butanoyl]pyrrolidin-2-yl]-1H-imidazol-4-yl]buta-1,3-diynyl]-1H-imidazol-2-yl]pyrrolidine-1-carbonyl]-2-methyl-propyl]carbamate (15)

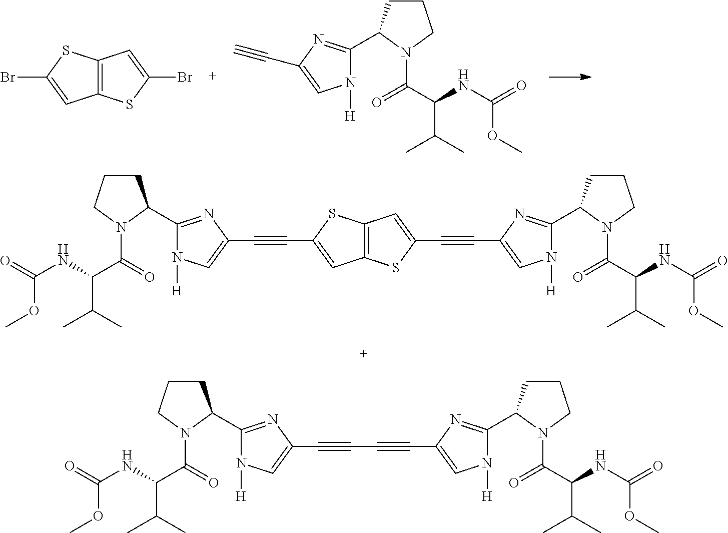 [SMILES: Brc1cc2sc(Br)cc2s1 ; C#Cc1c[nH]c([C@@H]2CCCN2C(=O)[C@@H](NC(=O)OC)C(C)C)n1 ; COC(=O)N[C@H](C(=O)N1CCC[C@H]1c1nc(C#Cc2cc3sc(C#Cc4c[nH]c([C@@H]5CCCN5C(=O)[C@@H](NC(=O)OC)C(C)C)n4)cc3s2)c[nH]1)C(C)C ; COC(=O)N[C@H](C(=O)N1CCC[C@H]1c1nc(C#CC#Cc2c[nH]c([C@@H]3CCCN3C(=O)[C@@H](NC(=O)OC)C(C)C)n2)c[nH]1)C(C)C]

2,5-Dibromothieno[3,2-b]thiophene (26 mg, 0.088 mmol), methyl N-[(1S)-1-[(2S)-2-(4-ethynyl-1H-imidazol-2-yl)pyrrolidine-1-carbonyl]-2-methyl-propyl]carbamate (70 mg, 0.22 mmol), Pd(dppf)Cl₂-DCM (57.1 mg, 0.008 mmol), TEA (24.5 uL, 0.17 mmol) and CuI (3.3 mg, 0.017 mmol) are dissolved in 3 mL of DMF. The system is flushed with nitrogen and the reaction mixture is heated at 70° C. overnight. After evaporation of the solvent under vacuum, the residue is purified by flash column chromatography on silica gel (0 to 7% MeOH in CH₂Cl₂) and is further purified by reverse phase HPLC using a gradient of CH₃CN/water to give methyl N-[(1S)-1-[(2S)-2-[4-[2-[2-[2-[2-[(2S)-1-[(2S)-2-(methoxycarbonylamino)-3-methyl-butanoyl]pyrrolidin-2-yl]-1H-imidazol-4-yl]ethynyl]thieno[3,2-b]thiophen-5-yl]ethynyl]-1H-imidazol-2-yl]pyrrolidine-1-carbonyl]-2-methyl-propyl]carbamate (16 mg)

¹H NMR (400 MHz, CD₃OD): δ [ppm] 7.41 (s, 2H), 7.25 (s, 2H), 5.08 (m, 2H), 4.18 (d, 2H), 3.80-3.94 (m, 4H), 3.56 (s, 6H), 1.97-2.27 (m, 10H), 0.82-0.96 (m, 12H).

LC/MS: m/z 773.53 (M+H⁺).

Methyl N-[(1S)-1-[(2S)-2-[4-[4-[2-[(2S)-1-[(2S)-2-(methoxycarbonylamino)-3-methyl-butanoyl]pyrrolidin-2-yl]-1H-imidazol-4-yl]buta-1,3-diynyl]-1H-imidazol-2-yl]pyrrolidine-1-carbonyl]-2-methyl-propyl]carbamate (5 mg) is also isolated.

¹H NMR (400 MHz, CD₃OD): δ [ppm] 7.33 (s, 2H), 5.08 (m, 2H), 4.18 (d, 2H), 3.77-3.91 (m, 4H), 3.56 (s, 6H), 1.92-2.22 (m, 10H), 0.86-0.96 (m, 12H).

LC/MS: m/z 635.48 (M+H⁺).

### EXAMPLE 16

### Methyl N-[(1S)-1-[(2S,4S)-2-[4-[2-[4-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]-2-methyl-phenyl]ethynyl]-1H-imidazol-2-yl]-4-methyl-pyrrolidine-1-carbonyl]-2-methyl-propyl]carbamate

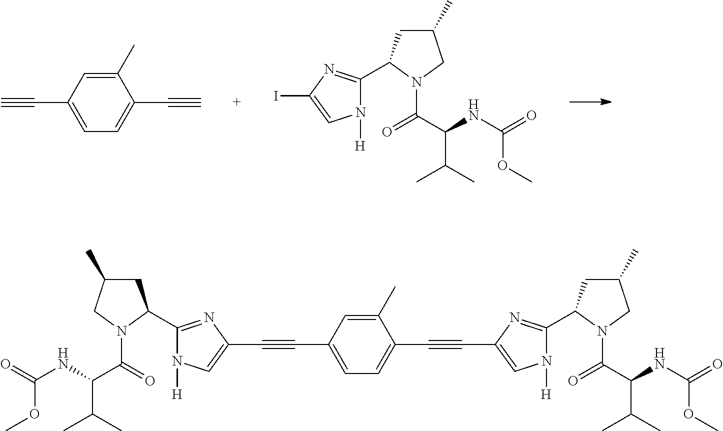 [SMILES: C#Cc1ccc(C#C)c(C)c1 ; COC(=O)N[C@H](C(=O)N1C[C@@H](C)C[C@H]1c1nc(I)c[nH]1)C(C)C ; COC(=O)N[C@H](C(=O)N1C[C@@H](C)C[C@H]1c1nc(C#Cc2ccc(C#Cc3c[nH]c([C@@H]4C[C@H](C)CN4C(=O)[C@@H](NC(=O)OC)C(C)C)n3)c(C)c2)c[nH]1)C(C)C]

Methyl N-[(1S)-1-[(2S,4S)-2-(4-iodo-1H midazol-2-yl)-4-methyl-pyrrolidine-1-carbonyl]-2-methyl-propyl]carbamate (60 mg, 0.14 mmol), 1,4-diethynyl-2-methyl-benzene (8.8 mg, 0.06 mmol), Pd(dppf)Cl₂-DCM (5.1 mg, 0.006 mmol), TEA (22 uL, 0.16 mmol) and CuI (2.4 mg, 0.012 mmol) are dissolved in 2 mL of DMF. The mixture is heated to 50° C. under N₂ overnight. After evaporation of the solvent under vacuum, the residue is purified by flash column chromatography on silica gel (0 to 7% MeOH in CH₂Cl₂) and is further purified by reverse phase HPLC using a gradient of CH₃CN/water to give methyl N-[(1S)-1-[(2S,4S)-2-[4-[2-[4-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]-2-methyl-phenyl]ethynyl]-1H-imidazol-2-yl]-4-methyl-pyrrolidine-1-carbonyl]-2-methyl-propyl]carbamate (10 mg) as a white solid.

¹H NMR (400 MHz, CD₃OD): δ [ppm] 7.42-7.82 (m, 5H), 5.10 (m, 2H), 4.30 (m, 2H), 4.18 (d, 2H), 3.63 (s, 6H), 2.45-2.65 (m, 9H), 1.70-1.96 (m, 4H), 1.19 (m, 6H). 0.86 (m, 12H).

LC/MS: m/z 753.56 (M+H⁺).

### EXAMPLE 17

### Methyl N-[(1S)-1-[(2S,4S)-2-[4-[2-[4-[2-[2-[(2S,4S)-1-[(2S)-2-(methoxy-carbonylamino)-3-methyl-butanoyl]-4-methyl-pyrrolidin-2-yl]-1H-imidazol-4-yl]ethyl]phenyl}ethyl]-1H-imidazol-2-yl]-4-methyl-pyrrolidine-1-carbonyl]-2-methyl-propyl]carbamate

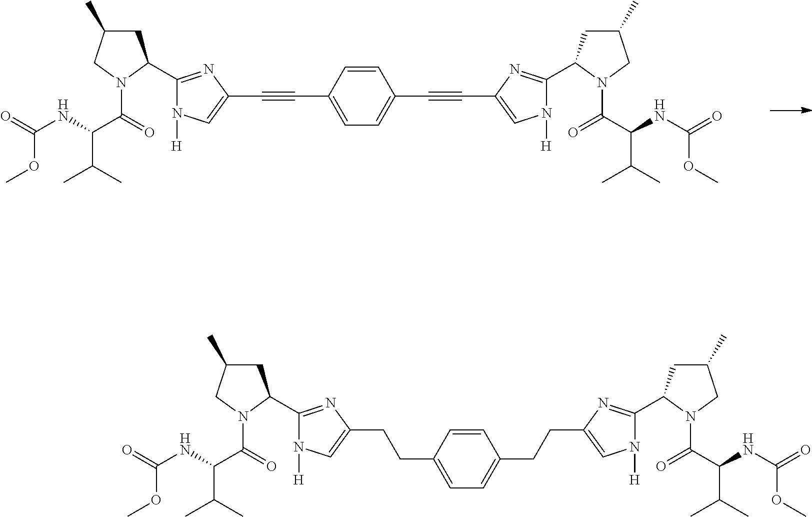 [SMILES: COC(=O)N[C@H](C(=O)N1C[C@@H](C)C[C@H]1c1nc(C#Cc2ccc(C#Cc3c[nH]c([C@@H]4C[C@H](C)CN4C(=O)[C@@H](NC(=O)OC)C(C)C)n3)cc2)c[nH]1)C(C)C ; COC(=O)N[C@H](C(=O)N1C[C@@H](C)C[C@H]1c1nc(CCc2ccc(CCc3c[nH]c([C@@H]4C[C@H](C)CN4C(=O)[C@@H](NC(=O)OC)C(C)C)n3)cc2)c[nH]1)C(C)C]

To a solution of methyl N-[(1S)-1-[(2S,4S)-2-[4-[2-[4-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]phenyl]ethynyl]-1H-imidazol-2-yl]-4-methyl-pyrrolidine-1-carbonyl]-2-methyl-propyl]carbamate (9.3 mg, 0.012 mmol) in 2 mL of methanol are added one drop of 1M HCl and a catalytic amount of 10% Pd/C. The reaction mixture is stirred at RT overnight under 1 atmosphere of hydrogen. The mixture is filtered and the filtrate is concentrated to dryness. The residue is purified by reverse phase HPLC using a gradient of CH₃CN/water to give methyl N-[(1S)-1-[(2S,4S)-2-[4-[2-[4-[2-[2-[(2S,4S)-1-[(2S)-2-(methoxy-carbonylamino)-3-methyl-butanoyl]-4-methyl-pyrrolidin-2-yl]-1H-imidazol-4-yl]ethyl]phenyl]ethyl]-1H-imidazol-2-yl]-4-methyl-pyrrolidine-1-carbonyl]-2-methyl-propyl]carbamate (2.8 mg).

¹H NMR (400 MHz, CD₃OD): [ppm] 7.07 (m, 6H), 5.06 (m, 2H), 4.16-4.27 (m, 4H), 3.60 (s, 6H), 2.89 (m, 6H), 2.45-2.60 (m, 4H), 1.70-1.96 (m, 4H), 1.19 (m, 10H). 0.86 (m, 12H).

LC/MS: m/z 747.56 (M+H⁺).

### EXAMPLE 18

### Methyl N-[(1S)-1-[(2S,4S)-2-[5-[2-[4-[2-[2-[(2S,4S)-1-[(2S)-2-(methoxycarbonylamino)-3-methyl-butanoyl]-4-methyl-pyrrolidin-2-yl]-4-methyl-1H-imidazol-5-yl]ethynyl]phenyl}ethynyl]-4-methyl-1H-imidazol-2-yl]-4-methyl-pyrrolidine-1-carbonyl]-2-methyl-propyl]carbamate

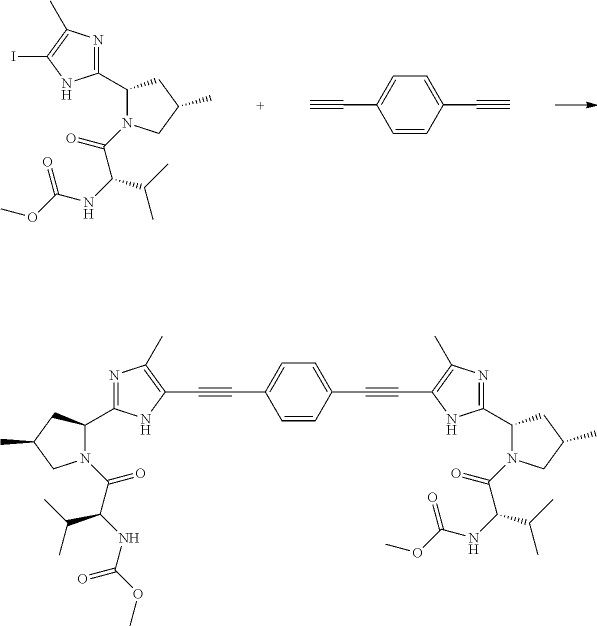

To a solution of methyl N-[(1S)-1-[(2S,4S)-2-(5-iodo-1H-imidazol-2-yl)-4-methyl-pyrrolidine-1-carbonyl]-2-methyl-propyl]carbamate (125 mg, 0.28 mmol), 1,4-diethynylbenzene (17.59 mg, 0.14 mmol), Pd(dppf)Cl₂-DCM (11.38 mg, 0.014 mmol), and CuI (5.31 mg, 0.028 mmol) in DMF (2.50 mL) is added TEA (70.53 mg, 97.15 μL, 0.70 mmol) and the reaction mixture is heated at 80° C. for 18 hours. The reaction mixture is diluted with water (10 mL) and is extracted with of EtOAc (5×10 mL). The combined organic layers are washed with water, dried over Na₂SO₄, and evaporated to dryness. The residue is purified by flash column chromatography on silica gel (0 to 15% MeOH in CH₂Cl₂) and is further purified by reverse phase HPLC using a gradient of CH₃CN/water to give methyl N-[(1R)-1-[(2S,4S)-2-[5-[2-[4-[2-[2-[(2S,4S)-1-[(2S)-2-(methoxycarbonylamino)-3-methyl-butanoyl]-4-methyl-pyrrolidin-2-yl]-4-methyl-1H-imidazol-5-yl]ethynyl]phenyl}ethynyl]-4-methyl-1H-imidazol-2-yl]-4-methyl-pyrrolidine-1-carbonyl]-2-methyl-propyl]carbamate (10.7 mg, 9%).

LC/MS: m/z 767.58 (M+W).

### EXAMPLE 19

### Methyl N-[(1S)-1-[(2S)-2-[5-[2-[4-[2-[2-[(2S)-1-[(2S)-2-(methoxycarbonylamino)-3-methyl-butanoyl]-pyrrolidin-2-yl]-4-methyl-1H-imidazol-5-yl]ethynyl]phenyl}ethynyl]-4-methyl-1H-imidazol-2-yl]-pyrrolidine-1-carbonyl}-2-methyl-propyl]carbamate

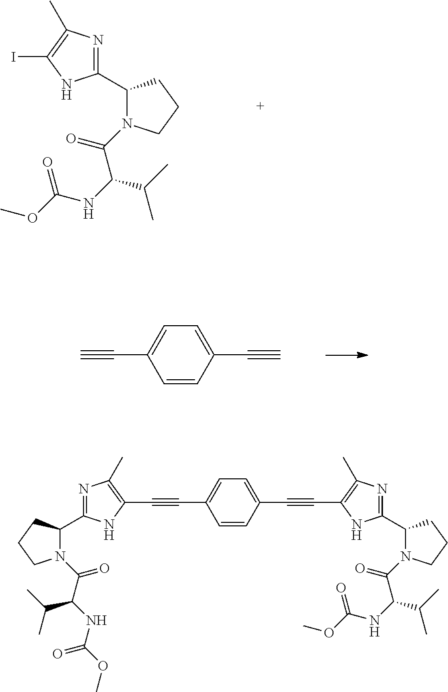

To a solution of methyl N-[(1S)-1-[(2S)-2-(5-iodo-1H-imidazol-2-yl)-pyrrolidine-1-carbonyl]-2-methyl-propyl]carbamate (129 mg, 0.296 mmol), 1,4-diethynylbenzene (18.7 mg, 0.148 mmol), Pd(dppf) (12.1 mg, 0.0148 mmol), and CuI (5.6 mg, 0.0296 mmol) in DMF (2.6 mL) is added TEA (103 μL, 0.740 mmol) and the mixture is heated at 80° C. for 18 hours. The reaction mixture is diluted with water (10 mL) and is extracted with of EtOAc (5×10 mL). The combined organic layers are washed with water, dried over Na₂SO₄, and evaporated to dryness. The residue is purified by flash column chromatography on silica gel (0 to 15% MeOH in CH₂Cl₂) and is further purified by reverse phase HPLC using a gradient of CH₃CN/water to give Methyl N-[(1R)-1-[(2S,4S)-2-[5-[2-[4-[2-[2-[(2S,4S)-1-[(2S)-2-(methoxycarbonylamino)-3-methyl-butanoyl]-pyrrolidin-2-yl}-4-methyl-1H-imidazol-5-yl]ethynyl]phenyl}ethynyl]-4-methyl-1H-imidazol-2-yl]-pyrrolidine-1-carbonyl}-2-methyl-propyl]carbamate (46 mg, 42%).

¹H NMR (400 MHz, DMSO-d₆) δ 12.00 (m, 2H), 7.44 (m, 4H), 7.25 (m, 2H), 4.92 (m, 2H), 3.98 (m, 2H), 3.74 (m, 2H), 3.50 (s, 6H), 2.22 (m, 6H), 2.12 (m, 6H), 1.91 (m, 6H), 0.82 (m, 12H).

### EXAMPLES 20 AND 21

### Methyl N-[(1S)-1-[(2S,4S)-2-[4-iodo-5-[2-[4-[2-[4-iodo-2-[(2S,4S)-1-[(2S)-2-(methoxycarbonylamino)-3-methyl-butanoyl]-4-methyl-pyrrolidin-2-yl]-1H-imidazol-5-yl]ethynyl]phenyl}ethynyl]-1H-imidazol-2-yl]-4-methyl-pyrrolidine-1-carbonyl]-2-methyl-propyl]carbamate (21)

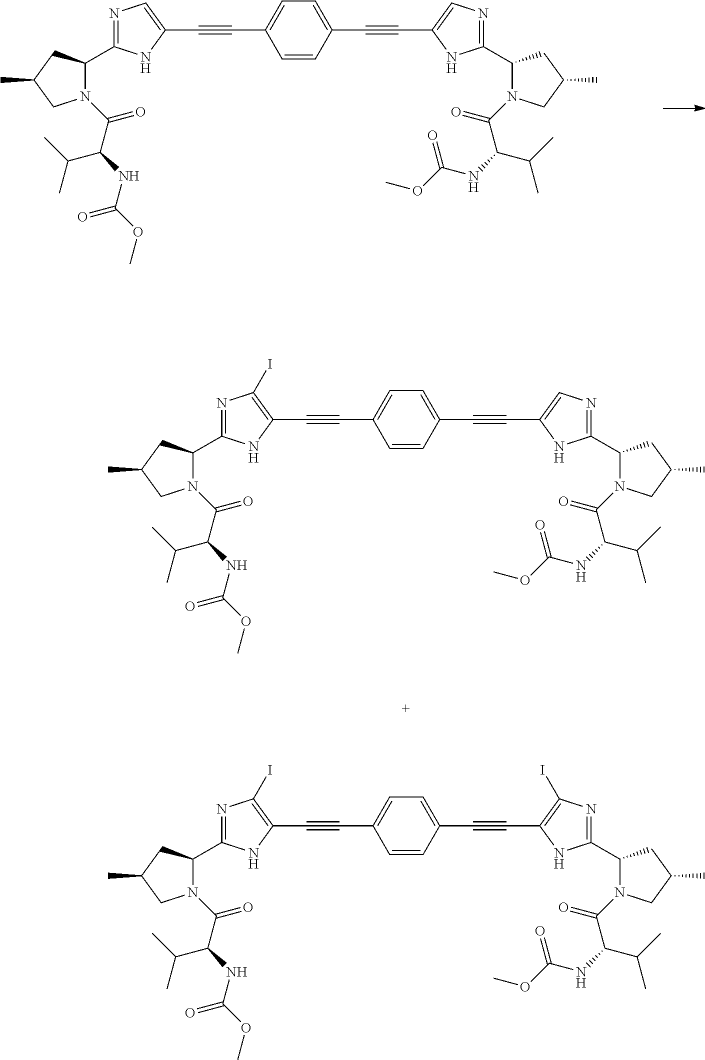

To a stirred suspension of Methyl N-[(1S)-1-[(2S,4S)-2-[5-[2-[4-[2-[2-[(2S,4S)-1-[(2S)-2-(methoxycarbonylamino)-3-methyl-butanoyl]-4-methyl-pyrrolidin-2-yl]-1H-imidazol-5-yl]ethynyl]phenyl]ethynyl]-1H-imidazol-2-yl]-4-methyl-pyrrolidine-1-carbonyl]-2-methyl-propyl]carbamate (0.127 g, 0.172 mmol) in CH₂Cl₂ (1.3 mL) at 0° C. is added N-iodosuccinimide (0.085 g, 0.378 mmol) under nitrogen atmosphere. The reaction mixture is stirred 1 hour at 0° C. and 1 hour at RT. The reaction mixture is concentrated and purified by reverse phase HPLC using a gradient of CH₃CN/water to afford methyl N-[(1S)-1-[(2S,4S)-2-[4-iodo-5-[2-[4-[2-[4-iodo-2-[(2S,4S)-1-[(2S)-2-(methoxycarbonylamino)-3-methyl-butanoyl]-4-methyl-pyrrolidin-2-yl]-1H-imidazol-5-yl]ethynyl]phenyl]ethynyl]-1H-imidazol-2-yl]-4-methyl-pyrrolidine-1-carbonyl]-2-methyl-propyl]carbamate (32 mg, 0.03114 mmol, 18.11%, purity, 96.4%)

LC/MS, m/z: 991.01 (M+H⁺)

and methyl N-[(2S)-1-[(2S,4S)-2-(5-[2-[4-(2-[4-iodo-2-[(2S,4S)-1-[(2S)-2-[(methoxycarbonyl)amino]-3-methylbutanoyl]-4-methylpyrrolidin-2-yl]-1H-imidazol-5-yl}ethynyl)phenyl]ethynyl}-1H-imidazol-2-yl)-4-methylpyrrolidin-1-yl]-3-methyl-1-oxobutan-2-yl]carbamate (7.5 mg, 0.0087 mmol 4.4%, purity, 95.9%)

LC/MS, m/z: 865.33 (M+H⁺).

### EXAMPLE 22

### Methyl N-[1-[(2S,4S)-2-[4-[2-[4-[2-[2-[(2S,4S)-1-[2-(methoxycarbonylamino)-3-methyl-butanoyl]-4-methyl-pyrrolidin-2-yl]-1H-imidazol-4-yl]ethynyl]-2-(trifluoromethyl)phenyl]ethynyl]-1H-imidazol-2-yl]-4-methyl-pyrrolidine-1-carbonyl]-2-methyl-propyl]carbamate

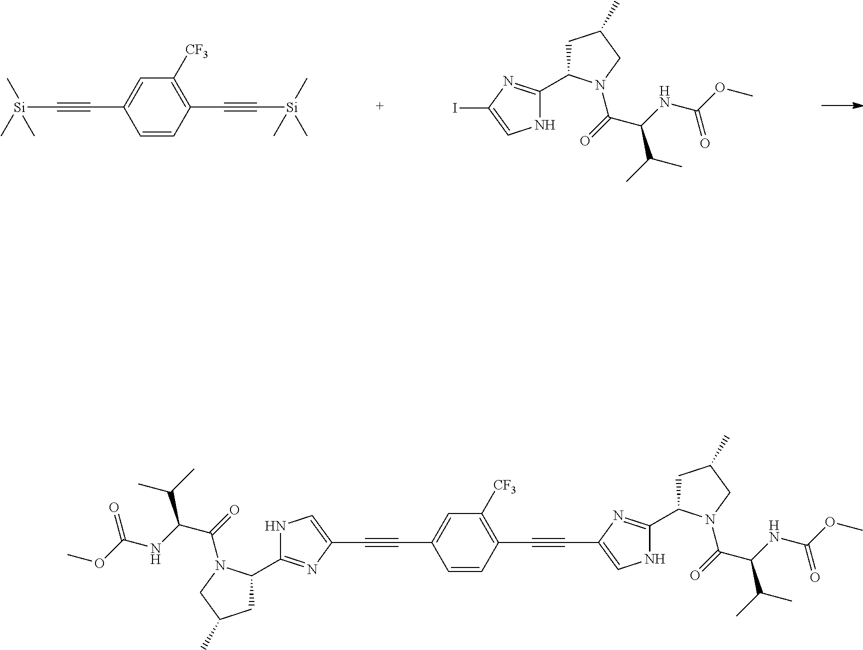

To a stirred solution of methyl N-[1-[(2S,4S)-2-(4-iodo-1H-imidazol-2-yl)-4-methyl-pyrrolidine-1-carbonyl]-2-methyl-propyl]carbamate (59.02 mg, 0.1359 mmol) in DMF (2 mL) are added trimethyl-[2-[2-(trifluoromethyl)-4-(2-trimethylsilylethynyl)phenyl]ethynyl]silane (20 mg, 0.05908 mmol), PdCl₂(dppf)-CH₂Cl₂ (4.825 mg, 0.005908 mmol), CuI (2.251 mg, 0.01182 mmol), H₂O (5.322 mg, 5.322 μL, 0.2954 mmol) and DBU (89.94 mg, 88.35 μL, 0.5908 mmol). The mixture is degassed and heated to 75° C. under nitrogen overnight. After removal of the solvent under reduced pressure, the residue is suspended in water, extracted with CH₂Cl₂ (3×10 mL). The combined organic extracts are washed with brine, dried over Na₂SO₄, concentrated, purified by silica gel column chromatography using MeOH/CH₂Cl₂ (0-6%), and the major fraction is further purified using reverse-phase prep-HPLC to provide methyl N-[1-[(2S,4S)-2-[4-[2-[4-[2-[2-[(2S,4S)-1-[2-(methoxycarbonylamino)-3-methyl-butanoyl]-4-methyl-pyrrolidin-2-yl]-1H-imidazol-4-yl]ethynyl]-2-(trifluoromethyl)phenyl]ethynyl]-1H-imidazol-2-yl]-4-methyl-pyrrolidine-1-carbonyl]-2-methyl-propyl]carbamate (6.9 mg, 0.008466 mmol, 14.33%) as a white solid. LC/MS: m/z=807.44 (M+H⁺). ¹H NMR (CD₃OD, 400 MHz): δ 7.20-7.7.80 (m, 5H), 4.98 (m, 2H), 4.20 (m, 4H), 3.62 (s, 6H), 2.46 (m, 4H), 1.96 (m, 4H), 1.15-1.30 (m, 8H), 0.84 (m, 12H).

### EXAMPLES 23 AND 24

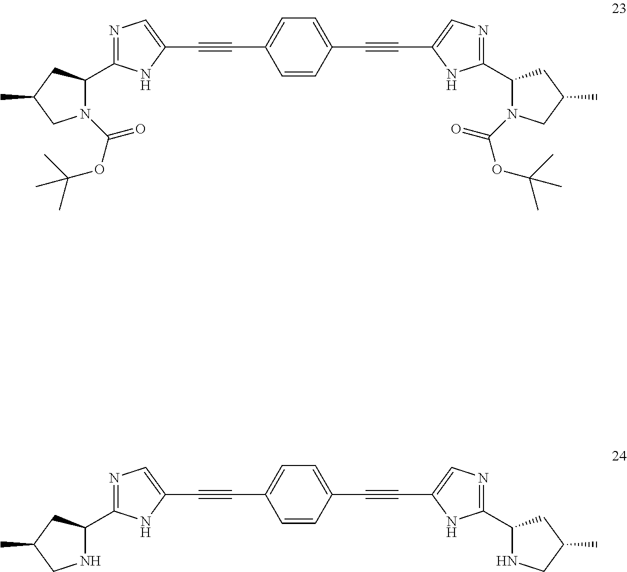

The above compounds were prepared according to the procedures disclosed herein.

### EXAMPLE 25

### Methyl N-[(1S)-1-[(2S)-2-[4-[2-[2-[(2S)-1-[(2S)-2-(methoxycarbonylamino)-3-methyl-butanoyl]pyrrolidin-2-yl]-1H-imidazol-4-yl]thieno[3,2-b]thiophen-5-yl]-1H-imidazol-2-yl]pyrrolidine-1-carbonyl]-2-methyl-propyl]carbamate

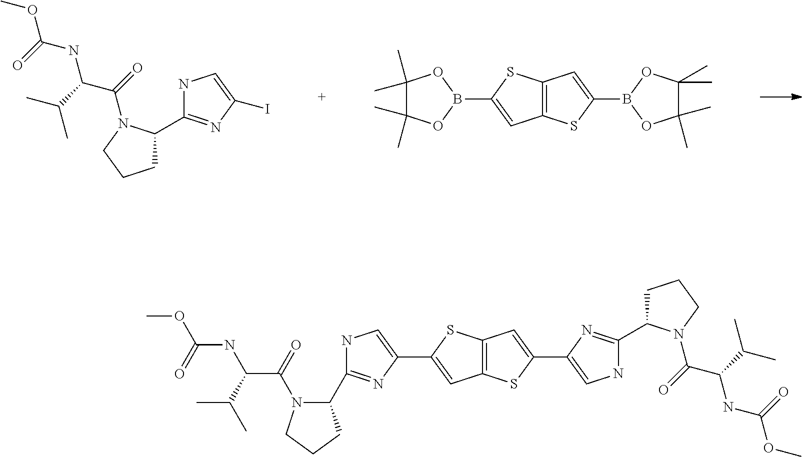

To a solution of methyl N-[(1S)-1-[(2S)-2-(4-iodo-1H-imidazol-2-yl)pyrrolidine-1-carbonyl]-2-methyl-propyl]carbamate (116.6 mg, 0.2775 mmol) and 4,4,5,5-tetramethyl-2-[5-(4,4,5,5-tetramethyl-1,3,2-dioxaborolan-2-yl)thieno[3,2-b]thiophen-2-yl]-1,3,2-dioxaborolane (47 mg, 0.08629 mmol) in acetonitrile (2.5 mL) in a microwave vial (10 mL) under nitrogen atmosphere are sequentially added Pd(dppf)Cl₂-CH₂Cl₂ (14.78 mg, 0.01810 mmol) and aq. sodium bicarbonate (603.3 μL of 1 M, 0.6033 mmol). The resultant suspension is heated in microwave at 150° C. for 10 minutes and concentrated. The residue is dissolved in 10% MeOH—CH₂Cl₂, filtered off salts and concentrated. The residue is purified by silica gel column chromatography using methanol-ethyl acetate (0:100 to 15:85) as eluent to afford a yellow compound (29 mg) which is repurified by reverse phase HPLC to afford methyl N-[(1S)-1-[(2S)-2-[4-[2-[2-[(2S)-1-[(2S)-2-(methoxycarbonylamino)-3-methyl-butanoyl]pyrrolidin-2-yl]-1H-imidazol-4-yl]thieno[3,2-b]thiophen-5-yl]-1H-imidazol-2-yl]pyrrolidine-1-carbonyl]-2-methyl-propyl]carbamate (6.90 mg, 0.009014 mmol, 9.893%) as a light yellow solid. Rf=0.29 (MeOH:EtOAc, 1:4).

LC/MS, m/z=725.5 (M+H⁺); Rt: 7.55 minutes.

HPLC (Rt)=18.1 minutes, Method: 10%-50% AcCN-water (0.01% TFA) for 40 min, Gemini C18 3 μm, 4.6 mm×250 mm.

### EXAMPLE 26

### Methyl N-[(1S)-1-[(2S,4S)-2-[4-[5-[2-[(2S,4S)-1-[(2S)-2-(methoxycarbonylamino)-3-methyl-butanoyl]-4-methyl-pyrrolidin-2-yl]-1H-imidazol-4-yl]thieno[3,2-b]thiophen-2-yl]-1H-imidazol-2-yl]-4-methyl-pyrrolidine-1-carbonyl]-2-methyl-propyl]carbamate

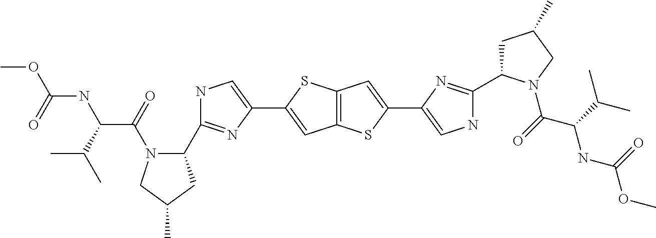

The title compound is prepared from methyl N-[(1S)-1-[(2S,4S)-2-(4-iodo-1H-imidazol-2-yl)-4-methyl-pyrrolidine-1-carbonyl]-2-methyl-propyl]carbamate as described for Example 25.

LC/MS, m/z=753.15 (M+H⁺); Rt: 7.01 minutes.

HPLC (Rt)=9.9 minutes, Method: 20%-60% AcCN-water (0.01% TFA) for 40 min, Gemini C18 3 μm, 4.6 mm×250 mm.

### EXAMPLES 27 TO 45

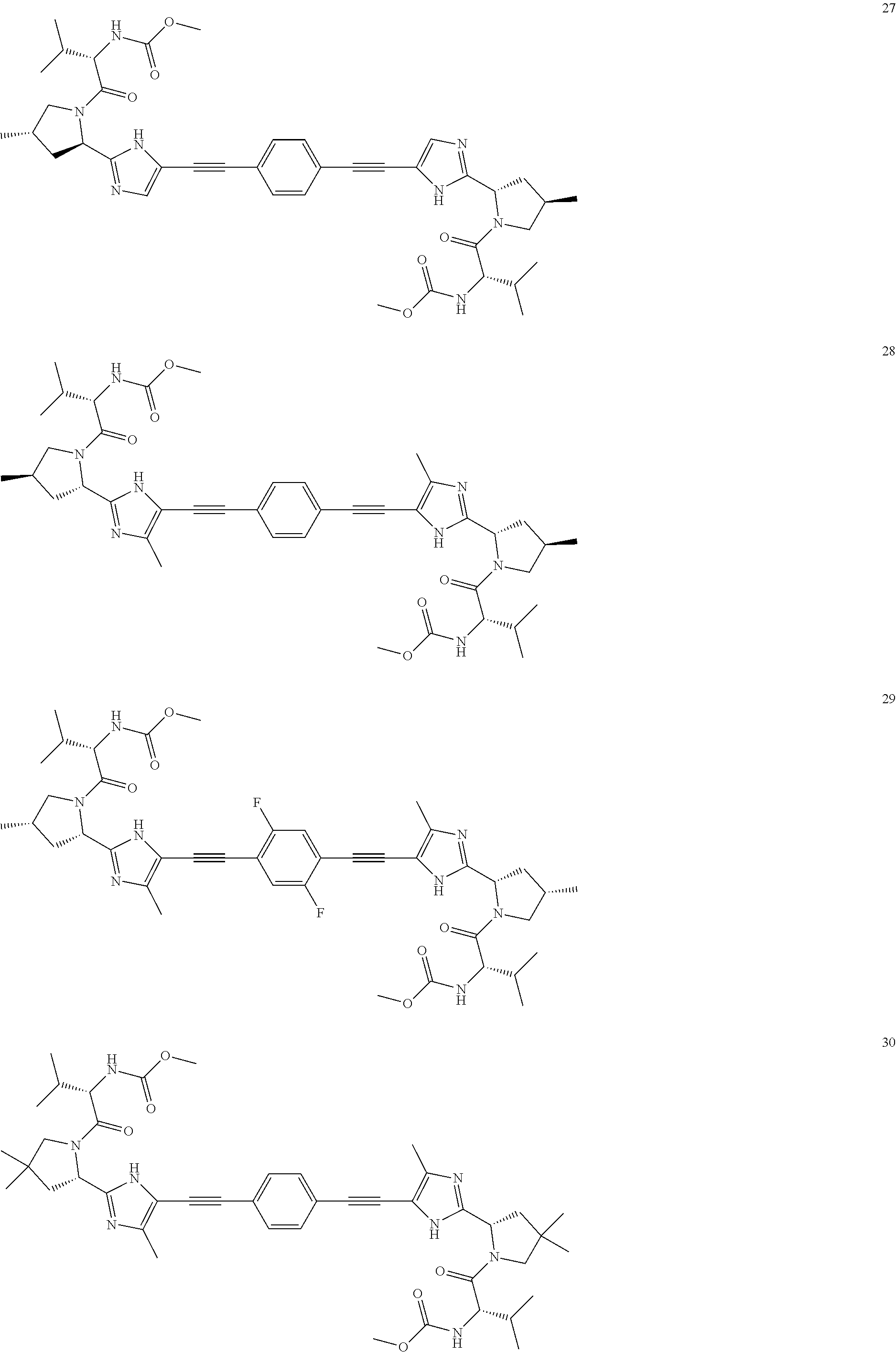

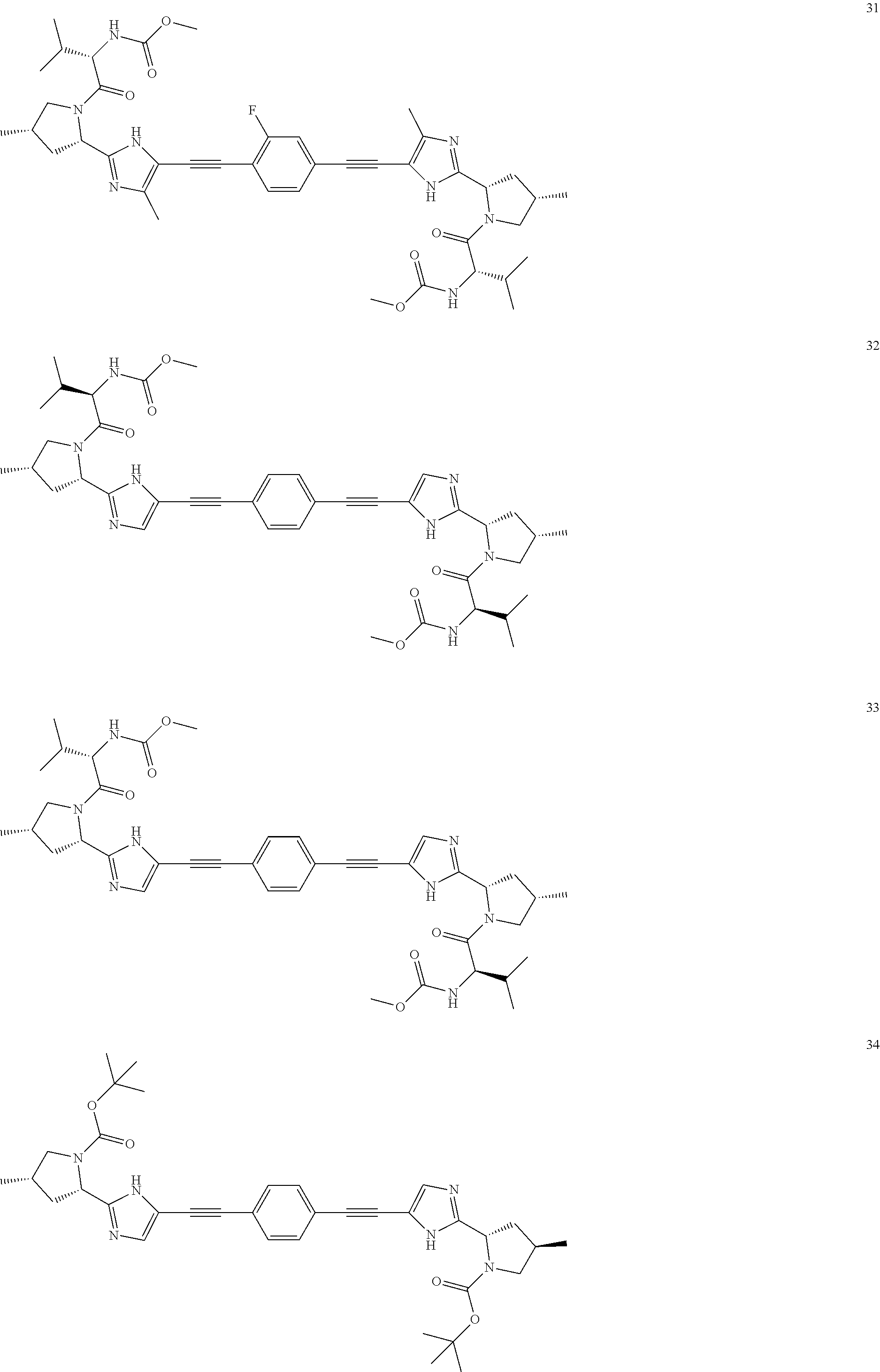

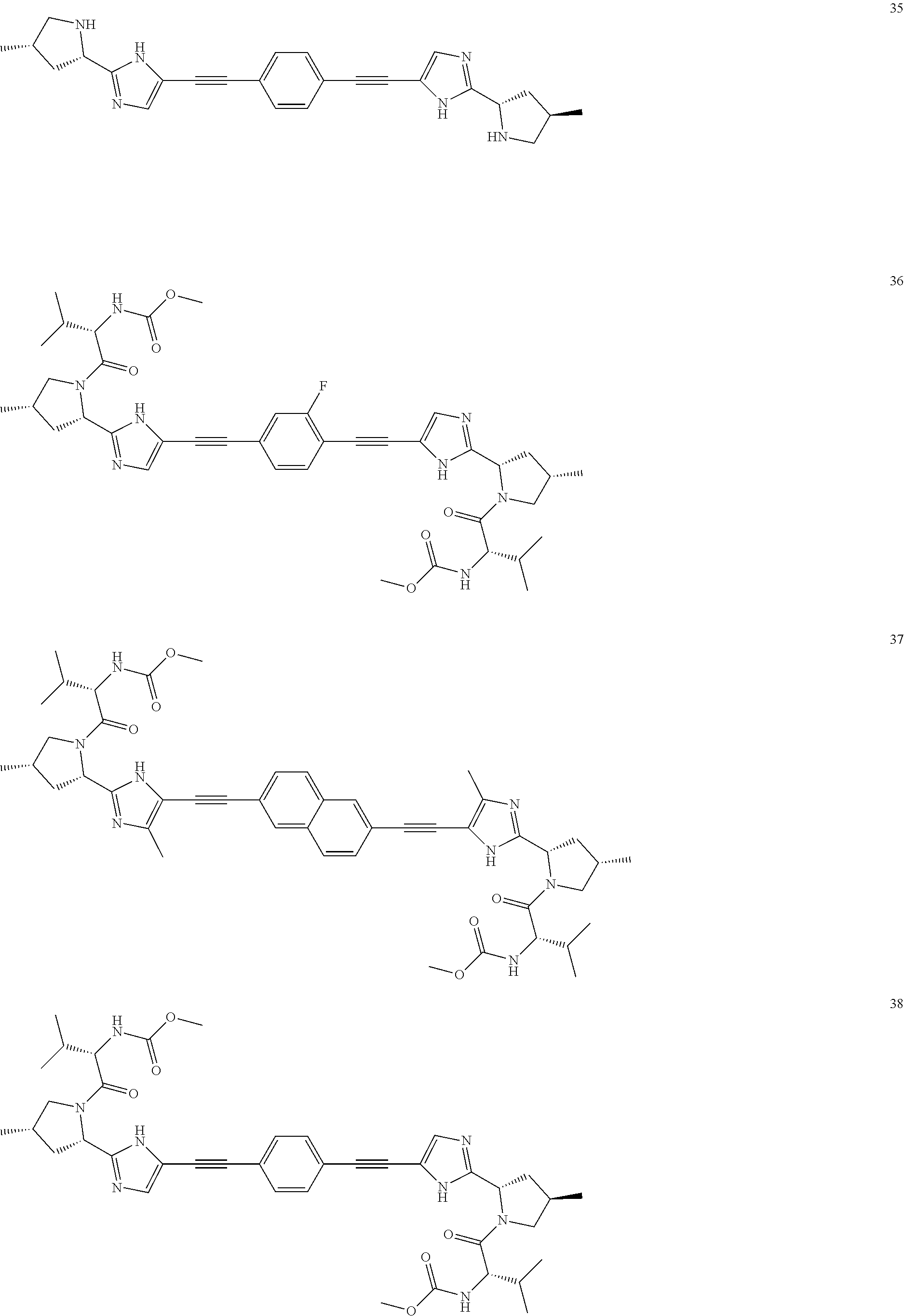

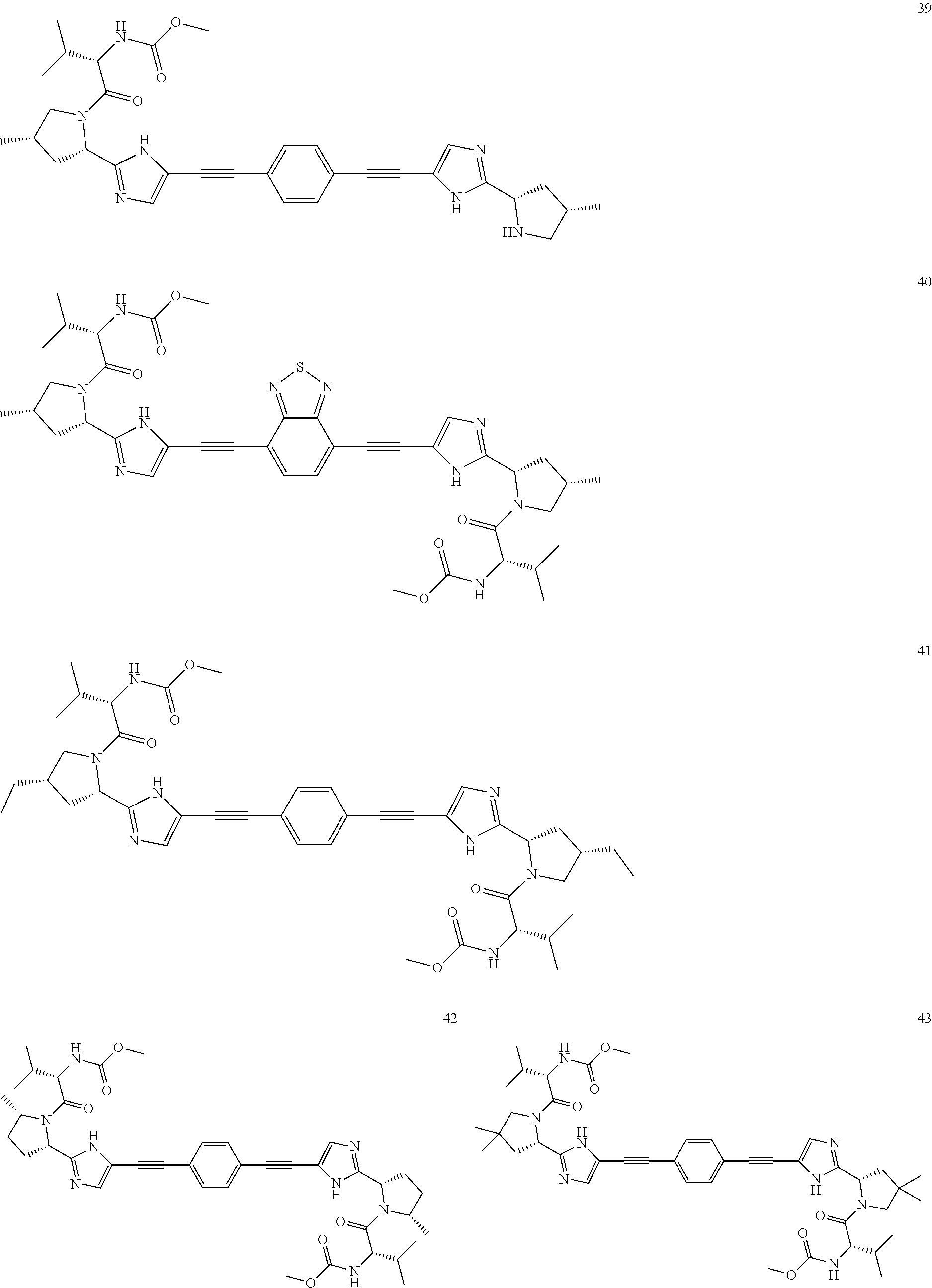

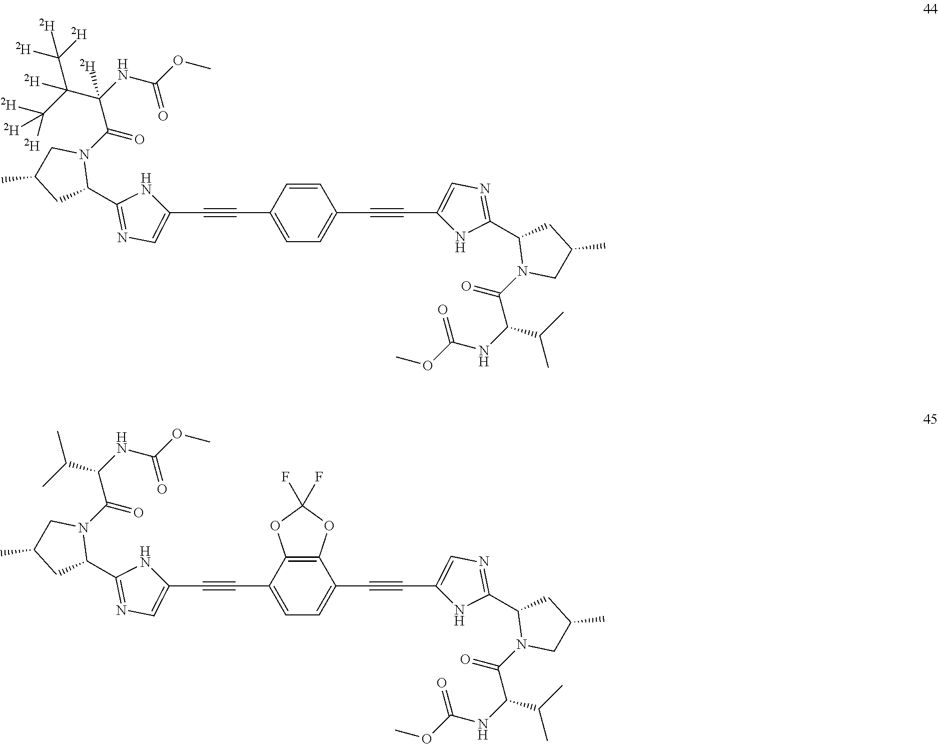

The above compounds were prepared according to the procedures disclosed herein.

### EXAMPLE 46

### Methyl N-[(1S)-1-[(2S,4S)-2-[5-[2-[4-[2-[2-[(2S,4S)-1-[(2S)-2-[[(2S)-2-(methoxycarbonylamino)-3-methyl-butanoyl]amino]-3-methyl-butanoyl]-4-methyl-pyrrolidin-2-yl]-1H-imidazol-5-yl]ethynyl]phenyl]ethynyl]-1H-imidazol-2-yl]-4-methyl-pyrrolidine-1-carbonyl]

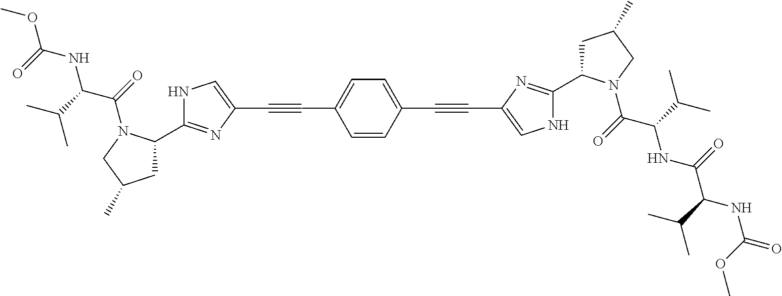 [SMILES: COC(=O)N[C@H](C(=O)N[C@H](C(=O)N1C[C@@H](C)C[C@H]1c1nc(C#Cc2ccc(C#Cc3c[nH]c([C@@H]4C[C@H](C)CN4C(=O)[C@@H](NC(=O)OC)C(C)C)n3)cc2)c[nH]1)C(C)C)C(C)C]

The same procedures are followed as mentioned for Example 4B.

LC/MS: m/z=838.56 (M+H⁺). HPLC (Rt)=43.34 minutes; Method: Phenomenex Gemini C18 3 um, 25% CH₃CN—H₂O (isocratic).

### EXAMPLE 47

### Methyl N-[(1S)-1-[(2S,4S)-2-[4-[5-[2-[(2S,4S)-1-[(2S)-2-(methoxycarbonylamino)-3-methyl-butanoyl]-4-methyl-pyrrolidin-2-yl]-5-methyl-1H-imidazol-4-yl]thieno[3,2-b]thiophen-2-yl]-5-methyl-1H-imidazol-2-yl]-4-methyl-pyrrolidine-1-carbonyl]-2-methyl-propyl]carbamate

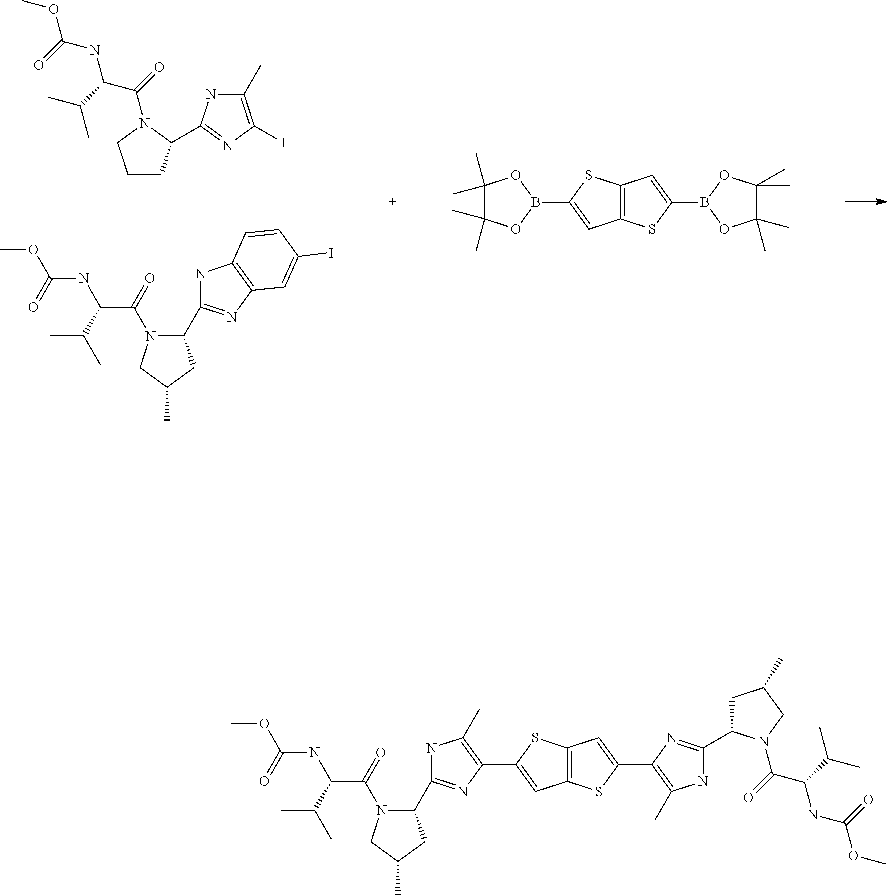

To a degassed (vacuum/nitrogen flush) mixture of methyl N-[(1S)-1-[(2S,4S)-2-(5-iodo-1H-benzimidazol-2-yl)-4-methyl-pyrrolidine-1-carbonyl]-2-methyl-propyl]carbamate (92.65 mg, 0.1913 mmol), methyl N-[(1S)-1-[(2S,4S)-2-(5-iodo-4-methyl-1H-imidazol-2-yl)-4-methyl-pyrrolidine-1-carbonyl]-2-methyl-propyl]carbamate (85.76 mg, 0.1913 mmol), 4,4,5,5-tetramethyl-2-[5-(4,4,5,5-tetramethyl-1,3,2-dioxaborolan-2-yl)thieno[3,2-b]thiophen-2-yl]-1,3,2-dioxaborolane (75 mg, 0.1913 mmol) and K₂CO₃ (132.2 mg, 0.9565 mmol) in degassed IPA (2.250 mL) and H₂O (750.0 μL) are added [3-(2-dicyclohexylphosphanylphenyl)-2,4-dimethoxy-phenyl]sulfonyloxysodium (15.69 mg, 0.03061 mmol) and Pd(OAc)₂ (1.718 mg, 0.007652 mmol). The reaction mixture is degassed twice and slowly heated at 90° C. for 16 hours, diluted with ethyl acetate (30 mL) and then the aqueous solution is discarded. The organic layer is washed with brine, dried (Na₂SO₄) and concentrated. The residue is purified by silica gel column chromatography using ethyl acetate to 10% MeOH-EtOAc as eluent to afford a mixture of products (110 mg) as yellow solid. LC-MS shows the presence of a mixture of methyl N-[(1S)-1-[(2S,4S)-2-[4-[5-[2-[(2S,4S)-1-[(2S)-2-(methoxycarbonylamino)-3-methyl-butanoyl]-4-methyl-pyrrolidin-2-yl]-5-methyl-1H-imidazol-4-yl]thieno[3,2-b]thiophen-2-yl]-5-methyl-1H-imidazol-2-yl]-4-methyl-pyrrolidine-1-carbonyl]-2-methyl-propyl]carbamate (HPLC Rt 8.42 minutes, M.Wt. 781), methyl N-[(1S)-1-[(2S,4S)-2-[4-[5-[2-[(2S,4S)-1-[(2S)-2-(methoxycarbonylamino)-3-methyl-butanoyl]-4-methyl-pyrrolidin-2-yl]-1H-benzimidazol-5-yl]thieno[3,2-b]thiophen-2-yl]-5-methyl-1H-imidazol-2-yl]-4-methyl-pyrrolidine-1-carbonyl]-2-methyl-propyl]carbamate (HPLC Rt, 13.6 minutes, M.Wt. 817) and methyl N-[(1S)-1-[(2S,4S)-2-[5-[5-[2-[(2S,4S)-1-[(2S)-2-(methoxycarbonylamino)-3-methyl-butanoyl]-4-methyl-pyrrolidin-2-yl]-1H-benzimidazol-5-yl]thieno[3,2-b]thiophen-2-yl]-1H-benzimidazol-2-yl]-4-methyl-pyrrolidine-1-carbonyl]-2-methyl-propyl]carbamate (HPLC Rt. 19.3 minutes, M.Wt. 853.06). Reverse phase HPLC purification of the mixture gives the title compound (18.3 mg, 0.02160 mmol, 45.17%) as a light yellow solid.

LC/MS, m/z=781.17 (M+H⁺); Rt: 6.92 minutes.

HPLC (Rt)=8.26 minutes, Method: 20%-60% AcCN-water (0.01% TFA) for 40 min, Gemini C18 3 μm, 4.6 mm×250 mm.

### EXAMPLE 48

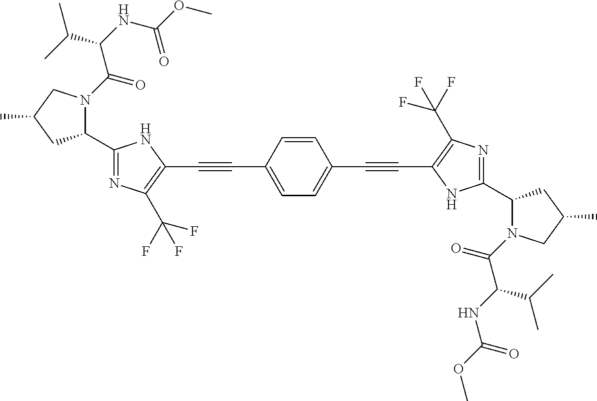

The above compound was prepared according to the procedures disclosed herein.

### EXAMPLE 49

### Methyl N-[(1S)-1-[(2S,4S)-2-[4-[6-[2-[(2S,4S)-1-[(2S)-2-(methoxycarbonylamino)-3-methyl-butanoyl]-4-methyl-pyrrolidin-2-yl]-1H-imidazol-4-yl]-2-naphthyl]-1H-imidazol-2-yl]-4-methyl-pyrrolidine-1-carbonyl]-2-methyl-propyl]carbamate

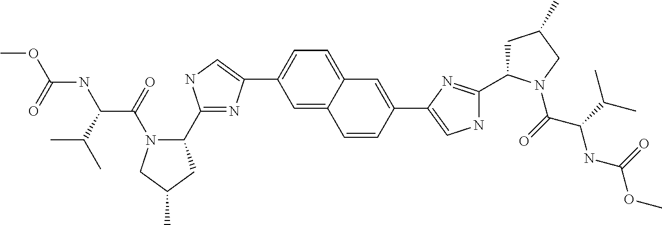

The title compound is prepared from methyl N-[(1S)-1-[(2S,4S)-2-(4-iodo-1H-imidazol-2-yl)-4-methyl-pyrrolidine-1-carbonyl]-2-methyl-propyl]carbamate, methyl N-[(1S)-1-[(2S,4S)-2-(5-iodo-1H-benzimidazol-2-yl)-4-methyl-pyrrolidine-1-carbonyl]-2-methyl-propyl]carbamate and 4,4,5,5-tetramethyl-2,6-(4,4,5,5-tetramethyl-1,3,2-dioxaborolan-2-yl)-2-naphthyl]-1,3,2-dioxaborolane as described for Example 47.

LC/MS, m/z=741.42 (M+H⁺); Rt: 6.46 minutes.

HPLC (Rt)=6.72 minutes, Method: 20%-60% AcCN-water (0.01% TFA) for 40 min, Gemini C18 3 μm, 4.6 mm×250 mm.

### EXAMPLES 50 AND 51

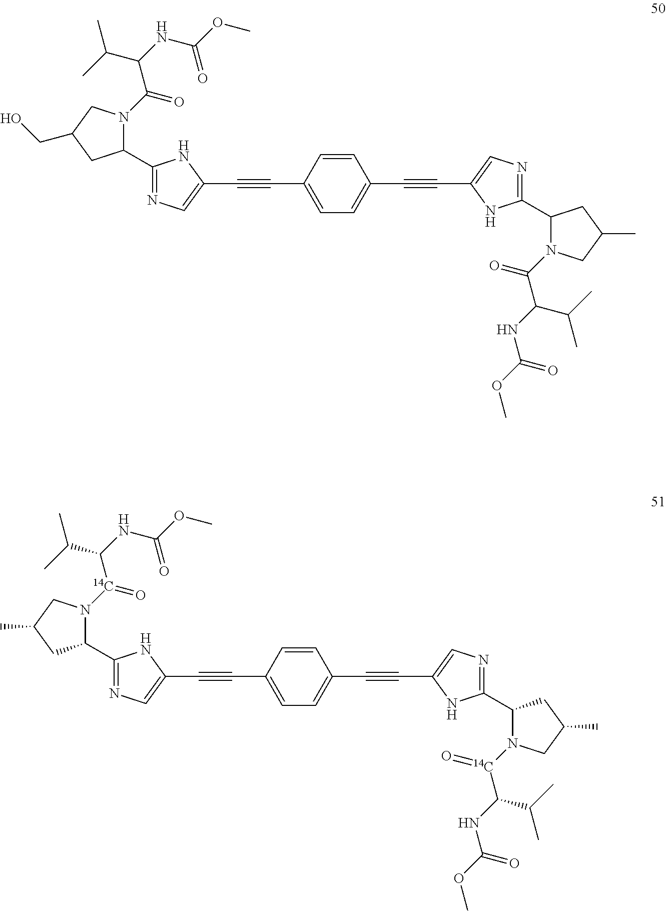

The above compounds were prepared according to the procedures disclosed herein.

### EXAMPLE 52

### Methyl N-[(1S)-1-[(2R,45)-2-[4-[2-[4-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]phenyl}ethynyl]-1H-imidazol-2-yl]-4-methyl-pyrrolidine-1-carbonyl]-2-methyl-propyl]carbamate

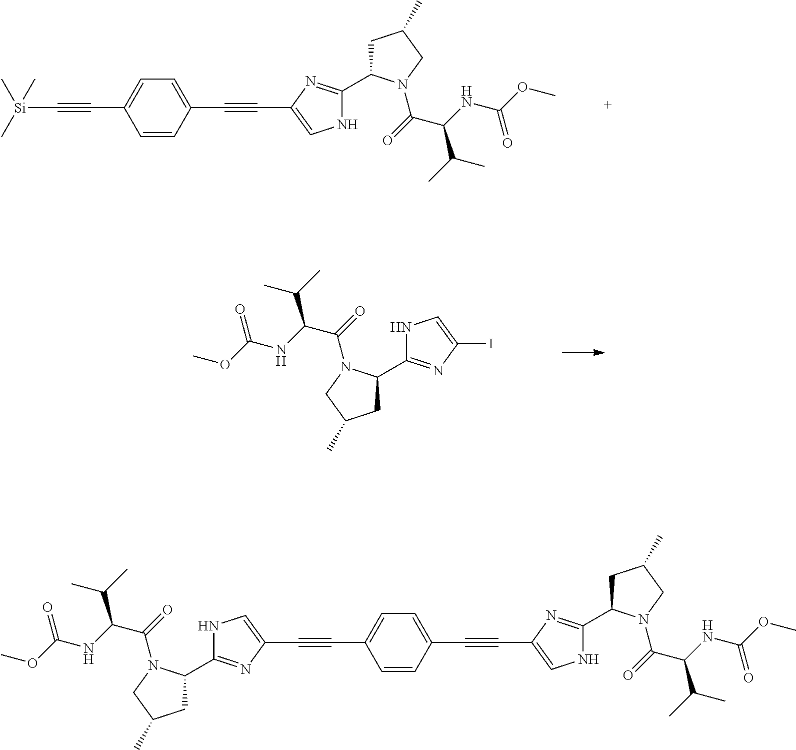

To a stirred solution of methyl N-[(1S)-2-methyl-1-[(2S,4S)-4-methyl-2-[4-[2-[4-(2-trimethylsilylethynyl)phenyl]ethynyl]-1H-imidazol-2-yl]pyrrolidine-1-carbonyl]propyl]carbamate (63.89 mg, 0.1266 mmol) in DMF (2 mL) are added methyl N-[(1S)-1-[(2R,4S)-2-(4-iodo-1H-imidazol-2-yl)-4-methyl-pyrrolidine-1-carbonyl]-2-methyl-propyl]carbamate (50 mg, 0.1151 mmol), PdCl₂(dppf)-CH₂Cl₂ (9.400 mg, 0.01151 mmol), CuI (2.192 mg, 0.01151 mmol), DBU (140.2 mg, 137.7 μL, 0.9208 mmol) and water (10.37 mg, 10.37 μL, 0.5755 mmol). The mixture is degassed and heated at 75° C. under nitrogen overnight. After removal of the solvent under reduced pressured, the residue is suspended in water, extracted with CH₂Cl₂ (3×10 mL). The combined organic extracts are washed with brine, dried over Na₂SO₄, concentrated, purified by silica gel column chromatography using MeOH (0-6%) in CH₂Cl₂ and the major fraction is further purified using reverse-phase prep-HPLC to provide methyl N-[(1S)-1-[(2R,4S)-2-[4-[2-[4-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]phenyl}ethynyl]-1H-imidazol-2-yl]-4-methyl-pyrrolidine-1-carbonyl]-2-methyl-propyl]carbamate (3.9 mg, 0.005199 mmol, 4.517%) as a white solid.

LC/MS: m/z=739.43 (M+H⁺). ¹H NMR (CD₃OD, 400 MHz): δ 7.55-7.65 (m, 6H), 5.25 (m, 1H), 5.10 (m, 1H), 4.20 (m, 4H), 3.62 (2×s, 6H), 2.50 (m, 4H), 2.00 (m, 4H), 1.15 (m, 8H), 0.84 (m, 12H).

### EXAMPLE 53

### Methyl N-[(1S)-1-[(2S,4S)-2-[4-[4-[2-[(2S,4S)-1-[(2S)-2-(methoxycarbonylamino)-3-methyl-butanoyl]-4-methyl-pyrrolidin-2-yl]-1H-imidazol-4-yl]buta-1,3-diynyl]-1H-imidazol-2-yl]-4-methyl-pyrrolidine-1-carbonyl]-2-methyl-propyl]carbamate

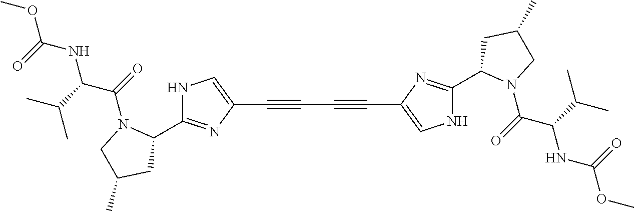

LC/MS, m/z=663.44 (M+H⁺); Rt: 7.61 minutes.

HPLC RT=14.47 minutes; Method: 20%-60% AcCN-water (0.01% TFA) for 40 min, Gemini C18 3 μm, 4.6 mm×250 mm.

### EXAMPLE 54

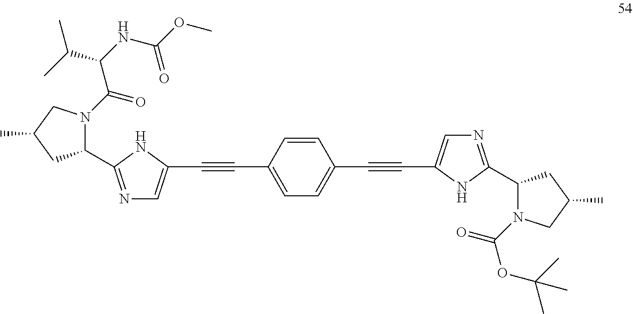

The above compound was prepared according to the procedures disclosed herein.

### EXAMPLE 55

### Methyl N-[(1S)-1-[(2S,4S)-2-[4-[2-[2-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]-6-quinolyl]ethynyl]-1H-imidazol-2-yl]-4-methyl-pyrrolidine-1-carbonyl]-2-methyl-propyl]carbamate

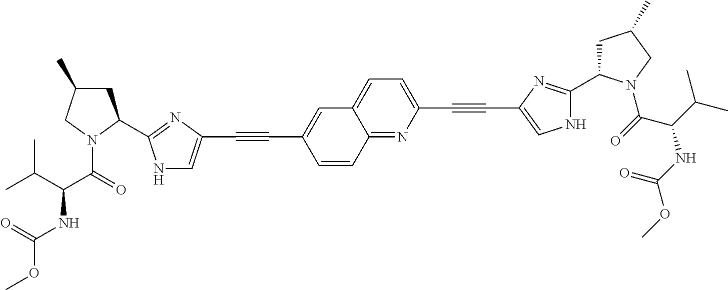 [SMILES: COC(=O)N[C@H](C(=O)N1C[C@@H](C)C[C@H]1c1nc(C#Cc2ccc3nc(C#Cc4c[nH]c([C@@H]5C[C@H](C)CN5C(=O)[C@@H](NC(=O)OC)C(C)C)n4)ccc3c2)c[nH]1)C(C)C]

The title compound is synthesized from methyl N-[(1S)-1-[(2S,4S)-2-(4-iodo-1H-imidazol-2-yl)-4-methyl-pyrrolidine-1-carbonyl]-2-methyl-propyl]carbamate and 2,6-diethynylquinoline as described in Example 6.

¹H NMR (400 MHz, CD₃OD): δ 8.30 (d, 1H), 8.03 (s, 1H), 7.92 (d, 1H), 7.79 (d, 1H), 7.63 (d, 1H), 7.45 (s, 1H), 7.32 (s, 1H), 5.01 (m, 2H), 4.19 (m, 2H), 4.17 (m, 2H), 3.62 (s, 6H), 2.46 (m, 2H), 2.34 (m, 2H), 1.99-1.86 (m, 4H), 1.36-1.27 (m, 2H), 1.17 (d, 6H), 0.89-0.82 (m, 12H).

LC/MS: m/z=790.3 (M+H⁺).

2,6-Diethynylquinoline is prepared from 2,6-dibromoquinoline according to the procedure reported for intermediate 12.

### EXAMPLE 56

### Methyl N-[(1S)-1-[(2S,4S)-2-[4-[2-[5-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]-2-pyridyl]ethynyl]-1H-imidazol-2-yl]-4-methyl-pyrrolidine-1-carbonyl]-2-methyl-propyl]carbamate

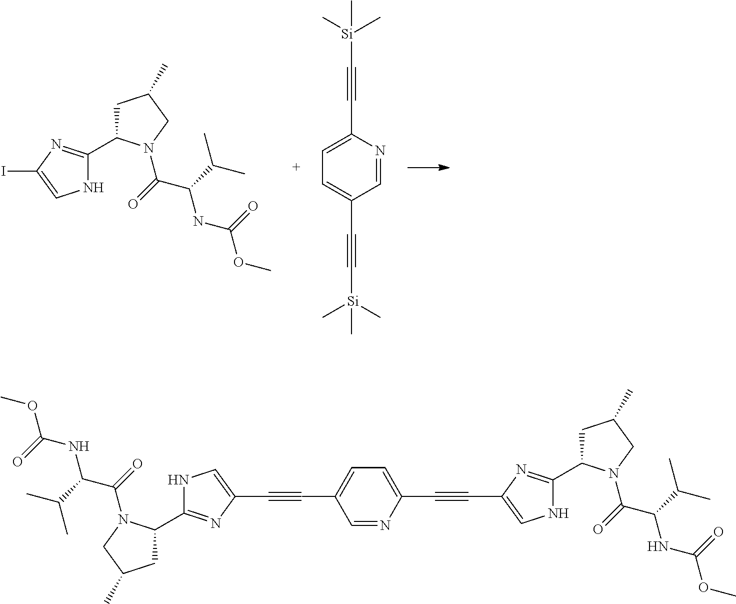

To a mixture of methyl N-[(1S)-1-[(2S,4S)-2-(4-iodo-1H-imidazol-2-yl)-4-methyl-pyrrolidine-1-carbonyl]-2-methyl-propyl]carbamate (102.4 mg, 0.2234 mmol), trimethyl-[2-[6-(2-trimethylsilylethynyl)-3-pyridyl]ethynyl]silane (31 mg, 0.1117 mmol), PdCl₂(dppf)-CH₂Cl₂ (13.69 mg, 0.01676 mmol) and copper Iodide (3.192 mg, 0.01676 mmol) is added DMF (1 mL) under nitrogen atmosphere. The reaction mixture is degassed thrice (vacuum and nitrogen gas) and DBU (204.0 mg, 200.4 μL, 1.340 mmol) is added. The reaction mixture is degassed three times, degassed water (6.037 mg, 6.037 μL, 0.3351 mmol) is added and heated at 60° C. for 8 hours. The reaction mixture is diluted with water (6 mL), extracted with methanol-CH₂Cl₂, and the combined extracts are washed with brine. The organic layer is dried (Na₂SO₄), concentrated and purified by silica gel column chromatography using methanol-CH₂Cl₂ (0 to 10%) as eluent to afford a crude product (43 mg) which is repurified by HPLC to afford methyl N-[(1S)-1-[(2S,4S)-2-[4-[2-[5-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]-2-pyridyl]ethynyl]-1H-imidazol-2-yl]-4-methyl-pyrrolidine-1-carbonyl]-2-methyl-propyl]carbamate (22.7 mg, 0.02985 mmol, 26.73%).

¹H NMR (400 MHz, CD₃OD): δ 8.49 (s, 1H), 7.78 (d, 1H), 7.46 (d, 1H), 7.3 (s, 1H), 7.25 (s, 1H), 4.95-4.86 (m, 2H), 4.15-4.05 (m, 4H), 3.54 (s, 6H), 3.3-3.5 (m, 2H), 2.44-2.16 (m, 4H), 1.94-1.66 (m, 4H), 1.08 (d, 6H), 0.77 (d, 6H), 0.74 (d, 6H).

LC/MS: m/z=740.45 (M+H⁺). Rₜ=7.75 minutes.

HPLC (Rt)=13.4 minutes, Method: 20%-60% AcCN-water (0.01% TFA) for 40 minutes, Gemini C18 3 μm, 4.6 mm×250 mm.

### EXAMPLE 57

### Methyl 2,5-bis[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]benzoate

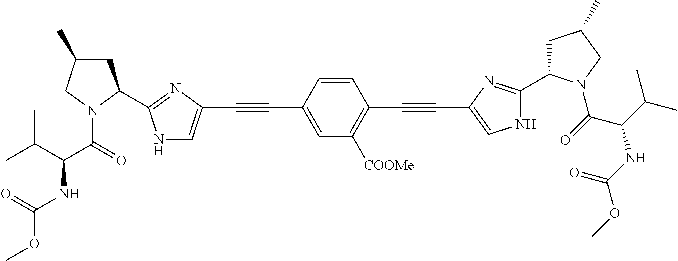 [SMILES: COC(=O)N[C@H](C(=O)N1C[C@@H](C)C[C@H]1c1nc(C#Cc2ccc(C#Cc3c[nH]c([C@@H]4C[C@H](C)CN4C(=O)[C@@H](NC(=O)OC)C(C)C)n3)c(C(=O)OC)c2)c[nH]1)C(C)C]

The title compound is synthesized from tert-butyl (2S,4S)-2-(4-iodo-1H-imidazol-2-yl)-4-methyl-pyrrolidine-1-carboxylate and methyl 2,5-diethynylbenzoate as described in Example 4B.

¹H NMR (400 MHz, CD₃OD): δ 7.97 (s, 1H), 7.58 (m, 2H), 7.29 (m, 2H), 4.98 (m, 2H), 4.21-4.15 (m, 4H), 3.92 (s, 3H), 3.62 (s, 6H), 2.48-2.42 (m, 2H), 2.36-2.31 (m, 2H), 1.97-1.85 (m, 4H), 1.16 (s, 6H), 0.81-0.88 (m, 12H).

LC/MS: m/z=797.4 (M+H⁺).

Methyl 2,5-diethynylbenzoate is prepared from methyl 2,5-diiodobenzoate according to the procedure reported for intermediate 12.

### EXAMPLES 58 AND 59

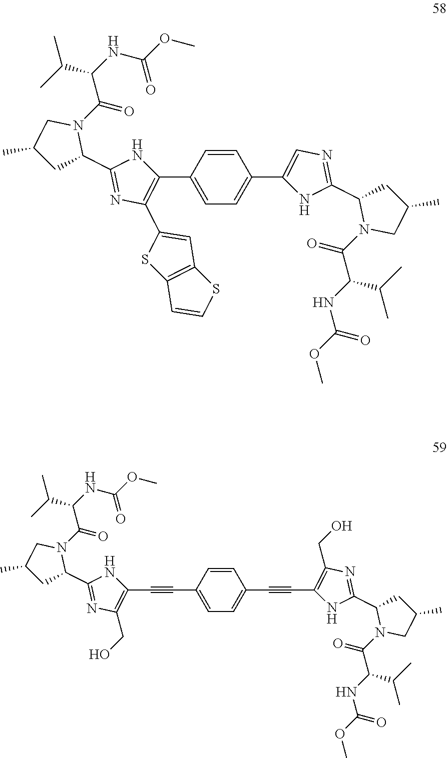

The above compounds were prepared according to the procedures disclosed herein.

### EXAMPLE 60

### Methyl N-[(1S)-1-[(2S,4S)-2-[4-[2-[5-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]-2-thienyl]ethynyl]-1H-imidazol-2-yl]-4-methyl-pyrrolidine-1-carbonyl]-2-methyl-propyl]carbamate

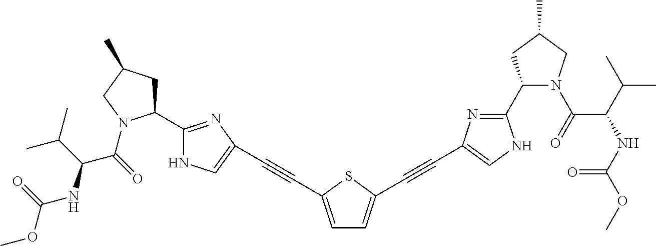 [SMILES: COC(=O)N[C@H](C(=O)N1C[C@@H](C)C[C@H]1c1nc(C#Cc2ccc(C#Cc3c[nH]c([C@@H]4C[C@H](C)CN4C(=O)[C@@H](NC(=O)OC)C(C)C)n3)s2)c[nH]1)C(C)C]

The title compound is synthesized from methyl N-[(1S)-1-[(2S)-2-(4-ethynyl-1Himidazol-2-yl)pyrrolidine-1-carbonyl]-2-methyl-propyl]carbamate and 2,5-dibromothiophene as described in Example 13.

¹H NMR (400 MHz, CD₃OD): δ 7.27 (m, 2H), 7.12 (m, 2H), 4.95 (m, 2H), 4.2-4.07 (m, 4H), 3.62 (s, 6H), 2.47-2.41 (m, 2H), 2.35-2.32 (m, 2H), 1.96-1.89 (m, 2H), 1.91-1.83 (m, 2H), 1.27 (m, 2H), 1.15 (d, 6H), 0.91-0.82 (m, 12H).

LC/MS: m/z=745.3 (M+H⁺).

### EXAMPLE 61

### Methyl N-[(1S)-1-[(2S,4S)-2-[4-[2-[2-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]benzothiophen-5-yl]ethynyl]-1H-imidazol-2-yl]-4-methyl-pyrrolidine-1-carbonyl]-2-methyl-propyl]carbamate

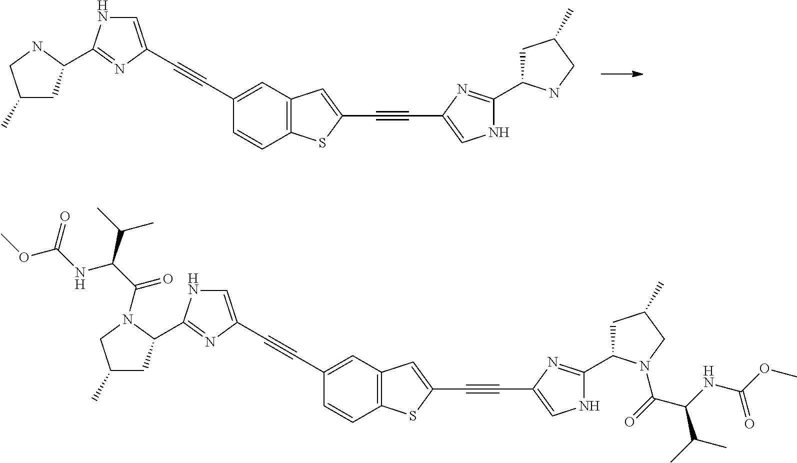

To a solution of 2-[(2S,4S)-4-methylpyrrolidin-2-yl]-4-[2-[2-[2-[2-[(2S,4S)-4-methylpyrrolidin-2-yl]-1H-imidazol-4-yl]ethynyl]benzothiophen-5-yl]ethynyl]-1H-imidazole (Hydrochloric Acid (4)) (30 mg, 0.04789 mmol) HCl salt in DMF (2 mL) are added (2S)-2-(methoxycarbonylamino)-3-methyl-butanoic acid (20.97 mg, 0.1197 mmol), HATU (45.51 mg, 0.1197 mmol) and DIPEA (61.89 mg, 83.41 μL, 0.4789 mmol). The mixture is stirred at rt overnight. After removal of the solvent under reduced pressure, the residue is purified by silica gel column chromatography using MeOH (0-6%) in CH₂Cl₂, and the major fraction is further purified using reverse-phase prep-HPLC to provide methyl N-[(1S)-1-[(2S,4S)-2-[4-[2-[2-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]benzothiophen-5-yl]ethynyl]-1H-imidazol-2-yl]-4-methyl-pyrrolidine-1-carbonyl]-2-methyl-propyl]carbamate (23 mg, 0.02841 mmol, 59.33%) as a white solid. LC/MS: m/z=795.40 (M+H⁺). ¹H NMR (CD₃OD, 400 MHz): δ 7.24-7.95 (m, 6H), 4.95 (m, 2H), 4.20 (m, 4H), 3.62 (s, 6H), 2.46 (m, 4H), 1.96 (m, 4H), 1.15 (m, 6H), 0.84 (m, 14H).

### EXAMPLE 62

### Methyl N-[(1S)-1-[(2S,4S)-2-[4-[2-[2-cyano-4-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]phenyl]ethynyl]-1H-imidazol-2-yl]-4-methyl-pyrrolidine-1-carbonyl]-2-methyl-propyl]carbamate

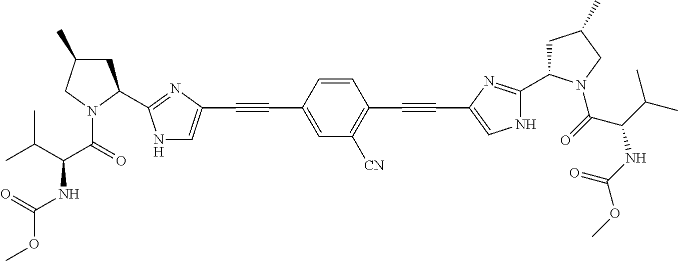 [SMILES: COC(=O)N[C@H](C(=O)N1C[C@@H](C)C[C@H]1c1nc(C#Cc2ccc(C#Cc3c[nH]c([C@@H]4C[C@H](C)CN4C(=O)[C@@H](NC(=O)OC)C(C)C)n3)c(C#N)c2)c[nH]1)C(C)C]

The title compound is synthesized from tert-butyl (2S,4S)-2-(4-iodo-1H-imidazol-2-yl)-4-methyl-pyrrolidine-1-carboxylate and 2,5-diethynylbenzonitrile as described in Example 4B.

¹H NMR (400 MHz, CD₃OD): δ 7.83 (s, 1H), 7.69 (d, 1H), 7.62 (d, 1H), 7.39 (s, 1H), 7.32 (s, 1H), 4.99-4.95 (m, 2H), 4.21-4.15 (m, 4H), 3.62 (s, 6H), 2.47-2.42 (m, 2H), 2.34 (m, 2H), 1.96-1.88 (m, 4H), 1.27 (m, 2H), 1.16 (d, 6H), 0.88-0.81 (m, 12H).

LC/MS: m/z=764.4 (M+H⁺).

2,5-Diethynylbenzonitrile is prepared from 2,5-diiodobenzonitrile according to the procedure reported for intermediate 12.

### EXAMPLE 63

### Dimethyl (2S,2′S)-1,1′-((3S,3′S,5S,5′S)-5,5′-(4,4′-(2-chloro-1,4-phenylene)bis(ethyne-2,1-diyl)bis(1H-imidazole-4,2-diyl))bis(3-methylpyrrolidine-5,1-diyl))bis(3-methyl-1-oxobutane-2,1-diyl)dicarbamate

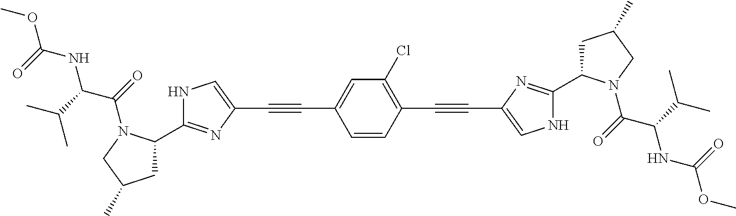

The same procedures are followed as mentioned for Example 4B.

LC/MS: m/z=659.30 (M+H⁺); RT=9.95 minutes.

### EXAMPLE 64

### ((S)-1-{(2S,4S)-4-Hydroxymethyl-2-[4-(4-{2-[(2S,4S)-1-((S)-2-methoxycarbonylamino-3-methyl-butyryl)-4-methyl-pyrrolidin-2-yl]-1H-imidazol-4-ylethynyl}-phenylethynyl)-1H-imidazol-2-yl]-pyrrolidine-1-carbonyl}-2-methyl-propyl)-carbamic acid methyl ester

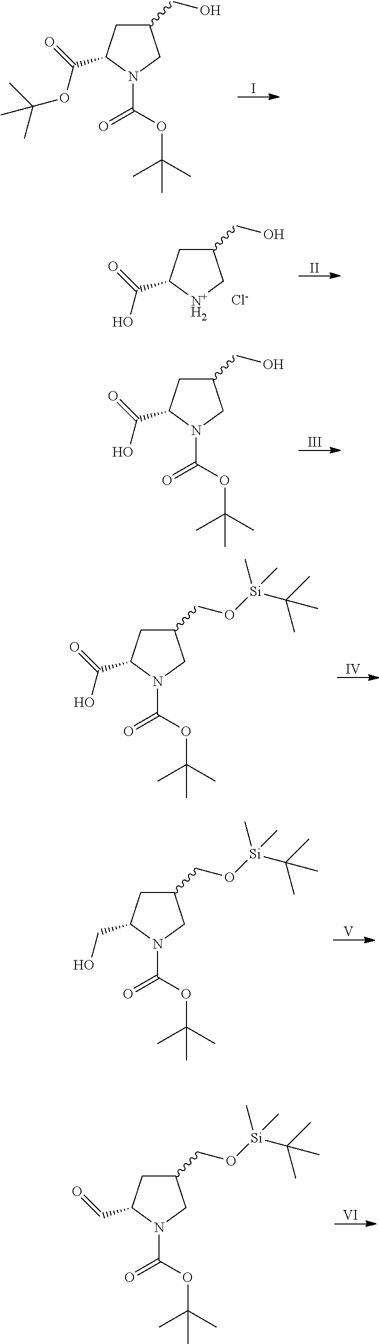

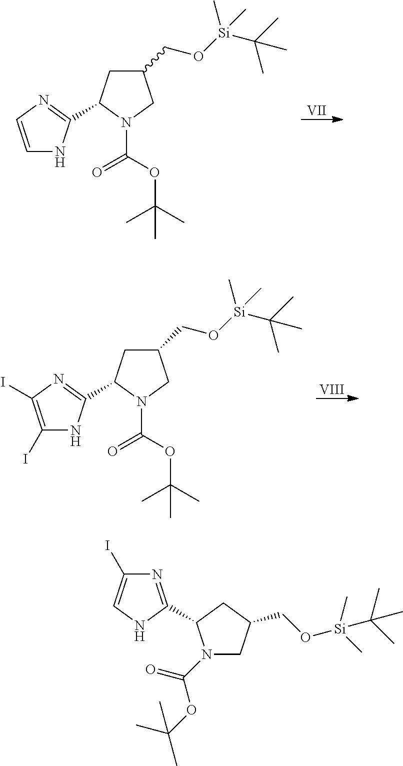

The starting material, (2S)-di-tert-butyl 4-(hydroxymethyl)pyrrolidine-1,2-dicarboxylate is obtained according to published literature procedures (Durand X., Hudhomme P., Khan J. A., Young D. W.; J. Chem. Soc., Perkin Trans. 1, 1996, 11, 1131).

### Step I

A suspension of (2S)-di-tert-butyl 4-(hydroxymethyl)pyrrolidine-1,2-dicarboxylate (3990 mg, 13.24 mmol) in HCl (4M in dioxane, 26.47 mL of 4 M, 105.9 mmol) is stirred for 90 minutes and evaporated to dryness. The sample is used as it is for the next step.

### Step II

To a solution of (2S)-2-carboxy-4-(hydroxymethyl)pyrrolidin-1-ium chloride (13.24 mmol) (from step I) in THF (20 mL) and water (6.6 mL) are sequentially added NaOH (7.408 mL of 2.5 M, 18.52 mmol) and tert-butoxycarbonyl tert-butyl carbonate (2.761 g, 2.906 mL, 12.65 mmol). The reaction mixture is stirred at room temperature overnight. NaOH (aqueous 2.5 M, 1.25 mL) is then added to bring the pH to 10-11. THF is evaporated and the residue is washed with ether (2×2 mL) and the residue is then acidified with 1M HCl to bring the pH to 1. The mixture is extracted by EtOAc (5×20 mL) and the combined organic layers are dried over Na₂SO₄, filtered and concentrated to dryness to give (2S)-1-(tert-butoxycarbonyl)-4-(hydroxymethyl)pyrrolidine-2-carboxylic acid (1.848 g, 84%).

### Step III

To a solution of (2S)-1-(tert-butoxycarbonyl)-4-(hydroxymethyl)pyrrolidine-2-carboxylic acid (1775 mg, 7.237 mmol) in DMF (17.75 mL) are sequentially added imidazole (1.084 g, 15.92 mmol) followed by TBDMSCl (2.291 g, 2.828 mL, 15.20 mmol). The mixture is stirred overnight at room temperature. The reaction mixture is extracted by EtOAc (5×70 mL) and the combined organic layers are washed with H₂O (3×35 mL), dried over Na₂SO₄, filtered and concentrated to dryness. The residue is purified by flash column chromatography on silica gel (0 to 20% methanol in CH₂Cl₂) to give a mixture of mono and bis-silylated product. The product is dissolved in 5:1 methanol (10 mL) water and stirred for 1 h. The mixture is concentrated to dryness and the residue is purified by flash column chromatography on silica gel (0 to 20% methanol in CH₂Cl₂) to give (2S)-1-(tert-butoxycarbonyl)-4-(((tert-butyldimethylsityl)oxy)methyl)pyrrolidine-2-carboxylic acid (1.239 g, 88%).

### Step IV

To a solution of (2S)-1-(tert-butoxycarbonyl)-4-(((tert-butyldimethylsilyl)oxy)methyl)pyrrolidine-2-carboxylic acid (1.080 g, 3.004 mmol) in THF (10.8 mL) is added Borane/THF (5.665 g, 6.308 mL of 1 M, 6.308 mmol) over 10 minutes in an ice bath. The mixture is stirred for 1 hour, warmed to room temperature and stirred for another 2 hours. The mixture is then cooled in an ice bath and diluted with 10% aqueous NH₄Cl (5 mL) followed by 10 mL of water. The reaction mixture is extracted by EtOAc (3×20 mL) and the combined organic layers are washed with H₂O (2×5 mL), dried over Na₂SO₄, filtered and concentrated to dryness. The residue is purified by flash column chromatography on silica gel (6 to 50% EtOAc in hexane) to give (2S)-tert-butyl 4-(((tert-butyldimethylsityl)oxy)methyl)-2-(hydroxymethyl)pyrrolidine-1-carboxylate (775 mg, 75%).

### Step V

To a solution of oxalyl chloride in CH₂Cl₂ (1.567 mL of 2 M, 3.134 mmol) in CH₂Cl₂ (3.6 mL) at −78° C. is added DMSO (489.7 mg, 444.8 μL, 6.267 mmol) and the mixture is stirred for 10 minutes. A solution of (2S)-4-Htert-butyl(dimethyl)silyl]oxymethyl]-2-(hydroxymethyl)pyrrolidine-1-carboxylate (722 mg, 2.089 mmol) in CH₂Cl₂ (8 mL) is then added and the mixture is stirred for another 30 minutes. DIPEA (1.619 g, 2.182 mL, 12.53 mmol) is added and the mixture is stirred for 30 minutes, warmed to room temperature, stirred again for 1 hour and diluted with 1M aqueous HCl (30 mL). The layers are separated and the aqueous layer is extracted by CH₂Cl₂ (2×20 mL). The combined organic layers are dried over Na₂SO₄, filtered, and concentrated to dryness. The residue is purified by flash column chromatography on silica gel (6 to 40% EtOAc in hexane) to give (2S)-tert-butyl 4-(((tert-butyldimethylsilyl)oxy)methyl)-2-formylpyrrolidine-1-carboxylate (647 mg, 90%).

### Step VI

Ammonia gas is bubbled in a solution of (2S)-4-Htert-butyl(dimethyl)silyl]oxymethyl]-2-formyl-pyrrolidine-1-carboxylate (581 mg, 1.691 mmol) in methanol (20 mL) at −20° C. for 15 minutes and glyoxal (aqueous) (1.718 g, 1.358 mL of 40% (w/w), 11.84 mmol) is added. The mixture is warmed gently to avoid strong bubbling and refluxed for 2 hours. The reaction mixture is then cooled to room temperature, methanol is evaporated and the residue is diluted with water (10 mL). The reaction mixture is extracted by CH₂Cl₂ (2×15 mL) and the combined organic layers are dried over Na₂SO₄, filtered and concentrated to dryness. The residue is purified by flash column chromatography on silica gel (0 to 15% methanol in CH₂Cl₂) to give (2S)-tert-butyl 4-(((tert-butyldimethylsityl)oxy)methyl)-2-(1H-imidazol-2-Apyrrolidine-1-carboxylate (454 mg, 70%).

### Step VII

To a solution of (2S)-tert-butyl 4-(((tert-butyldimethylsilyl)oxy)methyl)-2-(1H-imidazol-2-yl)pyrrolidine-1-carboxylate (450 mg, 1.179 mmol in CH₂Cl₂ (4.6 mL) is added N-iodosuccinimide (557.1 mg, 2.476 mmol). The reaction mixture is stirred at room temperature for 3 hours and diluted with water (5 mL). The layers are separated and aqueous portion is extracted by CH₂Cl₂ (2×5 mL) and the combined organic layers are dried over Na₂SO₄, filtered and concentrated to dryness. The residue is purified by flash column chromatography on silica gel (6 to 50% EtOAc in hexane) to give (2S,4S)-tert-butyl-4-(((tert-butyldimethylsilyl)oxy)methyl)-2-(4,5-diiodo-1H-imidazol-2-yl)pyrrolidine-1-carboxylate (230 mg, 31%) as a pure cis compound.

### Step VIII

To a solution of (2S,4S)-tert-butyl-4-(((tert-butyldimethylsilyl)oxy)methyl)-2-(4,5-diiodo-1H-imidazol-2-yl)pyrrolidine-1-carboxylate (225 mg, 0.355 mmol) and 0.5 M LiCl (710 μL) in THF at −20° C. is added iPrMgCl (177.6 μL of 2 M in THF, 0.3552 mmol) and the mixture is stirred for 15 minutes. Another portion of iPrMgCl (355.2 μL of 2 M in THF, 0.7104 mmol) is added and the mixture is warmed to room temperature and the mixture is stirred for another 2 hours. Another portion of iPrMgCl (150 μL of 2 M in THF) is added to complete the reaction and the mixture is stirred for another 30 minutes. The reaction mixture is diluted with 10% aqueous NH₄Cl (2 mL) and water (2 mL). The reaction mixture is extracted by AcOEt (3×10 mL), and the combined organic layers are dried over Na₂SO₄, filtered and concentrated to dryness. The residue is purified by flash column chromatography on silica gel (6 to 50% AcOEt in hexane) to give (2S,4S)-tert-butyl 4-(((tert-butyldimethylsilyl)oxy)methyl)-2-(4-iodo-1H-imidazol-2-yl)pyrrolidine-1-carboxylate (118 mg, 65%).

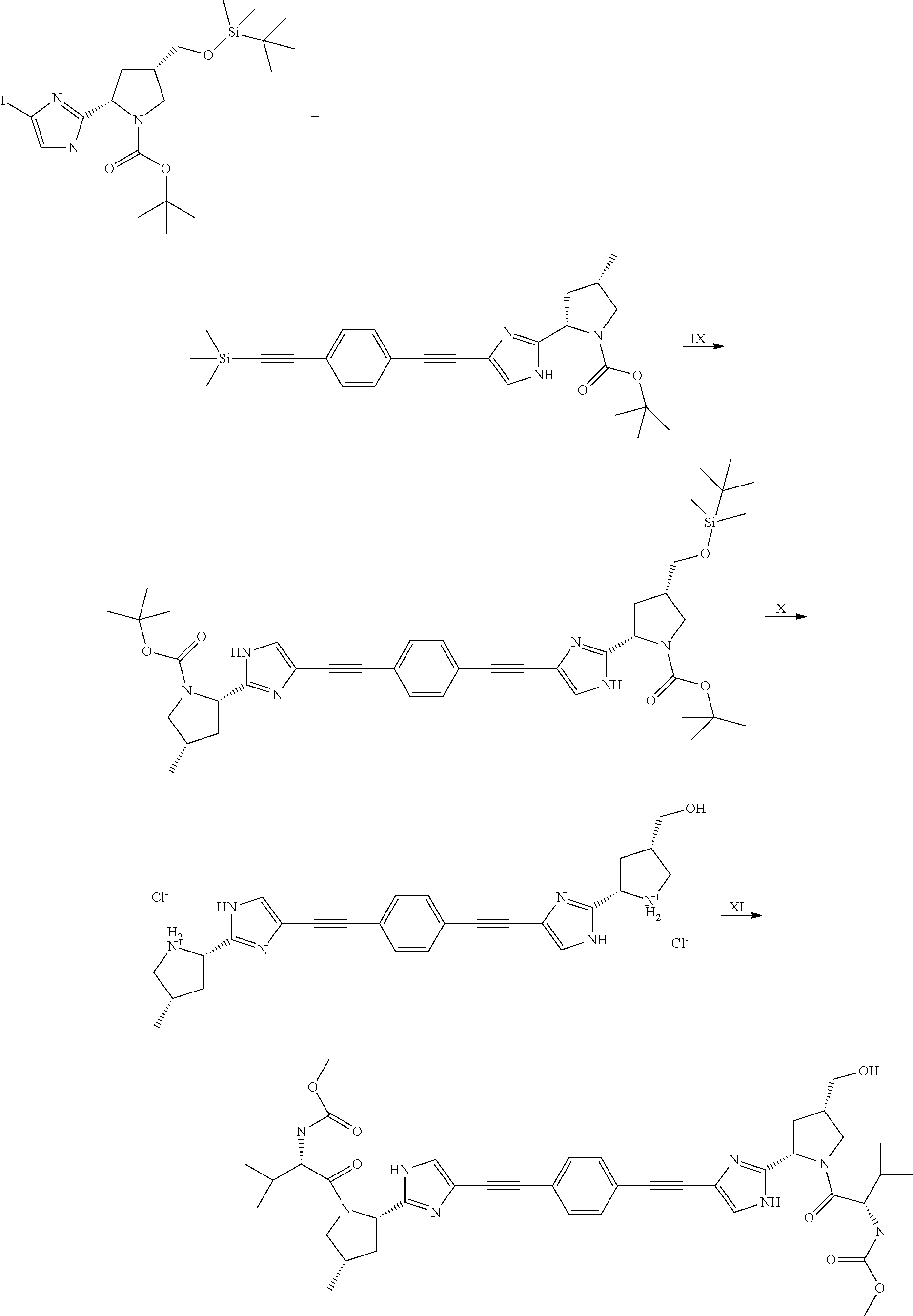

### Step IX

To a solution of tert-butyl (2S,4S)-4-Htert-butyl(dimethyl)silyl]oxymethyl]-2-(4-iodo-1H-imidazol-2-yl)pyrrolidine-1-carboxylate (66 mg, 0.1301 mmol), tert-butyl (2S,4S)-4-methyl-2-[4-[2-[4-(2-trimethylsilylethynyl)phenyl]ethynyl]-1H-imidazol-2-yl]pyrrolidine-1-carboxylate (58.24 mg, 0.1301 mmol), PdCl₂(dppf)-CH₂Cl₂ (5.312 mg, 0.006505 mmol) and CuI (1.239 mg, 0.006505 mmol) in DMF (2 mL) is added DBU (198.1 mg, 194.6 μL, 1.301 mmol) followed by one portion of water (1.2 μL, 0.5 eq.). The mixture is then warmed up to 60° C. and stirred at that temperature for 30 minutes before another portion of water is added (1.2 μL, 0.5 eq.) followed by another portion (1.2 μL, 0.5 eq.) after 30 minutes. The mixture is then stirred overnight at that temperature. The reaction is warmed to 100° C. and PdCl₂(dppf)-CH₂Cl₂ (4.6 mg, 0.0065 mmol) is added followed by CuI (1.2 mg, 0.0065 mmol). The mixture is stirred for another 3 hours at that temperature. The mixture is then cooled to room temperature and DMF is evaporated to dryness and the residue is purified by flash column chromatography on silica gel (0 to 15% methanol in CH₂Cl₂) to give (2S,4S)-tert-butyl 2-(4-((4-((2-((2S,4S)-1-(tert-butoxycarbonyl)-4-(((tert-butyldimethylsilyl)oxy)methyl)pyrrolidin-2-yl)-1H-imidazol-4-yl)ethynyl)phenyl)ethynyl)-1H-imidazol-2-yl)-4-methylpyrrolidine-1-carboxylate (26 mg, 26%).

### Step XI

A solution tert-butyl (2S,4S)-2-[4-[2-[4-[2-[2-[(2S,4S)-1-tert-butoxycarbonyl-4-[[tert-butyl(dimethyl)silyl]oxymethyl]pyrrolidin-2-yl]-1H-imidazol-4-yl]ethynyl]phenyl}ethynyl]-1H-imidazol-2-yl]-4-methyl-pyrrolidine-1-carboxylate (25 mg, 0.03311 mmol) in HCl (2 mL of 4 M in dioxane, 8.000 mmol) is stirred for 1 hour and evaporated to dryness. The sample is used as it is for the next step.

### Step XII

To a solution of [(3S,5S)-5-[4-[2-[4-[2-[2-[(2S,4S)-4-methylpyrrolidin-2-yl]-1H-imidazol-4-yl]ethynyl]phenyl]ethynyl]-1H-imidazol-2-yl]pyrrolidin-3-yl]methanol (Hydrochloric Acid (2)) (17 mg, 0.03311 mmol) and (2S)-2-(methoxycarbonylamino)-3-methyl-butanoic acid (11.60 mg, 0.06622 mmol) in DMF (2 mL) is cooled in an ice bath and are sequentially added HATU (26.44 mg, 0.06953 mmol) and DIPEA (25.68 mg, 34.61 μL, 0.1987 mmol) under nitrogen atmosphere. The reaction mixture is stirred at that temperature for 1.5 hours and evaporated to dryness. The residue is purified successively by flash column chromatography on silica gel (0 to 15% methanol in CH₂Cl₂) followed by reverse phase preparative HPLC to afford ((S)-1-[(2S,4S)-4-hydroxymethyl-2-[4-(4-{2-[(2S,4S)-1-((S)-2-methoxycarbonylamino-3-methyl-butyryl)-4-methyl-pyrrolidin-2-yl]-1H-imidazol-4-ylethynyl}-phenylethynyl)-1H-imidazol-2-yl]-pyrrolidine-1-carbonyl}-2-methyl-propyl)-carbamic acid methyl ester (6.2 mg, 26%).

¹H NMR (400 MHz, DMSO-d₆): δ 12.09 (broad s, 2H), 7.43 (m, 4H), 7.21 (m, 2H), 4.83 (m, 2H), 4.05 (m, 4H), 3.50 (s, 6H), 3.45 (m, 2H), 3.31 (s, 1H), 3.15 (m, 2H), 2.23 (m, 4H), 1.86 (m, 4H), 1.21 (m, 3H), 1.06 (m, 2H), 0.75 (m, 6H). LC/MS: m/z=755.51 (M+H⁺).

### EXAMPLES 65 AND 66

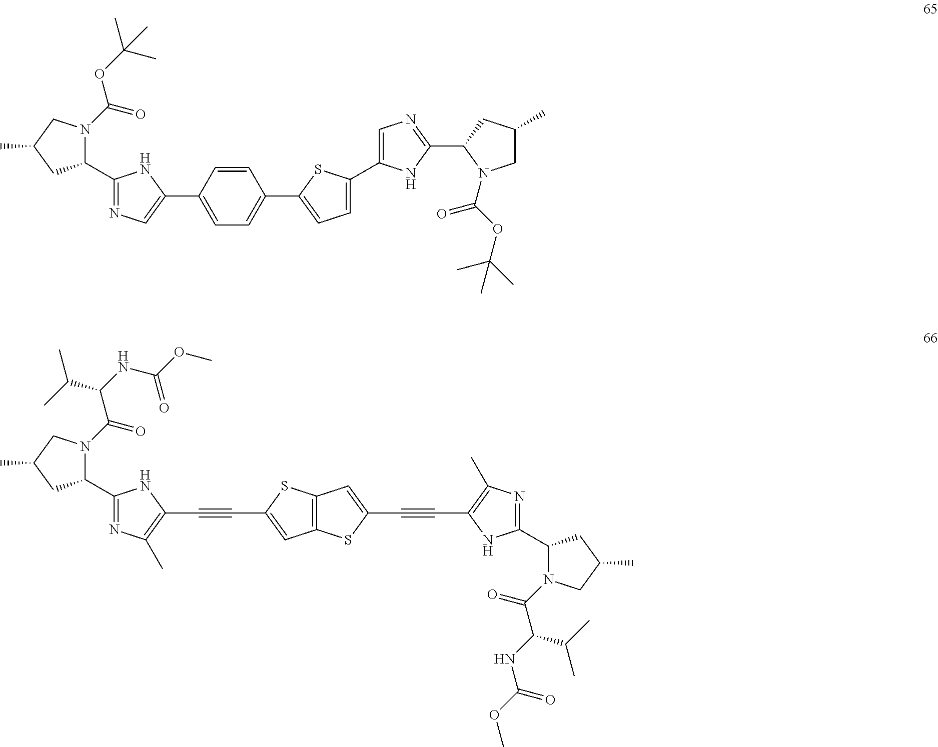

The above compounds were prepared according to the procedures disclosed herein.

### EXAMPLE 67

### Methyl N-[(1S)-1-[(2S,4S)-2-[4-[2-[2-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]benzothiophen-6-yl]ethynyl]-1H-imidazol-2-yl]-4-methyl-pyrrolidine-1-carbonyl]-2-methyl-propyl]carbamate

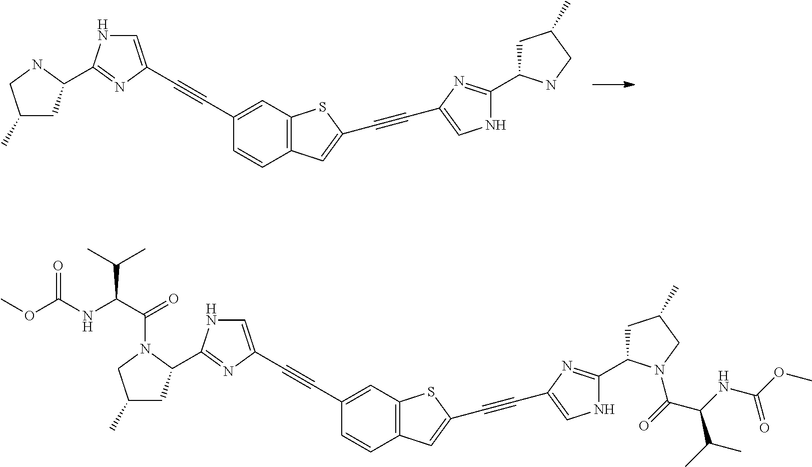

To a solution of 2-[(2S,4S)-4-methylpyrrolidin-2-yl]-4-[2-[2-[2-[2-[(2S,4S)-4-methylpyrrolidin-2-yl]-1H-imidazol-4-yl]ethynyl]benzothiophen-6-yl]ethynyl]-1H-imidazole (59.3 mg, 0.12 mmol) in DMF (2 mL) are added (2S)-2-(methoxycarbonylamino)-3-methyl-butanoic acid (47.5 mg, 0.27 mmol), HATU (112.6 mg, 0.29 mmol) and DIPEA (159.5 mg, 215.0 μL, 1.234 mmol). The mixture is stirred at rt overnight. After removal of the solvent under reduced pressure, the residue is purified by silica gel column chromatography using methanol (0-6%) in CH₂Cl₂, and the major fraction is further purified using revers-phase prep-HPLC to obtain methyl N-[(1S)-1-[(2S,4S)-2-[4-[2-[2-[2-[2-[(2S,4S)-1-[(2S)-2-(methoxycarbonylamino)-3-methyl-butanoyl]-4-methyl-pyrrolidin-2-yl]-1H-imidazol-4-yl]ethynyl]benzothiophen-6-yl]ethynyl]-1H-imidazol-2-yl]-4-methyl-pyrrolidine-1-carbonyl]-2-methyl-propyl]carbamate (46 mg, 0.05699 mmol, 46.18%) as an off-white solid. LC/MS: m/z=795.40 (M+H⁺). ¹H NMR (CD₃OD, 400 MHz): δ 7.24-7.95 (m, 6H), 4.95 (m, 2H), 4.20 (m, 4H), 3.62 (s, 6H), 2.46 (m, 4H), 1.96 (m, 4H), 1.15 (m, 6H), 0.84 (m, 14H).

### EXAMPLE 68

### Methyl N-[(1S)-1-[(2S,4S)-4-methoxy-2-[4-[2-[4-[2-[2-[(2S,4S)-4-methoxy-1-[(2S)-2-(methoxy-carbonylamino)-3-methyl-butanoyl]pyrrolidin-2-yl]-1H-imidazol-4-yl]ethynyl]phenyl]ethynyl]-1H-imidazol-2-yl]pyrrolidine-1-carbonyl]-2-methyl-propyl]carbamate

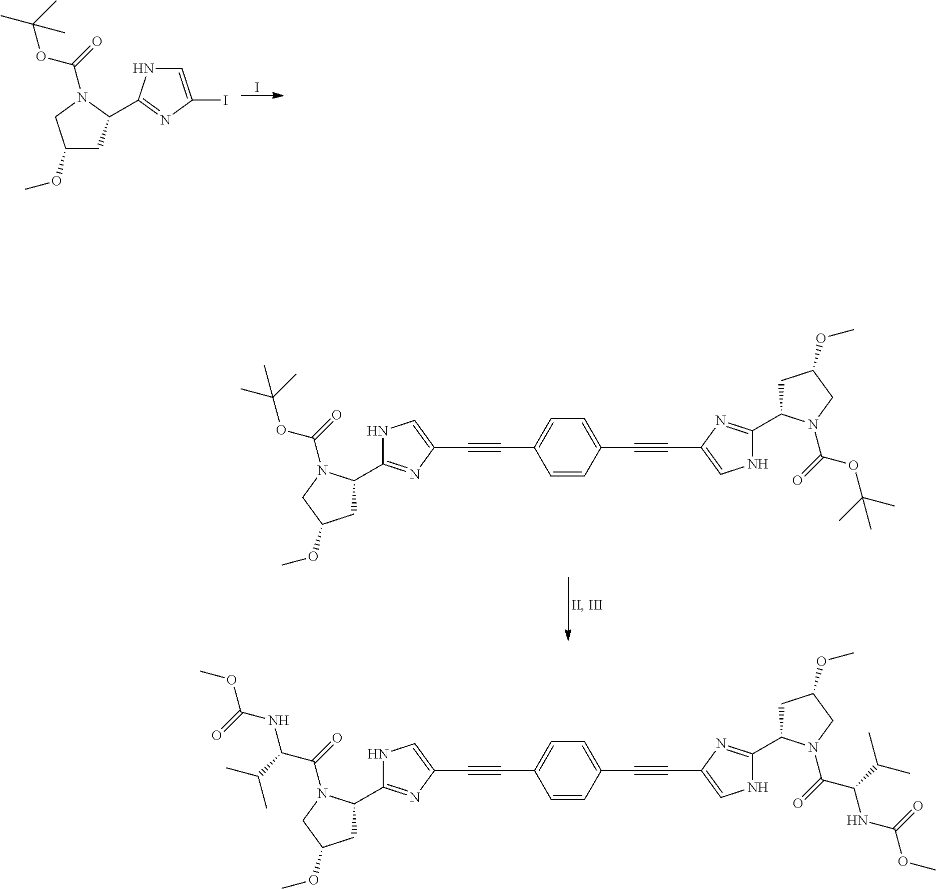

The title compound is prepared from tert-butyl (2S,4S)-2-(4-iodo-1H-imidazol-2-yl)-4-methoxy-pyrrolidine-1-carboxylate using the reactions described below.

### Step I:

To a mixture of tert-butyl (2S,4S)-2-(4-iodo-1H-imidazol-2-yl)-4-methoxy-pyrrolidine-1-carboxylate, trimethyl-[2-[4-(2-trimethylsilylethynyl)phenyl]ethynyl]-silane, PdCl₂(dppf)-CH₂Cl₂ and copper Iodide is added DMF under nitrogen atmosphere. The reaction mixture is degassed thrice (vacuum and nitrogen gas) and then DBU and water are added and heated at 60° C. for 8 hours. The reaction mixture is cooled to 0° C., diluted with water and filtered.

### Step II:

To a solution of the product from step I in dry MeOH (400 μL) is added HCl in Dioxane. The reaction mixture is stirred at rt for 2 hours and concentrated to afford the corresponding amine as the HCl salt. This material is used as such in the next step.

### Step III:

To a cold (−10 to −5° C.) stirred solution of the product from step II and HATU in DMF is added DIPEA dropwise. The resultant reaction mixture is slowly warmed up to rt and stirred overnight. The reaction mixture is diluted with water, the resultant white precipitate is collected by filtration and the filtrate is refiltered. The combined precipitates are washed with water and heptanes, dried under high vacuum and purified by reverse phase HPLC to afford the title compound.

¹H NMR (400 MHz, CD₃OD, Peaks for the major rotamer): δ 7.44 (s, 4H), 7.23 (s, 2H), 5.2-5.1 (m, 2H), 4.2-3.0 (m, 8H), 3.63 (s, 6H), 3.34 (s, 6H), 2.6-1.9 (m, 6H), 0.92-0.86 (doublets, 12H).

LC/MS, m/z=771.59 (M+H⁺); Rt: 7.25 minutes.

HPLC (RT)=11.70 minutes, Method: 20%-60% AcCN-water (0.01% TFA) for 40 min, Gemini C18 3 μm, 4.6 mm×250 mm.

### EXAMPLE 69

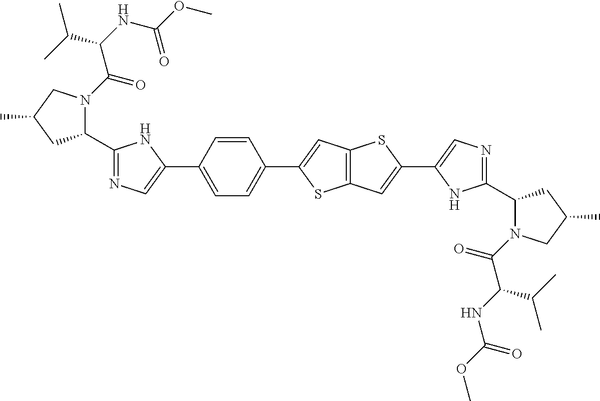

The above compound was prepared according to the procedures disclosed herein.

### EXAMPLE 70

### Activity Determination Using the ELISA and the Sub-Genomic Replicon 1a Cell Line

The cell line W11.8 containing the sub-genomic HCV replicon of genotype 1a is used to determine the potency of the drugs. The RNA replication in presence of different drug concentrations is indirectly measured in this cell line by the level of NS5A protein content upon drug treatment for four days. It is shown that the level of the NS5A protein correlates well with the level of HCV RNA in the replicon cell line. Cells are split twice a week in order to keep the confluence state below 85% of the culture flask surface area. The culture media used for cell passaging consists of DMEM-10% foetal bovine serum with 100 UI/mL penicillin, 100 μg/mL streptomycin, 2 mM glutamine, 1 mM sodium pyruvate, non-essential amino acids (1×) and 600 μg/mL of G418 final concentrations. Monolayer of the W11.8 cells is trypsinized and cells are counted. Cells are diluted at 50,000 cells/mL with complete DMEM without G418, then approximately 5,000 viable cells (100 μL) are plated per well in a white opaque 96-well microtiter plate. After an incubation period of 2-4 hours at 37° C. in a 5% CO2 incubator, compounds are added at various concentrations. Drugs are resuspended in DMSO at a stock concentration of 10 mM. Then, drugs are serially diluted at twice the final concentration in the same medium. One volume (100 μL) of each drug dilution is then added to each well that contains cells.

A control compound is used as an internal standard for each plate assay. Sixteen wells are used as control (0% inhibition) without drug. Eight wells are used as background control (100% inhibition) containing 2 μM (final concentration) of the control drug that was shown to inhibit the NS5A expression at ≈100% and is nontoxic to the cells. Values from 100% inhibited wells were averaged and used as the background value. Cells are further incubated for four days at 37° C. in a 5% CO₂ incubator. Following the incubation time of four days, the media is removed and wells are washed once with 150 μL of PBS at room temperature for five minutes. Cells are then fixed for five minutes using 150 μL per well of cold (−20° C.) fixative solution (50% methanol/50% acetone mix). Cells are then washed twice with 150 μL of PBS (phosphate buffered saline) per well, following the addition of 150 μL of blocking solution, cells are incubated for one hour at 37° C. to block non-specific sites. The blocking solution is removed and cells are washed twice with 150 μL of PBS per well and once with 150 μL of PBSTS solution (PBS/0.1% Triton X-100/0.02% SDS) per well. Then, 50 μL of mouse monoclonal anti-NS5A antibody (Santa Cruz, Cat. No. sc-52417) is added in each well, diluted 1/1,000 in the blocking solution and incubated at 4° C. overnight. Next day, media is removed and plates are washed five times with 150 μL of PBS per well with five-minute incubations at room temperature. Then 50 μL per well of peroxidase-conjugated donkey anti-mouse antibody (Jackson Immunoresearch, Cat. No. 715-036-150) diluted 1/10,000 in the blocking solution is added and incubated at room temperature for three hours on a shaker (500 rpm). Plates are washed four times with 150 μL of PBSTS solution per well and once with 150 μL of PBS. Then, substrate solution (100 μl, SuperSignal ELISA Pico Chemiluminescent Substrate, Fisher Cat. No. 37069) is added in each well and plates are incubated 60 minutes at room temperature prior to reading the luminescence (relative light units) on the Analyst HT plate reader. The percentage of inhibition at each drug concentration tested (in duplicate) is calculated. The concentration required to reduce viral replication by 50% (IC₅₀) is then determined from dose response curves using nonlinear regression analysis with the GraphPad Prism software, version 2.0 (GraphPad Software Inc., San Diego, Calif., USA).

### EXAMPLE 71

### Cell-Based Luciferase Reporter HCV (Ib) RNA Replication Assay Cell Culture

Replicon cell lines Huh-5.2 are derived from the Huh-7 hepatocarcinoma cell line are maintained in culture as generally described in Krieger, N; Lohmann, V; Bartenschlager, R. Enhancement of hepatitis C virus RNA replication by cell culture-adaptive mutations. J. Virol. 2001, 75, 4614-4624. The Huh-5.2 cells contain the highly cell culture-adapted replicon I₃₈₉ Luc-ubi-neo/NS3-375.1 construct that carries, in addition to the neomycin gene, an integrated copy to the firefly luciferase gene (Krieger, N; Lohmann, V; Bartenschlager, R. Enhancement of hepatitis C virus RNA replication by cell culture-adaptive mutations. J. Virol. 2001, 75, 4614-4624). This cell line allows measurement of HCV RNA replication and translation by measuring luciferase activity. It has been previously shown that the luciferase activity tightly follows the replicon RNA level in these cells (Krieger, N; Lohmann, V; Bartenschlager, R. Enhancement of hepatitis C virus RNA replication by cell culture-adaptive mutations. J. Virol. 2001, 75, 4614-4624). The Huh-ET cell line has the same features as those mentioned for Huh-5.2 cell line, except that ET cells are more robust and contain an adaptative mutation in the HCV NS4B gene instead of NS5A. Both cell lines are maintained in cultures at a sub-confluent level (<85%) as the level of replicon RNA is highest in actively proliferating cells. The culture media used for cell passaging consist of DMEM (Gibco BRL Laboratories, Mississauga, ON, Canada) supplemented with 10% foetal bovine serum with 1% penicilin/streptomycin, 1% glutamine, 1% sodium pyruvate, 1% non-essential amino acids, and 180 μg/ml of G418 final concentration. Cells are incubated at 37° C., in an atmosphere of 5% CO₂ and passaged twice a week to maintain sub-confluence.

Approximately 3000 viable Huh-ET cells (100 μl) are plated per well in a white opaque 96-well microtiter plate. The cell culture media used for the assay is the same as described above except that it contains no G418 and no phenol red. After an incubation period of 3-4 hours at 37° C. in a 5% CO₂ incubator, compounds (100 μl) are added at various concentrations. Cells are then further incubated for 4 days at 37° C. in a 5% CO₂ incubator. Thereafter, the culture media is removed and cells are lysed by the addition of 95 μL of the luciferase buffer (luciferin substrate in buffered detergent). Cell lysates are incubated at room temperature and protected from direct light for at least 10 minutes. Plates are read for luciferase counts using a luminometer (Wallac MicroBeta Trilux, Perkin Elmer™, MA, USA).

The 50% inhibitory concentrations (IC₅₀s) for inhibitory effect are determined from dose response curves using eleven concentrations per compound in duplicate. Curves are fitted to data points using nonlinear regression analysis, and IC₅₀s are interpolated from the resulting curve using GraphPad Prism software, version 2.0 (GraphPad Software Inc., San Diego, Calif., USA).

Table 1C shows comparative data for exemplary compounds of formula (I) (entries 1-3) and formula (IA) (entries 4-5). As is shown in the table, the compounds having a substituent at the 4-position of the pyrrolidine ring (i.e. compounds of the invention where R₄ and R_{4′} are methyl). Data shows IC₅₀ values against the sub-genomic replicon 1a cell line.

TABLE 1C

| Entry | Compound | Structure | IC₅₀ (pM) |
|---|---|---|---|
| 1 | 4 | 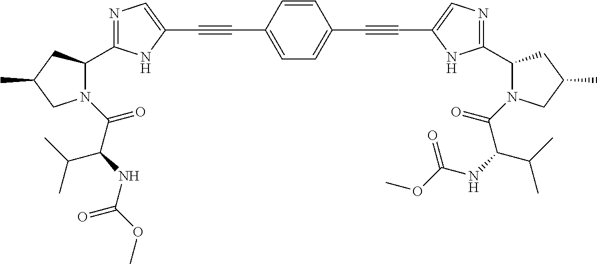 | 55 |
| 2 | | 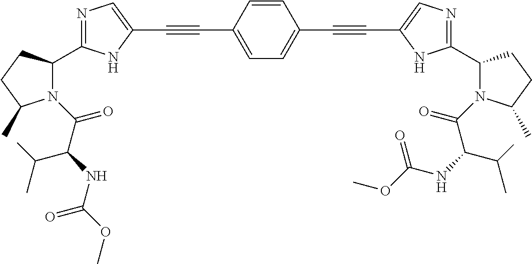 [SMILES: COC(=O)N[C@H](C(=O)N1[C@@H](C)CC[C@H]1c1ncc(C#Cc2ccc(C#Cc3cnc([C@@H]4CC[C@H](C)N4C(=O)[C@@H](NC(=O)OC)C(C)C)[nH]3)cc2)[nH]1)C(C)C] | 240 |
| 3 | | 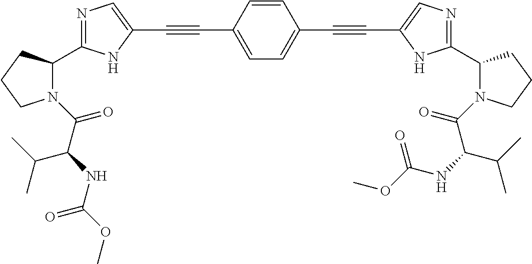 [SMILES: COC(=O)N[C@H](C(=O)N1CCC[C@H]1c1ncc(C#Cc2ccc(C#Cc3cnc([C@@H]4CCCN4C(=O)[C@@H](NC(=O)OC)C(C)C)[nH]3)cc2)[nH]1)C(C)C] | 4800 |
| 4 | 1 | 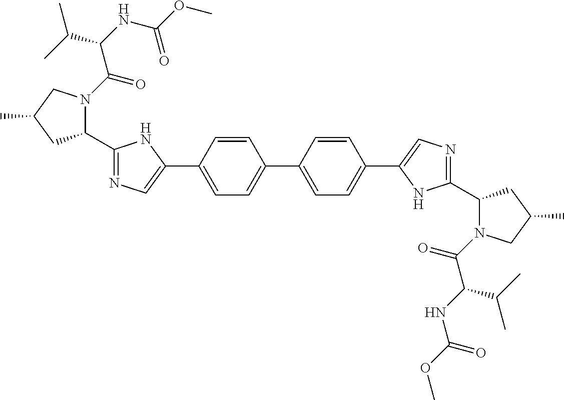 | 17 |
| 5 | | 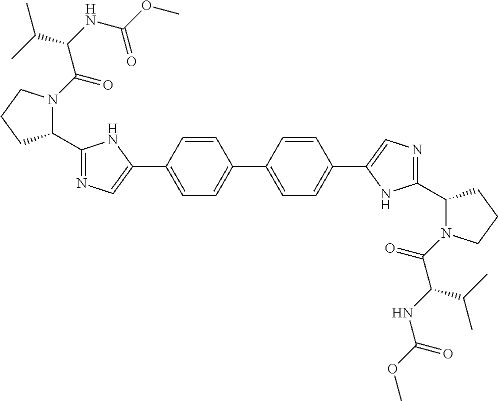 [SMILES: COC(=O)N[C@H](C(=O)N1CCC[C@H]1c1ncc(-c2ccc(-c3ccc(-c4cnc([C@@H]5CCCN5C(=O)[C@@H](NC(=O)OC)C(C)C)[nH]4)cc3)cc2)[nH]1)C(C)C] | 50 |

Table 1D shows compounds representative of the present invention and the EC50 values against the HCV 1b genotype. EC₅₀ ranges are presented in micromolar as follows: μM+++<=0.005<++<=5.0<+.

TABLE 1D

| | | Retention | | |
|---|---|---|---|---|
| | M + 1 | time | | IC₅₀ |
| Compound | (observed) | (minutes) | ¹H—NMR | (μM) |
| 1 | 767.69 | 7.14 | | +++ |
| 2 | 755.5 | 9.27 | | +++ |
| 3 | 717.5 | 8.17 | | +++ |
| 4 | 739.6 | 8.24 | | +++ |
| 5 | 743.44 | 8.11 | | +++ |
| 6 | 789.5 | 9.16 | | +++ |
| 7 | 771.58 | 7.5 | | +++ |
| 8 | 767.59 | 8.81 | | +++ |
| 9 | 739.51 | 8.89 | | +++ |
| 10 | 802.5 | 9.86 | | +++ |
| 11 | 767.4 | 9.22 | | +++ |
| 12 | 753.4 | 8.77 | | +++ |
| 13 | 752.58 | 9.14 | | +++ |
| 14 | 773.49 | 9.21 | | +++ |
| 15 | 635.48 | 7.12 | | ++ |
| 16 | 753.56 | 8.57 | | +++ |
| 17 | 747.57 | 6.13 | | ++ |
| 18 | 767.64 | 7.94 | | +++ |
| 19 | 739.65 | 7.31 | | +++ |
| 20 | 865.36 | 10.94 | | +++ |
| 21 | 991.17 | 14.56 | | +++ |
| 22 | 807.44 | 9.65 | | +++ |
| | | | 1H NMR (300 MHz, DMSO) d | |
| | | | 12.60 (s, 0.4H), 12.20 (s, | |
| | | | 1.6H), 7.48 (s, 5.5H), 7.20 (s, | |
| | | | 0.5H), 4.76 − 4.59 (m, 2H), | |
| | | | 3.75-3.59 (m, 2H), 3.04 − | |
| | | | 2.91 (m, 2H), 2.41 − 2.30 (m, | |
| | | | 2H), 2.29 − 2.13 (m, 2H), 1.72 − | |
| | | | 1.49 (m, 2H), 1.37 (s, 6H), | |
| 23 | 625.45 | 9.13 | 1.14 (s, 12H), 1.02 (d, J = 6.2 | ++ |
| | | | Hz, 6H). | |
| 24 | 425 | 5.08 | | ++ |
| 25 | 725.5 | 7.55 | | +++ |
| 26 | 753.45 | 7.14 | | +++ |
| 27 | 739.57 | 9.06 | | ++ |
| 28 | 767.53 | 8.11 | | +++ |
| 29 | 803.55 | 8.73 | | +++ |
| 30 | 795.6 | 8.47 | | +++ |
| 31 | 785.27 | 8.31 | | +++ |
| 32 | 739.49 | 8.39 | | ++ |
| 33 | 739.53 | 8.17 | | +++ |
| 34 | 625.41 | 8.91 | | |
| 35 | 424.99 | 4.94 | | |
| 36 | 757.4 | 17.58 | | +++ |
| 37 | 817.63 | 8.57 | | +++ |
| 38 | 739.47 | 8.32 | | +++ |
| 39 | 582.42 | 6.49 | | ++ |
| 40 | 797.53 | 8.7 | | +++ |
| 41 | 767.6 | 9.06 | | +++ |
| 42 | 739.4 | 9.49 | | +++ |
| 43 | 767.5 | 8.93 | | +++ |
| 44 | 747.49 | 8.05 | | +++ |
| 45 | 819.65 | 10.3 | | +++ |
| 46 | 838.4 | 8.49 | | +++ |
| 47 | 781.17 | 6.92 | | +++ |
| 48 | 875.45 | 16.08 | | +++ |
| 49 | 741.42 | 6.46 | | +++ |
| 50 | | | | |
| 51 | 743.4 | 2.79 | | |
| 52 | 739.43 | 8.62 | | ++ |
| 53 | 663.44 | 7.61 | | +++ |
| 54 | 682 | 8.55 | | +++ |
| 55 | 790.3 | 8.64 | | +++ |
| 56 | 740.45 | 7.75 | | +++ |
| 57 | 797.4 | 8.57 | | +++ |
| 58 | 829.84 | 2 | | +++ |
| 59 | 799.51 | 7.24 | | ++ |
| 60 | 745.3 | 8.74 | | +++ |
| 61 | 795.4 | 9.48 | | +++ |
| 62 | 764.45 | 9.01 | | +++ |
| 63 | 773.6 | 18.6 | | +++ |
| 64 | 755.51 | 7.45 | | +++ |
| 65 | 659.59 | 1.76 | | ++ |
| 66 | 829.55 | 16.6 | | +++ |
| 67 | 795.57 | 9.21 | | +++ |
| 68 | 771.59 | 7.25 | | +++ |
| 69 | 829.37 | 1.75 | | +++ |

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compound of Formula (III): 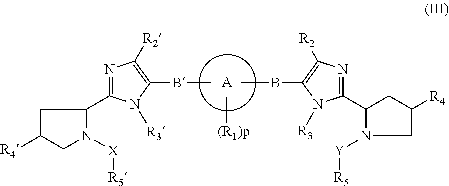
wherein
A is C₆₋₁₄ aryl, 4-12 membered heterocycle, C₃₋₁₀ cycloalkyl, 5-12 membered heteroaryl, or a bond;
B and B′ are each independently C₁₋₆ alkyl, C₂₋₆ alkenyl, or C₂₋₆ alkynyl;
R₁ is H, halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, —P(═O)ORₐOR_{b}, C₁₋₆ alkyl, C₂₋₆ alkenyl, C₂₋₆ alkynyl, or C₁₋₆ halogenated alkyl, or any two occurrences of R₁ can be taken together with the atoms to which they are attached to form a 5-7 cycloalkyl which is unsubstituted or substituted one or more times by R¹⁰ or a 5-7 membered heterocycle which is unsubstituted or substituted one or more times by R¹², wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
R₂ and R₂′ are each independently H, halogen, C₁₋₆ alkyl, —(CH₂)₁₋₃OH, —ORₐ, —C(═O)ORₐ, —C(O)NRₐR_{b}, —C(═O)OH, C₆₋₁₂ aryl, or 5-12 membered heteroaryl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
R₃ and R₃′ are each independently H, C₁₋₆ alkyl, C₂₋₆ alkenyl, or C₂₋₆ alkynyl;
R₄ and R₄′ are each independently halogen, C₁₋₆ alkyl, hydroxyl, C₆₋₁₄ aryl, or C₁₋₄ alkoxy;
X and Y are each independently 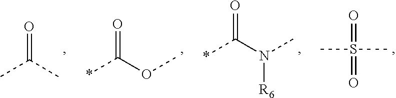
wherein the bond marked with an asterisk (*) indicates the attachment to the nitrogen of ring C or C′;
R₅ and R₅′ are each independently H, C₁₋₁₂ alkyl which is unsubstituted or substituted one or more times by R¹⁰, C₂₋₁₂ alkenyl which is unsubstituted or substituted one or more times by R¹⁰, C₂₋₁₂ alkynyl which is unsubstituted or substituted one or more times by R¹⁰, C₆₋₁₄ aryl which is unsubstituted or substituted one or more times by R¹¹, C₇₋₁₆ aralkyl which is unsubstituted or substituted one or more times by R¹¹, 5-12 membered heteroaryl which is unsubstituted or substituted one or more times by R¹¹, 6-18 membered heteroaralkyl which is unsubstituted or substituted one or more times by R¹¹, 3-12 membered heterocycle which is unsubstituted or substituted one or more times by R¹², or 4-18 membered heterocycle-alkyl which is unsubstituted or substituted one or more times by R₁₂;
R₆ is H, C₁₋₆ alkyl, or halogenated C₁₋₆ alkyl, or can be merged with R₆ or R₆′ to form a 3-12 membered heterocycle;
p is 0, 1, 2, 3 or 4;
R¹⁰ is halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
R¹¹ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
R¹² is halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}S O₂Rₐ, —NR_{b}S O₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
wherein as valency allows in B, B′, Rₐ, R_{b}, R_{c}, and R_{d}, R₁, R₂, R₂′, R₃, R₃′, R₄, R₄, R¹⁰, R₁₁ and R¹² each of alkyl, alkenyl, alkynyl, alkoxy, aryl, aralkyl, heteroaryl, heteroaralkyl, heterocycle, or heterocycle-alkyl is independently unsubstituted or substituted one or more times by halogen, —ORₐ′, oxo, —NR_{a′}R_{b′}, ═NO—R_{c}, —C(═O)ORₐ′, —C(O)NR_{a′}R_{b′}, —C(═O)OH, —C(═O)R_{a′}, —C(═NOR_{c′})R_{a′}, —C(═NR_{c′)NR}_{a′}R_{b′}, —NR_{d′}C(═O)NR_{a′}R_{b′}, —NR_{b′}C(═O)R_{a′}, —NR^{d′}C(═NR_{c′})NR_{a′}R_{b′}, —NR_{b′}C(═O)OR_{a′}, —OC(═O)NR_{a′}R_{b′}, —OC(═O)R_{a′}, —OC(═O)OR_{a′}, hydroxyl, nitro, azido, cyano, —S(O)₀₋₃R_{a′}, —SO₂NR_{a′}R_{b′}, or —NR_{b′}SO₂R_{a′}; wherein R_{a′}-R_{d′} are each independently H or C₁₋₁₂ alkyl; or
a pharmaceutically acceptable salt thereof.

2-48. (canceled)

49. The compound according to claim 1, wherein said compound is of formula (IV): 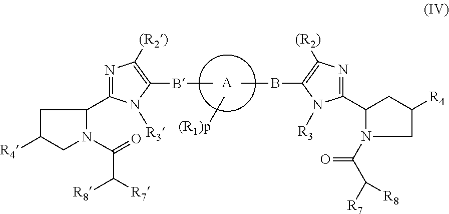
or a pharmaceutically acceptable salt thereof;
wherein
R₇ and R₇′ are each independently C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, benzyl which is unsubstituted or substituted one or more times by R¹¹, 5-6 membered heteroaryl which is unsubstituted or substituted one or more times by R¹¹, 6-7 membered heteroaralkyl which is unsubstituted or substituted one or more times by R¹¹, 3-6 membered heterocycle which is unsubstituted or substituted one or more times by R¹², or 4-7 membered heterocycle-alkyl which is unsubstituted or substituted one or more times by R¹²; and
R₈ and R₈′ are each independently —NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —NR_{b}SO₂Rₐ, —NR_{b}SO₂NRₐR_{b}, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

50-54. (canceled)

55. The compound according to claim 49, wherein said compound is of formula (V): 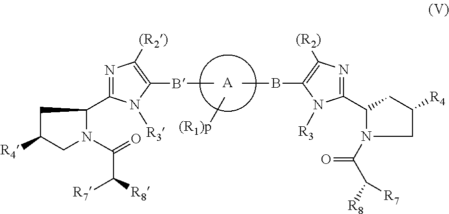
or a pharmaceutically acceptable salt thereof.

56-58. (canceled)

59. A compound of formula (IIIA): 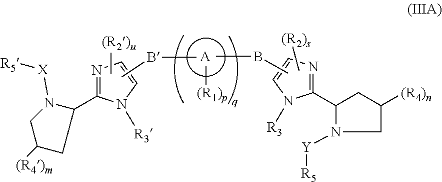
or a pharmaceutically acceptable salt thereof, wherein
each A is independently C₆₋₁₄ aryl, 4-12 membered heterocycle, C₃₋₁₀ cycloalkyl, or 5-12 membered heteroaryl;
B and B′ are each independently absent, C₁₋₆ alkyl, C₂₋₆ alkenyl, or C₂₋₆ alkynyl; wherein when q is 1 then at least one of B and B′ is absent;
R₁ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, —P(═O)ORₐOR_{b}, C₁₋₆ alkyl which is unsubstituted or substituted one or more times by R¹⁰, C₂₋₆ alkenyl which is unsubstituted or substituted one or more times by R¹⁰, C₂₋₆ alkynyl which is unsubstituted or substituted one or more times by R¹⁰, or any two occurrences of R₁ can be taken together with the atoms to which they are attached to form a 5-7 cycloalkyl which is unsubstituted or substituted one or more times by R¹¹ or a 5-7 membered heterocycle which is unsubstituted or substituted one or more times by R¹²;
Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
R_{2′} and R₂ are independently H, halogen, C₁₋₁₀ alkyl, C₁₋₆halogenated alkyl, —(CH₂)₁₋₆OH, —NR_{b}C(═O)RₐC₆₋₁₂ aryl, or 5-12 membered heteroaryl;
R₃ and R₃′ are each independently H, C₁₋₆ alkyl, —(CH₂)₁₋₆OH, C₂₋₆ alkenyl, or C₂₋₆ alkynyl;
R₄ and R₄′ are each independently —NRₐR_{b}, —C(O)NRₐR_{b}, —(CH₂)₁₋₆OH, C₁₋₆ alkyl, C₁₋₆ halogenated alkyl, C₆₋₁₄ aryl, or C₁₋₆alkoxy; wherein two occurrence of R₄ can be taken together with the atoms to which they are attached to form a C₁₋₆ alkenyl which is unsubstituted or substituted one or more times by R¹⁰, a 4-7 cycloalkyl which is unsubstituted or substituted one or more times by R¹¹ or a 4-7 membered heterocycle which is unsubstituted or substituted one or more times by R¹²; wherein two occurrence of R₄′ can be taken together with the atoms to which they are attached to form a C₁₋₆ alkenyl which is unsubstituted or substituted one or more times by R¹⁰, a 3-7 cycloalkyl which is unsubstituted or substituted one or more times by R¹¹ or a 4-7 membered heterocycle which is unsubstituted or substituted one or more times by R¹²; wherein Rₐ-R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
X and Y are each independently 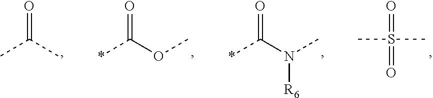
wherein the asterisk (*) indicates the point of attachment to the nitrogen of ring C or C′;
R₅ and R₅′ are each independently H, C₁₋₁₈ alkyl which is unsubstituted or substituted one or more times by R¹⁰, C₂₋₁₂ alkenyl which is unsubstituted or substituted one or more times by R¹⁰, C₂₋₁₂ alkynyl which is unsubstituted or substituted one or more times by R¹⁰, C₆₋₁₄ aryl which is unsubstituted or substituted one or more times by R¹¹, C₇₋₁₆ aralkyl which is unsubstituted or substituted one or more times by R¹¹, 5-12 membered heteroaryl which is unsubstituted or substituted one or more times by R¹¹, 6-18 membered heteroaralkyl which is unsubstituted or substituted one or more times by R¹¹, 3-12 membered heterocycle which is unsubstituted or substituted one or more times by R¹², or 4-18 membered heterocycle-alkyl which is unsubstituted or substituted one or more times by R¹²;
R₆ is H, C₁₋₆ alkyl, or halogenated C₁₋₆ alkyl;
m and n are a positive integer and when combined are 1, 2, 3, or 4, provided that each of m and n are not 3 or 4;
p is 0, 1, 2, 3 or 4;
q is 1 or 2;
u is 0 or 1;
s is 0 or 1;
R¹⁰ is halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b};
R¹¹ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and
R¹² is halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

60. (canceled)

61. (canceled)

62. A compound of formula (IIIB): 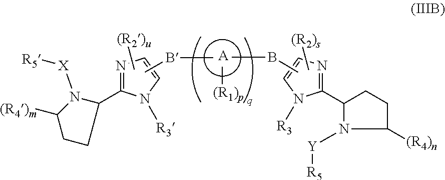
or a pharmaceutically acceptable salt thereof, wherein
each A is independently C₆₋₁₄ aryl, 4-12 membered heterocycle, C₃₋₁₀ cycloalkyl, or 5-12 membered heteroaryl wherein when q is 2 then both A rings are not phenyl;
B and B′ are each independently absent, C₁₋₆ alkyl, C₂₋₆ alkenyl, or C₂₋₆ alkynyl; wherein q is 1 then at least one of B and B′ is absent;
R₁ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}S O₂NRₐR_{b}, —P(═O)ORₐOR_{b}, C₁₋₆ alkyl which is unsubstituted or substituted one or more times by R¹⁰, C₂₋₆ alkenyl which is unsubstituted or substituted one or more times by R¹⁰, C₂₋₆ alkynyl which is unsubstituted or substituted one or more times by R¹⁰, or any two occurrences of R₁ can be taken together with the atoms to which they are attached to form a 5-7 cycloalkyl which is unsubstituted or substituted one or more times by R¹¹ or a 5-7 membered heterocycle which is unsubstituted or substituted one or more times by R¹²;
Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
R_{2′} and R₂ are independently H, halogen, C₁₋₁₀ alkyl, C₁₋₆ halogenated alkyl, —(CH₂)₁₋₆₀H, —NR_{b}C(═O)Rₐ C₆₋₁₂ aryl, or 5-12 membered heteroaryl;
R₃ and R₃′ are each independently H, alkyl, —(CH₂)₁₋₆OH, C₂₋₆ alkenyl, or C₂₋₆ alkynyl;
R₄ and R₄′ are each independently halogen, —NRₐR_{b}, —C(O)NRₐR_{b}, —(CH₂)₁₋₆ OH, C₁₋₆ alkyl, C₁₋₆ halogenated alkyl, hydroxyl, or C₁₋₆ alkoxy; wherein two occurrence of R₄ can be taken together with the atoms to which they are attached to form a C₁₋₆ alkenyl which is unsubstituted or substituted one or more times by R¹⁰, a 3-7 cycloalkyl which is unsubstituted or substituted one or more times by R¹¹ or a 4-7 membered heterocycle which is unsubstituted or substituted one or more times by R¹²; wherein two occurrence of R₄′ can be taken together with the atoms to which they are attached to form a C₁₋₆ alkenyl which is unsubstituted or substituted one or more times by R¹⁰, a 3-7 cycloalkyl which is unsubstituted or substituted one or more times by R¹¹ or a 4-7 membered heterocycle which is unsubstituted or substituted one or more times by R¹²; wherein Rₐ-R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
X and Y are each independently 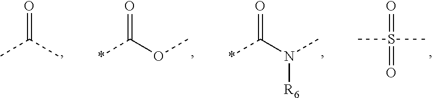
or a bond;
wherein the asterisk (*) indicates the point of attachment to the nitrogen of ring C or C′;
R₅ and R₅′ are each independently H, C₁₋₁₈ alkyl which is unsubstituted or substituted one or more times by R¹⁰, C₂₋₁₂ alkenyl which is unsubstituted or substituted one or more times by R¹⁰, C₂₋₁₂ alkynyl which is unsubstituted or substituted one or more times by R¹⁰, C₆₋₁₄ aryl which is unsubstituted or substituted one or more times by R¹¹, C₇₋₁₆ aralkyl which is unsubstituted or substituted one or more times by R¹¹, 5-12 membered heteroaryl which is unsubstituted or substituted one or more times by R¹¹, 6-18 membered heteroaralkyl which is unsubstituted or substituted one or more times by R¹¹, 3-12 membered heterocycle which is unsubstituted or substituted one or more times by R¹², or 4-18 membered heterocycle-alkyl which is unsubstituted or substituted one or more times by R¹²;
R₆ is H, C₁₋₆ alkyl, or halogenated C₁₋₆ alkyl;
m and n are a positive integer and when combined are 1, 2, 3, or 4, provided that each of m and n are not 3 or 4;
p is 0, 1, 2, 3 or 4;
q is 1 or 2;
u is 0 or 1;
s is 0 or 1;
R¹⁰ is halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b};
R¹¹ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and
R¹² is halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

63-68. (canceled)

69. The compound according to claim 1, wherein each A is independently a 5-12 membered heteroaryl wherein the heteroatom(s) are selected from the group consisting of oxygen and sulphur; wherein each A is independently substituted with (R₁)ₚ.

70-72. (canceled)

73. The compound according to claim 1,
wherein 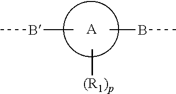
is selected from the group consisting of:

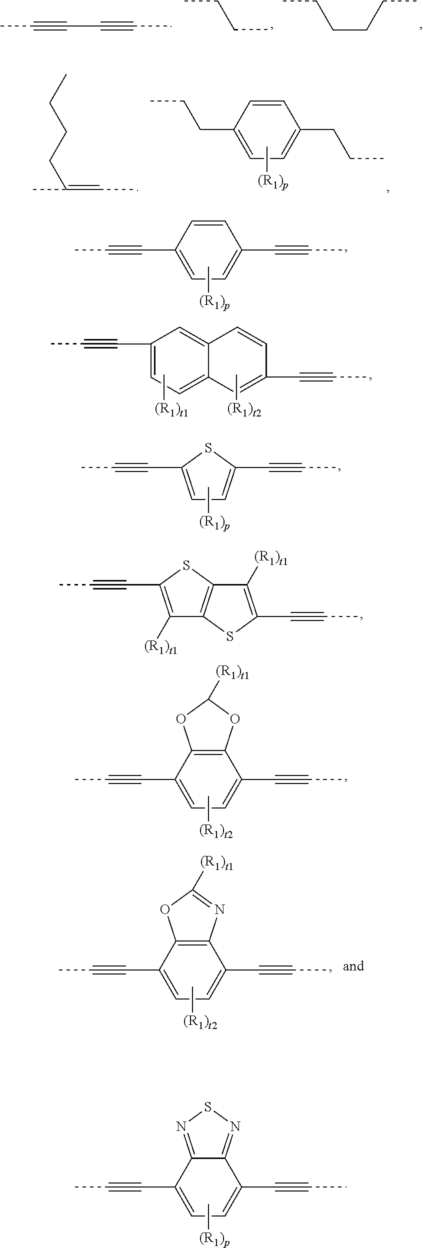

74. (canceled)

75. The compound according to claim59,
wherein 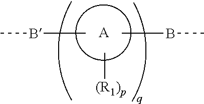
is selected from the group consisting of: 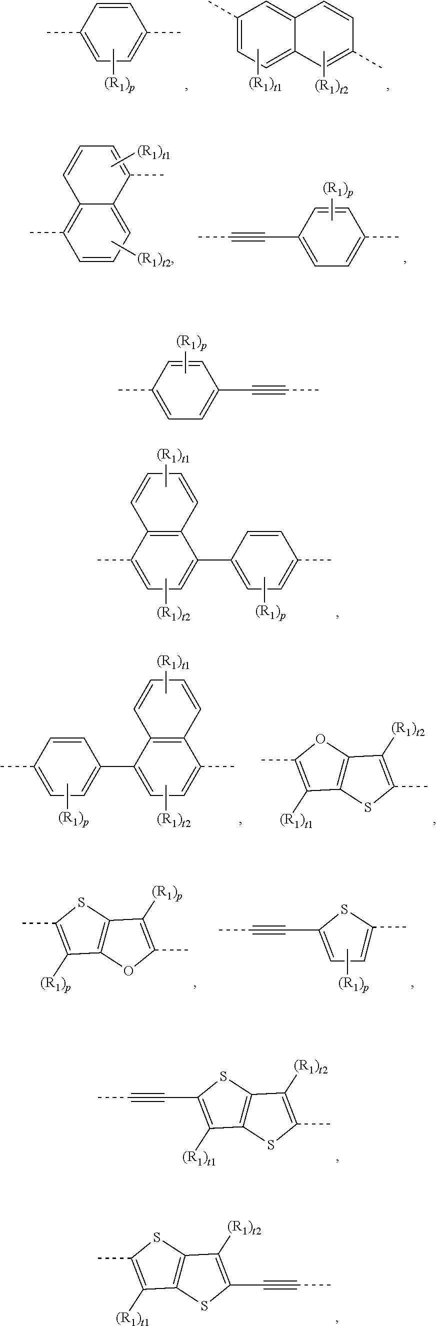 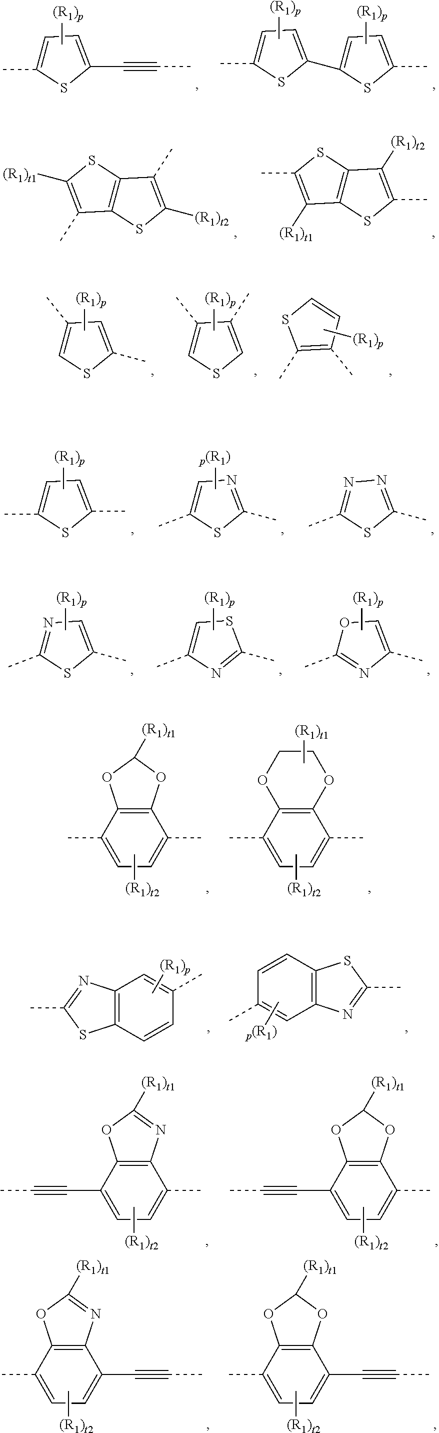 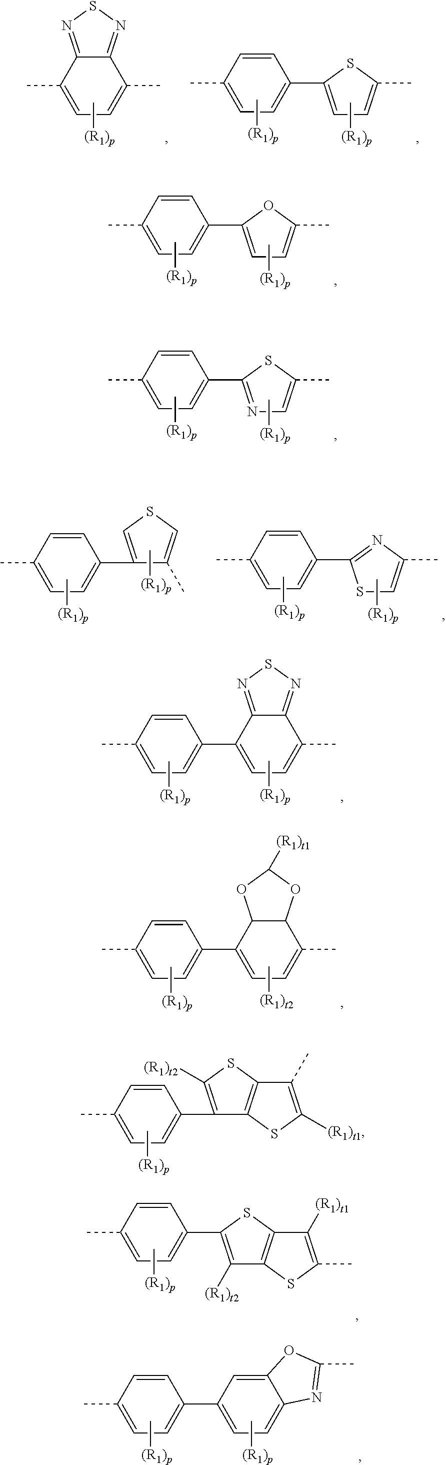 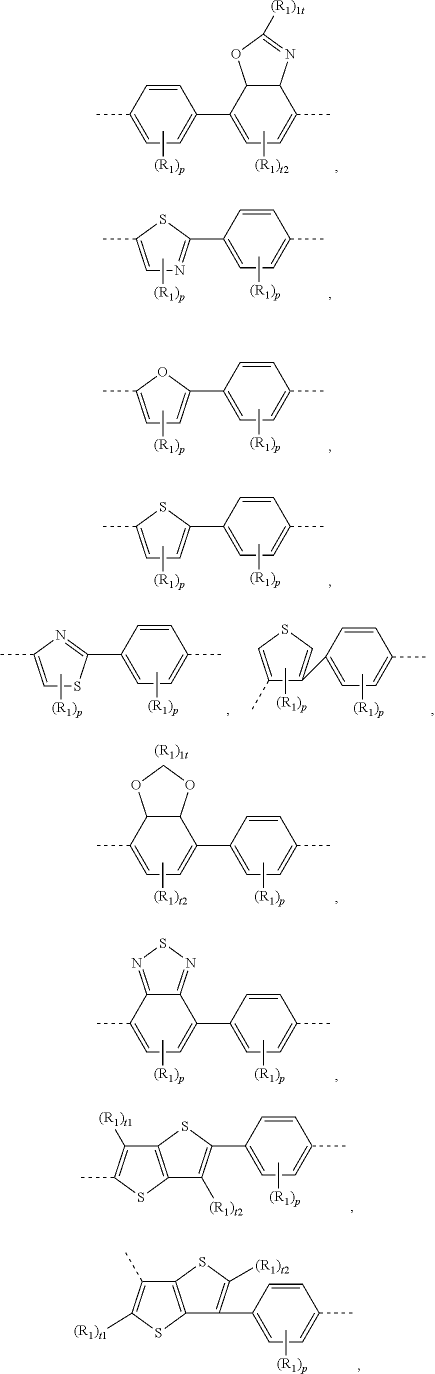 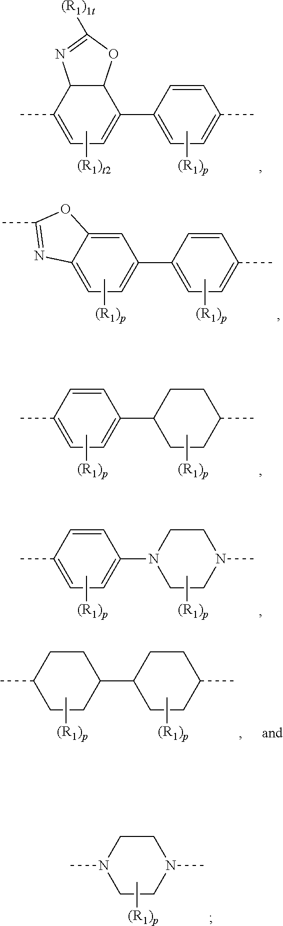
and
t1+t2=p.

76. The compound according to claim 75, wherein 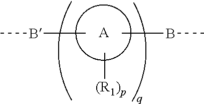
is selected from the group consisting of: 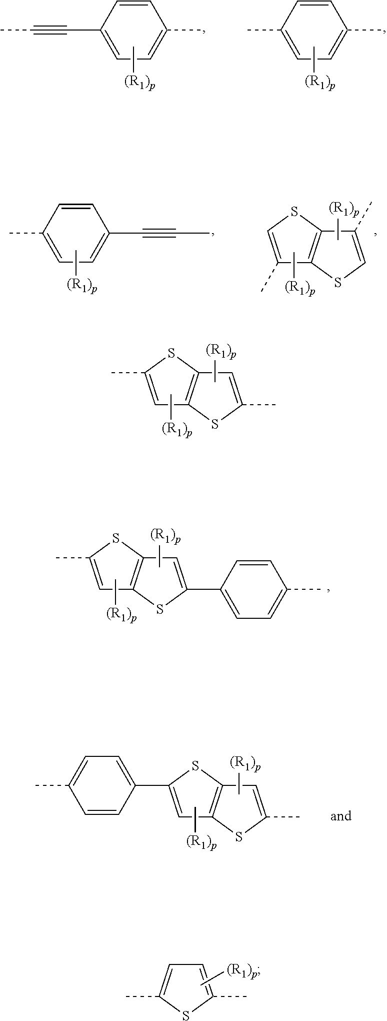
and
t1+t2=p.

77-112. (canceled)

113. The compound according to claim 59, wherein said compound is of formula (IVA): 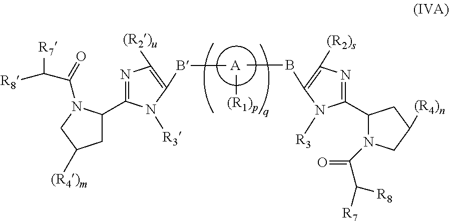
or a pharmaceutically acceptable salt thereof: wherein
R₇ and R₇′ are each independently C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, benzyl which is unsubstituted or substituted one or more times by R¹¹, 5-6 membered heteroaryl which is unsubstituted or substituted one or more times by R¹¹, 6-7 membered heteroaralkyl which is unsubstituted or substituted one or more times by R¹¹, 3-6 membered heterocycle which is unsubstituted or substituted one or more times by R¹², or 4-7 membered heterocycle-alkyl which is unsubstituted or substituted one or more times by R¹²;
R₈ and R₈′ are each independently —NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —NR_{b}S O₂Rₐ, or —NR_{b}SO₂NRₐR_{b}, wherein Rₐ, R_{b}, R_{c}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and
m and n are a positive integer and when combined are 1, 2, 3 or 4, provided that each of m and n are not 3 or 4.

114-119. (canceled)

120. The compound according to any claim 113, wherein said compound is of formula (VA): 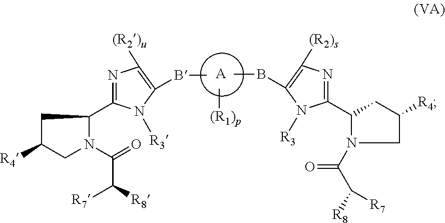
or a pharmaceutically acceptable salt thereof.

121. (canceled)

122. The compound of claim 1, wherein the compound is of formula: 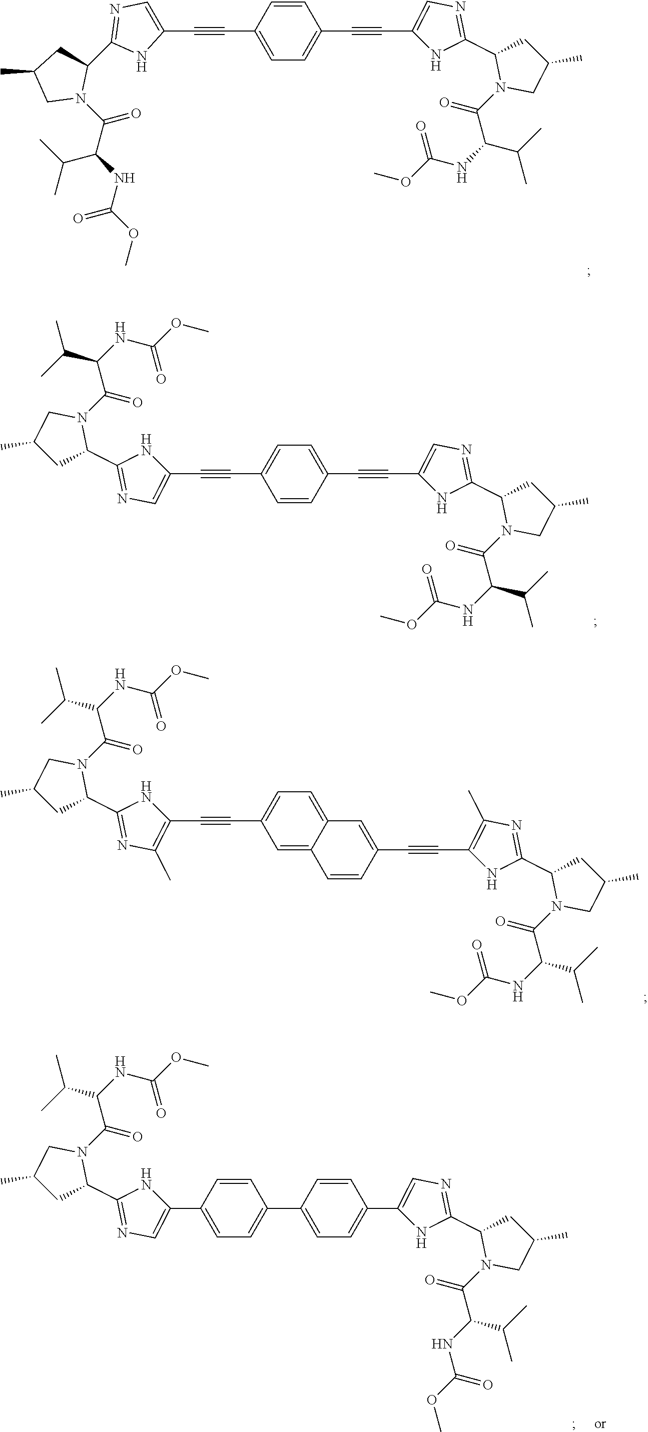 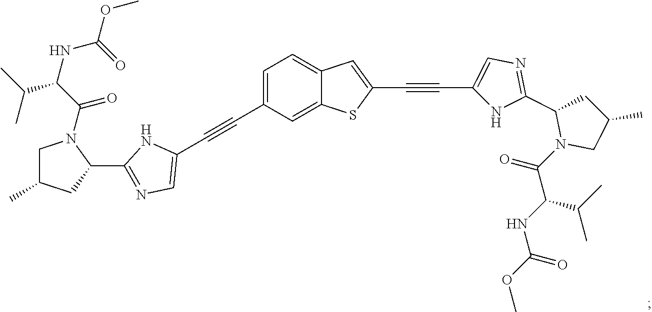 [SMILES: COC(=O)N[C@H](C(=O)N1C[C@@H](C)C[C@H]1c1ncc(C#Cc2ccc3cc(C#Cc4cnc([C@@H]5C[C@H](C)CN5C(=O)[C@@H](NC(=O)OC)C(C)C)[nH]4)sc3c2)[nH]1)C(C)C]
or a pharmaceutically acceptable salt thereof.

123-139. (canceled)

140. A method for preparing a compound of formula (IV) according to claim 49: 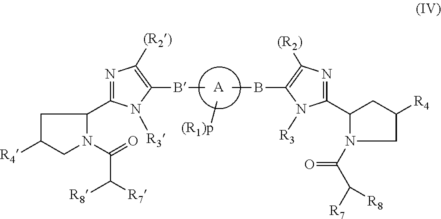
or a pharmaceutically acceptable salt thereof, wherein each of A, B, B′, R¹, p, R₂, R₂′, R₃, R_{3′}, R₄, R_{4′}, R₇, R_{7′}, R₈, and R_{8′}, are as defined in claim 49,
wherein said method comprises the steps of:
a) contacting a compound of formula (XXX): 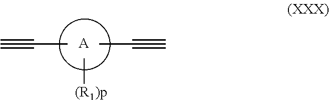
under coupling conditions with a compound of formula (XXXI) and a compound of formula (XXXII); and 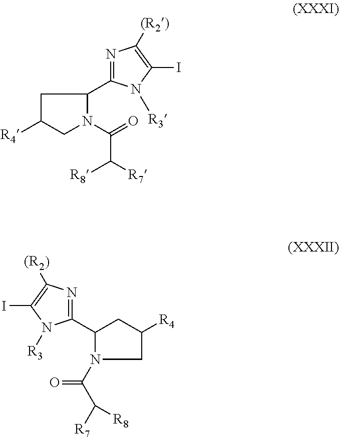
b) optionally hydrogenating the alkyne groups to provide a compound of formula (IV).

141-159. (canceled)

160. A method for preparing a compound of formula (III) 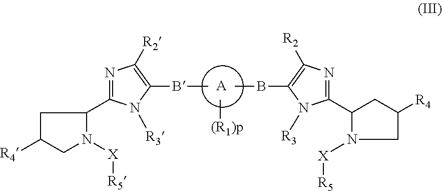
or a pharmaceutically acceptable salt thereof, wherein each of A, B, B′, R¹, p, R₂, R₂′, R₃, R_{3′}, R₄, R_{4′}, R₇, R_{7′}, R₈, and R_{8′}, are as defined herein in claim 1,
wherein said method comprises the steps of:
a) contacting the compound of formula (XXXIV) with a compound of formula R⁵—X—OH and/or R⁵—Y—OH: 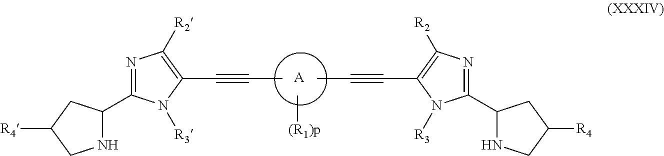
under reaction conditions to provide a compound of formula (III).

161-164. (canceled)

165. A pharmaceutical composition comprising at least one compound according to claim 1 and at least one pharmaceutically acceptable carrier or excipient.

166. A pharmaceutical composition comprising at least one compound according to claim 59 and at least one pharmaceutically acceptable carrier or excipient.

167. A pharmaceutical composition comprising at least one compound according to claim 62 and at least one pharmaceutically acceptable carrier or excipient.

168. A pharmaceutical composition comprising at least one compound according to claim 122 and at least one pharmaceutically acceptable carrier or excipient.

169. A method for treating or preventing a Hepatitis C viral infection in a human comprising administering to the human an effective amount of a compound according to claim 1.

170. A method for treating or preventing a Hepatitis C viral infection in a human comprising administering to the human an effective amount of a compound according to claim 59.

171. A method for treating or preventing a Hepatitis C viral infection in a human comprising administering to the human an effective amount of a compound according to claim 62.

172. A method for treating or preventing a Hepatitis C viral infection in a human comprising administering to the human an effective amount of a compound according to claim 122.
